SEQUENCE LISTING (11 sequences)

SEQ ID NO: 1 - protein, 150 aa, Homo sapiens
MKDEVALLAAVTLLGVLLQAYFSLQVISARRAFRVSPPLTTGPPEFERVYRAQVNCSEYFPLFLATLWVAGIFFHEGAAALCGLVYLFARLRYFQGYARSAQLRLAPLYASARALWLLVALAALGLLAHFLPAALRAALLGRLRTLLPWA

SEQ ID NO: 2 - protein, 150 aa, Mus musculus
MKDEVALLATVTLVGVLLQAYFSLQVISARRAFHVSPPLTSGPPEFERVFRAQVNCSEYFPLFLATLWVAGIFFHEGAAALCGLFYLFARLRYFQGYARSAQLRLTPLYASARALWLLVAMAALGLLVHFLPGTLRTALFRWLQMLLPMA

SEQ ID NO: 3 - protein, 150 aa, Rattus norvegicus
MKEETALLATVTLVGVLLQAYFSLQVISARRTFHVSPPLTSGPPEFERVFRAQVNCSEYFPLFLATLWVAGIFFHEGAAALCGLFYLFARLRYFQGYARSAQHRLDPLYASARALWLLVAMAALGLLVHFLPGTLRAALFRWLQVLLPMA

SEQ ID NO: 4 - protein, 133 aa, Bos taurus
MKDEVALLASVTLLGVLLQAYFSLQVISARRAFRVSPPLTTGPPEFERIYRAQVNCSEYFPLFLAMLWVAGIFFHEGAAALCGLVYLFARLRYFQGYARSAQQRLAPLYASARALWLLVALAALGLLAHFLPA

SEQ ID NO: 5 - protein, 145 aa, Gallus gallus
MLEQIHLLAAVTVLGVLEQAYFFLQVIHARRKFGISPPNISGPPEFERIFRAQVNSSEYFPIFVALLWQAGLFFHQGLAAALGLLYLYSRYCYFMGYRASSSDRLTPIYFSTGVLWVLIAAATLGLLHFFLSHYVGLNVLRLIAA

SEQ ID NO: 6 - protein, 147 aa, Homo sapiens
MAGNSILLAAVSILSACQQSYFALQVGKARLKYKVTPPAVTGSPEFERVFRAQQNCVEFYPIFIITLWMAGWYFNQVFATCLGLVYIYGRHLYFWGYSEAAKKRITGFRLSLGILALLTLLGALGIANSFLDEYLDLNIAKKLRRQF

SEQ ID NO: 7 - protein, 161 aa, Homo sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 8 - protein, 149 aa, Homo sapiens
MAVLSKEYGFVLLTGAASFIMVAHLAINVSKARKKYKVEYPIMYPENGHIFNCIQRAHQNTLEVYPPFLFFLAVGGVYHPRIASGLGLAWIVGRVLYAYGYYTGEPSKRSRGALGSIALLGLVGTTVCSAFQHLGWVKSGLGSGPKCCH

SEQ ID NO: 9 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 10 - protein, 155 aa, Homo sapiens
MVDLTQVMDDEVFMAFASYATIILSKMMLMSTATAFYRLTRKVFANPEDCVAFGKGENAKKYLRTDDRVERVRRAHLNDLENIIPFLGIGLLYSLSGPDPSTAILHFRLFVGARIYHTIAYLTPLPQPNRALSFFVGYGVTLSMAYRLLKSKLYL

SEQ ID NO: 11 - protein, 155 aa, Rattus norvegicus
MADLKQLMDNEVLMAFTSYATIILAKMMFLSSATAFQRLTNKVFANPEDCAGFGKGENAKKFLRTDEKVERVRRAHLNDLENIVPFLGIGLLYSLSGPDLSTALIHFRIFVGARIYHTIAYLTPLPQPNRGLAFFVGYGVTLSMAYRLLRSRLYL

(19) United States
(12) Patent Application Publication
(10) Pub. No.: US 2010/0216113 A1
(43) Pub. Date: Aug. 26, 2010

(54) Methods

(76) Inventors: Andreas Kohl, Stockholm (SE); Said Eshaghi, Solna (SE); Daniel Martinez Molina, Balsta (SE); Par Nordlund, Stockholm (SE); Anders Wetterholm, Stockholm (SE); Jesper Z. Haeggstrom, Stockholm (SE)

Correspondence Address: NIXON & VANDERHYE, PC, 901 NORTH GLEBE ROAD, 11TH FLOOR, ARLINGTON, VA, 22203, US

(21) Appl. No.: 12/451,394

(22) PCT Filed: May 7, 2008
(86) PCT No.: PCT/GB2008/001584

§ 371 Date: Apr. 29, 2010

Related U.S. Application Data
(60) Provisional application No. 60/924,521, filed on May 18, 2007.

(51) Int. Cl. C12Q 1/00; G06G 7/48; C12N 9/00

(52) U.S. Cl. 435/4; 703/11; 435/183

(57) ABSTRACT

A method for selecting or designing a compound expected to modulate the activity of Leukotriene C4 synthase (LTC4S), the method comprising the step of using molecular modelling means to select or design a compound that is predicted to interact with the catalytic site or a substrate binding region of LTC4S, wherein a three-dimensional structure of at least a part of the catalytic site or a substrate binding region of LTC4S is compared with a three-dimensional structure of a compound, and a compound that is predicted to interact with the said catalytic site or substrate binding region is selected. The selected compound may be predicted to bind to at least a part of a region of the structure termed the “GSH substrate binding cavity” (formed by residues including residues Arg51, Arg30, Arg104, Gln53, Asn55, Glu58, Tyr59, Tyr93, Tyr97, Ile27, Pro37, Leu108 of full length human LTC4S, or equivalent residues); the “lipophilic substrate binding crevice” (formed by residues including Ala20, Leu24, Ile27, Tyr59, Trp116, Ala112, Leu115, Leu108, Tyr109, Leu62, Val119, Thr66, Vall6 and Leu17, or equivalent residues); or the “catalytic site” (formed by residues including Arg104 or Arg31, or equivalent residues).

The present invention relates to methods for screening for modulators of LTC₄ synthase. It relates to the definition of a three-dimensional structure of LTC₄ synthase and methods based thereon.

Leukotriene C₄ (LTC₄) synthase (LTC4S) is a pivotal enzyme in the biosynthesis of leukotrienes, a family of paracrine hormones implicated in the pathophysiology of inflammatory and allergic disorders, in particular bronchial asthma (Samuelsson, B. Science 220, 568-75 (1983); and Lewis, R. A., Austen, K. F. & Soberman, R. J. N Engl J Med 323, 645-55 (1990)). Leukotrienes are formed by immunocompetent cells including neutrophils, eosinophils, basophils, mast cells, and macrophages, in response to a variety of immunological as well as non-immunological stimuli. These lipid mediators are divided into two major classes exemplified by the chemotaxin LTB₄, and the spasmogenic cysteinyl-leukotrienes (LTC₄, LTD₄, and LTE₄). Leukotriene biosynthesis is initiated by the enzyme 5-lipoxygenase (5-LO) which converts arachidonic acid into the unstable epoxide LTA₄, a central intermediate in the leukotriene cascade. LTA₄ may in turn be hydrolyzed into LTB₄ by the enzyme LTA₄ hydrolase, or conjugated with GSH to form LTC₄, a reaction catalyzed by the specific LTC4S. During cellular activation, all key enzymes in leukotriene biosynthesis, except LTA₄ hydrolase, form a biosynthetic complex assembled at the nuclear membrane, suggesting that leukotrienes may have unknown intranuclear functions related to gene regulation or cell growth (Serhan, C. N., Haeggstrom, J. Z. & Leslie, C. C. Faseb J 10, 1147-58 (1996)).

Leukotriene C₄, the natural product of LTC4S, may be cleaved by γ-glutamyl transpeptidase and a dipeptidase to yield LTD₄ and LTE₄, respectively. Together, these three leukotrienes constitute what was previously known as slow-reacting substance of anaphylaxis (SRS-A), a potent smooth muscle contracting agent with profound effects at only nM concentrations in the human respiratory system, where it elicits bronchoconstriction, and the microcirculation with increased leakage and oedema formation (Samuelsson, B. Science 220, 568-75 (1983)). Hence, cysteinyl-leukotrienes (cys-LT) are regarded as key mediators of inflammation and allergy, and have been implicated in a number of diseases, including nephritis, dermatitis, hay-fever, and in particular asthma and pulmonary fibrosis (Lewis, R. A., Austen, K. F. & Soberman, R. J. N Engl J Med 323, 645-55 (1990); Beller, T. C. et al. Proc Natl Acad Sci USA 101, 3047-52 (2004)). Furthermore, the role of cys-LT in inflammation and asthma has been well corroborated by the therapeutic potential and clinical use of molecules, which inhibit cys-LT biosynthesis as well as antagonists of the receptors for cys-LT (Drazen, J. M., Israel, E. & O'Byrne, P. Treatment of asthma with drugs modifying the leukotriene pathway. N. Engl. J. Med. 340, 197-206 (1999)). Moreover, reduced inflammatory reactions are observed in several animal models of leukotriene deficiency (Chen, X. S., Sheller, J. R., Johnson, E. N. & Funk, C. D. Nature 372, 179-182 (1994); Griffiths, R. J., et al. Proc Natl Acad Sci USA 92, 517-21 (1995); and Griffiths, R. J., et al. J Exp Med 185, 1123-9 (1997)). In addition, LTC₄ modulates the immune response, e.g., by interference with specific subsets of lymphocytes, production of cytokines, as well as liberation of immunoglobulins from B-lymphocytes (Payan, D. G., Missirian-Bastian, A. & Goetzl, E. J. Proc Natl Acad Sci USA 81, 3501-5 (1984); Rola-Pleszczynski, M. & Lemaire, I. J Immunol 135, 3958-61 (1985); and Yamaoka, K. A., Claesson, H. E. & Rosen, A. J Immunol 143, 1996-2000 (1989)).

LTC4S is a notoriously unstable, 18 kDa integral membrane enzyme, which has been purified to apparent homogeneity from KG-1 and THP-1 cells (Penrose, J. F. et al. Proc. Natl. Acad. Sci. USA 89, 11603-11606 (1992); Nicholson, D. W. et al. Proc. Natl. Acad. Sci. USA 90, 2015-2019 (1993). The cloning and molecular characterization of the enzyme unexpectedly revealed that LTC4S and FLAP are homologous proteins (Lam, B. K., et al. Proc. Natl. Acad. Sci. USA 91, 7663-7667 (1994); Welsch, D. J. et al. Proc. Natl. Acad. Sci. USA 91, 9745-9749 (1994)). Further studies showed that LTC4S is also distantly related to microsomal glutathione-5-transferases (MGST), in particular MGST2 and MGST3, and several functional links to LT metabolism have been established. Thus, human MGST2 and MGST3 both possess LTC₄ synthase activity and recent studies have shown that the main, if not the only, LTC₄ producing enzyme in human umbilical vein endothelial cells is MGST2, indicating that this enzyme plays a role in transcellular biosynthesis of LTC₄ in the vascular wall (Jakobsson, P.-J., et al. Prot. Sci. 8, 689-692 (1998)). Another enzyme homologous to LTC4S is microsomal prostaglandin (PG) E synthase type 1 (mPGES-1), which catalyzes the conversion of prostaglandin endoperoxides into the substance PGE2, an important mediator of inflammation, fever and pain (Jakobsson, P. J. et al. Proc. Natl. Acad. Sci. USA 96, 7220-7225 (1999)). Together, LTC4S, FLAP, MGST1, MGST2, MGST3, and microsomal prostaglandin (PG) E2 synthase, belong to a widespread protein superfamily designated MAPEG (membrane-associated proteins in eicosanoid and glutathione metabolism) (Jakobsson, P.-J., et al. Prot. Sci. 8, 689-692 (1998)).

Thus far, no detailed structural information is available for LTC4S or any other member of the MAPEG family. Some crude structural information has been obtained by electron microscopy, a technique which is very different from X-ray crystallography and which does not generate structures at atomic resolution. Thus, Schmidt-Krey et al. (Structure 12, 2009-14 (2004)) have described generation of 2-dimensional crystals of LTC4S from which a projection map could be calculated revealing a picture of its quaternary structure (trimer) and basic interrelationships of the transmembrane helices. A low-resolution (3.2 Å) structure of the detoxifying liver enzyme MGST-1 has also been determined (Holm et al (2006) J Mol Biol 360, 934-945. However, despite the well-recognized need thereof, the three-dimensional structure of LTC4S has not yet been disclosed.

More specifically, the problems that need to be overcome in order to provide such a determination may in brief be explained as follows. There are two major difficulties in obtaining a three-dimensional structure of a protein molecule, as discussed further below. The first one is to grow crystals of good quality that are reproducible and diffract to atomic resolution (beyond 2.5 Å). This means a thorough and cumbersome investigation of parameters that influence the crystal growth such as pH, temperature, nature of buffers, nature of precipitant, just to mention a few. The addition of ligands such as substrate analogues or inhibitors or the addition of other molecules can be important for obtaining good crystals. There is only little understanding of the physical background of the crystallisation process which means that the search for suitable crystallisation conditions for a certain protein is unique, requires creativity and intuition, and is governed by trial and error procedures. The purity of the protein is also a crucial parameter in the crystallisation and a suitable degree of purity can be hard, or even impossible, to achieve. Before suitable crystals have been obtained there is no guarantee that it will ever be possible to do so.

It should further be emphasized that all these problems are extremely hard to manage for membrane proteins, particularly integral membrane proteins, that are difficult to obtain in large and sufficiently pure amounts. Also, membrane proteins are hydrophobic, tend to aggregate, and have to be kept in solution by various detergents that interfere with the crystallisation process.

The second major difficulty is associated with overcoming the phase-problem which is inherent to X-ray diffraction methods. To be able to overcome this problem it is necessary to substitute the protein with suitable heavy atom substance such as e.g. mercury, gold or platinum compounds. Crystals often cannot withstand the treatment with these compounds and the search for suitable substitutions is not straight forward and may become very extensive. Another option is to substitute all methionines by seleno-methionine (Se-Met) residues. This method requires production of recombinant protein in special strains of E. coli under non-standard conditions, followed by a new purification and recrystallisation of the Se-Met containing protein.

It is important to note that although Schmidt-Krey et al reported the generation of 2-dimensional crystals of LTC4 synthase (ie a single layer or small number (for example less than 10) layers of LTC4 synthase homotrimers), these crystals can not be used for X-ray crystallography and determination of the three dimensional structure of the enzyme. For X-ray crystallography using current techniques it is necessary to have a 3-dimensional crystal ie with more than one layer of LTC4 synthase trimers, as is well known to those skilled in the art.

Thus, as a reliable definition of the three-dimensional structure of LTC4S would enable e.g. a display in visual form on a computer screen of the shape of the molecule, then, could the above mentioned problems be solved, a whole range of possibilities would be opened, such as rational structure-based drug design, e.g. in combination with combinatorial chemistry, aimed at production of novel medicaments useful in disorders associated with the leukotriene cascade, as well as protein-engineering to create novel variants of the enzyme with altered, but yet useful, properties.

As LTC4S is a recognized important drug target, some inhibitors thereof have been synthesized (Hutchinson, J. H. et al J. Med. Chem. 38, 4538-4547 (1995); Gupta, N., Nicholson, D. W., Ford-Hutchinson, A. W. Can. J. Physiol. Pharmacol. 75, 1212-1219 (1997)). Due to the absence of any available information regarding the three-dimensional structure of LTC4S, as discussed above, none of the previously described inhibitors have been designed based on the exact structure thereof. Accordingly, there is a need within this field of determining the three-dimensional structure of LTC4S in order to design more potent and selective inhibitors of LTC4S as well as modified structures exhibiting even more advantageous pharmaceutical properties.

The listing or discussion of a prior-published document in this specification should not necessarily be taken as an acknowledgement that the document is part of the state of the art or is common general knowledge.

We have crystallized and determined the three-dimensional structure of LTC4S complexed with the substrate glutathione. It is the first high-resolution three-dimensional structure of a member of the MAPEG family of proteins and enables a description of the structural basis and molecular mechanisms of catalysis. In addition, the structural information makes possible rational design of enzyme inhibitors, which may be developed into clinically useful anti-inflammatory drugs.

A first aspect of the invention provides a method for selecting or designing a compound expected to modulate the activity of Leukotriene C4 synthase (LTC4S), the method comprising the step of using molecular modelling means to select or design a compound that is predicted to interact with the catalytic site or a substrate binding region (together the active site) of LTC4S, wherein a three-dimensional structure of at least a part of the catalytic site or a substrate binding region of LTC4S is compared with a three-dimensional structure of a compound, and a compound that is predicted to interact with the said catalytic site or substrate binding region is selected.

The invention provides a computer-based method of rational drug design which comprises:

providing the structure of at least a part of the catalytic site or a substrate binding region of LTC4S (the protein) as defined by the coordinates of Table I or Table II ± the root mean square deviation from the backbone atoms of the protein of less than 2.0 Å, preferably less than 1.5 Å, 1.0 Å, or 0.5 Å;
providing the structure of a candidate modulator molecule; and
fitting the structure of the candidate modulator molecule to the structure of the protein.

By the term LTC4S is included the polypeptide termed LTC4S in Lam et al (1994) Expression cloning of a cDNA for human leukotriene C₄ synthase, an integral membrane protein conjugating reduced glutathione to leukotriene A₄ PNAS 91, 7663-7667 or Welsch et al (1994) Molecular cloning and expression of human leukotriene-C4 synthase PNAS 91, 9745-9749. LTC4S has EC number 4.4.1.20. The human LTC4S polypeptide sequence is presented below. The term “LTC4S” as used herein includes this polypeptide sequence as well as naturally occurring variants thereof. Further animal species also have equivalent polypeptides to LTC4S and are included within the scope of this term. Preferably, by LTC4S we mean the LTC4S polypeptide sequence shown below or a polypeptide sequence having at least 60, 65, 70, 75, 80, 85, 90, 95 or 98% identity thereto.

| | | |
|---|---|---|
| ..1 | Met Lys Asp Glu Val Ala Leu Leu Ala Ala | .10 |
| .11 | Val Thr Leu Leu Gly Val Leu Leu Gln Ala | .20 |
| .21 | Tyr Phe Ser Leu Gln Val Ile Ser Ala Arg | .30 |
| .31 | Arg Ala Phe Arg Val Ser Pro Pro Leu Thr | .40 |
| .41 | Thr Gly Pro Pro Glu Phe Glu Arg Val Tyr | .50 |
| .51 | Arg Ala Gln Val Asn Cys Ser Glu Tyr Phe | .60 |
| .61 | Pro Leu Phe Leu Ala Thr Leu Trp Val Ala . | .70 |
| .71 | Gly Ile Phe Phe His Glu Gly Ala Ala Ala | .80 |
| .81 | Leu Cys Gly Leu Val Tyr Leu Phe Ala Arg | .90 |
| .91 | Leu Arg Tyr Phe Gln Gly Tyr Ala Arg Ser | 100 |
| 101 | Ala Gln Leu Arg Leu Ala Pro Leu Tyr Ala | 110 |
| 111 | Ser Ala Arg Ala Leu Trp Leu Leu Val Ala | 120 |
| 121 | Leu Ala Ala Leu Gly Leu Leu Ala His Phe | 130 |
| 131 | Leu Pro Ala Ala Leu Arg Ala Ala Leu Leu | 140 |
| 141 | Gly Arg Leu Arg Thr Leu Leu Pro Trp Ala | 150 |

By the term LTC4S is included the polypeptide termed any mammalian or other LTC4S which has the same amino acid sequences as the human form with up to twenty, fifteen, ten, nine, eight, seven, six, five, four, three, two or one conservative or non-conservative substitutions therein. The amino acid sequences of mammalian LTC4S are about 90% identical. Thus, the three-dimensional structures are also expected to be identical to approximately the same extent. The term LTC4S does not encompass other members of the MAPEG family such as FLAP, MGST-1, MGST-2, MGST-3 or MPGES-1, as will be readily apparent to those skilled in the art.

In relation to polypeptides or structures used in screening methods or assays the term also includes fragments and fusions thereof that comprise the active site, as known to those skilled in the art. As LTC4S is a single-domain enzyme it is considered that fragments with extensive portions of the full length LTC4S sequence missing may not retain catalytic activity. However, fragments in which the C-terminal amino acids (for example C-terminal up to 20, 15, 10, 5, 4, 3, 2 or 1 amino acids) of full length LTC4S are missing are considered to retain catalytic activity. Since the active site of LTC4S is composed of amino acids from two adjacent monomers (Tables 1 and 2), a single polypeptide of LTC4S on its own is not sufficient for enzyme activity. Hence, in order to display catalytic activity, an LTC4S polypeptide must be complexed with another LTC4S polypeptide or a substitute polypeptide, for example a FLAP polypeptide, for example as described in Lam B K et al. (1997) J. Biol. Chem. 272(21):13923-8). Lam et al (1997) reports catalytic activity for a fusion of a LTC4S polypeptide and a FLAP polypeptide and for a fusion in which an internal segment of LTCS was replaced with a corresponding segment of FLAP. It is considered that wild-type LTC4S polypeptides spontaneously assemble into a catalytically active complex: it is preferred that the LTC4S fragment or fusion retains this ability.

The structure is typically (but not necessarily) a structure (or part of a structure) of an LTC4S polypeptide that retains LTC4S activity. For example, when in the form of a trimer or dimer, the LTC4S polypeptide is typically capable of conjugating glutathione with leukotriene A₄. Alternatively or in addition the LTC4S polypeptide retains fatty acid hydroperoxidase activity.

The following is an example of an assay that can be used in assessing the ability of a compound to modulate, for example inhibit, the action of LTC4S. In the assay LTC₄ synthase catalyses the reaction where the substrate LTA₄ methyl ester is converted to LTC₄ methyl ester. Purified recombinant human LTC₄ synthase (for example expressed in yeast) is dissolved in 25 mM Tris-buffer pH 7.8 and stored at −20° C. The assay is performed in phosphate buffered saline (PBS) pH 7.4, supplemented with 5 mM glutathione (GSH). The reaction is terminated by addition of acetonitrile/MeOH/acetic acid (50/50/1). The assay is performed at rt in 96-well plates. Analysis of the formed LTC₄ methyl ester is performed with reversed phase HPLC (Waters 2795 utilizing an Onyx Monolithic C18 column). The mobile phase consists of acetonitrile/MeOH/H₂O (32.5/30/37.5) with 1% acetic acid pH adjusted with NH₃ to pH 5.6, and absorbance measured at 280 nm with a Waters 2487 UV-detector.

The following is added sequentially to each well:
1. 50 μl assay buffer, PBS with 5 mM GSH.
2. 0.5 μl test compound in DMSO.
3. 2 μl LTC₄ synthase in PBS. The total protein concentration in this solution is 0.025 mg/ml. Incubation of the plate at room temperature for 10 minutes.
4. 0.5 μl LTA₄ methyl ester. Incubation of the plate at rt for 1 min.
5. 50 μl stop solution.
   80 μl of the incubation mixture is analysed with HPLC.

Alternatively, an assay of the fatty acid hydroperoxidase activity can be used. For example: incubate 0.1-0.2 μg LTC4S in 50 μl 0.1 M K-phosphate pH 7.5 containing 1.5 mM GSH with 250 pmol hydroperoxide (13-HPOD, or 5-HPETE) at RT for 10 min. Terminate the reaction by the addition of 150 μl stop solution (MeCN:H2O:HOAc, 50:25:0.2, v/v). Analyse the conversion of the hydroperoxide into the corresponding alcohol by RP-HPLC, as described above, using a mobile phase composed of MeCN:H2O:HOAc, 60:40:0.1, v/v and the UV detector set at 235 nm.

Variations on these assays or alternative assays may be used, as will be well known to the skilled person.

We have found that LTC4S with an N-terminal hexahistidine tag is particularly beneficial for determining a structure for LTC4S. This fusion polypeptide has, for example, LTC4S activity and beneficial solubility and stability characteristics which make it particularly suitable for structural studies, for example formation of crystals which may be analysed by X-ray crystallography methods. It is considered that a fusion polypeptide with a different type of tag or different length histidine tag (for example a pentahistidine tag or septahistidine tag) may still be useful but is unlikely to be as useful as the fusion polypeptide with a hexahistidine tag. The size and metal ion co-ordination properties of the hexahistidine tag are considered to be particularly beneficial in forming well-diffracting crystals of LTC4S. The hexahistidine tag is considered to co-ordinate divalent metal ions, for example Nickel or Cobalt ions. It is considered that the crystals comprise neighbouring hexahistidine tags from more than one LTC4S trimer coordinated with metal ions. Accordingly, the structure can be one determined for LTC4S having an N-terminal hexahistidine tag.

It is particularly preferred that the structure is one determinable by a method as described in Example 1, for example a structure obtainable by X-ray analysis from a crystal obtainable using a mother liquor solution comprising a detergent. An example of a suitable detergent is dodecyl maltoside (DDM). Examples of other suitable detergents include the following:
CYMAL®-4
4-Cyclohexyl-1-butyl-β-D-maltoside¹
CAS #: 181135-57-9
.CYMAL®-5
5-Cyclohexyl-1-pentyl-β-D-maltoside¹
Formula Weight: 494.5 C₂₃H₄₂O₁₁
n-NONYL-®-D-MALTOPYRANOSIDE, ANAGRADE®
n-Nonyl-®-D-maltoside
CAS #: 106402-05-5
n-DECYL-®-D-MALTOPYRANOSIDE, ANAGRADE®
n-Decyl-β-D-maltoside
(Low Alpha)
CAS #: 82494-09-5
Formula Weight: 482.6 C₂₂H₄₂O₁₁
n-UNDECYL-®-D-MALTOPYRANOSIDE, ANAGRADE®
n-Undecyl-β-D-maltoside (Low alpha)
CAS #: 253678-67-0
n-OCTYL-®-D-GLUCOPYRANOSIDE, ANAGRADE®
n-Octyl-®-D-glucoside
CAS #: 29836-26-8
Formula Weight: 292.4 C₁₄H₂₈O₆

Further preferred details of the crystallisation and X-ray analysis are described in Example 1.

It is particularly preferred that the structure is that represented by the structure co-ordinates shown in Table I (structure determined in the absence of glutathione; GSH) or II (structure determined in the presence of GSH), or a structure based or modelled on such a structure or co-ordinates, for example in which the root mean square deviation from the backbone atoms of the protein is less than 2.0 Å, preferably less than 1.5 Å, 1.0, or 0.5 Å.

The present application provides a listing illustrating the coordinates defining human LTC4S complexed to one of its substrates, glutathione, as well as a detergent molecule defining the binding site for the lipid substrate leukotriene A₄ (LTA₄). Based on their role in catalysis, the two binding sites occupied by glutathione and detergent define the active site of LTC4S and can be used as templates for design of molecules having desired properties. Methods for such design will be discussed in further detail below. The structural coordinates according to the invention are included in the present description as a separate section denoted “X-ray data”, as Tables I to II, immediately preceding the claims. These are co-ordinates for an LTC4S monomer and coordinated molecules. In Table I, atom no 1 to atom no 1263 define the LTC4S part of the complex. In table II, atom no 1 to atom no 1195 relate to the LTC4S whilst atom no 1233 to 1252 relate to glutathione. The atom numbers relating to the LTC4S are different in the two Tables because there are more residues visible in the first Table; tails aren't always visible in X-ray structures. Each active site within the homotrimer (or dimer) is formed by residues from two adjacent subunits within the homotrimer. The conditions prevailing at the determination thereof are described in detail in the Experimental section below.

As the skilled person in this field realises, such coordinates usually exhibit a certain degree of variation, due to e.g. thermal motion and slight differences in crystal packing. Thus, any references herein to Tables I to II in connection with the proteins and other molecules are merely intended to illustrate the coordinates defining the conformation of the molecules under identical conditions, as determined by use of the same apparatus and method.

The structure may be one determined following crystallisation in the presence of a known or potential interactor with LTC4S or modulator of LTC4S activity (as discussed further below), for example a known or potential inhibitor of LTC4S activity. The structure may, for example, be one determinable in the absence of glutathione (GSH) or in the presence of GSH. Examples of both are provided in Example 1. We have found that LTC4S crystallises in the presence of GSH. Alternatively, GSH can be absent during the initial crystallisation and then soaked into the crystal after it has formed.

For example, the structure may be one determined following crystallisation in the presence of a known LTC4S inhibitor, for example an inhibitor that is believed to bind to the GSH binding site; or an inhibitor that is believed to bind at the LTA4 binding site, such as cysteinyl-leukotrienes, LTC₄, LTD₄, or LTE₄, the 5-lipoxygenase inhibitors thiopyranol[2,3,4-c,d]indoles and L-699.333, the FLAP inhibitor MK886, or the CysLT receptor antagonist Montelukast. Co-crystals may form for inhibitors which are able respectively to displace GSH positioned in the GSH substrate binding cavity; or detergent positioned in the lipophilic substrate binding crevice; or at the catalytic part of the active site, as discussed in Example 1. The lipid substrate and GSH have different affinities for the active site. Thus, co-crystals may form with compounds targeted to the lipid binding site which have an IC50 of less than 100 μM, for example less than 10 μM or less than 1 μM (measured, for example, using LTA₄/GSH as the substrates), whereas compounds targeted to the GSH binding site may have an IC50 of less than 10 mM, for example less than 1 mM or less than 100 μM (measured, for example, using LTA₄/GSH as the substrate). It will be appreciated that some variation in crystallisation conditions (for example different mother liquors) may be required for co-crystallisation with different molecules. Techniques for investigating suitable crystallisation conditions in each case, starting from the information provided herein, will be well known to those skilled in the art. In one embodiment, co-crystallisation may be performed by diffusion of the co-crystallised molecule into a crystal of the polypeptide, for example a crystal obtained as set out in Example 1. This may be referred to as a “Soaking” procedure. If there is GSH or detergent positioned in the active site, as discussed in Example 1, co-crystallisation by diffusion/soaking may be easier to achieve, for example may require a lower concentration of the inhibitor, with an inhibitor with an IC50 of less than 10 μM, for example less than 1 μM or less than 100 nM.

It is considered that co-crystallisation may also be possible and useful with molecules with lower affinity for LTC4S, for example with IC50s in the millimolar range. For example, co-crystallisation may be useful with small molecules (“fragments”) considered to interact with part only of the active site. These small molecules may be useful as modules in designing/building a larger molecule with a lower IC50 for LTC4S inhibitor activity.

A further aspect of the invention provides a three-dimensional crystalline form of an LTC4S polypeptide (ie with multiple layers, for example more than 10 layers, preferably more than 100 layers of LTC4S homotrimers) as defined in relation to any one of the preceding aspects of the invention, for example a polypeptide consisting of full length human LTC4S with an N-terminal hexahistidine tag. The three dimensional crystalline form may belong to space group F23. A unit cell may contain 48 LTC4S chains and/or comprise multiple adjacent histidine tags coordinated by metal ions, as discussed further in Example 1 (for example three for each LTC4S trimer or twelve for each unit cell). The term “three-dimensional crystalline form” will be well known to those skilled in the art and does not encompass a two-dimensional (ie single or up to about 10 layer of LTC4S homotrimers) crystal form such as that described in Schmidt-Krey et al (1994) supra.

The crystalline form may further comprise a co-crystallised molecule, for example GSH or a detergent or other known or potential interactor with LTC4S or modulator of LTC4S activity, or a test compound (for example a small molecule considered to interact with only a part of the active site) whose properties vis a vis LTC4S may not be known. For example, the co-crystallised molecule, for example test compound, may be a molecule that is known to modulate LTC4S or other MAPEG family member activity; or may be an LTC₄ mimic or an LTA₄ mimic, or LTC₄ receptor agonist or antagonist; or a fatty acid hydroperoxide or mimic thereof, or another aliphatic compound (Thorén S and Jakobsson P J, Eur. J. Biochem. (2000) 267(21):6428-34; Schroder O et al., Biochem. Biophys. Res Commun. (2003), 312(2):271-6). Known LTC4 receptors include CysLT1, CysLT2 and GPR-17 (Ciana P et al. EMBO J. (2006), 25(19):4615-27). For example, the co-crystallised molecule may be a compound with an IC50 for LTC4S of less than 100 μM, typically less than 10 μM, 1 μM or 100 nM. The co-crystallised molecule may be a compound identified by a screening/design method of the invention, as discussed further below. The co-crystallised molecule may be a small compound thought to interact with only a part of the active site and having an IC50 in the millimolar range.

A further aspect of the invention accordingly provides a method for preparing a crystalline form of the invention, or for attempting to prepare a crystalline form of the invention, comprising 1) providing an LTC4S polypeptide as defined in relation to any of the preceding aspects of the invention; 2) providing a compound selected using a selection/design method of the invention (typically but not necessarily with a LTC4S IC50 of less than 100 μM, 10 μM, 1 μM or 100 nM; and 3) carrying out crystallisation trials on a composition comprising the polypeptide and the selected compound.

A further aspect of the invention provides the use of a polypeptide as defined in relation to any of the preceding aspects of the invention in generating a three-dimensional crystal or a structure of the active site or a substrate binding region (or at least a part any thereof) of LTC4S; or a three-dimensional crystal or a structure of the active site or a substrate binding region (or at least a part any thereof) of LTC4S bound to a test compound. Preferences for the test compound are as indicated above.

The crystalline form may be useful in generating X-ray diffraction data and a structure, as well known to those skilled in the art, for example using techniques similar to those described in Example 1. The structure determined for LTC4S, for example as described herein, may be used in structure solution and refinement, for example as described in Example 1.

Such co-crystallisation and structures determined from co-crystallised molecules may be useful in molecular modelling and in determining features of the polypeptide and compound that are important for interaction. This may be useful in designing or selecting further test compounds.

In one embodiment it is preferred that the modelled molecule is predicted to bind to a region of the structure termed the “GSH substrate binding cavity” (considered to be formed by residues including residues Arg51, Arg30, Arg104, Gln53, Asn55, Glu58, Tyr59, Tyr93, Tyr97, Ile27, Pro37, Leu108 of full length human LTC4S, or equivalent residues of other LTC4S polypeptide); the “lipophilic substrate binding crevice” (formed by residues including Ala20, Leu24, Ile27, Tyr59, Trp116, Ala112, Leu115, Leu108, Tyr109, Leu62, Val119, Thr66, Val16 and Leu17, or equivalent residues of other LTC4S polypeptide); or the “catalytic site” (residues including Arg104 or Arg31, or equivalent residues of other LTC4S polypeptide). Accordingly, the method may comprise comparing the structure of the compound with the structure of one or more of the regions mentioned above (or regions interacting with those regions).

TABLE 1
Residues lining the glutathione binding cavity

| Monomer1 | Monomer2 |
|---|---|
| Arg51 | Ser23 |
| Asn55 | Val26 |
| Glu58 | Ile27 |
| Tyr59 | Arg30 |
| Tyr93 | Arg31 |
| Tyr97 | Pro37 |
| Arg104 | Tyr50 |
| Leu108 | Gln53 |

In Table 1, Arg51, Asn55, Glu58, Tyr59, Tyr93, Tyr97, Arg104, Arg30, and Gln 53 are highly conserved amino acids among members of the MAPEG family.

At this site, GSH is bound deep in a polar pocket at the interface between helix 1 and 2 from one monomer and 3 and 4 from a neighbouring monomer. The GSH molecule makes polar interactions to residues from both monomers constituting the active site. The carboxylate moieties of GSH make salt bridges to Arg51′ and Arg30 at the base of the binding pocket, effectively bending GSH and directing its thiol group towards the membrane interface where it interacts with Arg104′. Additional polar interactions to GSH are made by Gln53, Asn55′, Glu58′, Tyr59′, Tyr93′ and Tyr97′. Several non-polar interactions are also made (Ile27, Pro37 and Leu108′) providing an optimal fit for GSH into its binding pocket.

TABLE 2
Amino acids in the leukotriene binding site

| Monomer1 | Monomer2 |
|---|---|
| Tyr59 | Val16 |
| Leu62 | Ala20 |
| Ala101 | Ser23 |
| Gln102 | Val26 |
| Arg104 | Ile27 |
| Leu105 | Ser28 |
| Leu108 | Arg30 |
| Tyr109 | Arg31 |
| Ala112 | Val35 |
| Leu115 | Ser36 |
| Trp116 | Pro37 |
| Val119 | Pro38 |

The present amino acids define the site binding the aliphatic side chain of the detergent DDM, a good mimic of LTA₄.

Here, Trp 116 forms the roof of the pocket, Tyr59, Ala20, and Leu62 form the floor and side walls, whereas Leu115 creates a bottom that restricts further intrusion of the ω-end of LTA₄ into the protein. At the opposite end of LTA₄, the carboxyl group is positioned in a wide section of the substrate binding cleft.

TABLE 3
Catalytic domain for LTC4S

| Monomer1 | Monomer2 |
|---|---|
| Arg51 | Val16 |
| Asn55 | Ala20 |
| Glu58 | Ser23 |
| Tyr59 | Val26 |
| Leu62 | Ile27 |
| Tyr93 | Ser28 |
| Tyr97 | Arg30 |
| Ala101 | Arg31 |
| Gln102 | Val35 |
| Arg104 | Ser36 |
| Leu105 | Pro37 |
| Leu108 | Pro38 |
| Tyr109 | Tyr50 |
| Ala112 | Gln53 |
| Leu115 | |
| Trp116 | |
| Val119 | |

In Tables 1-3 above, the numbering of the amino acid sequence of LTC4S begin with the initial Met and is thus identical to the numbering in SEQ ID NO 1. The residues listed in Tables 1-3 are likely to define a common active site for homologues enzymes, i.e., other members of the MAPEG family.

It is preferred that the three-dimensional structure of at least a part of the active to site or a substrate binding region of LTC4S is a three-dimensional structure of at least a part of the “GSH substrate binding cavity”; the “lipophilic substrate binding crevice”; and/or the “catalytic site” or interacting regions, all as defined above, and a compound that is predicted to interact with the said “GSH substrate binding cavity”; “lipophilic substrate binding crevice”; and/or “catalytic site” or interacting regions of LTC4S is selected. Alternatively, the compound may bind to a portion of said LTC4S polypeptide that is not the “GSH substrate binding cavity”; “lipophilic substrate binding crevice”; and/or “catalytic site” or interacting regions of LTC4S, for example so as to interfere with the binding of a substrate molecule or its access to the catalytic site. In a still further example, the compound may bind to a portion of LTC4S so as to decrease said polypeptide's activity by an allosteric effect. This allosteric effect may be an allosteric effect that is involved in the natural regulation of LTC4S′ s activity.

The compound may bind to a portion of LTC4S that is involved in interaction between the subunits of the homotrimer. Residues considered to be involved in interaction between subunits are indicated in the following Tables. The subunits are indicated as subunits A, B and C, with the interactions of A with B and A with C indicated.

TABLE 4

| | | |
|---|---|---|
| Residue Number | Residue Name | Chain ID |

Protein Residue Contacts A B

| | | |
|---|---|---|
| −4 | HIS | A |
| −2 | HIS | A |
| −1 | HIS | A |
| 0 | HIS | A |
| 1 | HIS | A |
| 3 | ASP | A |
| 4 | GLU | A |
| 44 | PRO | A |
| 47 | GLU | A |
| 48 | ARG | A |
| 51 | ARG | A |
| 58 | GLU | A |
| 59 | TYR | A |
| 61 | PRO | A |
| 62 | LEU | A |
| 65 | ALA | A |
| 66 | THR | A |
| 69 | VAL | A |
| 72 | ILE | A |
| 73 | PHE | A |
| 97 | TYR | A |
| 101 | ALA | A |
| 104 | ARG | A |
| 108 | LEU | A |
| 115 | LEU | A |
| 116 | TRP | A |
| 119 | VAL | A |
| 122 | ALA | A |
| 123 | ALA | A |
| 126 | LEU | A |
| 129 | HIS | A |
| 130 | PHE | A |

Protein Residue Contacts B A

| | | |
|---|---|---|
| −4 | HIS | B |
| −3 | HIS | B |
| −1 | HIS | B |
| 0 | HIS | B |
| 1 | HIS | B |
| 2 | LYS | B |
| 3 | ASP | B |
| 5 | VAL | B |
| 6 | ALA | B |
| 7 | LEU | B |
| 9 | ALA | B |
| 10 | ALA | B |
| 12 | THR | B |
| 13 | LEU | B |
| 16 | VAL | B |
| 17 | LEU | B |
| 19 | GLN | B |
| 20 | ALA | B |
| 23 | SER | B |
| 24 | LEU | B |
| 27 | ILE | B |
| 30 | ARG | B |
| 37 | PRO | B |
| 38 | PRO | B |
| 39 | LEU | B |
| 40 | THR | B |
| 50 | TYR | B |
| 53 | GLN | B |
| 54 | VAL | B |
| 57 | SER | B |
| 60 | PHE | B |
| 61 | PRO | B |
| 64 | LEU | B |
| 68 | TRP | B |

Protein Residue Contacts A C

| | | |
|---|---|---|
| −4 | HIS | A |
| −3 | HIS | A |
| −1 | HIS | A |
| 0 | HIS | A |
| 1 | HIS | A |
| 2 | LYS | A |
| 3 | ASP | A |
| 5 | VAL | A |
| 6 | ALA | A |
| 7 | LEU | A |
| 9 | ALA | A |
| 10 | ALA | A |
| 12 | THR | A |
| 13 | LEU | A |
| 16 | VAL | A |
| 17 | LEU | A |
| 19 | GLN | A |
| 20 | ALA | A |
| 23 | SER | A |
| 24 | LEU | A |
| 26 | VAL | A |
| 27 | ILE | A |
| 30 | ARG | A |
| 37 | PRO | A |
| 38 | PRO | A |
| 39 | LEU | A |
| 40 | THR | A |
| 50 | TYR | A |
| 53 | GLN | A |
| 54 | VAL | A |
| 57 | SER | A |
| 60 | PHE | A |
| 61 | PRO | A |
| 64 | LEU | A |
| 68 | TRP | A |

Protein Residue Contacts C A

| | | |
|---|---|---|
| 1 | GTT | C |
| −1 | HIS | C |
| 0 | HIS | C |
| 1 | HIS | C |
| 3 | ASP | C |
| 4 | GLU | C |
| 44 | PRO | C |
| 47 | GLU | C |
| 48 | ARG | C |
| 51 | ARG | C |
| 58 | GLU | C |
| 59 | TYR | C |
| 61 | PRO | C |
| 62 | LEU | C |
| 65 | ALA | C |
| 66 | THR | C |
| 69 | VAL | C |
| 72 | ILE | C |
| 73 | PHE | C |
| 97 | TYR | C |
| 101 | ALA | C |
| 104 | ARG | C |
| 108 | LEU | C |
| 115 | LEU | C |
| 116 | TRP | C |
| 119 | VAL | C |
| 122 | ALA | C |
| 123 | ALA | C |
| 126 | LEU | C |
| 129 | HIS | C |
| 130 | PHE | C |

Thus, a further aspect of the invention provides a method for selecting or designing a compound expected to modulate the activity of Leukotriene C4 synthase (LTC4S), the method comprising the step of using molecular modelling means to select or design a compound that is predicted to interact with a subunit interaction region of LTC4S, wherein a three-dimensional structure of at least a part of a subunit interaction region of LTC4S is compared with a three-dimensional structure of a compound, and a compound that is predicted to interact with the said substrate interaction region is selected. Residues involved in subunit interaction regions are indicated in the preceding Table 4.

It will be appreciated that a compound may have component parts that are predicted to interact with more than one part of LTC4S, for example more than one part of the LTC4S active site. For example, a compound may have a component part that interacts with the GSH substrate binding cavity of the LTC4S, as discussed above; and another component part that interacts with a different part of the LTC4S, for example with the “lipophilic substrate binding crevice”; and/or the “catalytic site” ie with other parts of the active site. A compound for further testing may be “assembled” from component parts (which may individually be very small) that are predicted to bind to different parts of the LTC4S, for example different parts of the LTC4S active site.

The three-dimensional structures may be displayed by a computer in a two-dimensional form, for example on a computer screen. The comparison may be performed using such two-dimensional displays.

The following relate to molecular modelling techniques: Blundell et at (1996) Structure-based drug design Nature 384, 23-26; Bohm (1996) Computational tools for structure-based ligand design Prog Biophys Mol Biol 66(3), 197-210; Cohen et al (1990) J Med Chem 33, 883-894; Navia et al (1992) Curr Opin Struct Biol 2, 202-210.

The following computer programs [wes1], for example, may be useful in carrying out the method of this aspect of the invention: GRID (Goodford (1985) J Med Chem 28, 849-857; available from Molecular Discovery, Pinner, UK); MOE (Chemical Computing Group, Montreal, Quebec, Canada); AUTODOCK (Goodsell et al (1990) Proteins: Structure, Function and Genetics 8, 195-202; available from Scripps Research Institute, La Jolla, Calif., USA); DOCK (Kuntz et al (1982) J Mol Biol 161, 269-288; available from the University of California, San Francisco, Calif.); LUDI (Bohm (1992) J Comp Aid Molec Design 6, 61-78; available from Accelrys, San Diego, Calif., USA); Sybyl (Tripos Associates, St Louis, Mo., USA); Gaussian 03, for example revision D (Gaussian, Inc., Pittsburgh, Pa., USA); AMBER (University of California at San Francisco, San Francisco, Calif., USA); QUANTA (Accelrys, San Diego, Calif., USA); and Insight II (Accelrys, San Diego, Calif., USA). Programs may be run on, for example, a Silicon Graphics™, an IBM RISC/6000™, or a Red Hat Enterprise Linux workstation.

Several in silico methods could be employed, for example as described in to Example 2, or via a substructure search for new ligands using programmes such as [wes2] Unity (Tripos Associates, St Louis, Mo., USA) or ChemFinder (CambridgeSoft, Cambridge, Mass., USA). The basic structure of the natural ligand (LTA₄ or GSH) or part thereof capable of binding to LTC4S is taken (or predicted) and various structural features of it (for example the hydrophobic and charged entities) are submitted to a programme which will search a set of chemical company catalogues for chemicals containing this substructure. For example the structure of the ends of GSH may be useful in searching for compounds which may interact with the GSH binding pocket or catalytic site, whilst the structure of LTA₄, for example its length, may be useful in searching for compounds which interact with the lipophilic substrate binding crevice.

These compounds are then screened, either using a computer or by eye, for groups that could not interact with portions of the catalytic site, for example the GSH substrate binding cavity (or other portion, as discussed above) because, for example, they are too large or have steric or charge hindrance, and those are discarded. The remaining chemicals are submitted to a PRODRG server and topologies/co-ordinates for these chemicals are created. These chemicals are modelled into the structure, from which chemicals that are possibly able to bind to the GSH substrate binding cavity/lipophilic substrate binding crevice/catalytic site/interacting regions are selected. Further details of the PRODRG programme are available at http://davapc1.bioch.dundee.ac.uk/programs/prodrg/prodrg.html.

An alternative approach is to use PRODRG: a tool for generating GROMOS/MOL2/WHATIF topologies and hydrogen atom positions from small molecule PDB files. Starting from the natural ligand the skilled person can computationally vary all possible groups at each site on the ligand, with a variety of new groups while the protein co-ordinates and the ligand back-bone co-ordinates remain fixed. The results can then be screened for hindrance, repulsion and attraction.

A starting compound may initially be selected by screening for an effect on LTC4S enzyme activity (for example using LTA₄ as a substrate); then compared with the structure; used as the basis for designing further compounds which may then be tested by further modelling and/or synthesis and assessment, as discussed further below.

The selected compounds may then be ordered or synthesised and assessed, for one or more of ability to bind to and/or modulate LTC4S activity. The compounds may be crystallised with the LTC4S polypeptide and the structure of any complex determined.

The method of the invention may further comprise the steps of providing, synthesising, purifying and/or formulating a compound selected using computer modelling, as described above; and of assessing whether the compound modulates the activity of LTC4S. The compound may be formulated for pharmaceutical use, for example for use in in vivo trials in animals or humans.

Thus, the present invention provides methods of structure-based design of LTC4S inhibitors. Such methods are based, for example, on the use of the present coordinates, or preferably the coordinates defining a selected region, as templates in order to synthesize advantageous inhibitors with strong and specific binding properties. More specifically, such methods can first use a conventional organic synthesis, alone or combined with combinatorial chemistry, wherein the structure of the product of the synthesis is then further refined by cycles of crystallisation of enzyme and inhibitor, followed by another chemical synthesis, the product of which is again refined, etc.

A compound that modulates the activity of LTC4S may be selected. For example, a compound that increases the activity of LTC4S may be selected, or a compound that decreases the activity of LTC4S may be selected. Situations in which each type of compound may be useful (or for which such a compound is a starting point for further investigations or compound design) are indicated below.

The ability of the compound to modulate the activity of LTC4S towards LTA₄ (5S,5,6-oxido-7,9-trans-11,14-cis eicosatetraenoic acid) may be assessed. Such assessment may also be carried out in a microtitre plate format or other format suitable for high throughput screening. The assessment may be carried out using enzyme assay techniques well known to those skilled in the art and as described below. The LTC4S polypeptide used in such an assay may be a LTC4S polypeptide that retains LTC4S activity, as discussed above. For example, a polypeptide comprising full length human LTC4S or comprising a fragment of human LTC4S, for example a fragment lacking up to the C-terminal 20, 10, 5, 4, 3, 2 or 1 amino acids may be used, as will be apparent to those skilled in the art. Any such competent fragment has to be present together with a complementary fragment to form an active dimer or trimer since the active site is composed of residues from two adjacent subunits: it is considered that assembly into such a dimmer or trimer occurs spontaneously, but they may also be connected by a linker, using standard techniques in protein engineering.

An example of an assay that can be used in assessing the ability of a compound to modulate, for example inhibit, the action of LTC4S is described above.

Alternatively, the ability of the compound to modulate the fatty acid hydroperoxidase activity of LTC4S may be measured. An example of a suitable assay is described above.

Whilst it is anticipated that a compound binding to the active site of LTC4S will modulate the LTC4S activity of LTC4S (for example as assessed by action on LTA₄), it is possible that other activities or properties of LTC4S may be modulated, for example subunit interactions or interactions with other polypeptides (for example other MAPEG family members, for example FLAP), phosphorylation (Gupta N et al. FEBS Lett. (1999) 449(1):66-70), or effects of divalent cations (Nicholson D W et al. Eur. J. Biochem. (1992) 209(2):725-34), or intramolecular interactions or interactions with other LTC4S or FLAP monomers or dimmers (Mandal A K et al Proc. Natl. Acad. Sci. USA (2004) 101(17):6587-92).

As noted above, the selected or designed compound may be synthesised (if not already synthesised) or purified and tested for its effect on LTC4S (or a fragment, variant or fusion with LTC4S activity), for example its effect on the LTC4S activity. The compound may be tested in an in vitro screen for its effect on a LTC4S polypeptide or on a cell or tissue in which LTC4S is present. The cell or tissue may contain endogenous LTC4S and/or may contain exogenous LTC4S (including LTC4S expressed as a result of manipulation of endogenous nucleic acid encoding LTC4S). The compound may be tested in an ex vivo or in vivo screen, which may use a transgenic animal or tissue. The compound may also be tested, for comparison, in a cell, tissue or organism that does not contain LTC4S (or contains reduced amounts of LTC4S), for example due to a knock-out or knock-down of one or more copies of the LTC4S gene. Suitable tests will be apparent to those skilled in the art and examples include assessment of effects in an animal or ex vivo model of inflammation. Example of suitable models include Zymosan induced peritonitis and ovalbumin-sensitized mice as an allergic asthma model. The compound may be tested in a human ex vivo model of inflammation, for example on human peripheral blood or human umbilical cord blood, or on cells isolated from human peripheral blood or human umbilical cord blood, for example on leukocytes, for example neutrophils, eosinophils, or mast cells.

Compounds may also be subjected to other tests, for example toxicology or metabolism tests, as is well known to those skilled in the art.

A compound which binds to the lipophilic substrate binding crevice may be a compound which is also capable of binding to the receptor for the product of an LTC4S, i.e. an LTC₄ receptor, e.g. on a cell, such as a mast cell. Examples of LTC₄ receptors include CysLT1, CysLT2 and GPR-17. Thus, such a compound may be useful as an LTC₄ antagonist or agonist. Appropriate tests may also be conducted to determine whether this is the case.

It is preferred that the LTC4S is a polypeptide which consists of the amino acid sequence of the LTC4S sequence referred to above or naturally occurring allelic variants thereof. It is preferred that the naturally occurring allelic variants are mammalian, preferably human. The LTC4S may be a fusion polypeptide, for example with an N-terminal hexahistidine tag or FLAP, as discussed above.

It is particularly preferred, although not essential, that the variant or fragment or derivative or fusion of the LTC4S, or the fusion of the variant or fragment or derivative has at least 30% of the enzyme activity of full-length human LTC4S with respect to the glutathione conjugation of LTA₄. It is more preferred if the variant or fragment or derivative or fusion of the said LTC4S, or the fusion of the variant or fragment or derivative has at least 50%, preferably at least 70% and more preferably at least 90% of the enzyme activity of LTC4S with respect to the glutathione conjugation of LTA₄. However, it will be appreciated that variants or fusions or derivatives or fragments which are devoid of enzymatic activity may nevertheless be useful, for example by interacting with another polypeptide. Thus, variants or fusions or derivatives or fragments which are devoid of enzymatic activity may be useful in a binding assay, which may be used, for example, in a method of the invention in which modulation of an interaction of a mutated LTC4S of the invention and a compound is measured.

By “variants” of a polypeptide we include insertions, deletions and substitutions, either conservative or non-conservative. In particular we include variants of the polypeptide where such changes do not substantially alter the activity of the said polypeptide, for example the LTC4S activity of LTC4S, as described above.

By “conservative substitutions” is intended combinations such as Gly, Ala; Val, Ile, Leu; Asp, Glu; Asn, Gln; Ser, Thr; Lys, Arg; and Phe, Tyr.

The one and three-letter amino acid code of the IUPAC-IUB Biochemical Nomenclature Commission is used herein. In particular, Xaa represents any amino acid. It is preferred that Xaa represents a naturally occurring amino acid. It is preferred that the amino acids are L-amino acids.

It is particularly preferred if the LTC4S variant has an amino acid sequence which has at least 65% identity with the amino acid sequence of LTC4S referred to above (eg in Lam et al (1994) supra), more preferably at least 70%, 71%, 72%, 73% or 74%, still more preferably at least 75%, yet still more preferably at least 80%, in further preference at least 85%, in still further preference at least 90% and most preferably at least 95% or 97% identity with the amino acid sequence defined above.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al (1994) Nucl Acid Res 22, 4673-4680). The parameters used may be as follows:

Fast pairwise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

It is preferred that the LTC4S has identical or conserved residues that are equivalent to Arg104 or Arg31; and/or Arg51, Arg30, Arg104, Gln53, Asn55, Glu58, Tyr59, Tyr93, Tyr97, Ile27, Pro37, Leu108 (GSH substrate binding cavity residues); and optionally also Ala20, Leu24, Ile27, Tyr59, Trp116, Ala112, Leu115, Leu108, Tyr109, Leu62, Val119, Thr66, Val16 and Leu17 (lipophilic substrate binding crevice residues). Other residues for which it may be desirable for the residues to be identical or conserved include other residues listed in tables 1, 2, 3 or 4 above.

A further aspect of the invention provides a mutated LTC4S polypeptide, wherein one or more residues equivalent to Arg51, Arg30, Arg104, Gln53, Asn55, Glu58, Tyr59, Tyr93, Tyr97, Ile27, Pro37, Leu 108, Ala20, Leu24, Ile27, Tyr59, Trp 116, Ala112, Leu115, Leu108, Tyr109, Leu62, Val119, Thr66, Val16 and Leu17 or Arg31 of full length human LTC4S is mutated.

The present invention relates to a mutated form of LTC4S, which mutated form comprises one or more of the mutations defined in the following Tables 5-7, wherein amino acids are given in single letter code. Thus, R51G/AN/L/I/S/T/D/E/N/Q/H/K/P/C/M/F/Y/W indicates that residue arginine 51, using the LTC4S numbering scheme, is modified to an alanine, valine, a leucine and so forth.

TABLE 5
Mutations in the active site (GSH binding site)

| | |
|---|---|
| R30G/A/V/L/I/S/T/D/E/N/Q/H/K/P/C/M/F/Y/W | 5(1) |
| R51G/A/V/L/I/S/T/D/E/N/Q/H/K/P/C/M/F/Y/W | 5(2) |
| Q53G/A/V/L/I/S/T/D/E/N/R/H/K/P/C/M/F/Y/W | 5(3) |
| N55G/A/V/L/I/S/T/D/E/R/Q/H/K/P/C/M/F/Y/W | 5(4) |
| E58G/A/V/L/I/S/T/D/N/Q/R/H/K/P/C/M/F/Y/W | 5(5) |
| Y59G/A/V/L/I/S/T/D/E/N/Q/R/H/K/P/C/M/F/W | 5(6) |
| Y93G/A/V/L/I/S/T/D/E/N/Q/R/H/K/P/C/M/F/W | 5(7) |
| Y97G/A/V/L/I/S/T/D/E/N/Q/R/H/K/P/C/M/F/W | 5(8) |
| R104G/A/V/L/I/S/T/D/E/N/Q/H/K/P/C/M/F/Y/W | 5(9) |

TABLE 6
Mutations in the active site (LTA4 binding site)

| | |
|---|---|
| A20G/V/L/I/S/T/D/E/N/Q/R/H/K/P/C/M/F/Y/W | 6(1) |
| Y59G/A/V/L/I/S/T/D/E/N/Q/R/H/K/P/C/M/F/W | 6(2) |
| L62G/A/V/I/S/T/D/E/N/Q/R/H/K/P/C/M/F/Y/W | 6(3) |
| L115G/A/V/I/S/T/D/E/N/Q/R/H/K/P/C/M/F/Y/W | 6(4) |
| W116G/A/V/L/I/S/T/D/E/N/Q/R/H/K/P/C/M/F/Y | 6(5) |

More specifically, this embodiment relates to a mutant comprising any combination of at least two mutated amino acids, or any one of the above mentioned sequences of mutations, or any separate one amino acid mutation selected from the group consisting of sequences nos (5)1-9, 6(1-5).

The mutated LTC4S may be useful in determining where on the LTC4S a polypeptide or compound of interest interacts. For example, the abilities of a compound (including polypeptide) to bind to the mutated and unmutated LTC4S, or to modulate the activity of the LTC4S may be measured and compared.

A further aspect of the invention provides a polynucleotide encoding a mutated LTC4S polypeptide of the invention. A still further aspect of the invention provides a recombinant polynucleotide suitable for expressing a mutated LTC4S of the invention. A yet further aspect of the invention provides a host cell comprising a polynucleotide of the invention.

A further aspect of the invention provides a method of making a mutated LTC4S of the invention, the method comprising culturing a host cell of the invention which expresses said mutated LTC4S and isolating said mutated LTC4S.

A further aspect of the invention provides a mutated LTC4S obtainable by the above method.

Examples of these aspects of the invention may be prepared using routine methods by those skilled in the art.

For example, the above mutated LTC4S may be made by methods well known in the art, for example using molecular biology methods or automated chemical peptide synthesis methods.

It will be appreciated that peptidomimetic compounds may also be useful. Thus, by “polypeptide” or “peptide” we include not only molecules in which amino acid residues are joined by peptide (—CO—NH—) linkages but also molecules in which the peptide bond is reversed. Methods of designing and making peptidomimetic compounds will be known to those skilled in the art.

The invention further provides a method of identifying or characterising a compound that modulates the activity of LTC4S, comprising the step of determining the effect of the compound on the LTC4S activity of, or ability of the compound to bind to, the said mutated LTC4S of the invention.

The method may further comprise determining the effect of the compound on the LTC4S activity of, or ability of the compound to bind to, the LTC4S which is not mutated at the said residue.

For example, as will be apparent to the skilled person it may be helpful to compare the effect of a compound on wild-type (unmutated) LTC4S and a mutated LTC4S in which residue(s) (for example residue(s) which a test compound is expected, for example from computer modelling studies, to interact with) are mutated, in order to confirm or otherwise that the compound is binding as expected.

It will be appreciated that the LTC4S or mutated LTC4S may be a fusion protein comprising a tag, for example to aid purification or crystallisation, for example a hexahistidine tag, as described in Example 1.

A further aspect of the invention provides a kit of parts useful in carrying out a method according to the preceding aspect of the invention, comprising (1) a mutated LTC4S of the invention and (2) the corresponding LTC4S which is not so mutated.

The capability of the said LTC4S polypeptide with regard to interacting with or binding to a compound (which may be a polypeptide) may be measured by any method of detecting/measuring a protein/protein interaction or other compound/protein interaction, as discussed further below. Suitable methods include methods such as, for example, yeast two-hybrid interactions, co-purification, ELISA, co-immunoprecipitation and surface plasmon resonance methods. Thus, the LTC4S polypeptide may be considered capable of binding to or interacting with a polypeptide or other compound If an interaction may be detected between the LTC4S polypeptide and the compound or polypeptide by ELISA, co-immunoprecipitation or surface plasmon resonance methods or by a yeast two-hybrid interaction or copurification method. It is preferred that the interaction can be detected using a surface plasmon resonance method. Surface plasmon resonance methods are well known to those skilled in the art. Techniques are described in, for example, O'Shannessy D J Determination of kinetic rate and equilibrium binding constants for macromolecular interactions: a critique of the surface plasmon resonance literature. Curr Opin Biotechnol. 1994 February; 5(1):65-71; Fivash M, Towler E M, Fisher R J BIAcore for macromolecular interaction. Curr Opin Biotechnol. 1998 February; 9(1):97-101; Malmqvist M BIACORE: an affinity biosensor system for characterization of biomolecular interactions. Biochem Soc Trans. 1999 February; 27(2):335-40.

The effect of the compound on the LTC4S activity of LTC4S may be assessed, as indicated above. A compound may be selected that decreases the LTC4S activity of LTC4S. Such compounds may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III, thereby inhibiting or decreasing the formation of LTD₄, LTE₄ or, especially, LTC₄.

Such compounds are thus expected to be useful in the treatment of disorders that may benefit from inhibition of production (i.e. synthesis and/or biosynthesis) of leukotrienes (such as LTC₄), for example a respiratory disorder and/or inflammation. Further tests may be performed to assess the suitability of the compound for the treatment of such a disorder and/or inflammation, as will be well known to those skilled in the art.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, such compounds may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulo-nephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyroiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Such compounds may be useful for either the therapeutic and/or prophylactic treatment of the above-mentioned conditions.

A compound that increases the LTC₄ synthase activity of LTC4S may be useful in situations in which enhanced production of LTC₄, LTD₄ and/or LTE₄ is useful, for example in enhancing an immune response, for example in patients with an impaired immune response.

It will be appreciated that the invention provides screening assays for use in trying to identify drugs which may be useful in modulating, for example either enhancing or inhibiting, the LTC4S activity of LTC4S. Compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and more preferably less than 1000, 750 or 500 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

It will be understood that it will be desirable to identify compounds that may modulate the activity of LTC4S in vivo. Thus it will be understood that reagents and conditions used in the method may be chosen such that the interactions between, for example, the LTC4S and the substrate, are similar to those between the human LTC4S and a naturally occurring substrate (for example LTA₄). As well known to those skilled in the art, different assay systems may be used to assess a compound, in some of which the convenience of the assay or the specificity for an effect on LTC4S may be optimised, whilst in others the in vivo relevance may be optimised, for example by assessing the effect of the compound in a whole cell.

The compounds that are tested in the screening methods of the assay or in other assays in which the ability of a compound to modulate the LTC4S activity of LTC4S may be measured, may be compounds that have been selected and/or designed (including modified) using molecular modelling techniques, for example using computer techniques.

The invention also provides a means for homology modelling of related proteins (referred to below as target proteins). By “homology modelling” is meant the prediction of related MAPEG family member structures based either on x-ray crystallographic data or computer-assisted de novo prediction of structure, based upon manipulation of the coordinate data of Tables I to II.

“Homology modelling” extends to target MAPEG proteins, which are related the human LTC4S protein whose structure has been determined in the accompanying examples. It also extends to LTC4S mutants.

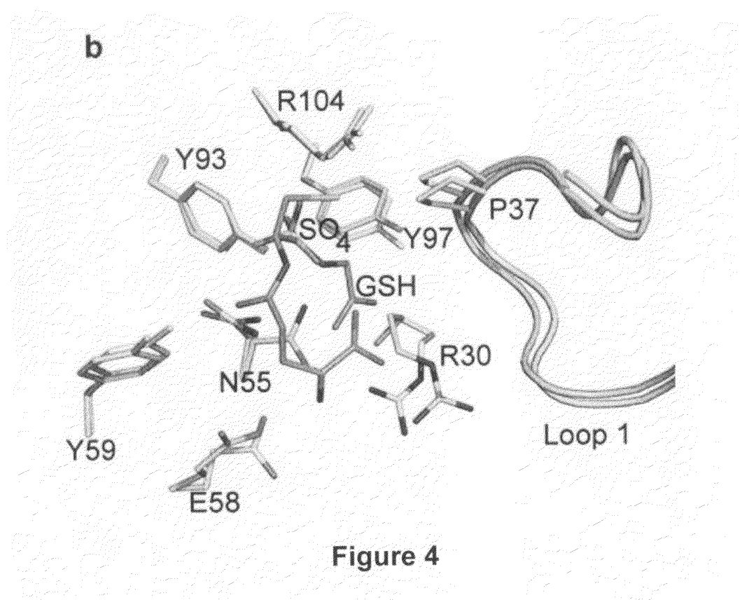

In general, the method involves comparing the amino acid sequences of the LTC4S protein of Table I or II with a target MAPEG family member protein by aligning the amino acid sequences. Amino acids in the sequences are then compared and groups of amino acids that are homologous (referred to as “corresponding regions”) are grouped together. This method identifies conserved regions of the polypeptides and accounts for amino acid insertions or deletions. Alignment of MAPEG family sequences in view of the structural information obtained from LTC4S is discussed in Example 1 and an alignment is shown in FIG. 6.

Homology between amino acid sequences can alternatively or in addition be determined using commercially available algorithms, as discussed above. Once the amino acid sequences of the polypeptides with known and unknown structures are aligned, the structures of the conserved amino acids in a computer representation of the polypeptide with known structure are transferred to the corresponding amino acids of the target protein. For example, a tyrosine in the amino acid sequence of known structure may be replaced by a phenylalanine, the corresponding homologous amino acid in the amino acid sequence of the target protein.

The structures of amino acids located in non-conserved regions may be assigned manually by using standard peptide geometries or by molecular simulation techniques, such as molecular dynamics. The final step in the process can be accomplished by refining the entire structure using molecular dynamics and/or energy minimization.

Homology modelling as such is a technique that is well known to those skilled in the art (see e.g. Greer, Science, Vol. 228, (1985), 1055, and Blundell et al., Eur. J. Biochem, Vol. 172, (1988), 513). The techniques described in these references, as well as other homology modelling techniques generally available in the art, may be used in performing the present invention.

Thus, a further aspect of the invention provides a method of predicting a three dimensional structure of a target LTC4S protein or other MAPEG family member protein or homo- or heteromultimer thereof (the target protein), the method comprises the steps of: aligning a representation of an amino acid sequence of the target protein with the amino acid sequence of the LTC4S of Table I or II, optionally varied by a root mean square deviation of not more than 2.0 Å, preferably less than 1.5 Å, 1.0, 0.5 Å, or selected coordinates thereof, to match homologous regions of the amino acid sequences; modelling the structure of the matched homologous regions of said target protein on the corresponding regions of the LTC4S structure as defined by Table I or II, optionally varied by a root mean square deviation of not more than 2, 1.5 or 1 Å, or selected coordinates thereof; and determining a conformation for said target protein which substantially preserves the structure of said matched homologous regions.

Preferably the method is performed using computer modelling.

The MAPEG family member may be FLAP, MGST1, MGST2, MGST3, MPGES-1 or an LTC4S protein. Typically, the structure of the MAPEG family member may be unknown or known only at low resolution, for example at less than 2.5 Å resolution. The predicted structure may, for example, be a predicted structure for a heteromultimer (heterodimer) of a LTC4S polypeptide with a FLAP polypeptide, or a fusion in which an internal segment of LTCS is replaced with a corresponding segment of FLAP, as noted above.

A further aspect of the invention provides a method of obtaining a structure of a target LTC4S protein or other MAPEG family member protein or homo- or heteromultimer thereof (the target protein), the method comprises the steps of: providing a crystal of said target protein; obtaining an X-ray diffraction pattern of said crystal; calculating a three-dimensional atomic coordinate structure of said target protein, by modelling the structure of said target protein on the LTC4S structure of Table I or II ± the root mean square deviation from the backbone atoms of the protein of less than 2.0 Å, preferably less than 1.5 Å, 1.0, or 0.5 Å, or selected coordinates thereof.

Thus, the present LTC4S structure may be useful in interpreting X-ray diffraction data from a related polypeptide.

In a further aspect of the invention, the 3D structure of LTC4S, as provided herein, can be used to interpret electron crystallographic data to generate a structure from 2D crystals. Accordingly, a further aspect of the invention provides a method of obtaining a structure of a target LTC4S protein or other MAPEG family member protein or homo- or heteromultimer thereof (the target protein), the method comprises the steps of: providing a crystal of said target protein; obtaining an electron diffraction pattern of said crystal; calculating a three-dimensional atomic coordinate structure of said target protein, by modelling the structure of said target protein on the LTC4S structure of Table I or II ± the root mean square deviation from the backbone atoms of the protein of less than 2.0 Å, preferably less than 1.5 Å, 1.0, or 0.5 Å, or selected coordinates thereof. The crystal can be a 2D crystal.

A further aspect of the invention provides a method for selecting or designing a compound expected to modulate the activity of a MAPEG family member protein or homo- or heteromultimer thereof, the method comprising the step of using molecular modelling means to select or design a compound that is predicted to interact with the catalytic site or a substrate binding region of the MAPEG family member protein or homo- or heteromultimer thereof, wherein a three-dimensional structure of at least a part of the catalytic site or a substrate binding region of the MAPEG family member protein or homo- or heteromultimer thereof is compared with a three-dimensional structure of a compound, and a compound that is predicted to interact with the said catalytic site or substrate binding region is selected, wherein the three-dimensional structure of at least a part of the catalytic site or a substrate binding region of the MAPEG protein or complex thereof is a three-dimensional structure (or part thereof) predicted or obtained by a method according to the preceding three aspects of the invention.

In the methods of the invention for selecting or designing a compound, the molecular structure to be fitted may be in the form of a model of a pharmacophore.

A further aspect of the invention provides a computer-based method of rational drug design comprising: (a) providing the coordinates of a LTC4S structure as defined in Table I or II optionally varied by a root mean square deviation of backbone atoms of the protein of less than 2.0, 1.5, 1.0 or 0.5 Å, or selected coordinates thereof; (b) providing the structures of a plurality of molecular fragments; (c) fitting the structure of each of the molecular fragments to the selected coordinates; and (d) assembling the molecular fragments into a single molecule to form a candidate modulator molecule.

The method may further comprise the step of: (a) obtaining or synthesising the molecular fragment or modulator molecule; and (b) contacting the molecular fragment or modulator molecule with LTC4S to determine the ability of the molecular fragment or modulator molecule to interact with LTC4S.

The selected coordinates may be coordinates defining the active site, for example substrate binding regions or catalytic site, as discussed above. Fragments may, for example, be fitted to different parts of the active site and may then be assembled together, using techniques well known to those skilled in the art. See, for example, Hajduk & Greer (2007) Nature Reviews Drug Discovery 6, 211-219.

A further aspect of the invention provides a method of obtaining a representation of the three dimensional structure of LTC4S, which method comprises providing the data of Table I or II optionally varied by a root mean square deviation of backbone atoms of the protein of less than 2.0, 1.5, 1.0 or 0.5 Å, or selected coordinates thereof, and constructing a three-dimensional structure representing said coordinates.

The structure may be presented as, for example, (a) a wire-frame model; (b) a chicken-wire model; (c) a ball-and-stick model; (d) a space-filling model; (e) a stick-model; (f) a ribbon model; (g) a snake model; (h) an arrow and cylinder model; (i) an electron density map; (j) a molecular surface model.

A further aspect of the invention provides computer readable storage medium or a computer system, intended to generate structures and/or perform optimisation of compounds which interact with LTC4S or other MAPEG family member protein or homo- or heteromultimer thereof, complexes of LTC4S or other MAPEG family member protein or homo- or heteromultimer thereof with compounds, the storage medium or system containing computer-readable data comprising one or more of: (a) LTC4S co-ordinate data of Tables I or II optionally varied by a root mean square deviation of backbone atoms of the protein of less than 2.0, 1.5, 1.0 or 0.5 Å, or selected coordinates thereof, said data defining the three-dimensional structure of LTC4S or said selected coordinates thereof; (b) atomic coordinate data of a target MAPEG family member protein or homo- or heteromultimer thereof generated by homology modelling of the target based on the coordinate data of Table I or II optionally varied by a root mean square deviation of backbone atoms of the protein of less than 2.0, 1.5, 1.0 or 0.5 Å, or selected coordinates thereof; (c) atomic coordinate data of a target MAPEG family member protein or homo- or heteromultimer thereof generated by interpreting X-ray crystallographic data or NMR data by reference to the co-ordinate data of Tables I or II optionally varied by a root mean square deviation of backbone atoms of the protein of less than 2.0, 1.5, 1.0 or 0.5 Å, or selected coordinates thereof; (d) structure factor data derivable from the atomic coordinate data of (b) or (c); and (e) atomic coordinate data of Table I or II optionally varied by a root mean square deviation of backbone atoms of the protein of less than 2.0, 1.5, 1.0 or 0.5 Å, or selected coordinates thereof.

Such a computer system may be useful in performing a selection or design method of the invention.

The term “computer-readable storage medium” will be well known to those skilled in the art and includes any medium or media which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

The term “a computer system” will also be well known to those skilled in the art and includes the hardware means, software means and data storage means used to analyse the atomic coordinate data of the present invention. The minimum hardware means of the computer-based systems of the present invention typically comprises a central processing unit (CPU), a working memory and data storage means, and e.g. input means, output means etc. A monitor may also be provided to visualize structure data. The data storage means may be RAM or means for accessing computer readable media of the invention. Examples of such systems are microcomputer workstations available from Silicon Graphics Incorporated and Sun Microsystems running Unix based, Linux based, Windows NT or IBM OS/2 operating systems. It will be appreciated that the methods of the invention may be performed by remote access to the atomic coordinate data of the present invention, for example using the internet.

All documents referred to herein are hereby incorporated by reference.

The invention is now described in more detail by reference to the following, non-limiting, Figures and Examples. All the references given below, and previously in this specification, are hereby included herein by reference.

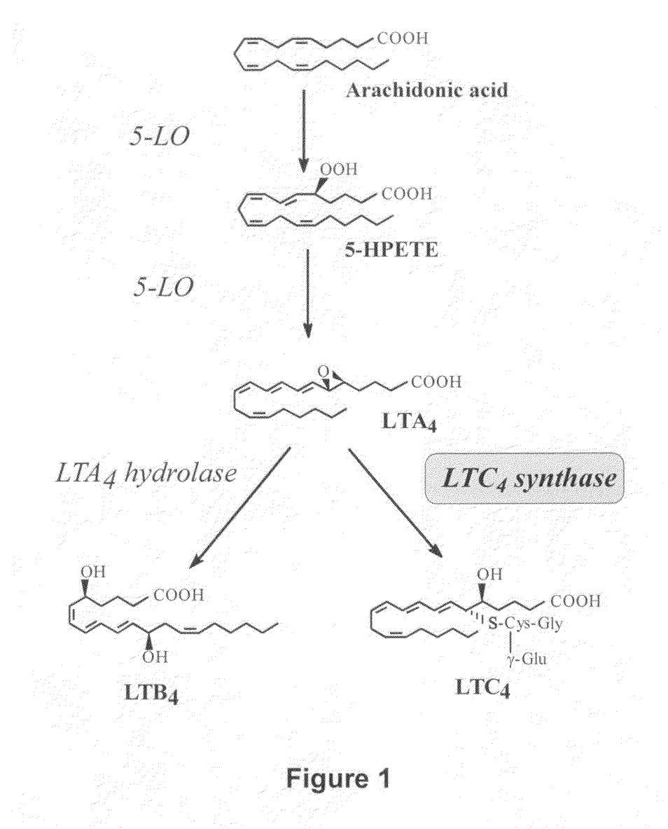

### FIG. 1 Key Enzymes and Intermediates in Leukotriene Biosynthesis.

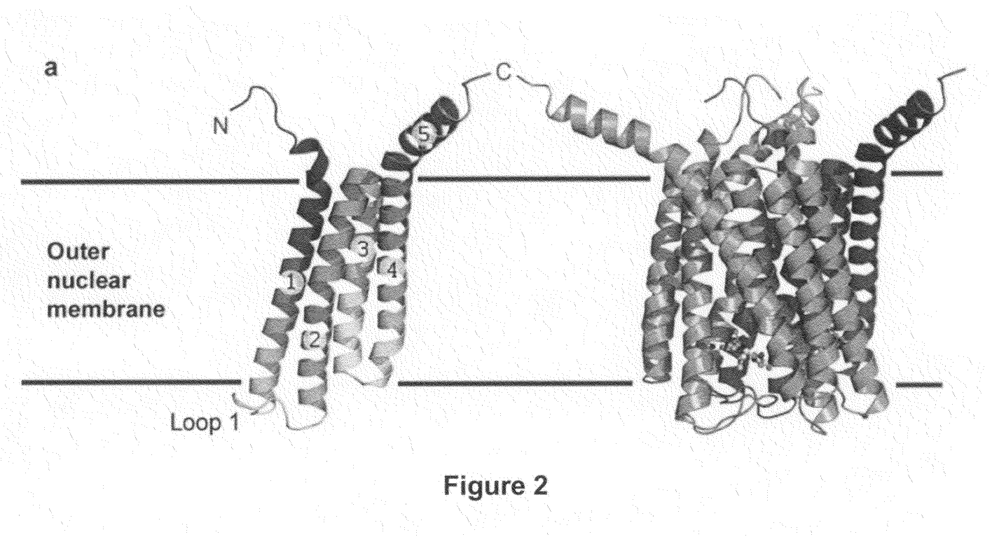

FIG. 2 LTC4 synthase overall structure. a) Left, front view of LTC4 synthase protomer in ribbon representation showing helices 1 through 5. The entire LTC4 synthase polypeptide, except for the last residue (the last four in the GSH-complexed structure), can be traced in the electron density maps. Right, the homotrimer. Bound glutathione, indicating the position of the active site, in ball and stick representation. Approximate membrane positions indicated by black lines. b) Cytosolic view of the trimer, showing the position of the three active sites with loop 1 covering each binding pocket. Glutathione shown in ball and stick representation together with modelled detergent.

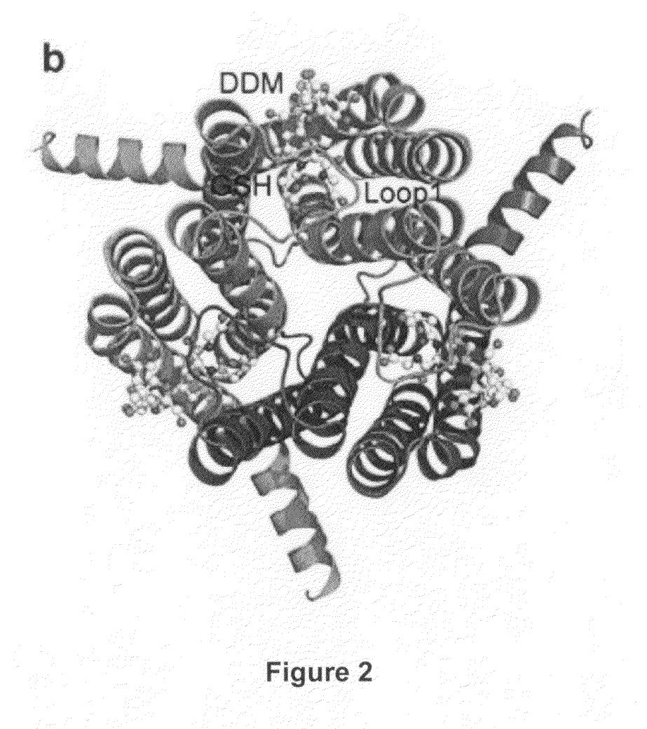

FIG. 3| Substrate- and lipid-protein interactions. a) Surface representation of the trimeric protein. There are ball and stick representations of carbon chains lining the protein surface. The detergent molecule is shown and underneath, a glimpse of the bound glutathione is seen. b) Cross-section from the cytosolic side of LTC4 synthase, as indicated in a), revealing the polar binding pocket of GSH and the cleft where the aliphatic co-substrate binds. Dashed bonds highlight the partial occupancy of the second pyranoside of the detergent molecule.

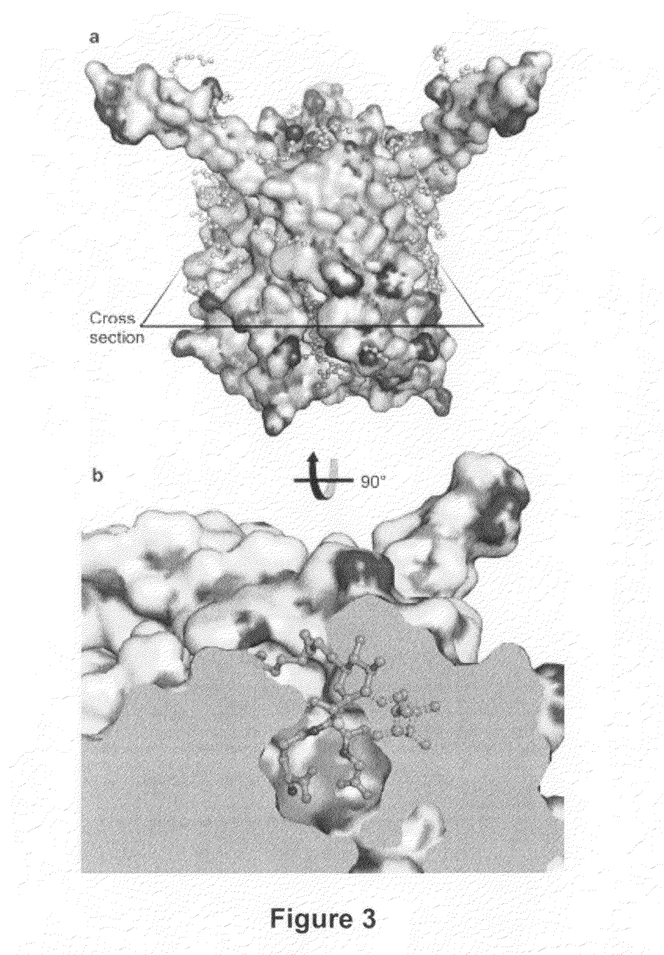

FIG. 4| Glutathione binding. a) Electron density map (2Fo-Fc, contoured at 3σ and phased with the apo structure before refinement) for bound glutathione shown in ball and stick representation. Interacting side chains are labeled accordingly. Chemical bonds to glutathione are drawn as dashed lines. A coordinated water molecule is shown as a sphere. b) Superposition of the active sites in the apo- and glutathione bound structures. Glutathione is shown in stick representation and the sulphate molecule from the apo-structure is indicated.

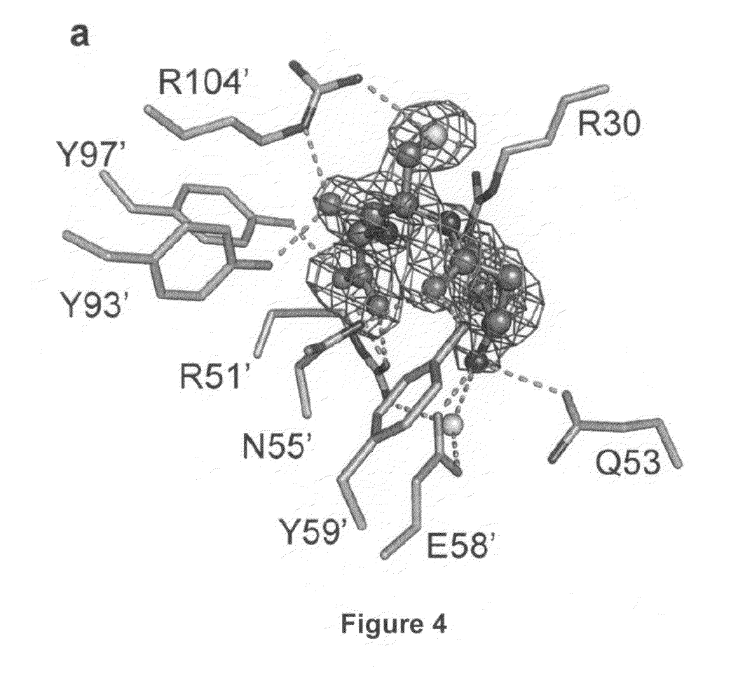

FIG. 5| The hydrophobic binding crevice and proposed schematic mechanism of substrate binding and catalysis. a) The interface between two monomers shown with interacting and bordering residues shown as sticks. A sphere shows the area where the cysteinyl sulphur is located. The bound detergent is depicted as a ball and stick model. b) Side view of the substrate binding region rotated 90° counter clockwise with respect to a). For clarity, only the monomer harbouring the Trp116′ crevice, is depicted. The removed monomer contributes hydrophobic residues to the lipid binding cleft and polar residues to glutathione. c) Schematic view of the proposed substrate binding and conjugation.

FIG. 6 Sequence alignment of the MAPEG family. Primary sequence of human (h) LTC4S aligned to other MAPEG members from mouse (m), rat (r), cow (c) and chicken (ch). Capital G indicates residues binding GSH in the structure. The conservation of these residues is highlighted. Helices indicated with corresponding helix numbers.

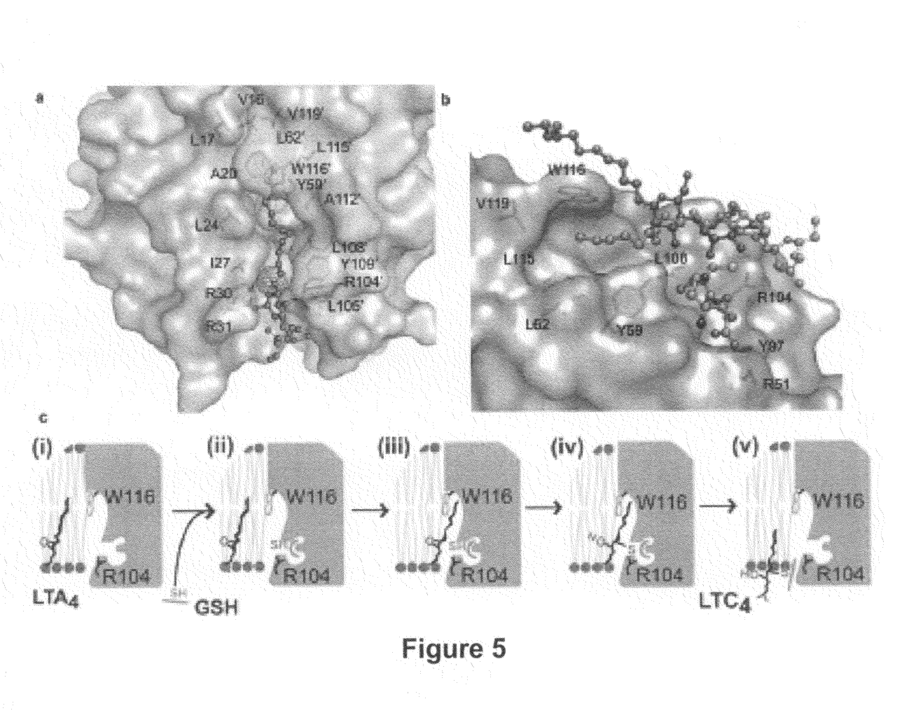

FIG. 7 Schematic drawing of the reaction catalyzed by LTC₄ synthase. LTA₄ and glutathione, on the left, are conjugated to form LTC₄ in a non-reversible reaction.

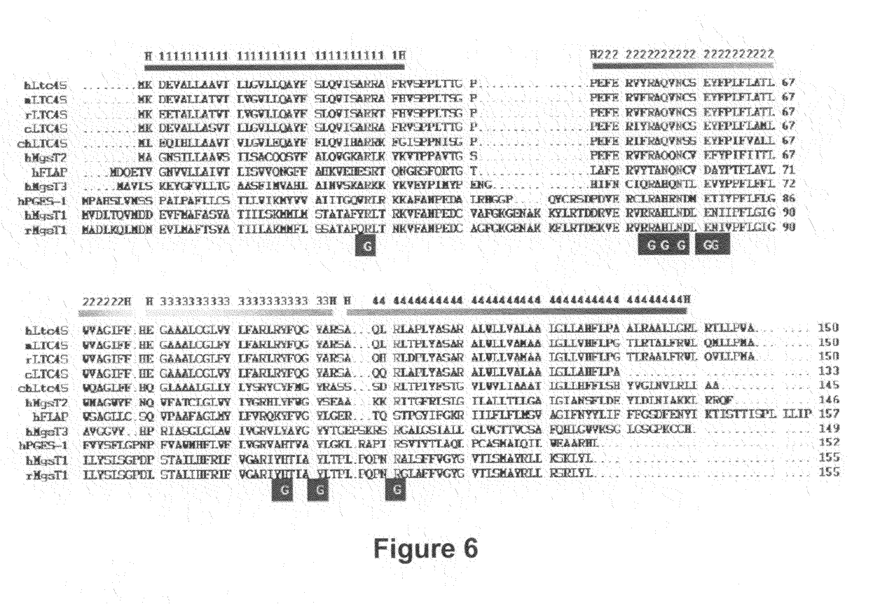

### FIG. 8 Metal Coordination and Crystal Packing

a) Perinuclear view showing full coordination by one trimer of one metal and partial coordination of 3 metals (dots) by the hexa-histidine tag. b) Crystal symmetry, largely governed by metal clusters of in total 8 metals per dodecamer coordinating the N-terminal as shown in a).

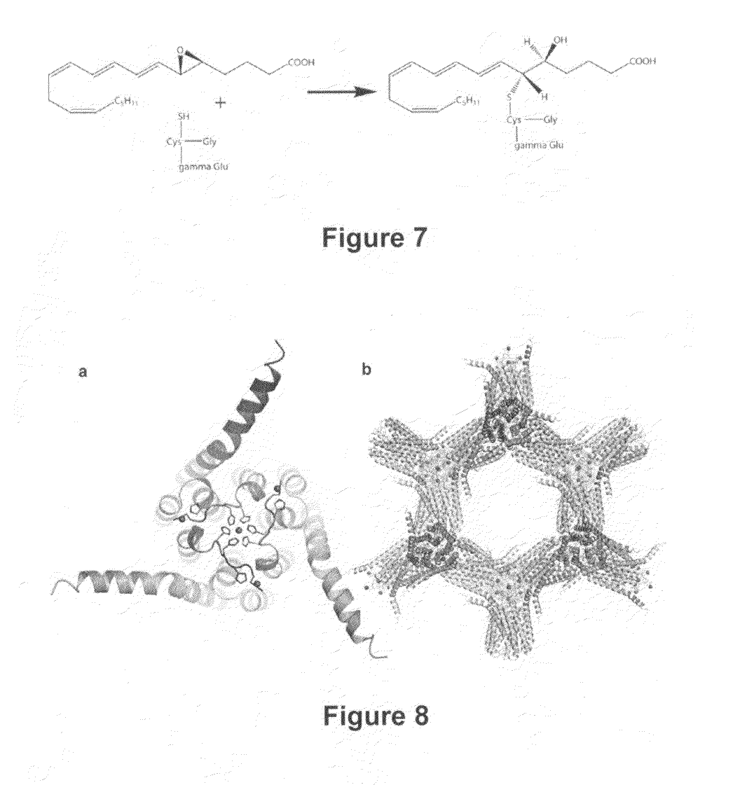

### FIG. 9 Glutathione Binding

Stereo view of active site showing electron density (2FO—FC, contoured at 3σ, phased with the apo structure before refinement) for bound glutathione shown in stick and ball representation. Interacting side chains, coloured by monomer and labelled accordingly. Chemical bonds to glutathione drawn as dashed lines.

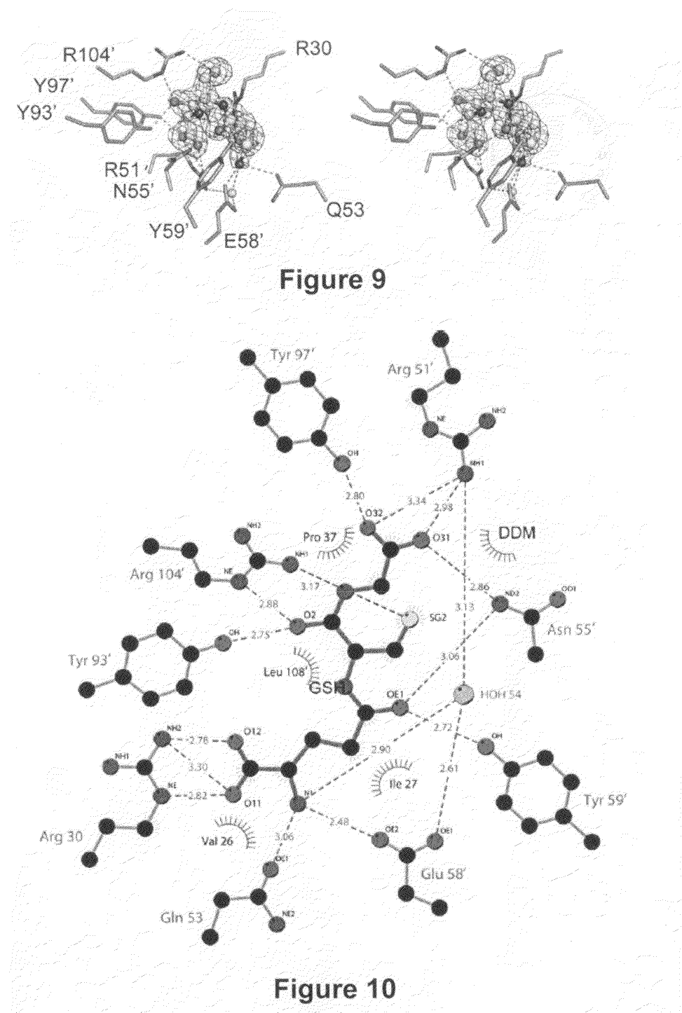

FIG. 10 Schematic diagram of protein ligand interactions Ligplot of glutathione and interacting residues showing bond lengths and interactions as dashed lines. A dark dashed line indicates the interaction between Arg104′ and the cysteinyl sulphur.

### EXAMPLE 1

### Crystallisation and Structure Determination for LTC4S

### Materials and Methods

Imidazole, Tris base, NaCl, KCl, Triton X-100, sodium deoxycholate, S-hexylglutathione agarose, probenecid, reduced glutathione (GSH), and 2-mercaptoethanol were obtained from Sigma. Dodecyl maltoside was obtained from Anatrace.

Human LTC4S was expressed as a hexa-histidine construct in yeast. Extraction from the membrane was performed with a Triton X-100 and Triton-DOC mixture. The protein was purified using two affinity chromatography steps and finalized with a gel filtration, allowing for detergent exchange to n-dodecyl β-D-maltoside.

### Cloning and Plasmid Construction

The human LTC4S cDNA (I.M.A.G.E. cDNA clone 5277851, MRC geneservice, Cambridge, UK) was subcloned into pPICZA (Invitrogen). Both the cDNA, supplemented with an Nterminal sequence encoding a His6 tag, and the vector were PCR amplified and the products were co-transformed into CaCl₂-competent E. coli (TOP10, Invitrogen), utilizing the endogenous recombinase activity of E. coli to recombine the fragments. The protein coding part of the resulting plasmid, pPICZ-hisLTC4S, was verified by DNA sequencing.

### Protein Expression and Purification

The expression vector was transformed into P. pastoris KM71H cells using the Pichia EasyComp Transformation kit (Invitrogen). Recombinant cells were cultivated in baffled flasks in 2.5 L minimal yeast medium with glycerol (Invitrogen) at 27° C. When OD600 reached 8-10, the cells were resuspended in 0.5 L minimal yeast medium with 0.5% methanol. The cells were harvested after 72 h by centrifugation (2500×g, 7 min) and resuspended in 50 mM Tris-HCl, pH 7.8, 100 mM KCl and 10% glycerol. The cells were homogenised with glass beads (0.5 mm) and the slurry was filtered through nylon net filters (180 μm, Millipore) and centrifuged (1500×g, 10 min). Membrane bound proteins in the supernatant were solubilized with Triton X-100 (1%, v/v) and sodium deoxycholate (0.5%, w/v) for 1 h with stirring on ice. After centrifugation (10 000×g, 10 min) the supernatant was supplemented with 10 mM imidazole and loaded on Ni-Sepharose Fast Flow (GE Healthcare). The column was washed with buffer A (25 mM Tris-HCl, pH 7.8, 10% glycerol, 0.1% Triton X-100 and 5 mM 2-mercaptoethanol) supplemented with 20 mM imidazole and 0.1 M NaCl, followed by additional wash with buffer A containing 40 mM imidazole and 0.5 M NaCl. LTC4 synthase was eluted with 300 mM imidazole, 0.5 M NaCl and 0.1 mM GSH in buffer A. The final step of purification was performed on a column packed with 3 mL S-hexylglutathione agarose. The column was washed with buffer A, supplemented with 0.5 M NaCl and 0.1 mM GSH. Pure LTC4 synthase was eluted with 25 mM Tris-HCl, pH 7.8, 0.1% Triton X-100, 30 mM probenecid, 5 mM 2-mercaptoethanol and 0.1 mM GSH. The purified protein was either stored frozen at −20° C. or directly further polished in a buffer exchange step on a Superdex 200 16/60 (GE Healthcare.) equilibrated with 0.03% w/v DDM (w/v), 20 mM Tris pH 8.0, 300 mM NaCl and 0.5 mM TCEP. Fractions containing LTC4 synthase were concentrated to 3.1 mg ml−1 by ultrafiltration.

### Crystallisation

Crystals were grown either at 4° C. or 20° C. from a 3.1 mg ml⁻¹ membrane protein solution, using sitting drop vapour diffusion technique. Crystals typically appeared after 3-4 days, reaching optimal size after approximately 7 days.

Protein solution containing 20 mM Tris pH 8.0, 300 mM NaCl, 0.03% (w/v) DDM was mixed (1:1) with reservoir solution containing either 200 mM NaCl, 100 mM Na cacodylate pH 6.5, 2 M Ammonium Sulphate (AmSO₄) for native protein; 2% PEG 400, 100 mM HEPES Na pH 7.5, 2M AmSO₄ for GSH derivative or 100 mM bis-Tris pH 5.5, 2M AmSO₄ for heavy atom soaks. For the latter, crystals were grown at 20° C. and soaked for 2 hours in 2.5 mM PtCN₄, dissolved in artificial mother liquor.

GSH derivatives were obtained by mixing equal volumes of 6 mM GSH, dissolved in mother liquor, with protein and left to soak for 24 hours at 4° C.

All crystals were transferred to their corresponding reservoir solution supplemented with 25% glycerol for cryo protection, then flash frozen in liquid nitrogen.

All X-ray data were collected on beam lines ID14-4 and ID23-2 at the European Synchrotron Radiation Facility (ESRF). Diffraction data of native and GSH soaked crystals was processed and scaled using Mosflm and SCALA while the XDS suite was used for processing and scaling of heavy atom MAD data sets.

Three highly redundant datasets to 3.2 Å were collected around the Pt LIII edge based on an X-ray fluorescence spectrum collected from the crystal. A single platinum site was located on the basis of anomalous differences observed in the peak dataset using SHELXD after local scaling in XPREP and SHELXE₂₈ confirmed the correct hand. Good experimental MAD (multiwavelength anomalous diffraction) phases were obtained using the program SHARP₂₉, resulting in an overall figure of merit (FOM) of 0.41/0.26 for the acentric/centric reflections respectively. These phases were further improved using a 75% solvent content in SOLOMON₃₀, resulting in an overall figure of merit of 0.88.

The initial map was used to model α-helices, which were in turn used together with high resolution data to assign residues with ARP/wARP₃₁ which managed to build ˜80% of the sequence. The model was further built using Coot 32 and used for molecular replacement with Phaser ₃₃ of a higher resolution apo structure and the GSH bound structure. Both protein models were built in Coot and refined using Refmac₃₄ to an R_{work}/R_{free} of 17.6/21.2% for the apo model and to 18.3/22.0% for the GSH bound structure. All crystals belonged to space group F23 with cell dimensions of approx a=b=c=170 Å and α=β=γ=90° containing one monomer per asymmetric unit having a solvent content of 80%.

All images were prepared using Pymol ₃₅ except FIG. 10 which was generated with LIGPLOT ₃₆

TABLE 8
Data collection, phasing and refinement statistics.

| | | Native 2 | Native 3 | Crystal 1 | | |
|---|---|---|---|---|---|---|
| | Native 1 | Apo | GSH | Pt(CN)2 | | |
| Data collection | | | | | | |
| Space group | F23 | F23 | F23 | F23 | | |
| Cell | | | | | | |
| dimensions | | | | | | |
| a = b = c (Å) | 170.260 | 169.670 | 169.940 | 170.78 | | |
| a = b = g (°) | 90.00 | 90.00 | 90.00 | 90.00 | | |
| | | | | Peak | Inflection | Remote |
| Wavelength | 0.93924 | 0.9390 | 0.8733 | 1.07150 | 1.07200 | 1.06888 |
| Resolution (Å) | 30.0-2.2 | 51.16-2.0 | 51.232-2.15 | 30.0-3.2 | 30.0-3.2 | 30.0-3.2 |
| R_{merge} | 11.2/(51.4) | 8.5/(81.5) | 14.1/(66.7) | 15.5/(53.4) | 15.4/(52.6) | 14.8/(50.9) |
| I/sI | 15.6/(2.8) | 16.1/(2.2) | 8.8/(2.3) | 14.0/(2.5) | 14.6/(2.6) | 13.9/(2.65) |
| Completeness | 99.8/(100) | 99.8/(100) | 99.8/(100) | 99.5/(99.8) | 99.5/(99.6) | 99.5/(99.5) |
| (%) | | | | | | |
| Redundancy | 7.4 | 7.2 | 5.0 | 6.4 | 6.3 | 6.3 |
| Refinement | | | | | | |
| Resolution (Å) | | 2.0-28.3 | 2.15-49.03 | | | |
| No. reflections | | 25914 | 20920 | | | |
| R_{work/}R_{free} | | 0.1756/0.2117 | 0.1830/0.2202 | | | |
| No. atoms | | | | | | |
| Protein | | 1267 | 1194 | | | |
| Ligand/ion | | | 53 | | | |
| Water | | 113 | 126 | | | |
| B-factors | | — | | | | |
| Protein | | 23.7 | 28.0 | | | |
| Ligand/ion | | — | 33.1 | | | |
| Water R.m.s | | 59.2 | 40.2 | | | |
| deviations | | | | | | |
| Bond lengths | | 0.022 | 0.025 | | | |
| (Å) | | | | | | |
| Bond angles | | 1.987 | 2.485 | | | |
| (°) | | | | | | |

### Results and Discussion

### LTC4 Architecture and Membrane Interactions

We have determined the crystal structure of the human LTC4 synthase in its apo and GSH-complexed forms, to 2.0 and 2.15 Å resolution respectively. The entire LTC4S polypeptide, except for the last residue (four last in the GSH-complexed structure), can be traced in the electron density maps. In addition, the N-terminal 6-His tag used for purification is clearly visible, and forms a unique metal coordinated cluster composed of 12 crystallographically related hexa-histidine tags (FIG. 8) coordinating 8 metals. A number of extended densities, likely to originate from ordered detergents or lipids, are seen in the maps and have been introduced into the crystallographic refinement as the detergent dodecyl maltoside (DDM) used in the purification or as tentative aliphatic chains. A notable cluster of such aliphatic chains pack against helices 1, 3 and 5, underscoring the high hydrophobicity of helix 5, where the first half is almost exclusively composed of hydrophobic residues (FIG. 3a). In its natural membrane environment, it is plausible that helix 5 slightly changes direction to be embedded into the lipid bilayer as an interfacial surface helix.

LTC4S is composed of five long α-helices—the first four (helix 1-4) forming the transmembrane segments, while helix 5 extends out of the membrane plane (FIG. 2). The crystal structure reveals a compact trimeric protein, where a crystallographic three-fold axis relates the three subunits. Two of the three TM helix connecting loops are short (loop 2 and 3), while loop 1, connecting helix 1 and 2, is longer, constituted by 11 residues. Loop 1 folds on top of the neighbouring monomer and contributes to the subunit interaction in the trimer (FIG. 2). Helix 4 and 5 are connected by a short proline containing turn and helix 5 could also be seen as an extension of helix 4.

The active site of LTC4S, identified by a bound GSH, is buried at the interface of two adjacent monomers close to the membrane face where loop 1 is positioned. It is likely that this part of the protein faces the cytoplasmic side of the outer nuclear membrane. This would facilitate delivery and release of substrates and product, since both the preceding and following steps in the synthesis pathway are conducted on the cytosolic side of the membrane. Crystal contacts are mediated by the C-terminal helix, the N-terminal 6-His tag on the perinuclear side of the protein and also by loop 1 and 3 on the cytosolic side. The fact that the crystals contain only ˜20% protein, but still diffract to high resolution is unusual, but might be explained by the high symmetry space group (F23) and the intriguing 6-His polynuclear metal cluster which contains an internal crystallographic 322 symmetry. (FIG. 8).

The electron density maps reveals a number of extended electron densities that are likely to originate from bound detergent and lipid molecules (FIG. 2a). In the GSH soaked structure one tentative DDM molecule is modelled close to the bound GSH, marking the active site (see below), in the crevice formed between helix 1 from one subunit and helix 3 from a neighbouring subunit. In the apo-LTC4S structure, two DDM molecules are modelled in this region.

Most of the additional aliphatic chains are also seen on the perinuclear half of the TM region and include one extended chain found in the centre of the trimer that might be important for stabilising the trimeric structure. In general, the cytoplasmic half of the molecule containing the active site pocket is more polar. The location of the active site on the cytosolic half of the enzyme is consistent with that of 5-LO, the enzyme producing the co-substrate for LTC4S, which is found in the cytosol. The topology is also consistent with that protein kinase C (Gupta N et al. FEBS Lett. (1999) 449(1):66-70) known to phosphorylate S28 of LTC4S is found in the cytosol.

The four-helix TM topology of the LTA4S is also supported by a low resolution electron diffraction projection image of LTC4S (Schmidt-Krey et al., 2004, supra). A four helix TM structure was also seen in the low resolution electron crystallography structure of the distantly related MGST1 (Holm et al 2006, supra).

### Active Site Structure and Substrate Binding

The location of the active site of LTC4 synthase was revealed by a clearly bound GSH. The active site of the enzyme is buried at the interface of two adjacent monomers close to the membrane face where loop 1 is positioned, between helix 1 and 2 from one monomer and 3 and 4 from a neighbouring monomer. It is likely that this part of the protein faces the cytoplasmic side of the outer nuclear membrane. This would facilitate delivery and release of substrates and product, since both the preceding and following steps in the synthesis pathway are conducted on the cytosolic side of the membrane. GSH adopts a horseshoe shaped conformation deep in a polar pocket at the interface between helix 1 and 2 from one monomer and 3 and 4 from a neighbouring monomer (FIGS. 2b, 3b). The carboxylate moieties of GSH face the protein matrix, while the thiol group is directed towards the membrane layer where a DDM molecule is bound (FIG. 2b). Polar interactions are made with GSH by residues from both subunits constituting the active site (FIGS. 4a,b). Arg51′ and Arg30 make salt bridges to the two carboxylates of GSH, at the base of the binding pocket, effectively bending GSH and directing its thiol group towards the membrane interface where it interacts with Arg 104′ and is positioned close to a bound DDM molecule (FIGS. 3b, 4a). Additional polar interactions to GSH are made by Gln53, Asn55′, Glu58′, Tyr59′, Tyr93′ and Tyr97′. Several non-polar interactions are also made (Ile 27, Pro37, Leu 108′) providing a good fit for GSH into its binding pocket. For a detailed overview of the GSH-binding see FIG. 10.

The structure of LTC4S has also been determined in a GSH free apo form, where a tentative sulphate ion is found in the GSH pocket. Comparison of the GSH bound LTC4S structure with the apo-LTC4S structure reveals that only local adjustments of polar amino acid side chains are made upon GSH binding (FIG. 4b). While the hydrophobic and aromatic residues providing interactions with GSH are in very similar positions in the two structures, most of the polar residues change conformation upon GSH binding. Loop 1 appears to at least partially cover the access of GSH to its binding pocket (FIGS. 2b, 4b). Therefore some flexibility of loop1 might be required during the reaction cycle, consistent with structural rearrangement of this loop upon GSH binding (FIG. 4).

### Recognition of the Lipophilic LTA4 Substrate

Structures of enzymes in complexes with lipophilic substrates are rare and in fact, except for electron carriers, no direct structural information is available in the literature of an integral membrane enzymes in complex with its lipid substrate, or a good analog thereof (http://blanco.biomol.uci.edu/Membrane_Proteins_xtal.html). Crystallographic studies of LTC4S with its co-substrate LTA₄ are challenging due to the short lifetime of this epoxide containing compound (˜10 seconds). In the GSH complexed LTC4S structure, however, a lipophilic molecule is already bound in the active site region, and based on the length of the aliphatic chain and on the apparent structure of its head group, this molecule has been assigned as DDM, although the terminal sugar group is mainly disordered (FIG. 4a). The 12-carbon chain and the first sugar group are, however, well defined in the electron density and we propose that this bound detergent might serve as a good model for the binding of LTA₄ to the enzyme as it has important structural similarities to LTA₄. The aliphatic chain binds in an elongated cavity on the enzyme lipid interface and the binding mode of this lipophilic compound positions atom 15, counted from the ω-end, on top of the GSH thiol group, consistent with LTA₄ being conjugated with GSH at this position, C6 of the LTA₄ substrate. The proposed binding canyon for LTA4 is hence constituted by a narrow elongated crevice formed by hydrophobic residues (FIG. 4a). The hydrophobic tail of the detergent is lined by Ala20, Leu 24, Ile27 from one subunit and Tyr59′, Trp116′, Ala 112′, Leu 115′, Leu108′ and Tyr 109′, from the neighbouring subunit. Trp116′ play a key role in positioning the aliphatic chain when it forms a lid over the ω-end of the substrate. The Trp116′ pocket constitute an ingenious mode for fixing the position of the ω-end of the lipid, effectively serving as a ruler to allow the appropriate positioning of C 6 of LTA₄ at the GSH thiol. In the GSH free apo-LTC4S structure, a DDM molecule is also bound in this region of the protein but the detergent molecule is translated further down into the lipid bilayer, as compared to the DDM in the GSH complexed structure (FIG. 5b). The effect of this translation is that the position of the detergent atom corresponding to C6 of LTA4, is now found more than 8 Å from where the GSH thiol is located in the GSH structure. Furthermore, the detergent in the apo-LTC4S structure is not wedged into the Trp 116 pocket with its ω-termini, but this termini instead sits outside the pocket. This gives further support for the fact that Trp 116 plays an important role in the alignment of the aliphatic chain of LTA₄ in the active site of LTC4S. It also suggests that GSH binding assists in the formation of an appropriate lipid binding crevice, presumably by covering charged groups in the active site and extending the interaction surface for the lipid, thereby allowing LTA₄ to enter into a productive binding mode in the active site of LTC4S.

In order to promote the GSH conjugation reaction, the nucleophilicity of the GSH thiol is most likely enhanced by the enzyme. Arg104 is ideally positioned to promote the pKa shift of the GSH thiol through its positive charge, where one of the η-nitrogens is able to mediate a polar interaction with the GSH sulphur (3.2 Å). This unusually short sulphur-nitrogen distance together with the lack of additional hydrogen bond acceptors suggest that the enzyme-bound thiol group may in fact be an anionic thiolate in the crystal structure, as suggested for the distantly related MGST-1₂₃. The GSH thiol is well positioned for a nucleophilic attack on the allylic C6 of the oxirane ring of LTA_{4 37}. There are no obvious acid residues situated in the active site that could provide a proton to the evolving C—O group of the substrate. It is therefore likely that the substrate oxyanion is stabilised by hydrogen bonding with the enzyme. Arg104 is located close to the expected position of the C5 of the substrate and could therefore also assist in stabilising the substrate anion. In addition, Arg31 is located on the distal side of the substrate and although it is flexible in the present structure, this residue may also assist in the stabilisation of the substrate anion. The chirality of the resulting SN2 mechanism will be defined by the productive binding of the epoxide in the active site. In the reaction, the opening of the epoxide will result in chiral inversion such that the 6S stereochemistry of the epoxide oxygen of LTA₄ will be completely converted to the 6R configuration of the resulting glutathionyl moiety of the product LTC₄.

FIG. 5c depicts a schematic summary of the proposed mechanism for substrate binding and product generation for LTC₄ synthase. In the proposed binding scheme, GSH enters its binding pocket from the cytosol and by doing so, enables binding of LTA₄ that previously resided in the lipid membrane. The hydrophilic addition to LTA₄ enables LTC₄ to migrate out in the cytosol, perhaps depending on the flexibility of loop 1.

The structural information on LTC₄ synthase, especially on the location of the TM segments and the position of the active site residues, allows for a structure based alignment of the MAPEG family (FIG. 6). Previous sequence alignments suggest a common evolutionary origin of the human MAPEG members, where sequence conservation is low but significant in the two central TM segments, helix 2 and helix 3₁₄. Sequences in the terminal TM segments, helix 1 and 4, however, are more diverged and clear similarities can only be seen within subfamilies. In the structure based alignment, further conservation is added to the active site region of the MAPEG family by the likely alignment of helix 1. The alignment of the first three helices strongly supports that the majority of the GSH coordinating residues are conserved in all the GSH dependent MAPEG members (All MAPEG members except FLAP are known to catalyse GSH dependent reactions). Out of the 8 polar residues coordinating GSH from these helices, 5 are fully conserved in the GSH dependent enzymes, while the remaining 3 have mainly conservative amino acid substitutions. Our alignment of helix 4 is more tentative. Arg104, the critical catalytic residue proposed here is aligned with arginines predicted to reside in the N-terminal end of helix 4 of other GSH dependent MAPEG members. The position of this potential catalytic Arg in MGST-1 (Arg130) appears to be consistent with a low-resolution structure of MGST-1₁₉. While the amino acids coordinating GSH are highly conserved in the MAPEG family, the residues lining the proposed LTA₄ binding crevice are not. Although most of the corresponding residues are hydrophobic in the other families, their size and aromatic composition vary. This may indicate how the MAPEG members have evolved to recognize different lipid substrates binding in the same pocket. However, it could also suggest that lipid recognition occurs differently in the other family members, for example, MGST-1 has evolved to be highly unspecific. In addition, the nature of the head group of LTA₄ has been shown to be variable without loss of enzymatic activity_{38,39}. This lack of major specificity for the head group can presumably be explained by the rather wide area in this region, which does not supply any distinct binding pocket for the carboxylate (FIGS. 3a, 5a).

In conclusion, it appears that the horseshoe-shaped binding mode of GSH is conserved throughout the GSH dependent MAPEG family members and, potentially, that the structural basis for GSH activation may be related. In LTC₄ synthase, the arginine at position 104 is likely to play a key role in activating the GSH thiol for a nucleophilic attack on the LTA₄ epoxide. The intriguing lipid binding pocket of LTC₄ synthase allows LTA₄ to fit into the active site by the Trp116 crevice, effectively positioning the C6 of the substrate for conjugation with GSH. It is plausible that this principle of lipid substrate recognition is primarily based on the aliphatic chain length rather than the nature of the head group. This may have some bearing also for substrate recognition in other integral membrane enzymes.

### EXAMPLE 2

### Computer-Based Compound Screening

Structure-based drug design utilizes the three-dimensional structure of a known target as a guide to rationally design molecules which may eventually lead to disease modifying agents, drugs. The structure of the target may be obtained through X-ray crystallography or another three-dimensional structure determination method.

Preferably, the crystals are comprised of target-ligand complexes depicting relevant binding modes and desired interactions of the putative drugs with the target. Most favorably, a series of target-ligand complexes is prepared where the complexed ligands are members of one or more series of lead compounds directed against the target. This also includes the design of ligands to diminish binding to another molecule or molecules (for example another enzyme or enzymes that shares a substrate with a target enzyme) to improve specificity to one or more desired targets.

Examination of the three-dimensional structure of one or more target-ligand complexes may lead an enhanced understanding of the binding mode of the ligands and the binding cavity of the target in the presence of the ligands. This information can be harnessed by one or more tools in medicinal chemistry or computational chemistry to form hypotheses for the modifications to the ligands which may lead to the modulation of binding affinity or improve other characteristics favorable for use as a drug.

The tools of medicinal chemistry and computational chemistry of structure-based drug design include, but are not limited to, molecular modelling, virtual screening and docking, design of focused combinatorial chemistry libraries, de novo ligand design, guides to additional candidates for three-dimensional structure determination, and rationalization of observed structure-activity relationships.

Potential modifications directed or inspired through the application of medicinal and computational tools include elaboration of a ligand to establish or modulate specific interactions with the target, removal of groups from the ligands which are deemed unimportant or detracting from desired binding affinity to the target, modification of ligand to modulate relative specificity against other targets, and elaboration of a ligand to improve its drug-like properties without producing unacceptable effects to the binding affinity to the target.

In an example, structures of small molecule compounds identified as having inhibitory activity in an enzyme activity screen can be modelled using Sybyl (Tripos Inc., 1699 South Hanley Rd., St. Louis, Mo., 63144, USA) molecular modelling software and compared with the X-ray crystallographic structure of the same or similar molecules in the active site region of the enzyme using Coot (Emsley and Cowtan, Acta Crystallographica D, 2004, 60, 2126-2132) crystallographic model-building software. Based on the comparison, changes can be made to the modelled compounds in Sybyl which are expected, on the basis of the structure comparison, to either enhance or weaken the inhibition of the enzyme. New compounds based on these models can then be synthesised and their effect in an enzyme activity screen measured. This process can be repeated, for example possibly focusing on different parts of the molecule/enzyme or different properties of the compound, as discussed above, in different rounds.

### REFERENCES

1. Samuelsson, B. Leukotrienes: Mediators of immediate hypersensitivity reactions and inflammation. Science 220, 568-575 (1983).
2. Funk, C. D. Prostaglandins and leukotrienes: Advances in eicosanoid biology. Science 294, 1871-1875 (2001).
3. Samuelsson, B., Dahlén, S.-E., Lindgren, J.-Å., Rouzer, C. A. & Serhan, C. N. Leukotrienes and lipoxins: Structures, biosynthesis, and biological effects. Science 237, 1171-1176 (1987).
4. Hanna, C. J., Bach, M. K., Pare, P. D. & Schellenberg, R. R. Slow-Reacting Substances (Leukotrienes) Contract Human Airway and Pulmonary Vascular Smooth Muscle Invitro. Nature 290, 343-344 (1981).
5. Dahlén, S.-E., Hedqvist, P., Hammarström, S. & Samuelsson, B. Leukotrienes are potent constrictors of human bronchi. Nature 288, 484-486 (1980).
6. Dahlén, S.-E. et al. Leukotrienes promote plasma leakage and leukocyte adhesion in postcapillary venules: In vivo effects with relevance to the acute inflammatory response. Proc. Natl. Acad. Sci. USA 78, 3887-3891 (1981).
7. Weiss, J. W. et al. Bronchoconstrictor Effects of Leukotriene-C in Humans. Science 216, 196-198 (1982).
8. Smedegard, G. et al. Leukotriene-C4 Affects Pulmonary and Cardiovascular Dynamics in Monkey. Nature 295, 327-329 (1982).
9. Beller, T. C. et al. Cysteinyl leukotriene 1 receptor controls the severity of chronic pulmonary inflammation and fibrosis. Proc. Natl. Acad. Sci. U.S.A. 101, 3047-3052 (2004).
10. Robbiani, D. F. et al. The leukotriene C4 transporter MRP1 regulates CCL19 (MIP-3beta, ELC)-dependent mobilization of dendritic cells to lymph nodes. Cell 103, 757-68 (2000).
11. Drazen, J. F., Israel, E. & O'Byrne, P. Treatment of asthma with drugs modifying the leukotriene pathway. N. Engl. J. Med. 340, 197-206 (1999).
12. Lam, B. K. & Austen, K. F. Leukotriene C-4 synthase: a pivotal enzyme in cellular biosynthesis of the cysteinyl leukotrienes. Prostaglandins & Other Lipid Mediators 68-9, 511-520 (2002).
13. Penrose, J. F. et al. Purification of human leukotriene C₄ synthase. Proc. Natl. Acad. Sci. USA 89, 11603-11606 (1992).
14. Nicholson, D. W. et al. Purification to homogeneity and the N-terminal sequence of human leukotriene C4 synthase: A homodimeric glutathione S-transferase composed of 18-kDa subunits. Proc. Natl. Acad. Sci. USA 90, 2015-2019 (1993).
15. Lam, B. K., Penrose, J. F., Freeman, G. J. & Austen, K. F. Expression cloning of a cDNA for human leukotriene C₄ synthase, an integral membrane protein conjugating reduced glutathione to leukotriene A₄. Proc. Natl. Acad. Sci. U.S.A. 91, 7663-7 (1994).
16. Welsch, D. J. et al. Molecular cloning end expression of human leukotriene C₄ synthase. Proc. Natl. Acad. Sci. U.S.A. 91, 9745-9749 (1994).
17. Jakobsson, P. J., Morgenstern, R., Mancini, J., Ford-Hutchinson, A. & Persson, B. Common structural features of MAPEG—a widespread superfamily of membrane associated proteins with highly divergent functions in eicosanoid and glutathione metabolism. Protein Sci. 8, 689-92. (1999).
18. Schmidt-Krey, I. et al. Human leukotriene C(4) synthase at 4.5 A resolution in projection. Structure 12, 2009-14 (2004).
19. Holm, P. J. et al. Structural basis for detoxification and oxidative stress protection in membranes. J. Mol. Biol. 360, 934-945 (2006).
20. White, S. H. Membrane Proteins of Known 3D Structure. http://blanco.biomol.uci.edu/Membrane_Proteins_xtal.html (2007).
21. Morisseau, C. & Hammock, B. D. Epoxide hydrolases: Mechanisms, inhibitor designs, and biological roles. Annu. Rev. Pharmacol. Toxicol. 45, 311-+(2005).
22. Corey, E. J., Hopkins, P. B., Munroe, J. E., Marfat, A. & Hashimoto, S. Total Synthesis of 6-Trans, 10-Cis and (+/−)-6-Trans, 8-Cis Isomers of Leukotriene-B. J. Am. Chem. Soc. 102, 7986-7987 (1980).
23. Morgenstern, R., Svensson, R., Bernat, B. A. & Armstrong, R. N. Kinetic analysis of the slow ionization of glutathione by microsomal glutathione transferase MGST1. Biochemistry (Mosc.) 40, 3378-3384 (2001).
24. Lam, B. K., Penrose, J. F., Xu, K. Y., Baldasaro, M. H. & Austen, K. F. Site-directed mutagenesis of human leukotriene C₄ synthase. J. Biol. Chem. 272, 13923-13928 (1997).
25. Kojima, F., Kato, S. & Kawai, S. Prostaglandin E synthase in the pathophysiology of arthritis. Fundamental & Clinical Pharmacology 19, 255-261 (2005).
26. Mabuchi, T. et al. Membrane-associated prostaglandin E synthase-I is required for neuropathic pain. Neuroreport 15, 1395-1398 (2004).
27. Abramovitz, M. et al. 5-lipoxygenase-activating protein stimulates the utilization of arachidonic acid by 5-lipoxygenase. Eur. J. Biochem. 215, 105-111 (1993).
28. Sheldrick, G. M. & Schneider, T. R. in Methods Enzymol. (ed. Sweet, C. W. C. J. a. R. M.) 319-343 (Academic Press, 1997).
29. de La Fortelle, E. & Bricogne, G. in Methods Enzymol. (ed. Charles W. Carter, J.) 472-494 (Academic Press, 1997).
30. Abrahams, J. P. & Leslie, A. G. W. Methods used in the structure determination of bovine mitochondrial F1 ATPase. Acta Crystallogr. D Biol. Crystallogr. 52, 30-42 (1996).
31. Perrakis, A., Morris, R. & Lamzin, V. S. Automated protein model building combined with iterative structure refinement. Nat. Struct. Biol. 6, 458-463 (1999).
32. Emsley, P. & Cowtan, K. Coot: model-building tools for molecular graphics. Acta Crystallogr. D Biol. Crystallogr. 60, 2126-2132 (2004).
33. McCoy, A. J., Grosse-Kunstleve, R. W., Storoni, L. C. & Read, R. J. Likelihood enhanced fast translation functions. Acta Crystallogr. D Biol. Crystallogr. 61, 458-464 (2005).
34. Murshudov, G. N., Vagin, A. A. & Dodson, E. J. Refinement of Macromolecular Structures by the Maximum-Likelihood Method. Acta Crystallogr. D Biol. Crystallogr. 53, 240-255 (1997).
35. DeLano, W. L. The PyMOL Molecular Graphics System. DeLano Scientific, Palo Alto, Calif., USA. (2002).
36. Wallace, A. C., Laskowski, R. A. & Thornton, J. M. Ligplot—a Program to Generate Schematic Diagrams of Protein Ligand Interactions. Protein Eng. 8, 127-134 (1995).
37. Corey, E. J. et al. Stereospecific total synthesis of a “Slow Reacting Substance” of anaphylaxis, leukotriene C4. J. Am. CHem Soc. 102, 1436-1439 (1980).
38. Izumi T. et al. Solubilization and partial purification of leukotriene C4 synthase from guinea-pig lung: a microsomal enzyme with high specificity towards 5,6-epoxide leukotriene A4. Biochim Biophys Acta. 959, 305-15 (1988).
39. Yoshimoto T, Soberman R J, Spur B, Austen K F. Properties of highly purified leukotriene C4 synthase of guinea pig lung. J Clin Invest. March; 81, 866-871 (1988).

### X Ray Data

TABLE I
without GSH

| | | |
|---|---|---|
| HEADER | ---XX-XXX-9- | dmm |

| | |
|---|---|
| COMPND | --- |

| | | | |
|---|---|---|---|
| REMARK | 3 | | |
| REMARK | 3 | REFINEMENT. | |
| REMARK | 3 | PROGRAM | : REFMAC 5.2.0019 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.00 |
| REMARK | 3 | RESOLUTION RANGE LOW | (ANGSTROMS) | : 28.27 |
| REMARK | 3 | DATA CUTOFF | (SIGMA(F)) | : NONE |
| REMARK | 3 | COMPLETENESS FOR RANGE | (%) | : 99.76 |
| REMARK | 3 | NUMBER OF REFLECTIONS | | : 25914 |
| REMARK | 3 | | | |

| REMARK | 3 | FIT TO DATA IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.17737 |
| REMARK | 3 | R VALUE | (WORKING SET) | : 0.17563 |

| | | | | |
|---|---|---|---|---|
| REMARK | 3 | FREE R VALUE | | : 0.21171 |
| REMARK | 3 | FREE R VALUE TEST SET SIZE | (%) | : 5.0 |
| REMARK | 3 | FREE R VALUE TEST SET COUNT | | : 1376 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 2.000 |
| REMARK | 3 | BIN RESOLUTION RANGE LOW | | : 2.052 |
| REMARK | 3 | REFLECTION IN BIN | (WORKING SET) | : 1882 |
| REMARK | 3 | BIN COMPLETENESS | (WORKING + TEST) (%) | : 100.00 |
| REMARK | 3 | BIN R VALUE | (WORKING SET) | : 0.247 |

| | | | |
|---|---|---|---|
| REMARK | 3 | BIN FREE R VALUE SET COUNT | : 89 |
| REMARK | 3 | BIN FREE R VALUE | : 0.269 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 1596 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 30.196 |

| REMARK | 3 | OVERALL ANISOTROPIC B VALUE. | | |
|---|---|---|---|---|
| REMARK | 3 | B11 (A**2) : NULL | | |
| REMARK | 3 | B22 (A**2) : NULL | | |
| REMARK | 3 | B33 (A**2) : NULL | | |
| REMARK | 3 | B12 (A**2) : NULL | | |
| REMARK | 3 | B13 (A**2) : NULL | | |
| REMARK | 3 | B23 (A**2) : NULL | | |
| REMARK | 3 | | | |
| REMARK | 3 | ESTIMATED OVERALL COORDINATE ERROR. | | |
| REMARK | 3 | ESU BASED ON R VALUE | (A): | 0.102 |
| REMARK | 3 | ESU BASED ON FREE R VALUE | (A): | 0.107 |
| REMARK | 3 | ESU BASED ON MAXIMUM LIKELIHOOD | (A): | 0.072 |
| REMARK | 3 | ESU FOR B VALUES BASED ON MAXIMUM LIKELIHOOD | (A**2): | 5.087 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.957 |
| REMARK | 3 | CORRELATION COEFFICIENT FO-FC FREE | : 0.945 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 1514 | ; 0.022 | ; 0.021 |
| REMARK | 3 | BOND ANGLES REFINED ATOMS | (DEGREES): | 2024 | ; 1.987 | ; 2.075 |
| REMARK | 3 | TORSION ANGLES, PERIOD 1 | (DEGREES): | 177 | ; 7.169 | ; 5.000 |
| REMARK | 3 | TORSION ANGLES, PERIOD 2 | (DEGREES): | 54 | ;30.135 | ;20.000 |
| REMARK | 3 | TORSION ANGLES, PERIOD 3 | (DEGREES): | 197 | ;16.738 | ;15.000 |
| REMARK | 3 | TORSION ANGLES, PERIOD 4 | (DEGREES): | 13 | ;20.706 | ;15.000 |
| REMARK | 3 | CHIRAL-CENTER RESTRAINTS | (A**3): | 230 | ; 0.183 | ; 0.200 |

| | | | | | | |
|---|---|---|---|---|---|---|
| REMARK | 3 | GENERAL PLANES REFINED ATOMS | (A): | 1021 | ; 0.009 | ; 0.020 |
| REMARK | 3 | NON-BONDED CONTACTS REFINED ATOMS | (A): | 720 | ; 0.241 | ; 0.200 |
| REMARK | 3 | NON-BONDED TORSION REFINED ATOMS | (A): | 957 | ; 0.317 | ; 0.200 |
| REMARK | 3 | H-BOND (X...Y) REFINED ATOMS | (A): | 79 | ; 0.257 | ; 0.200 |
| REMARK | 3 | SYMMETRY VDW REFINED ATOMS | (A): | 159 | ; 0.250 | ; 0.200 |
| REMARK | 3 | SYMMETRY H-BOND REFINED ATOMS | (A): | 20 | ; 0.321 | ; 0.200 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 834 | ; 1.257 | ; 1.500 |
| REMARK | 3 | MAIN-CHAIN ANGLE REFINED ATOMS | (A**2): | 1289 | ; 1.865 | ; 2.000 |
| REMARK | 3 | SIDE-CHAIN BOND REFINED ATOMS | (A**2): | 753 | ; 3.016 | ; 3.000 |
| REMARK | 3 | SIDE-CHAIN ANGLE REFINED ATOMS | (A**2): | 723 | ; 4.123 | ; 4.500 |
| REMARK | 3 | | | | | |

| REMARK | 3 | NCS RESTRAINTS STATISTICS | | | | | |
|---|---|---|---|---|---|---|---|
| REMARK | 3 | NUMBER OF NCS GROUPS : NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS : 4 | | | | | |
| REMARK | 3 | ATOM RECORD CONTAINS RESIDUAL B FACTORS ONLY | | | | | |
| REMARK | 3 | | | | | | |
| REMARK | 3 | TLS GROUP : 1 | | | | | |
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | −5 | | A | 5 |

| REMARK | 3 | ORIGIN FOR THE GROUP (A): 38.5653 −48.1143 −30.3275 | | | | | |
|---|---|---|---|---|---|---|---|
| REMARK | 3 | T TENSOR | | | | | |
| REMARK | 3 | T11: | 0.0725 | T22: | 0.1052 | | |
| REMARK | 3 | T33: | 0.1085 | T12: | −0.0606 | | |
| REMARK | 3 | T13: | 0.0711 | T23: | 0.0830 | | |
| REMARK | 3 | L TENSOR | | | | | |
| REMARK | 3 | L11: | 17.8061 | L22: | 2.2778 | | |
| REMARK | 3 | L33: | 10.5462 | L12: | 0.0569 | | |
| REMARK | 3 | L13: | −12.6791 | L23: | 1.8189 | | |
| REMARK | 3 | S TENSOR | | | | | |
| REMARK | 3 | S11: | 0.0667 | S12: | −0.6593 | S13: | −0.5618 |
| REMARK | 3 | S21: | −0.0684 | S22: | 0.0140 | S23: | −0.4595 |
| REMARK | 3 | S31: | −0.0809 | S32: | 0.6802 | S33: | −0.0807 |
| REMARK | 3 | | | | | | |

| REMARK | 3 | TLS GROUP : 2 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 6 | | A | 112 |

| REMARK | 3 | ORIGIN FOR THE GROUP (A): 15.2468 −57.8968 −10.8106 | | | | | |
|---|---|---|---|---|---|---|---|
| REMARK | 3 | T TENSOR | | | | | |
| REMARK | 3 | T11: | 0.1017 | T22: | 0.1079 | | |
| REMARK | 3 | T33: | 0.0651 | T12: | −0.0195 | | |
| REMARK | 3 | T13: | 0.0851 | T23: | 0.0219 | | |
| REMARK | 3 | L TENSOR | | | | | |
| REMARK | 3 | L11: | 1.7350 | L22: | 1.3482 | | |
| REMARK | 3 | L33: | 2.1370 | L12: | 0.4369 | | |
| REMARK | 3 | L13: | −0.9101 | L23: | −0.5741 | | |
| REMARK | 3 | S TENSOR | | | | | |
| REMARK | 3 | S11: | −0.0534 | S12: | −0.2815 | S13: | −0.0727 |
| REMARK | 3 | S21: | 0.2539 | S22: | −0.0172 | S23: | 0.1277 |
| REMARK | 3 | S31: | 0.1175 | S32: | −0.0823 | S33: | 0.0706 |
| REMARK | 3 | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 113 | | A | 142 |

| REMARK | 3 | ORIGIN FOR THE GROUP (A): 12.8365 −48.9997 −38.6599 | | | | | |
|---|---|---|---|---|---|---|---|
| REMARK | 3 | T TENSOR | | | | | |
| REMARK | 3 | T11: | 0.0974 | T22: | 0.1247 | | |
| REMARK | 3 | T33: | 0.1388 | T12: | −0.0022 | | |
| REMARK | 3 | T13: | −0.0214 | T23: | 0.0643 | | |
| REMARK | 3 | L TENSOR | | | | | |
| REMARK | 3 | L11: | 0.4772 | L22: | 1.7385 | | |
| REMARK | 3 | L33: | 15.5152 | L12: | 0.5613 | | |
| REMARK | 3 | L13: | −1.8713 | L23: | −1.4990 | | |
| REMARK | 3 | S TENSOR | | | | | |
| REMARK | 3 | S11: | −0.1136 | S12: | 0.1208 | S13: | 0.0559 |
| REMARK | 3 | S21: | −0.2355 | S22: | 0.0525 | S23: | 0.2362 |
| REMARK | 3 | S31: | −0.0451 | S32: | −0.6552 | S33: | 0.0611 |
| REMARK | 3 | | | | | | |

| REMARK | 3 | TLS GROUP : 4 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 143 | | A | 149 |

| REMARK | 3 | ORIGIN FOR THE GROUP (A): 11.2731 −46.1923 −66.6381 | | | | | |
|---|---|---|---|---|---|---|---|
| REMARK | 3 | T TENSOR | | | | | |
| REMARK | 3 | T11: | 0.4358 | T22: | 0.4581 | | |
| REMARK | 3 | T33: | 0.4564 | T12: | 0.0018 | | |
| REMARK | 3 | T13: | −0.0002 | T23: | −0.0096 | | |
| REMARK | 3 | L TENSOR | | | | | |
| REMARK | 3 | L11: | 22.3722 | L22: | 31.1048 | | |
| REMARK | 3 | L33: | 224.2959 | L12: | −26.3796 | | |
| REMARK | 3 | L13: | 70.8378 | L23: | −83.5266 | | |
| REMARK | 3 | S TENSOR | | | | | |
| REMARK | 3 | S11: | 0.2737 | S12: | 2.4714 | S13: | 1.2687 |
| REMARK | 3 | S21: | −5.2175 | S22: | −3.9374 | S23: | −3.9650 |
| REMARK | 3 | S31: | 3.7824 | S32: | 1.4196 | S33: | 3.6637 |
| REMARK | 3 | | | | | | |
| REMARK | 3 | | | | | | |

| REMARK | 3 | BULK SOLVENT MODELLING. | | | | | | |
|---|---|---|---|---|---|---|---|---|
| REMARK | 3 | METHOD USED : MASK | | | | | | |
| REMARK | 3 | PARAMETERS FOR MASK CALCULATION | | | | | | |
| REMARK | 3 | VDW PROBE RADIUS | | | | : 1.20 | | |
| REMARK | 3 | ION PROBE RADIUS | | | | : 0.80 | | |
| REMARK | 3 | SHRINKAGE RADIUS | | | | : 0.80 | | |
| REMARK | 3 | | | | | | | |
| REMARK | 3 | OTHER REFINEMENT REMARKS: | | | | | | |
| REMARK | 3 | HYDROGENS HAVE BEEN ADDED IN THE RIDING POSITIONS | | | | | | |
| REMARK | 3 | | | | | | | |
| CISPEP | 1 | HIS | A | −4 | HIS | A | −3 | 0.00 |
| CISPEP | 2 | PRO | A | 37 | PRO | A | 38 | 0.00 |
| CISPEP | 3 | LEU | A | 143 | ARG | A | 144 | 0.00 |
| CISPEP | 4 | ARG | A | 144 | THR | A | 145 | 0.00 |

| | | | | | |
|---|---|---|---|---|---|
| CRYST1 | 169.670 | 169.670 | 169.670 | 90.00 90.00 90.00 | F 2 3 |

| | | | | |
|---|---|---|---|---|
| SCALE1 | 0.005894 | 0.000000 | 0.000000 | 0.00000 |
| SCALE2 | 0.000000 | 0.005894 | 0.000000 | 0.00000 |
| SCALE3 | 0.000000 | 0.000000 | 0.005894 | 0.00000 |

| | | | | | | | | | | | |
|---|---|---|---|---|---|---|---|---|---|---|---|
| ATOM | 1 | N | ALA | A | −5 | 43.417 | −56.043 | −31.929 | 1.00 | 40.01 | N |
| ATOM | 2 | CA | ALA | A | −5 | 44.846 | −56.114 | −31.509 | 1.00 | 40.26 | C |
| ATOM | 3 | CB | ALA | A | −5 | 45.609 | −57.239 | −32.274 | 1.00 | 39.53 | C |
| ATOM | 4 | C | ALA | A | −5 | 45.458 | −54.750 | −31.798 | 1.00 | 39.87 | C |
| ATOM | 5 | O | ALA | A | −5 | 46.544 | −54.674 | −32.349 | 1.00 | 40.49 | O |
| ATOM | 6 | N | HIS | A | −4 | 44.731 | −53.685 | −31.435 | 1.00 | 39.62 | N |
| ATOM | 7 | CA | HIS | A | −4 | 45.061 | −52.308 | −31.812 | 1.00 | 37.34 | C |
| ATOM | 8 | CB | HIS | A | −4 | 46.450 | −51.924 | −31.223 | 1.00 | 36.68 | C |
| ATOM | 9 | CG | HIS | A | −4 | 47.559 | −51.830 | −32.233 | 1.00 | 33.52 | C |
| ATOM | 10 | ND1 | HIS | A | −4 | 48.441 | −52.864 | −32.484 | 1.00 | 31.74 | N |
| ATOM | 11 | CE1 | HIS | A | −4 | 49.312 | −52.492 | −33.406 | 1.00 | 31.37 | C |
| ATOM | 12 | NE2 | HIS | A | −4 | 49.019 | −51.258 | −33.773 | 1.00 | 30.66 | N |
| ATOM | 13 | CD2 | HIS | A | −4 | 47.931 | −50.816 | −33.047 | 1.00 | 28.76 | C |
| ATOM | 14 | C | HIS | A | −4 | 45.004 | −52.310 | −33.341 | 1.00 | 37.04 | C |
| ATOM | 15 | O | HIS | A | −4 | 45.628 | −53.147 | −33.953 | 1.00 | 36.99 | O |
| ATOM | 16 | N | HIS | A | −3 | 44.287 | −51.438 | −34.034 | 1.00 | 36.79 | N |
| ATOM | 17 | CA | HIS | A | −3 | 43.501 | −50.265 | −33.676 | 1.00 | 35.24 | C |
| ATOM | 18 | CB | HIS | A | −3 | 42.020 | −50.578 | −33.563 | 1.00 | 34.83 | C |
| ATOM | 19 | CG | HIS | A | −3 | 41.652 | −51.796 | −34.356 | 1.00 | 36.60 | C |
| ATOM | 20 | ND1 | HIS | A | −3 | 41.549 | −51.785 | −35.734 | 1.00 | 35.20 | N |
| ATOM | 21 | CE1 | HIS | A | −3 | 41.284 | −53.008 | −36.163 | 1.00 | 37.38 | C |
| ATOM | 22 | NE2 | HIS | A | −3 | 41.227 | −53.817 | −35.114 | 1.00 | 35.02 | N |
| ATOM | 23 | CD2 | HIS | A | −3 | 41.496 | −53.092 | −33.976 | 1.00 | 32.96 | C |
| ATOM | 24 | C | HIS | A | −3 | 44.037 | −48.993 | −32.985 | 1.00 | 34.06 | C |
| ATOM | 25 | O | HIS | A | −3 | 44.487 | −48.964 | −31.825 | 1.00 | 34.68 | O |
| ATOM | 26 | N | HIS | A | −2 | 43.985 | −47.955 | −33.798 | 1.00 | 30.69 | N |
| ATOM | 27 | CA | HIS | A | −2 | 44.283 | −46.597 | −33.397 | 1.00 | 29.38 | C |
| ATOM | 28 | CB | HIS | A | −2 | 45.218 | −46.005 | −34.412 | 1.00 | 28.22 | C |
| ATOM | 29 | CG | HIS | A | −2 | 46.542 | −46.677 | −34.358 | 1.00 | 26.13 | C |
| ATOM | 30 | ND1 | HIS | A | −2 | 47.513 | −46.312 | −33.449 | 1.00 | 27.41 | N |
| ATOM | 31 | CE1 | HIS | A | −2 | 48.563 | −47.107 | −33.584 | 1.00 | 29.67 | C |
| ATOM | 32 | NE2 | HIS | A | −2 | 48.267 | −48.026 | −34.490 | 1.00 | 26.72 | N |
| ATOM | 33 | CD2 | HIS | A | −2 | 46.995 | −47.795 | −34.964 | 1.00 | 24.20 | C |
| ATOM | 34 | C | HIS | A | −2 | 43.025 | −45.771 | −33.151 | 1.00 | 29.66 | C |
| ATOM | 35 | O | HIS | A | −2 | 43.067 | −44.525 | −33.162 | 1.00 | 29.63 | O |
| ATOM | 36 | N | HIS | A | −1 | 41.928 | −46.484 | −32.937 | 1.00 | 28.29 | N |
| ATOM | 37 | CA | HIS | A | −1 | 40.722 | −45.901 | −32.363 | 1.00 | 29.36 | C |
| ATOM | 38 | CB | HIS | A | −1 | 39.833 | −45.269 | −33.435 | 1.00 | 29.37 | C |
| ATOM | 39 | CG | HIS | A | −1 | 39.189 | −46.282 | −34.328 | 1.00 | 29.87 | C |
| ATOM | 40 | ND1 | HIS | A | −1 | 39.843 | −46.855 | −35.395 | 1.00 | 27.36 | N |
| ATOM | 41 | CE1 | HIS | A | −1 | 39.044 | −47.730 | −35.976 | 1.00 | 26.36 | C |
| ATOM | 42 | NE2 | HIS | A | −1 | 37.900 | −47.747 | −35.318 | 1.00 | 26.52 | N |
| ATOM | 43 | CD2 | HIS | A | −1 | 37.978 | −46.878 | −34.261 | 1.00 | 23.85 | C |
| ATOM | 44 | C | HIS | A | −1 | 39.979 | −47.029 | −31.664 | 1.00 | 29.58 | C |
| ATOM | 45 | O | HIS | A | −1 | 40.235 | −48.223 | −31.897 | 1.00 | 27.14 | O |
| ATOM | 46 | N | HIS | A | 0 | 39.084 | −46.641 | −30.765 | 1.00 | 30.33 | N |
| ATOM | 47 | CA | AHIS | A | 0 | 38.210 | −47.644 | −30.156 | 0.50 | 31.50 | C |
| ATOM | 48 | CA | BHIS | A | 0 | 38.246 | −47.574 | −30.012 | 0.50 | 31.25 | C |
| ATOM | 49 | CB | AHIS | A | 0 | 38.709 | −48.065 | −28.776 | 0.50 | 31.91 | C |
| ATOM | 50 | CB | BHIS | A | 0 | 38.708 | −47.583 | −28.555 | 0.50 | 31.55 | C |
| ATOM | 51 | CG | AHIS | A | 0 | 40.193 | −48.215 | −28.683 | 0.50 | 34.15 | C |
| ATOM | 52 | CG | BHIS | A | 0 | 38.350 | −46.341 | −27.780 | 0.50 | 31.51 | C |
| ATOM | 53 | ND1 | AHIS | A | 0 | 41.012 | −47.196 | −28.255 | 0.50 | 36.59 | N |
| ATOM | 54 | ND1 | BHIS | A | 0 | 37.046 | −45.965 | −27.524 | 0.50 | 32.11 | N |
| ATOM | 55 | CE1 | AHIS | A | 0 | 42.269 | −47.606 | −28.279 | 0.50 | 37.93 | C |
| ATOM | 56 | CE1 | BHIS | A | 0 | 37.031 | −44.870 | −26.789 | 0.50 | 29.93 | C |
| ATOM | 57 | NE2 | AHIS | A | 0 | 42.288 | −48.862 | −28.688 | 0.50 | 36.79 | N |
| ATOM | 58 | NE2 | BHIS | A | 0 | 38.284 | −44.520 | −26.551 | 0.50 | 35.90 | N |
| ATOM | 59 | CD2 | AHIS | A | 0 | 41.003 | −49.268 | −28.948 | 0.50 | 36.50 | C |
| ATOM | 60 | CD2 | BHIS | A | 0 | 39.128 | −45.422 | −27.162 | 0.50 | 34.28 | C |
| ATOM | 61 | C | HIS | A | 0 | 36.803 | −47.073 | −30.063 | 1.00 | 31.25 | C |
| ATOM | 62 | O | HIS | A | 0 | 36.595 | −45.843 | −30.221 | 1.00 | 34.09 | O |
| ATOM | 63 | N | HIS | A | 1 | 35.841 | −47.958 | −29.894 | 1.00 | 30.33 | N |
| ATOM | 64 | CA | HIS | A | 1 | 34.441 | −47.590 | −29.816 | 1.00 | 29.97 | C |
| ATOM | 65 | CB | HIS | A | 1 | 33.627 | −48.418 | −30.808 | 1.00 | 29.50 | C |
| ATOM | 66 | CG | HIS | A | 1 | 34.011 | −48.156 | −32.223 | 1.00 | 27.56 | C |
| ATOM | 67 | ND1 | HIS | A | 1 | 33.499 | −47.103 | −32.952 | 1.00 | 23.70 | N |
| ATOM | 68 | CE1 | HIS | A | 1 | 34.037 | −47.102 | −34.155 | 1.00 | 23.80 | C |
| ATOM | 69 | NE2 | HIS | A | 1 | 34.871 | −48.123 | −34.244 | 1.00 | 25.91 | N |
| ATOM | 70 | CD2 | HIS | A | 1 | 34.888 | −48.794 | −33.037 | 1.00 | 20.73 | C |
| ATOM | 71 | C | HIS | A | 1 | 33.851 | −47.679 | −28.403 | 1.00 | 30.10 | C |
| ATOM | 72 | O | HIS | A | 1 | 32.636 | −47.579 | −28.252 | 1.00 | 30.73 | O |
| ATOM | 73 | N | LYS | A | 2 | 34.727 | −47.765 | −27.395 | 1.00 | 29.69 | N |
| ATOM | 74 | CA | LYS | A | 2 | 34.376 | −47.810 | −25.963 | 1.00 | 29.87 | C |
| ATOM | 75 | CB | LYS | A | 2 | 35.618 | −47.776 | −25.043 | 1.00 | 29.27 | C |
| ATOM | 76 | CG | LYS | A | 2 | 36.413 | −48.999 | −25.019 | 1.00 | 28.73 | C |
| ATOM | 77 | CD | LYS | A | 2 | 37.425 | −48.905 | −23.891 | 1.00 | 27.66 | C |
| ATOM | 78 | CE | LYS | A | 2 | 38.601 | −48.087 | −24.330 | 1.00 | 36.28 | C |
| ATOM | 79 | NZ | LYS | A | 2 | 39.767 | −48.393 | −23.499 | 1.00 | 40.53 | N |
| ATOM | 80 | C | LYS | A | 2 | 33.570 | −46.603 | −25.587 | 1.00 | 29.58 | C |
| ATOM | 81 | O | LYS | A | 2 | 32.575 | −46.712 | −24.829 | 1.00 | 27.29 | O |
| ATOM | 82 | N | ASP | A | 3 | 33.973 | −45.452 | −26.126 | 1.00 | 27.69 | N |
| ATOM | 83 | CA | ASP | A | 3 | 33.322 | −44.230 | −25.730 | 1.00 | 28.51 | C |
| ATOM | 84 | CB | ASP | A | 3 | 34.195 | −42.983 | −26.035 | 1.00 | 29.24 | C |
| ATOM | 85 | CG | ASP | A | 3 | 34.690 | −42.927 | −27.498 | 1.00 | 34.80 | C |
| ATOM | 86 | OD1 | ASP | A | 3 | 34.424 | −43.836 | −28.373 | 1.00 | 32.19 | O |
| ATOM | 87 | OD2 | ASP | A | 3 | 35.424 | −41.943 | −27.755 | 1.00 | 42.71 | O |
| ATOM | 88 | C | ASP | A | 3 | 31.928 | −44.116 | −26.312 | 1.00 | 27.85 | C |
| ATOM | 89 | O | ASP | A | 3 | 31.212 | −43.201 | −25.976 | 1.00 | 28.12 | O |
| ATOM | 90 | N | GLU | A | 4 | 31.526 | −45.044 | −27.185 | 1.00 | 26.80 | N |
| ATOM | 91 | CA | GLU | A | 4 | 30.165 | −45.057 | −27.694 | 1.00 | 24.98 | C |
| ATOM | 92 | CB | GLU | A | 4 | 30.151 | −45.459 | −29.168 | 1.00 | 26.58 | C |
| ATOM | 93 | CG | GLU | A | 4 | 31.122 | −44.591 | −29.949 | 1.00 | 33.17 | C |
| ATOM | 94 | CD | GLU | A | 4 | 30.987 | −44.795 | −31.407 | 1.00 | 40.28 | C |
| ATOM | 95 | OE1 | GLU | A | 4 | 31.710 | −45.652 | −31.941 | 1.00 | 41.27 | O |
| ATOM | 96 | OE2 | GLU | A | 4 | 30.154 | −44.079 | −32.011 | 1.00 | 47.02 | O |
| ATOM | 97 | C | GLU | A | 4 | 29.233 | −45.994 | −26.888 | 1.00 | 23.81 | C |
| ATOM | 98 | O | GLU | A | 4 | 28.010 | −45.994 | −27.128 | 1.00 | 23.71 | O |
| ATOM | 99 | N | VAL | A | 5 | 29.768 | −46.806 | −25.976 | 1.00 | 20.73 | N |
| ATOM | 100 | CA | VAL | A | 5 | 28.898 | −47.787 | −25.321 | 1.00 | 21.18 | C |
| ATOM | 101 | CB | VAL | A | 5 | 29.162 | −49.221 | −25.939 | 1.00 | 21.98 | C |
| ATOM | 102 | CG1 | VAL | A | 5 | 28.872 | −49.207 | −27.488 | 1.00 | 22.13 | C |
| ATOM | 103 | CG2 | VAL | A | 5 | 30.626 | −49.683 | −25.679 | 1.00 | 20.83 | C |
| ATOM | 104 | C | VAL | A | 5 | 29.156 | −47.790 | −23.847 | 1.00 | 19.14 | C |
| ATOM | 105 | O | VAL | A | 5 | 28.709 | −48.668 | −23.159 | 1.00 | 19.28 | O |
| ATOM | 106 | N | ALA | A | 6 | 29.943 | −46.817 | −23.364 | 1.00 | 17.79 | N |
| ATOM | 107 | CA | ALA | A | 6 | 30.304 | −46.851 | −21.945 | 1.00 | 16.48 | C |
| ATOM | 108 | CB | ALA | A | 6 | 31.438 | −45.830 | −21.604 | 1.00 | 15.49 | C |
| ATOM | 109 | C | ALA | A | 6 | 29.116 | −46.694 | −20.974 | 1.00 | 16.52 | C |
| ATOM | 110 | O | ALA | A | 6 | 29.153 | −47.257 | −19.877 | 1.00 | 14.62 | O |
| ATOM | 111 | N | LEU | A | 7 | 28.129 | −45.868 | −21.330 | 1.00 | 16.27 | N |
| ATOM | 112 | CA | LEU | A | 7 | 26.955 | −45.723 | −20.470 | 1.00 | 17.57 | C |
| ATOM | 113 | CB | LEU | A | 7 | 26.023 | −44.567 | −20.942 | 1.00 | 16.67 | C |
| ATOM | 114 | CG | LEU | A | 7 | 26.613 | −43.149 | −20.994 | 1.00 | 21.48 | C |
| ATOM | 115 | CD1 | LEU | A | 7 | 25.575 | −42.165 | −21.651 | 1.00 | 21.87 | C |
| ATOM | 116 | CD2 | LEU | A | 7 | 26.972 | −42.660 | −19.583 | 1.00 | 21.78 | C |
| ATOM | 117 | C | LEU | A | 7 | 26.155 | −47.054 | −20.494 | 1.00 | 17.71 | C |
| ATOM | 118 | O | LEU | A | 7 | 25.635 | −47.466 | −19.474 | 1.00 | 17.10 | O |
| ATOM | 119 | N | LEU | A | 8 | 26.069 | −47.688 | −21.655 | 1.00 | 17.54 | N |
| ATOM | 120 | CA | LEU | A | 8 | 25.386 | −48.983 | −21.746 | 1.00 | 16.81 | C |
| ATOM | 121 | CB | LEU | A | 8 | 25.364 | −49.503 | −23.209 | 1.00 | 16.66 | C |
| ATOM | 122 | CG | LEU | A | 8 | 24.740 | −48.526 | −24.258 | 1.00 | 17.84 | C |
| ATOM | 123 | CD1 | LEU | A | 8 | 24.734 | −49.147 | −25.668 | 1.00 | 15.12 | C |
| ATOM | 124 | CD2 | LEU | A | 8 | 23.292 | −48.130 | −23.831 | 1.00 | 16.84 | C |
| ATOM | 125 | C | LEU | A | 8 | 26.131 | −50.021 | −20.895 | 1.00 | 17.77 | C |
| ATOM | 126 | O | LEU | A | 8 | 25.519 | −50.873 | −20.247 | 1.00 | 16.05 | O |
| ATOM | 127 | N | ALA | A | 9 | 27.462 | −49.991 | −20.960 | 1.00 | 16.99 | N |
| ATOM | 128 | CA | ALA | A | 9 | 28.263 | −50.932 | −20.203 | 1.00 | 16.14 | C |
| ATOM | 129 | CB | ALA | A | 9 | 29.737 | −50.855 | −20.583 | 1.00 | 15.06 | C |
| ATOM | 130 | C | ALA | A | 9 | 28.086 | −50.698 | −18.672 | 1.00 | 15.93 | C |
| ATOM | 131 | O | ALA | A | 9 | 28.076 | −51.692 | −17.894 | 1.00 | 15.13 | O |
| ATOM | 132 | N | ALA | A | 10 | 28.006 | −49.434 | −18.252 | 1.00 | 15.67 | N |
| ATOM | 133 | CA | ALA | A | 10 | 27.823 | −49.094 | −16.834 | 1.00 | 16.01 | C |
| ATOM | 134 | CB | ALA | A | 10 | 28.010 | −47.530 | −16.585 | 1.00 | 15.78 | C |
| ATOM | 135 | C | ALA | A | 10 | 26.428 | −49.568 | −16.296 | 1.00 | 16.10 | C |
| ATOM | 136 | O | ALA | A | 10 | 26.337 | −50.132 | −15.228 | 1.00 | 16.67 | O |
| ATOM | 137 | N | VAL | A | 11 | 25.386 | −49.304 | −17.066 | 1.00 | 15.68 | N |
| ATOM | 138 | CA | VAL | A | 11 | 24.012 | −49.773 | −16.758 | 1.00 | 16.30 | C |
| ATOM | 139 | CB | VAL | A | 11 | 22.972 | −49.208 | −17.703 | 1.00 | 14.94 | C |
| ATOM | 140 | CG1 | VAL | A | 11 | 21.554 | −49.864 | −17.528 | 1.00 | 16.14 | C |
| ATOM | 141 | CG2 | VAL | A | 11 | 22.882 | −47.575 | −17.552 | 1.00 | 15.49 | C |
| ATOM | 142 | C | VAL | A | 11 | 24.015 | −51.290 | −16.733 | 1.00 | 16.92 | C |
| ATOM | 143 | O | VAL | A | 11 | 23.349 | −51.890 | −15.882 | 1.00 | 16.60 | O |
| ATOM | 144 | N | THR | A | 12 | 24.685 | −51.911 | −17.702 | 1.00 | 16.32 | N |
| ATOM | 145 | CA | THR | A | 12 | 24.817 | −53.378 | −17.708 | 1.00 | 17.42 | C |
| ATOM | 146 | CB | THR | A | 12 | 25.647 | −53.859 | −18.903 | 1.00 | 18.02 | C |
| ATOM | 147 | OG1 | THR | A | 12 | 24.987 | −53.519 | −20.137 | 1.00 | 16.93 | O |
| ATOM | 148 | CG2 | THR | A | 12 | 26.036 | −55.370 | −18.851 | 1.00 | 14.01 | C |
| ATOM | 149 | C | THR | A | 12 | 25.433 | −53.910 | −16.409 | 1.00 | 16.73 | C |
| ATOM | 150 | O | THR | A | 12 | 24.910 | −54.894 | −15.816 | 1.00 | 15.64 | O |
| ATOM | 151 | N | LEU | A | 13 | 26.552 | −53.313 | −15.996 | 1.00 | 15.79 | N |
| ATOM | 152 | CA | LEU | A | 13 | 27.221 | −53.737 | −14.800 | 1.00 | 16.01 | C |
| ATOM | 153 | CB | LEU | A | 13 | 28.590 | −53.047 | −14.609 | 1.00 | 16.41 | C |
| ATOM | 154 | CG | LEU | A | 13 | 29.393 | −53.565 | −13.388 | 1.00 | 19.68 | C |
| ATOM | 155 | CD1 | LEU | A | 13 | 29.771 | −55.020 | −13.556 | 1.00 | 19.44 | C |
| ATOM | 156 | CD2 | LEU | A | 13 | 30.713 | −52.725 | −13.200 | 1.00 | 19.13 | C |
| ATOM | 157 | C | LEU | A | 13 | 26.337 | −53.506 | −13.570 | 1.00 | 15.89 | C |
| ATOM | 158 | O | LEU | A | 13 | 26.323 | −54.352 | −12.672 | 1.00 | 14.48 | O |
| ATOM | 159 | N | LEU | A | 14 | 25.632 | −52.378 | −13.502 | 1.00 | 15.35 | N |
| ATOM | 160 | CA | ALEU | A | 14 | 24.701 | −52.134 | −12.396 | 0.50 | 15.61 | C |
| ATOM | 161 | CA | BLEU | A | 14 | 24.708 | −52.130 | −12.403 | 0.50 | 17.49 | C |
| ATOM | 162 | CB | ALEU | A | 14 | 23.965 | −50.809 | −12.565 | 0.50 | 14.81 | C |
| ATOM | 163 | CB | BLEU | A | 14 | 24.010 | −50.795 | −12.601 | 0.50 | 17.49 | C |
| ATOM | 164 | CG | ALEU | A | 14 | 22.907 | −50.423 | −11.509 | 0.50 | 14.03 | C |
| ATOM | 165 | CG | BLEU | A | 14 | 24.340 | −49.618 | −11.680 | 0.50 | 23.49 | C |
| ATOM | 166 | CD1 | ALEU | A | 14 | 23.472 | −50.464 | −10.087 | 0.50 | 14.69 | C |
| ATOM | 167 | CD1 | BLEU | A | 14 | 23.836 | −48.354 | −12.361 | 0.50 | 26.02 | C |
| ATOM | 168 | CD2 | ALEU | A | 14 | 22.393 | −49.016 | −11.824 | 0.50 | 13.57 | C |
| ATOM | 169 | CD2 | BLEU | A | 14 | 23.635 | −49.803 | −10.304 | 0.50 | 24.03 | C |
| ATOM | 170 | C | LEU | A | 14 | 23.638 | −53.234 | −12.357 | 1.00 | 16.89 | C |
| ATOM | 171 | O | LEU | A | 14 | 23.233 | −53.656 | −11.277 | 1.00 | 16.93 | O |
| ATOM | 172 | N | GLY | A | 15 | 23.162 | −53.637 | −13.546 | 1.00 | 17.46 | N |
| ATOM | 173 | CA | GLY | A | 15 | 22.179 | −54.730 | −13.683 | 1.00 | 16.39 | C |
| ATOM | 174 | C | GLY | A | 15 | 22.728 | −56.068 | −13.167 | 1.00 | 17.25 | C |
| ATOM | 175 | O | GLY | A | 15 | 22.008 | −56.795 | −12.478 | 1.00 | 16.27 | O |
| ATOM | 176 | N | VAL | A | 16 | 23.985 | −56.388 | −13.474 | 1.00 | 15.54 | N |
| ATOM | 177 | CA | VAL | A | 16 | 24.635 | −57.585 | −12.942 | 1.00 | 16.05 | C |
| ATOM | 178 | CB | VAL | A | 16 | 26.081 | −57.768 | −13.534 | 1.00 | 16.60 | C |
| ATOM | 179 | CG1 | VAL | A | 16 | 26.889 | −58.786 | −12.790 | 1.00 | 13.19 | C |
| ATOM | 180 | CG2 | VAL | A | 16 | 26.005 | −58.062 | −15.097 | 1.00 | 14.81 | C |
| ATOM | 181 | C | VAL | A | 16 | 24.700 | −57.497 | −11.408 | 1.00 | 16.94 | C |
| ATOM | 182 | O | VAL | A | 16 | 24.384 | −58.486 | −10.737 | 1.00 | 15.38 | O |
| ATOM | 183 | N | LEU | A | 17 | 25.153 | −56.345 | −10.865 | 1.00 | 16.09 | N |
| ATOM | 184 | CA | LEU | A | 17 | 25.217 | −56.158 | −9.422 | 1.00 | 17.41 | C |
| ATOM | 185 | CB | LEU | A | 17 | 25.774 | −54.767 | −9.072 | 1.00 | 17.39 | C |
| ATOM | 186 | CG | LEU | A | 17 | 27.252 | −54.609 | −9.426 | 1.00 | 18.79 | C |
| ATOM | 187 | CD1 | LEU | A | 17 | 27.647 | −53.153 | −9.176 | 1.00 | 23.01 | C |
| ATOM | 188 | CD2 | LEU | A | 17 | 28.127 | −55.576 | −8.560 | 1.00 | 17.11 | C |
| ATOM | 189 | C | LEU | A | 17 | 23.847 | −56.346 | −8.730 | 1.00 | 16.71 | C |
| ATOM | 190 | O | LEU | A | 17 | 23.767 | −56.949 | −7.656 | 1.00 | 14.14 | O |
| ATOM | 191 | N | LEU | A | 18 | 22.801 | −55.774 | −9.314 | 1.00 | 16.77 | N |
| ATOM | 192 | CA | ALEU | A | 18 | 21.424 | −55.941 | −8.796 | 0.50 | 16.35 | C |
| ATOM | 193 | CA | BLEU | A | 18 | 21.461 | −55.960 | −8.738 | 0.50 | 17.28 | C |
| ATOM | 194 | CB | ALEU | A | 18 | 20.433 | −55.107 | −9.625 | 0.50 | 16.20 | C |
| ATOM | 195 | CB | BLEU | A | 18 | 20.446 | −55.068 | −9.426 | 0.50 | 17.28 | C |
| ATOM | 196 | CG | ALEU | A | 18 | 18.964 | −55.058 | −9.160 | 0.50 | 15.46 | C |
| ATOM | 197 | CG | BLEU | A | 18 | 20.350 | −53.591 | −9.062 | 0.50 | 19.31 | C |
| ATOM | 198 | CD1 | ALEU | A | 18 | 18.839 | −54.376 | −7.795 | 0.50 | 11.79 | C |
| ATOM | 199 | CD1 | BLEU | A | 18 | 19.355 | −52.998 | −10.042 | 0.50 | 16.93 | C |
| ATOM | 200 | CD2 | ALEU | A | 18 | 18.013 | −54.427 | −10.216 | 0.50 | 14.53 | C |
| ATOM | 201 | CD2 | BLEU | A | 18 | 19.894 | −53.345 | −7.599 | 0.50 | 18.66 | C |
| ATOM | 202 | C | LEU | A | 18 | 20.989 | −57.425 | −8.810 | 1.00 | 17.19 | C |
| ATOM | 203 | O | LEU | A | 18 | 20.348 | −57.916 | −7.890 | 1.00 | 16.76 | O |
| ATOM | 204 | N | GLN | A | 19 | 21.295 | −58.128 | −9.897 | 1.00 | 17.14 | N |
| ATOM | 205 | CA | GLN | A | 19 | 20.958 | −59.554 | −9.983 | 1.00 | 17.18 | C |
| ATOM | 206 | CB | GLN | A | 19 | 21.254 | −60.163 | −11.377 | 1.00 | 16.60 | C |
| ATOM | 207 | CG | GLN | A | 19 | 20.347 | −59.642 | −12.516 | 1.00 | 16.76 | C |
| ATOM | 208 | CD | GLN | A | 19 | 18.827 | −60.028 | −12.267 | 1.00 | 21.75 | C |
| ATOM | 209 | OE1 | GLN | A | 19 | 17.959 | −59.196 | −12.194 | 1.00 | 21.27 | O |
| ATOM | 210 | NE2 | GLN | A | 19 | 18.581 | −61.276 | −12.126 | 1.00 | 18.28 | N |
| ATOM | 211 | C | GLN | A | 19 | 21.729 | −60.361 | −8.946 | 1.00 | 18.44 | C |
| ATOM | 212 | O | GLN | A | 19 | 21.171 | −61.281 | −8.360 | 1.00 | 16.40 | O |
| ATOM | 213 | N | ALA | A | 20 | 23.004 | −60.028 | −8.737 | 1.00 | 17.03 | N |
| ATOM | 214 | CA | ALA | A | 20 | 23.788 | −60.698 | −7.729 | 1.00 | 18.65 | C |
| ATOM | 215 | CB | ALA | A | 20 | 25.240 | −60.148 | −7.704 | 1.00 | 18.11 | C |
| ATOM | 216 | C | ALA | A | 20 | 23.144 | −60.456 | −6.357 | 1.00 | 19.31 | C |
| ATOM | 217 | O | ALA | A | 20 | 23.092 | −61.366 | −5.503 | 1.00 | 18.87 | O |
| ATOM | 218 | N | TYR | A | 21 | 22.699 | −59.225 | −6.130 | 1.00 | 18.33 | N |
| ATOM | 219 | CA | TYR | A | 21 | 22.041 | −58.885 | −4.880 | 1.00 | 19.08 | C |
| ATOM | 220 | CB | TYR | A | 21 | 21.742 | −57.387 | −4.855 | 1.00 | 19.27 | C |
| ATOM | 221 | CG | TYR | A | 21 | 20.869 | −56.985 | −3.692 | 1.00 | 22.24 | C |
| ATOM | 222 | CD1 | TYR | A | 21 | 21.394 | −56.910 | −2.401 | 1.00 | 23.86 | C |
| ATOM | 223 | CE1 | TYR | A | 21 | 20.586 | −56.526 | −1.340 | 1.00 | 25.39 | C |
| ATOM | 224 | CZ | TYR | A | 21 | 19.262 | −56.203 | −1.595 | 1.00 | 24.30 | C |
| ATOM | 225 | OH | TYR | A | 21 | 18.446 | −55.826 | −0.571 | 1.00 | 27.49 | O |
| ATOM | 226 | CE2 | TYR | A | 21 | 18.739 | −56.245 | −2.867 | 1.00 | 25.76 | C |
| ATOM | 227 | CD2 | TYR | A | 21 | 19.539 | −56.628 | −3.892 | 1.00 | 22.26 | C |
| ATOM | 228 | C | TYR | A | 21 | 20.750 | −59.717 | −4.647 | 1.00 | 18.54 | C |
| ATOM | 229 | O | TYR | A | 21 | 20.509 | −60.207 | −3.535 | 1.00 | 18.86 | O |
| ATOM | 230 | N | PHE | A | 22 | 19.959 | −59.901 | −5.699 | 1.00 | 18.09 | N |
| ATOM | 231 | CA | PHE | A | 22 | 18.702 | −60.656 | −5.615 | 1.00 | 17.87 | C |
| ATOM | 232 | CB | PHE | A | 22 | 17.942 | −60.678 | −6.944 | 1.00 | 16.55 | C |
| ATOM | 233 | CG | PHE | A | 22 | 17.414 | −59.362 | −7.367 | 1.00 | 18.68 | C |
| ATOM | 234 | CD1 | PHE | A | 22 | 17.160 | −58.359 | −6.431 | 1.00 | 16.52 | C |
| ATOM | 235 | CE1 | PHE | A | 22 | 16.657 | −57.094 | −6.817 | 1.00 | 17.66 | C |
| ATOM | 236 | CZ | PHE | A | 22 | 16.413 | −56.823 | −8.173 | 1.00 | 18.35 | C |
| ATOM | 237 | CE2 | PHE | A | 22 | 16.700 | −57.824 | −9.142 | 1.00 | 19.95 | C |
| ATOM | 238 | CD2 | PHE | A | 22 | 17.186 | −59.104 | −8.729 | 1.00 | 18.06 | C |
| ATOM | 239 | C | PHE | A | 22 | 19.062 | −62.094 | −5.227 | 1.00 | 17.94 | C |
| ATOM | 240 | O | PHE | A | 22 | 18.402 | −62.692 | −4.375 | 1.00 | 16.29 | O |
| ATOM | 241 | N | SER | A | 23 | 20.119 | −62.627 | −5.841 | 1.00 | 17.48 | N |
| ATOM | 242 | CA | ASER | A | 23 | 20.609 | −63.977 | −5.526 | 0.50 | 18.22 | C |
| ATOM | 243 | CA | BSER | A | 23 | 20.607 | −63.979 | −5.524 | 0.50 | 18.14 | C |
| ATOM | 244 | CB | ASER | A | 23 | 21.743 | −64.373 | −6.496 | 0.50 | 18.07 | C |
| ATOM | 245 | CB | BSER | A | 23 | 21.764 | −64.390 | −6.459 | 0.50 | 17.96 | C |
| ATOM | 246 | OG | ASER | A | 23 | 21.172 | −64.782 | −7.725 | 0.50 | 17.88 | O |
| ATOM | 247 | OG | BSER | A | 23 | 21.969 | −65.784 | −6.334 | 0.50 | 17.40 | O |
| ATOM | 248 | C | SER | A | 23 | 21.052 | −64.132 | −4.074 | 1.00 | 18.29 | C |
| ATOM | 249 | O | SER | A | 23 | 20.715 | −65.143 | −3.407 | 1.00 | 18.63 | O |
| ATOM | 250 | N | LEU | A | 24 | 21.797 | −63.150 | −3.578 | 1.00 | 18.16 | N |
| ATOM | 251 | CA | LEU | A | 24 | 22.211 | −63.166 | −2.191 | 1.00 | 20.24 | C |
| ATOM | 252 | CB | LEU | A | 24 | 23.208 | −62.051 | −1.887 | 1.00 | 21.40 | C |
| ATOM | 253 | CG | LEU | A | 24 | 24.537 | −62.161 | −2.669 | 1.00 | 25.52 | C |
| ATOM | 254 | CD1 | LEU | A | 24 | 25.348 | −60.826 | −2.669 | 1.00 | 27.61 | C |
| ATOM | 255 | CD2 | LEU | A | 24 | 25.433 | −63.346 | −2.181 | 1.00 | 28.16 | C |
| ATOM | 256 | C | LEU | A | 24 | 20.993 | −63.080 | −1.279 | 1.00 | 20.87 | C |
| ATOM | 257 | O | LEU | A | 24 | 20.974 | −63.698 | −0.204 | 1.00 | 19.35 | O |
| ATOM | 258 | N | GLN | A | 25 | 19.970 | −62.331 | −1.688 | 1.00 | 20.20 | N |
| ATOM | 259 | CA | GLN | A | 25 | 18.769 | −62.243 | −0.834 | 1.00 | 20.86 | C |
| ATOM | 260 | CB | GLN | A | 25 | 17.844 | −61.147 | −1.343 | 1.00 | 20.63 | C |
| ATOM | 261 | CG | GLN | A | 25 | 18.363 | −59.767 | −1.108 | 1.00 | 26.30 | C |
| ATOM | 262 | CD | GLN | A | 25 | 18.336 | −59.415 | 0.352 | 1.00 | 31.82 | C |
| ATOM | 263 | OE1 | GLN | A | 25 | 19.368 | −59.242 | 0.972 | 1.00 | 36.59 | O |
| ATOM | 264 | NE2 | GLN | A | 25 | 17.145 | −59.354 | 0.922 | 1.00 | 37.03 | N |
| ATOM | 265 | C | GLN | A | 25 | 18.014 | −63.599 | −0.763 | 1.00 | 19.55 | C |
| ATOM | 266 | O | GLN | A | 25 | 17.436 | −63.957 | 0.273 | 1.00 | 19.83 | O |
| ATOM | 267 | N | VAL | A | 26 | 18.004 | −64.344 | −1.865 | 1.00 | 18.97 | N |
| ATOM | 268 | CA | VAL | A | 26 | 17.426 | −65.679 | −1.853 | 1.00 | 19.01 | C |
| ATOM | 269 | CB | VAL | A | 26 | 17.356 | −66.299 | −3.264 | 1.00 | 19.68 | C |
| ATOM | 270 | CG1 | VAL | A | 26 | 16.870 | −67.745 | −3.190 | 1.00 | 18.47 | C |
| ATOM | 271 | CG2 | VAL | A | 26 | 16.433 | −65.468 | −4.130 | 1.00 | 19.44 | C |
| ATOM | 212 | C | VAL | A | 26 | 18.209 | −66.598 | −0.922 | 1.00 | 19.18 | C |
| ATOM | 273 | O | VAL | A | 26 | 17.603 | −67.351 | −0.176 | 1.00 | 17.84 | O |
| ATOM | 274 | N | ILE | A | 27 | 19.540 | −66.574 | −0.994 | 1.00 | 18.77 | N |
| ATOM | 275 | CA | ILE | A | 27 | 20.368 | −67.361 | −0.081 | 1.00 | 18.90 | C |
| ATOM | 276 | CB | ILE | A | 27 | 21.872 | −67.186 | −0.396 | 1.00 | 19.36 | C |
| ATOM | 277 | CG1 | ILE | A | 27 | 22.168 | −67.738 | −1.801 | 1.00 | 19.09 | C |
| ATOM | 278 | CD1 | ILE | A | 27 | 23.527 | −67.372 | −2.345 | 1.00 | 21.97 | C |
| ATOM | 279 | CG2 | ILE | A | 27 | 22.768 | −67.923 | 0.683 | 1.00 | 16.77 | C |
| ATOM | 280 | C | ILE | A | 27 | 20.088 | −66.972 | 1.381 | 1.00 | 20.52 | C |
| ATOM | 281 | O | ILE | A | 27 | 20.019 | −67.814 | 2.279 | 1.00 | 18.92 | O |
| ATOM | 282 | N | SER | A | 28 | 19.923 | −65.676 | 1.611 | 1.00 | 21.21 | N |
| ATOM | 283 | CA | SER | A | 28 | 19.598 | −65.189 | 2.930 | 1.00 | 22.97 | C |
| ATOM | 284 | CB | SER | A | 28 | 19.706 | −63.658 | 2.950 | 1.00 | 22.02 | C |
| ATOM | 285 | OG | SER | A | 28 | 19.162 | −63.196 | 4.183 | 1.00 | 29.00 | O |
| ATOM | 286 | C | SER | A | 28 | 18.203 | −65.679 | 3.388 | 1.00 | 22.89 | C |
| ATOM | 287 | O | SER | A | 28 | 18.035 | −66.082 | 4.523 | 1.00 | 22.64 | O |
| ATOM | 288 | N | ALA | A | 29 | 17.214 | −65.659 | 2.493 | 1.00 | 23.91 | N |
| ATOM | 289 | CA | ALA | A | 29 | 15.865 | −66.183 | 2.797 | 1.00 | 23.51 | C |
| ATOM | 290 | CB | ALA | A | 29 | 14.866 | −65.824 | 1.680 | 1.00 | 22.99 | C |
| ATOM | 291 | C | ALA | A | 29 | 15.858 | −67.681 | 3.051 | 1.00 | 24.00 | C |
| ATOM | 292 | O | ALA | A | 29 | 15.073 | −68.156 | 3.864 | 1.00 | 23.89 | O |
| ATOM | 293 | N | ARG | A | 30 | 16.697 | −68.436 | 2.340 | 1.00 | 24.05 | N |
| ATOM | 294 | CA | ARG | A | 30 | 16.889 | −69.872 | 2.601 | 1.00 | 24.83 | C |
| ATOM | 295 | CB | ARG | A | 30 | 17.953 | −70.466 | 1.666 | 1.00 | 24.79 | C |
| ATOM | 296 | CG | ARG | A | 30 | 17.442 | −71.056 | 0.403 | 1.00 | 23.58 | C |
| ATOM | 297 | CD | ARG | A | 30 | 18.611 | −71.522 | −0.501 | 1.00 | 25.95 | C |
| ATOM | 298 | NE | ARG | A | 30 | 18.140 | −71.735 | −1.879 | 1.00 | 31.89 | N |
| ATOM | 299 | CZ | ARG | A | 30 | 17.282 | −72.709 | −2.245 | 1.00 | 32.74 | C |
| ATOM | 300 | NH1 | ARG | A | 30 | 16.809 | −73.552 | −1.329 | 1.00 | 26.90 | N |
| ATOM | 301 | NH2 | ARG | A | 30 | 16.886 | −72.852 | −3.520 | 1.00 | 26.35 | N |
| ATOM | 302 | C | ARG | A | 30 | 17.296 | −70.172 | 4.053 | 1.00 | 26.73 | C |
| ATOM | 303 | O | ARG | A | 30 | 16.859 | −71.188 | 4.631 | 1.00 | 25.68 | O |
| ATOM | 304 | N | ARG | A | 31 | 18.136 | −69.312 | 4.642 | 1.00 | 28.09 | N |
| ATOM | 305 | CA | ARG | A | 31 | 18.486 | −69.426 | 6.072 | 1.00 | 29.61 | C |
| ATOM | 306 | CB | ARG | A | 31 | 19.806 | −68.732 | 6.380 | 1.00 | 30.78 | C |
| ATOM | 307 | CG | ARG | A | 31 | 20.959 | −69.299 | 5.571 | 1.00 | 35.56 | C |
| ATOM | 308 | CD | ARG | A | 31 | 21.813 | −68.181 | 4.962 | 1.00 | 44.64 | C |
| ATOM | 309 | NE | ARG | A | 31 | 23.081 | −68.659 | 4.386 | 1.00 | 49.92 | N |
| ATOM | 310 | CZ | ARG | A | 31 | 24.024 | −67.860 | 3.879 | 1.00 | 54.18 | C |
| ATOM | 311 | NE1 | ARG | A | 31 | 23.844 | −66.530 | 3.854 | 1.00 | 55.03 | N |
| ATOM | 312 | NH2 | ARG | A | 31 | 25.143 | −68.386 | 3.373 | 1.00 | 55.84 | N |
| ATOM | 313 | C | ARG | A | 31 | 17.375 | −68.914 | 6.995 | 1.00 | 29.48 | C |
| ATOM | 314 | O | ARG | A | 31 | 16.919 | −69.643 | 7.879 | 1.00 | 28.43 | O |
| ATOM | 315 | N | ALA | A | 32 | 16.911 | −67.680 | 6.783 | 1.00 | 29.12 | N |
| ATOM | 316 | CA | ALA | A | 32 | 15.800 | −67.176 | 7.593 | 1.00 | 29.17 | C |
| ATOM | 317 | CB | ALA | A | 32 | 15.349 | −65.806 | 7.126 | 1.00 | 28.95 | C |
| ATOM | 318 | C | ALA | A | 32 | 14.622 | −68.163 | 7.628 | 1.00 | 29.78 | C |
| ATOM | 319 | O | ALA | A | 32 | 14.097 | −68.471 | 8.699 | 1.00 | 29.40 | O |
| ATOM | 320 | N | PHE | A | 33 | 14.234 | −68.696 | 6.468 | 1.00 | 30.14 | N |
| ATOM | 321 | CA | PHE | A | 33 | 13.040 | −69.536 | 6.403 | 1.00 | 30.08 | C |
| ATOM | 322 | CB | PHE | A | 33 | 12.127 | −69.061 | 5.289 | 1.00 | 30.34 | C |
| ATOM | 323 | CG | PHE | A | 33 | 11.816 | −67.626 | 5.409 | 1.00 | 31.02 | C |
| ATOM | 324 | CD1 | PHE | A | 33 | 11.058 | −67.178 | 6.483 | 1.00 | 32.95 | C |
| ATOM | 325 | CE1 | PHE | A | 33 | 10.794 | −65.818 | 6.646 | 1.00 | 34.66 | C |
| ATOM | 326 | CZ | PHE | A | 33 | 11.288 | −64.893 | 5.742 | 1.00 | 33.75 | C |
| ATOM | 327 | CE2 | PHE | A | 33 | 12.076 | −65.336 | 4.648 | 1.00 | 34.48 | C |
| ATOM | 328 | CD2 | PHE | A | 33 | 12.334 | −66.702 | 4.504 | 1.00 | 32.19 | C |
| ATOM | 329 | C | PHE | A | 33 | 13.322 | −71.010 | 6.344 | 1.00 | 29.65 | C |
| ATOM | 330 | O | PHE | A | 33 | 12.400 | −71.813 | 6.127 | 1.00 | 29.19 | O |
| ATOM | 331 | N | ARG | A | 34 | 14.579 | −71.366 | 6.615 | 1.00 | 29.06 | N |
| ATOM | 332 | CA | ARG | A | 34 | 14.995 | −72.774 | 6.706 | 1.00 | 28.59 | C |
| ATOM | 333 | CB | ARG | A | 34 | 14.435 | −73.427 | 7.996 | 1.00 | 29.80 | C |
| ATOM | 334 | CG | ARG | A | 34 | 14.347 | −72.512 | 9.225 | 1.00 | 33.40 | C |
| ATOM | 335 | CD | ARG | A | 34 | 15.642 | −72.504 | 10.040 | 1.00 | 40.97 | C |
| ATOM | 336 | NE | ARG | A | 34 | 15.448 | −71.941 | 11.394 | 1.00 | 46.03 | N |
| ATOM | 337 | CZ | ARG | A | 34 | 15.778 | −70.690 | 11.743 | 1.00 | 48.13 | C |
| ATOM | 338 | NH1 | ARG | A | 34 | 16.318 | −69.853 | 10.843 | 1.00 | 46.71 | N |
| ATOM | 339 | NH2 | ARG | A | 34 | 15.567 | −70.270 | 12.991 | 1.00 | 49.30 | N |
| ATOM | 340 | C | ARG | A | 34 | 14.549 | −73.586 | 5.482 | 1.00 | 27.84 | C |
| ATOM | 341 | O | ARG | A | 34 | 13.721 | −74.488 | 5.613 | 1.00 | 27.01 | O |
| ATOM | 342 | N | VAL | A | 35 | 15.062 | −73.239 | 4.285 | 1.00 | 26.72 | N |
| ATOM | 343 | CA | VAL | A | 35 | 14.850 | −74.044 | 3.081 | 1.00 | 25.93 | C |
| ATOM | 344 | CB | VAL | A | 35 | 14.109 | −73.224 | 1.967 | 1.00 | 25.70 | C |
| ATOM | 345 | CG1 | VAL | A | 35 | 13.853 | −74.088 | 0.734 | 1.00 | 24.14 | C |
| ATOM | 346 | CG2 | VAL | A | 35 | 12.807 | −72.636 | 2.511 | 1.00 | 26.15 | C |
| ATOM | 347 | C | VAL | A | 35 | 16.180 | −74.571 | 2.530 | 1.00 | 26.42 | C |
| ATOM | 348 | O | VAL | A | 35 | 17.033 | −73.785 | 2.055 | 1.00 | 28.00 | O |
| ATOM | 349 | N | SER | A | 36 | 16.358 | −75.879 | 2.485 | 1.00 | 25.58 | N |
| ATOM | 350 | CA | SER | A | 36 | 17.659 | −76.397 | 2.045 | 1.00 | 27.44 | C |
| ATOM | 351 | CB | SER | A | 36 | 18.135 | −77.585 | 2.923 | 1.00 | 27.60 | C |
| ATOM | 352 | OG | SER | A | 36 | 17.100 | −78.531 | 3.105 | 1.00 | 31.58 | O |
| ATOM | 353 | C | SER | A | 36 | 17.644 | −76.781 | 0.566 | 1.00 | 27.25 | C |
| ATOM | 354 | O | SER | A | 36 | 16.652 | −77.349 | 0.110 | 1.00 | 26.82 | O |
| ATOM | 355 | N | PRO | A | 37 | 18.722 | −76.440 | −0.183 | 1.00 | 27.91 | N |
| ATOM | 356 | CA | PRO | A | 37 | 18.980 | −76.878 | −1.579 | 1.00 | 27.78 | C |
| ATOM | 357 | CB | PRO | A | 37 | 20.368 | −76.318 | −1.862 | 1.00 | 27.43 | C |
| ATOM | 358 | CG | PRO | A | 37 | 20.467 | −75.142 | −0.958 | 1.00 | 27.76 | C |
| ATOM | 359 | CD | PRO | A | 37 | 19.786 | −75.539 | 0.296 | 1.00 | 27.85 | C |
| ATOM | 360 | C | PRO | A | 37 | 18.976 | −78.431 | −1.697 | 1.00 | 28.57 | C |
| ATOM | 361 | O | PRO | A | 37 | 19.380 | −79.080 | −0.755 | 1.00 | 28.21 | O |
| ATOM | 362 | N | PRO | A | 38 | 18.481 | −79.006 | −2.827 | 1.00 | 29.15 | N |
| ATOM | 363 | CA | PRO | A | 38 | 17.946 | −78.317 | −4.032 | 1.00 | 29.69 | C |
| ATOM | 364 | CB | PRO | A | 38 | 17.825 | −79.449 | −5.044 | 1.00 | 29.10 | C |
| ATOM | 365 | CG | PRO | A | 38 | 17.599 | −80.684 | −4.242 | 1.00 | 29.23 | C |
| ATOM | 366 | CD | PRO | A | 38 | 18.331 | −80.478 | −2.915 | 1.00 | 29.05 | C |
| ATOM | 367 | C | PRO | A | 38 | 16.579 | −77.858 | −3.557 | 1.00 | 31.15 | C |
| ATOM | 368 | O | PRO | A | 38 | 16.329 | −77.953 | −2.357 | 1.00 | 31.21 | O |
| ATOM | 369 | N | LEU | A | 39 | 15.629 | −77.422 | −4.358 | 1.00 | 31.64 | N |
| ATOM | 370 | CA | LEU | A | 39 | 14.314 | −77.307 | −3.600 | 1.00 | 31.36 | C |
| ATOM | 371 | CB | LEU | A | 39 | 14.358 | −78.099 | −2.298 | 1.00 | 32.57 | C |
| ATOM | 372 | CG | LEU | A | 39 | 13.205 | −78.765 | −1.538 | 1.00 | 33.34 | C |
| ATOM | 373 | CD1 | LEU | A | 39 | 13.595 | −79.057 | −0.071 | 1.00 | 37.71 | C |
| ATOM | 374 | CD2 | LEU | A | 39 | 12.846 | −80.105 | −2.226 | 1.00 | 37.19 | C |
| ATOM | 375 | C | LEU | A | 39 | 14.044 | −75.881 | −3.248 | 1.00 | 30.76 | C |
| ATOM | 376 | O | LEU | A | 39 | 14.863 | −75.168 | −2.653 | 1.00 | 30.37 | O |
| ATOM | 377 | N | THR | A | 40 | 12.889 | −75.453 | −3.697 | 1.00 | 30.19 | N |
| ATOM | 378 | CA | THR | A | 40 | 12.610 | −74.057 | −3.883 | 1.00 | 29.53 | C |
| ATOM | 379 | CB | THR | A | 40 | 12.688 | −73.773 | −5.367 | 1.00 | 30.60 | C |
| ATOM | 380 | OG1 | THR | A | 40 | 11.937 | −74.791 | −6.056 | 1.00 | 31.63 | O |
| ATOM | 381 | CG2 | THR | A | 40 | 14.141 | −73.849 | −5.799 | 1.00 | 29.67 | C |
| ATOM | 382 | C | THR | A | 40 | 11.203 | −73.800 | −3.360 | 1.00 | 28.92 | C |
| ATOM | 383 | O | THR | A | 40 | 10.597 | −72.784 | −3.659 | 1.00 | 29.15 | O |
| ATOM | 384 | N | THR | A | 41 | 10.704 | −74.765 | −2.583 | 1.00 | 27.72 | N |
| ATOM | 385 | CA | THR | A | 41 | 9.417 | −74.650 | −1.909 | 1.00 | 27.48 | C |
| ATOM | 386 | CB | THR | A | 41 | 8.502 | −75.882 | −2.175 | 1.00 | 27.00 | C |
| ATOM | 387 | OG1 | THR | A | 41 | 9.214 | −77.087 | −1.892 | 1.00 | 27.66 | O |
| ATOM | 388 | CG2 | THR | A | 41 | 8.072 | −75.910 | −3.621 | 1.00 | 29.36 | C |
| ATOM | 389 | C | THR | A | 41 | 9.699 | −74.492 | −0.431 | 1.00 | 25.77 | C |
| ATOM | 390 | O | THR | A | 41 | 10.661 | −75.046 | 0.065 | 1.00 | 25.46 | O |
| ATOM | 391 | N | GLY | A | 42 | 8.905 | −73.683 | 0.252 | 1.00 | 24.99 | N |
| ATOM | 392 | CA | GLY | A | 42 | 8.945 | −73.595 | 1.708 | 1.00 | 23.67 | C |
| ATOM | 393 | C | GLY | A | 42 | 7.875 | −72.582 | 2.070 | 1.00 | 23.48 | C |
| ATOM | 394 | O | GLY | A | 42 | 6.894 | −72.444 | 1.352 | 1.00 | 22.56 | O |
| ATOM | 395 | N | PRO | A | 43 | 8.072 | −71.841 | 3.160 | 1.00 | 23.65 | N |
| ATOM | 396 | CA | PRO | A | 43 | 7.110 | −70.819 | 3.581 | 1.00 | 24.08 | C |
| ATOM | 397 | CB | PRO | A | 43 | 7.832 | −70.125 | 4.742 | 1.00 | 24.25 | C |
| ATOM | 398 | CG | PRO | A | 43 | 8.783 | −71.149 | 5.257 | 1.00 | 25.08 | C |
| ATOM | 399 | CD | PRO | A | 43 | 9.233 | −71.924 | 4.060 | 1.00 | 23.63 | C |
| ATOM | 400 | C | PRO | A | 43 | 6.783 | −69.793 | 2.479 | 1.00 | 24.09 | C |
| ATOM | 401 | O | PRO | A | 43 | 7.610 | −69.534 | 1.594 | 1.00 | 23.76 | O |
| ATOM | 402 | N | PRO | A | 44 | 5.575 | −69.205 | 2.535 | 1.00 | 24.85 | N |
| ATOM | 403 | CA | PRO | A | 44 | 5.142 | −68.124 | 1.623 | 1.00 | 24.39 | C |
| ATOM | 404 | CB | PRO | A | 44 | 3.905 | −67.536 | 2.312 | 1.00 | 24.28 | C |
| ATOM | 405 | CG | PRO | A | 44 | 3.355 | −68.696 | 3.171 | 1.00 | 24.88 | C |
| ATOM | 406 | CD | PRO | A | 44 | 4.533 | −69.582 | 3.522 | 1.00 | 24.64 | C |
| ATOM | 407 | C | PRO | A | 44 | 6.208 | −67.048 | 1.468 | 1.00 | 25.03 | C |
| ATOM | 408 | O | PRO | A | 44 | 6.509 | −66.629 | 0.337 | 1.00 | 24.73 | O |
| ATOM | 409 | N | GLU | A | 45 | 6.785 | −66.592 | 2.577 | 1.00 | 25.54 | N |
| ATOM | 410 | CA | GLU | A | 45 | 7.791 | −65.505 | 2.508 | 1.00 | 26.37 | C |
| ATOM | 411 | CB | GLU | A | 45 | 8.148 | −65.013 | 3.911 | 1.00 | 26.87 | C |
| ATOM | 412 | CG | GLU | A | 45 | 7.986 | −66.063 | 5.051 | 1.00 | 33.53 | C |
| ATOM | 413 | CD | GLU | A | 45 | 6.539 | −66.308 | 5.538 | 1.00 | 35.80 | C |
| ATOM | 414 | OE1 | GLU | A | 45 | 5.895 | −65.375 | 6.067 | 1.00 | 40.36 | O |
| ATOM | 415 | OE2 | GLU | A | 45 | 6.050 | −67.448 | 5.397 | 1.00 | 35.30 | O |
| ATOM | 416 | C | GLU | A | 45 | 9.019 | −65.920 | 1.707 | 1.00 | 25.19 | C |
| ATOM | 417 | O | GLU | A | 45 | 9.577 | −65.130 | 0.919 | 1.00 | 26.91 | O |
| ATOM | 418 | N | PHE | A | 46 | 9.416 | −67.180 | 1.840 | 1.00 | 24.09 | N |
| ATOM | 419 | CA | PHE | A | 46 | 10.514 | −67.686 | 1.041 | 1.00 | 22.77 | C |
| ATOM | 420 | CB | PHE | A | 46 | 11.040 | −69.058 | 1.513 | 1.00 | 22.24 | C |
| ATOM | 421 | CG | PHE | A | 46 | 12.039 | −69.620 | 0.560 | 1.00 | 21.92 | C |
| ATOM | 422 | CD1 | PHE | A | 46 | 13.354 | −69.131 | 0.550 | 1.00 | 22.58 | C |
| ATOM | 423 | CE1 | PHE | A | 46 | 14.297 | −69.571 | −0.403 | 1.00 | 24.84 | C |
| ATOM | 424 | CZ | PHE | A | 46 | 13.918 | −70.488 | −1.397 | 1.00 | 21.95 | C |
| ATOM | 425 | CE2 | PHE | A | 46 | 12.570 | −70.965 | −1.407 | 1.00 | 24.69 | C |
| ATOM | 426 | CD2 | PHE | A | 46 | 11.648 | −70.513 | −0.432 | 1.00 | 22.55 | C |
| ATOM | 427 | C | PHE | A | 46 | 10.116 | −67.746 | −0.438 | 1.00 | 21.60 | C |
| ATOM | 428 | O | PHE | A | 46 | 10.870 | −67.304 | −1.291 | 1.00 | 21.09 | O |
| ATOM | 429 | N | GLU | A | 47 | 8.937 | −68.283 | −0.736 | 1.00 | 20.53 | N |
| ATOM | 430 | CA | GLU | A | 47 | 8.510 | −68.514 | −2.110 | 1.00 | 21.35 | C |
| ATOM | 431 | CB | GLU | A | 47 | 7.255 | −69.428 | −2.217 | 1.00 | 20.97 | C |
| ATOM | 432 | CG | GLU | A | 47 | 7.552 | −70.837 | −1.600 | 1.00 | 24.76 | C |
| ATOM | 433 | CD | GLU | A | 47 | 6.517 | −71.960 | −1.915 | 1.00 | 25.54 | C |
| ATOM | 434 | OE1 | GLU | A | 47 | 5.538 | −71.723 | −2.666 | 1.00 | 31.53 | O |
| ATOM | 435 | OE2 | GLU | A | 47 | 6.720 | −73.111 | −1.429 | 1.00 | 30.90 | O |
| ATOM | 436 | C | GLU | A | 47 | 8.346 | −67.187 | −2.869 | 1.00 | 20.28 | C |
| ATOM | 437 | O | GLU | A | 47 | 8.696 | −67.121 | −4.063 | 1.00 | 20.94 | O |
| ATOM | 438 | N | ARG | A | 48 | 7.895 | −66.129 | −2.182 | 1.00 | 18.14 | N |
| ATOM | 439 | CA | ARG | A | 48 | 7.808 | −64.822 | −2.830 | 1.00 | 18.29 | C |
| ATOM | 440 | CB | ARG | A | 48 | 7.068 | −63.815 | −1.947 | 1.00 | 17.43 | C |
| ATOM | 441 | CG | ARG | A | 48 | 5.519 | −64.068 | −1.806 | 1.00 | 18.36 | C |
| ATOM | 442 | CD | ARG | A | 48 | 4.802 | −62.867 | −1.200 | 1.00 | 19.00 | C |
| ATOM | 443 | NE | ARG | A | 48 | 5.470 | −62.390 | 0.030 | 1.00 | 17.46 | N |
| ATOM | 444 | CZ | ARG | A | 48 | 5.165 | −62.827 | 1.253 | 1.00 | 17.53 | C |
| ATOM | 445 | NH1 | ARG | A | 48 | 4.236 | −63.780 | 1.421 | 1.00 | 14.56 | N |
| ATOM | 446 | NH2 | ARG | A | 48 | 5.825 | −62.346 | 2.290 | 1.00 | 18.95 | N |
| ATOM | 447 | C | ARG | A | 48 | 9.216 | −64.278 | −3.237 | 1.00 | 17.89 | C |
| ATOM | 448 | O | ARG | A | 48 | 9.375 | −63.673 | −4.285 | 1.00 | 16.60 | O |
| ATOM | 449 | N | VAL | A | 49 | 10.203 | −64.442 | −2.354 | 1.00 | 18.24 | N |
| ATOM | 450 | CA | VAL | A | 49 | 11.547 | −63.897 | −2.571 | 1.00 | 18.62 | C |
| ATOM | 451 | CB | VAL | A | 49 | 12.432 | −64.045 | −1.288 | 1.00 | 18.71 | C |
| ATOM | 452 | CG1 | VAL | A | 49 | 13.930 | −63.668 | −1.646 | 1.00 | 18.08 | C |
| ATOM | 453 | CG2 | VAL | A | 49 | 11.927 | −63.102 | −0.190 | 1.00 | 18.68 | C |
| ATOM | 454 | C | VAL | A | 49 | 12.171 | −64.709 | −3.724 | 1.00 | 18.95 | C |
| ATOM | 455 | O | VAL | A | 49 | 12.825 | −64.140 | −4.621 | 1.00 | 18.18 | O |
| ATOM | 456 | N | TYR | A | 50 | 11.986 | −66.033 | −3.654 | 1.00 | 17.64 | N |
| ATOM | 457 | CA | TYR | A | 50 | 12.406 | −66.948 | −4.717 | 1.00 | 19.43 | C |
| ATOM | 458 | CB | TYR | A | 50 | 12.079 | −68.396 | −4.342 | 1.00 | 19.93 | C |
| ATOM | 459 | CG | TYR | A | 50 | 12.435 | −69.317 | −5.462 | 1.00 | 22.07 | C |
| ATOM | 460 | CD1 | TYR | A | 50 | 13.766 | −69.562 | −5.777 | 1.00 | 23.24 | C |
| ATOM | 461 | CE1 | TYR | A | 50 | 14.114 | −70.378 | −6.836 | 1.00 | 24.35 | C |
| ATOM | 462 | CZ | TYR | A | 50 | 13.114 | −70.971 | −7.580 | 1.00 | 25.00 | C |
| ATOM | 463 | OH | TYR | A | 50 | 13.447 | −71.822 | −8.637 | 1.00 | 27.98 | O |
| ATOM | 464 | CE2 | TYR | A | 50 | 11.782 | −70.758 | −7.284 | 1.00 | 22.26 | C |
| ATOM | 465 | CD2 | TYR | A | 50 | 11.448 | −69.920 | −6.225 | 1.00 | 22.60 | C |
| ATOM | 466 | C | TYR | A | 50 | 11.821 | −66.571 | −6.080 | 1.00 | 19.23 | C |
| ATOM | 467 | O | TYR | A | 50 | 12.557 | −66.461 | −7.110 | 1.00 | 19.32 | O |
| ATOM | 468 | N | ARG | A | 51 | 10.513 | −66.341 | −6.099 | 1.00 | 19.18 | N |
| ATOM | 469 | CA | ARG | A | 51 | 9.817 | −66.052 | −7.324 | 1.00 | 19.97 | C |
| ATOM | 470 | CB | ARG | A | 51 | 8.293 | −66.173 | −7.181 | 1.00 | 21.05 | C |
| ATOM | 471 | CG | ARG | A | 51 | 7.498 | −65.862 | −8.506 | 1.00 | 26.10 | C |
| ATOM | 472 | CD | ARG | A | 51 | 7.675 | −67.005 | −9.547 | 1.00 | 31.39 | C |
| ATOM | 473 | NE | ARG | A | 51 | 7.726 | −68.297 | −8.867 | 1.00 | 31.38 | N |
| ATOM | 474 | CZ | ARG | A | 51 | 8.413 | −69.344 | −9.311 | 1.00 | 34.69 | C |
| ATOM | 475 | NH1 | ARG | A | 51 | 9.100 | −69.263 | −10.450 | 1.00 | 22.44 | N |
| ATOM | 476 | NH2 | ARG | A | 51 | 8.410 | −70.486 | −8.607 | 1.00 | 35.43 | N |
| ATOM | 477 | C | ARG | A | 51 | 10.232 | −64.688 | −7.880 | 1.00 | 20.18 | C |
| ATOM | 478 | O | ARG | A | 51 | 10.372 | −64.567 | −9.088 | 1.00 | 18.68 | O |
| ATOM | 479 | N | ALA | A | 52 | 10.427 | −63.683 | −7.002 | 1.00 | 17.84 | N |
| ATOM | 480 | CA | ALA | A | 52 | 10.853 | −62.354 | −7.440 | 1.00 | 17.20 | C |
| ATOM | 481 | CB | ALA | A | 52 | 10.935 | −61.413 | −6.249 | 1.00 | 15.92 | C |
| ATOM | 482 | C | ALA | A | 52 | 12.237 | −62.461 | −8.187 | 1.00 | 15.68 | C |
| ATOM | 483 | O | ALA | A | 52 | 12.430 | −61.854 | −9.232 | 1.00 | 15.58 | O |
| ATOM | 484 | N | GLN | A | 53 | 13.135 | −63.289 | −7.650 | 1.00 | 16.26 | N |
| ATOM | 485 | CA | GLN | A | 53 | 14.501 | −63.483 | −8.212 | 1.00 | 17.49 | C |
| ATOM | 486 | CB | GLN | A | 53 | 15.395 | −64.268 | −7.244 | 1.00 | 17.15 | C |
| ATOM | 487 | CG | GLN | A | 53 | 16.792 | −64.600 | −7.845 | 1.00 | 19.66 | C |
| ATOM | 488 | CD | GLN | A | 53 | 16.789 | −65.983 | −8.427 | 1.00 | 24.83 | C |
| ATOM | 489 | OE1 | GLN | A | 53 | 16.560 | −66.937 | −7.715 | 1.00 | 33.39 | O |
| ATOM | 490 | NE2 | GLN | A | 53 | 16.992 | −66.106 | −9.732 | 1.00 | 30.94 | N |
| ATOM | 491 | C | GLN | A | 53 | 14.383 | −64.212 | −9.519 | 1.00 | 17.09 | C |
| ATOM | 492 | O | GLN | A | 53 | 14.971 | −63.794 | −10.524 | 1.00 | 15.97 | O |
| ATOM | 493 | N | VAL | A | 54 | 13.587 | −65.286 | −9.542 | 1.00 | 16.48 | N |
| ATOM | 494 | CA | VAL | A | 54 | 13.379 | −66.003 | −10.791 | 1.00 | 18.24 | C |
| ATOM | 495 | CB | VAL | A | 54 | 12.432 | −67.201 | −10.631 | 1.00 | 17.35 | C |
| ATOM | 496 | CG1 | VAL | A | 54 | 12.072 | −67.761 | −12.023 | 1.00 | 19.79 | C |
| ATOM | 497 | CG2 | VAL | A | 54 | 13.117 | −68.271 | −9.764 | 1.00 | 17.59 | C |
| ATOM | 498 | C | VAL | A | 54 | 12.842 | −65.065 | −11.901 | 1.00 | 18.53 | C |
| ATOM | 499 | O | VAL | A | 54 | 13.329 | −65.148 | −13.050 | 1.00 | 18.60 | O |
| ATOM | 500 | N | ASN | A | 55 | 11.817 | −64.241 | −11.594 | 1.00 | 17.49 | N |
| ATOM | 501 | CA | AASN | A | 55 | 11.243 | −63.380 | −12.616 | 0.50 | 17.51 | C |
| ATOM | 502 | CA | BASN | A | 55 | 11.191 | −63.292 | −12.587 | 0.50 | 18.24 | C |
| ATOM | 503 | CB | AASN | A | 55 | 9.941 | −62.756 | −12.090 | 0.50 | 16.84 | C |
| ATOM | 504 | CB | BASN | A | 55 | 10.002 | −62.467 | −11.963 | 0.50 | 18.04 | C |
| ATOM | 505 | CG | AASN | A | 55 | 8.828 | −63.784 | −11.879 | 0.50 | 16.39 | C |
| ATOM | 506 | CG | BASN | A | 55 | 8.947 | −61.909 | −13.033 | 0.50 | 20.63 | C |
| ATOM | 507 | OD1 | AASN | A | 55 | 8.862 | −64.899 | −12.396 | 0.50 | 16.04 | O |
| ATOM | 508 | OD1 | BASN | A | 55 | 8.830 | −60.662 | −13.333 | 0.50 | 20.83 | O |
| ATOM | 509 | ND2 | AASN | A | 55 | 7.843 | −63.406 | −11.110 | 0.50 | 15.88 | N |
| ATOM | 510 | ND2 | BASN | A | 55 | 8.163 | −62.839 | −13.587 | 0.50 | 22.68 | N |
| ATOM | 511 | C | ASN | A | 55 | 12.249 | −62.307 | −13.108 | 1.00 | 17.59 | C |
| ATOM | 512 | O | ASN | A | 55 | 12.361 | −62.023 | −14.346 | 1.00 | 18.17 | O |
| ATOM | 513 | N | CYS | A | 56 | 12.999 | −61.726 | −12.179 | 1.00 | 16.96 | N |
| ATOM | 514 | CA | CYS | A | 56 | 14.022 | −60.745 | −12.544 | 1.00 | 18.32 | C |
| ATOM | 515 | CB | CYS | A | 56 | 14.714 | −60.116 | −11.337 | 1.00 | 16.70 | C |
| ATOM | 516 | SG | CYS | A | 56 | 13.524 | −59.032 | −10.424 | 1.00 | 20.36 | S |
| ATOM | 517 | C | CYS | A | 56 | 15.054 | −61.423 | −13.451 | 1.00 | 18.71 | C |
| ATOM | 518 | O | CYS | A | 56 | 15.399 | −60.855 | −14.454 | 1.00 | 19.05 | O |
| ATOM | 519 | N | SER | A | 57 | 15.461 | −62.652 | −13.114 | 1.00 | 17.88 | N |
| ATOM | 520 | CA | SER | A | 57 | 16.442 | −63.388 | −13.912 | 1.00 | 19.79 | C |
| ATOM | 521 | CB | SER | A | 57 | 16.829 | −64.712 | −13.222 | 1.00 | 19.31 | C |
| ATOM | 522 | OG | SER | A | 57 | 17.644 | −64.400 | −12.105 | 1.00 | 23.46 | O |
| ATOM | 523 | C | SER | A | 57 | 15.954 | −63.681 | −15.313 | 1.00 | 19.97 | C |
| ATOM | 524 | O | SER | A | 57 | 16.719 | −63.627 | −16.256 | 1.00 | 18.51 | O |
| ATOM | 525 | N | GLU | A | 58 | 14.686 | −64.063 | −15.430 | 1.00 | 19.38 | N |
| ATOM | 526 | CA | GLU | A | 58 | 14.071 | −64.300 | −16.712 | 1.00 | 22.71 | C |
| ATOM | 527 | CB | GLU | A | 58 | 12.650 | −64.809 | −16.454 | 1.00 | 22.29 | C |
| ATOM | 528 | CG | GLU | A | 58 | 11.610 | −64.725 | −17.615 | 1.00 | 28.06 | C |
| ATOM | 529 | CD | GLU | A | 58 | 10.119 | −64.921 | −17.129 | 1.00 | 29.82 | C |
| ATOM | 530 | OE1 | GLU | A | 58 | 9.855 | −65.434 | −15.974 | 1.00 | 37.40 | O |
| ATOM | 531 | OE2 | GLU | A | 58 | 9.194 | −64.583 | −17.921 | 1.00 | 40.41 | O |
| ATOM | 532 | C | GLU | A | 58 | 14.054 | −63.071 | −17.636 | 1.00 | 20.72 | C |
| ATOM | 533 | O | GLU | A | 58 | 14.265 | −63.194 | −18.856 | 1.00 | 20.19 | O |
| ATOM | 534 | N | TYR | A | 59 | 13.719 | −61.914 | −17.084 | 1.00 | 18.16 | N |
| ATOM | 535 | CA | TYR | A | 59 | 13.655 | −60.716 | −17.892 | 1.00 | 19.28 | C |
| ATOM | 536 | CB | TYR | A | 59 | 12.731 | −59.697 | −17.263 | 1.00 | 18.76 | C |
| ATOM | 537 | CG | TYR | A | 59 | 11.300 | −59.947 | −17.621 | 1.00 | 23.86 | C |
| ATOM | 538 | CD1 | TYR | A | 59 | 10.738 | −59.328 | −18.728 | 1.00 | 26.67 | C |
| ATOM | 539 | CE1 | TYR | A | 59 | 9.406 | −59.580 | −19.092 | 1.00 | 27.52 | C |
| ATOM | 540 | CZ | TYR | A | 59 | 8.641 | −60.410 | −18.323 | 1.00 | 27.28 | C |
| ATOM | 541 | OH | TYR | A | 59 | 7.343 | −60.578 | −18.686 | 1.00 | 30.09 | O |
| ATOM | 542 | CE2 | TYR | A | 59 | 9.149 | −61.038 | −17.165 | 1.00 | 26.90 | C |
| ATOM | 543 | CD2 | TYR | A | 59 | 10.517 | −60.823 | −16.857 | 1.00 | 23.94 | C |
| ATOM | 544 | C | TYR | A | 59 | 15.046 | −60.078 | −18.139 | 1.00 | 17.75 | C |
| ATOM | 545 | O | TYR | A | 59 | 15.174 | −59.191 | −18.944 | 1.00 | 18.12 | O |
| ATOM | 546 | N | PHE | A | 60 | 16.030 | −60.482 | −17.366 | 1.00 | 16.65 | N |
| ATOM | 547 | CA | PHE | A | 60 | 17.314 | −59.788 | −17.398 | 1.00 | 17.08 | C |
| ATOM | 548 | CB | PHE | A | 60 | 18.228 | −60.264 | −16.249 | 1.00 | 16.84 | C |
| ATOM | 549 | CG | PHE | A | 60 | 19.545 | −59.491 | −16.175 | 1.00 | 21.47 | C |
| ATOM | 550 | CD1 | PHE | A | 60 | 19.551 | −58.134 | −15.840 | 1.00 | 24.35 | C |
| ATOM | 551 | CE1 | PHE | A | 60 | 20.732 | −57.409 | −15.771 | 1.00 | 23.58 | C |
| ATOM | 552 | CZ | PHE | A | 60 | 21.932 | −58.048 | −16.025 | 1.00 | 20.33 | C |
| ATOM | 553 | CE2 | PHE | A | 60 | 21.965 | −59.374 | −16.383 | 1.00 | 24.02 | C |
| ATOM | 554 | CD2 | PHE | A | 60 | 20.725 | −60.106 | −16.465 | 1.00 | 22.80 | C |
| ATOM | 555 | C | PHE | A | 60 | 18.020 | −59.878 | −18.804 | 1.00 | 17.03 | C |
| ATOM | 556 | O | PHE | A | 60 | 18.512 | −58.852 | −19.285 | 1.00 | 16.38 | O |
| ATOM | 557 | N | PRO | A | 61 | 18.004 | −61.050 | −19.474 | 1.00 | 16.53 | N |
| ATOM | 558 | CA | PRO | A | 61 | 18.582 | −60.994 | −20.837 | 1.00 | 18.85 | C |
| ATOM | 559 | CB | PRO | A | 61 | 18.604 | −62.474 | −21.297 | 1.00 | 17.13 | C |
| ATOM | 560 | CG | PRO | A | 61 | 18.397 | −63.332 | −19.981 | 1.00 | 16.10 | C |
| ATOM | 561 | CD | PRO | A | 61 | 17.627 | −62.422 | −19.073 | 1.00 | 15.15 | C |
| ATOM | 562 | C | PRO | A | 61 | 17.781 | −60.119 | −21.831 | 1.00 | 18.43 | C |
| ATOM | 563 | O | PRO | A | 61 | 18.359 | −59.572 | −22.792 | 1.00 | 18.09 | O |
| ATOM | 564 | N | LEU | A | 62 | 16.476 | −60.001 | −21.599 | 1.00 | 18.07 | N |
| ATOM | 565 | CA | ALEU | A | 62 | 15.622 | −59.205 | −22.475 | 0.50 | 18.05 | C |
| ATOM | 566 | CA | BLEU | A | 62 | 15.629 | −59.215 | −22.477 | 0.50 | 18.30 | C |
| ATOM | 567 | CB | ALEU | A | 62 | 14.116 | −59.376 | −22.165 | 0.50 | 17.27 | C |
| ATOM | 568 | CB | BLEU | A | 62 | 14.129 | −59.446 | −22.183 | 0.50 | 17.43 | C |
| ATOM | 569 | CG | ALEU | A | 62 | 13.359 | −60.517 | −22.828 | 0.50 | 17.98 | C |
| ATOM | 570 | CG | BLEU | A | 62 | 13.720 | −60.919 | −22.126 | 0.50 | 18.62 | C |
| ATOM | 571 | CD1 | ALEU | A | 62 | 13.903 | −61.893 | −22.336 | 0.50 | 17.72 | C |
| ATOM | 572 | CD1 | BLEU | A | 62 | 12.234 | −61.075 | −21.700 | 0.50 | 16.30 | C |
| ATOM | 573 | CD2 | ALEU | A | 62 | 11.819 | −60.393 | −22.550 | 0.50 | 17.18 | C |
| ATOM | 574 | CD2 | BLEU | A | 62 | 14.022 | −61.581 | −23.482 | 0.50 | 17.70 | C |
| ATOM | 575 | C | LEU | A | 62 | 15.992 | −57.756 | −22.289 | 1.00 | 18.54 | C |
| ATOM | 576 | O | LEU | A | 62 | 16.108 | −57.011 | −23.263 | 1.00 | 17.70 | O |
| ATOM | 577 | N | PHE | A | 63 | 16.141 | −57.360 | −21.022 | 1.00 | 16.90 | N |
| ATOM | 578 | CA | PHE | A | 63 | 16.551 | −56.048 | −20.688 | 1.00 | 17.56 | C |
| ATOM | 579 | CB | PHE | A | 63 | 16.629 | −55.862 | −19.186 | 1.00 | 17.32 | C |
| ATOM | 580 | CG | PHE | A | 63 | 17.464 | −54.659 | −18.799 | 1.00 | 21.52 | C |
| ATOM | 581 | CD1 | PHE | A | 63 | 17.041 | −53.382 | −19.147 | 1.00 | 23.75 | C |
| ATOM | 582 | CE1 | PHE | A | 63 | 17.791 | −52.293 | −18.805 | 1.00 | 25.51 | C |
| ATOM | 583 | CZ | PHE | A | 63 | 18.946 | −52.456 | −18.076 | 1.00 | 19.58 | C |
| ATOM | 584 | CE2 | PHE | A | 63 | 19.398 | −53.627 | −17.761 | 1.00 | 24.95 | C |
| ATOM | 585 | CD2 | PHE | A | 63 | 18.619 | −54.804 | −18.113 | 1.00 | 21.83 | C |
| ATOM | 586 | C | PHE | A | 63 | 17.943 | −55.717 | −21.350 | 1.00 | 17.30 | C |
| ATOM | 587 | O | PHE | A | 63 | 18.081 | −54.698 | −22.037 | 1.00 | 18.03 | O |
| ATOM | 588 | N | LEU | A | 64 | 18.922 | −56.588 | −21.158 | 1.00 | 17.10 | N |
| ATOM | 589 | CA | LEU | A | 64 | 20.249 | −56.352 | −21.725 | 1.00 | 19.18 | C |
| ATOM | 590 | CB | LEU | A | 64 | 21.157 | −57.518 | −21.361 | 1.00 | 20.25 | C |
| ATOM | 591 | CG | LEU | A | 64 | 21.639 | −57.357 | −19.858 | 1.00 | 24.98 | C |
| ATOM | 592 | CD1 | LEU | A | 64 | 22.854 | −58.226 | −19.728 | 1.00 | 30.26 | C |
| ATOM | 593 | CD2 | LEU | A | 64 | 22.032 | −55.828 | −19.381 | 1.00 | 26.42 | C |
| ATOM | 594 | C | LEU | A | 64 | 20.205 | −56.251 | −23.257 | 1.00 | 17.32 | C |
| ATOM | 595 | O | LEU | A | 64 | 20.827 | −55.367 | −23.826 | 1.00 | 15.36 | O |
| ATOM | 596 | N | ALA | A | 65 | 19.530 | −57.205 | −23.908 | 1.00 | 14.59 | N |
| ATOM | 597 | CA | ALA | A | 65 | 19.500 | −57.230 | −25.382 | 1.00 | 16.84 | C |
| ATOM | 598 | CB | ALA | A | 65 | 18.605 | −58.408 | −25.903 | 1.00 | 15.91 | C |
| ATOM | 599 | C | ALA | A | 65 | 18.894 | −55.878 | −25.861 | 1.00 | 17.29 | C |
| ATOM | 600 | O | ALA | A | 65 | 19.428 | −55.269 | −26.783 | 1.00 | 16.12 | O |
| ATOM | 601 | N | THR | A | 66 | 17.787 | −55.436 | −25.245 | 1.00 | 16.24 | N |
| ATOM | 602 | CA | ATHR | A | 66 | 17.085 | −54.275 | −25.751 | 0.50 | 16.49 | C |
| ATOM | 603 | CA | BTHR | A | 66 | 17.091 | −54.262 | −25.768 | 0.50 | 18.16 | C |
| ATOM | 604 | CB | ATHR | A | 66 | 15.639 | −54.219 | −25.219 | 0.50 | 16.54 | C |
| ATOM | 605 | CB | BTHR | A | 66 | 15.623 | −54.159 | −25.288 | 0.50 | 18.45 | C |
| ATOM | 606 | OG1 | ATHR | A | 66 | 15.054 | −55.525 | −25.308 | 0.50 | 14.40 | O |
| ATOM | 607 | OG1 | BTHR | A | 66 | 15.598 | −53.940 | −23.872 | 0.50 | 23.96 | O |
| ATOM | 608 | CG2 | ATHR | A | 66 | 14.840 | −53.304 | −26.054 | 0.50 | 13.47 | C |
| ATOM | 609 | CG2 | BTHR | A | 66 | 14.850 | −55.451 | −25.654 | 0.50 | 17.71 | C |
| ATOM | 610 | C | THR | A | 66 | 17.852 | −52.983 | −25.432 | 1.00 | 17.60 | C |
| ATOM | 611 | O | THR | A | 66 | 17.928 | −52.039 | −26.281 | 1.00 | 18.07 | O |
| ATOM | 612 | N | LEU | A | 67 | 18.452 | −52.955 | −24.258 | 1.00 | 17.19 | N |
| ATOM | 613 | CA | LEU | A | 67 | 19.299 | −51.826 | −23.859 | 1.00 | 16.58 | C |
| ATOM | 614 | CB | LEU | A | 67 | 20.005 | −52.135 | −22.551 | 1.00 | 16.20 | C |
| ATOM | 615 | CG | LEU | A | 67 | 21.108 | −51.166 | −22.030 | 1.00 | 17.33 | C |
| ATOM | 616 | CD1 | LEU | A | 67 | 20.488 | −49.743 | −21.679 | 1.00 | 16.06 | C |
| ATOM | 617 | CD2 | LEU | A | 67 | 21.873 | −51.749 | −20.853 | 1.00 | 18.05 | C |
| ATOM | 618 | C | LEU | A | 67 | 20.384 | −51.580 | −24.949 | 1.00 | 17.71 | C |
| ATOM | 619 | O | LEU | A | 67 | 20.625 | −50.431 | −25.353 | 1.00 | 17.60 | O |
| ATOM | 620 | N | TRP | A | 68 | 21.107 | −52.644 | −25.319 | 1.00 | 15.96 | N |
| ATOM | 621 | CA | TRP | A | 68 | 22.205 | −52.529 | −26.278 | 1.00 | 16.78 | C |
| ATOM | 622 | CB | TRP | A | 68 | 23.062 | −53.830 | −26.262 | 1.00 | 17.05 | C |
| ATOM | 623 | CG | TRP | A | 68 | 23.991 | −53.781 | −25.072 | 1.00 | 18.39 | C |
| ATOM | 624 | CD1 | TRP | A | 68 | 23.699 | −54.162 | −23.769 | 1.00 | 19.58 | C |
| ATOM | 625 | NE1 | TRP | A | 68 | 24.777 | −53.912 | −22.967 | 1.00 | 18.86 | N |
| ATOM | 626 | CE2 | TRP | A | 68 | 25.775 | −53.323 | −23.697 | 1.00 | 17.20 | C |
| ATOM | 627 | CD2 | TRP | A | 68 | 25.299 | −53.181 | −25.031 | 1.00 | 18.42 | C |
| ATOM | 628 | CE3 | TRP | A | 68 | 26.151 | −52.601 | −26.019 | 1.00 | 20.65 | C |
| ATOM | 629 | CZ3 | TRP | A | 68 | 27.431 | −52.170 | −25.618 | 1.00 | 19.83 | C |
| ATOM | 630 | CH2 | TRP | A | 68 | 27.866 | −52.310 | −24.257 | 1.00 | 17.97 | C |
| ATOM | 631 | CZ2 | TRP | A | 68 | 27.036 | −52.854 | −23.279 | 1.00 | 15.13 | C |
| ATOM | 632 | C | TRP | A | 68 | 21.691 | −52.135 | −27.683 | 1.00 | 17.12 | C |
| ATOM | 633 | O | TRP | A | 68 | 22.282 | −51.250 | −28.355 | 1.00 | 17.75 | O |
| ATOM | 634 | N | VAL | A | 69 | 20.611 | −52.755 | −28.134 | 1.00 | 17.13 | N |
| ATOM | 635 | CA | VAL | A | 69 | 20.089 | −52.422 | −29.466 | 1.00 | 15.80 | C |
| ATOM | 636 | CB | VAL | A | 69 | 18.978 | −53.437 | −29.958 | 1.00 | 16.41 | C |
| ATOM | 637 | CG1 | VAL | A | 69 | 18.389 | −52.940 | −31.309 | 1.00 | 14.45 | C |
| ATOM | 638 | CG2 | VAL | A | 69 | 19.587 | −54.918 | −30.115 | 1.00 | 14.25 | C |
| ATOM | 639 | C | VAL | A | 69 | 19.558 | −50.995 | −29.473 | 1.00 | 16.34 | C |
| ATOM | 640 | O | VAL | A | 69 | 19.897 | −50.190 | −30.373 | 1.00 | 16.09 | O |
| ATOM | 641 | N | ALA | A | 70 | 18.795 | −50.620 | −28.457 | 1.00 | 16.18 | N |
| ATOM | 642 | CA | ALA | A | 70 | 18.322 | −49.236 | −28.359 | 1.00 | 16.59 | C |
| ATOM | 643 | CB | ALA | A | 70 | 17.292 | −49.022 | −27.202 | 1.00 | 15.43 | C |
| ATOM | 644 | C | ALA | A | 70 | 19.524 | −48.259 | −28.250 | 1.00 | 17.63 | C |
| ATOM | 645 | O | ALA | A | 70 | 19.488 | −47.171 | −28.847 | 1.00 | 16.15 | O |
| ATOM | 646 | N | GLY | A | 71 | 20.552 | −48.617 | −27.476 | 1.00 | 17.57 | N |
| ATOM | 647 | CA | GLY | A | 71 | 21.633 | −47.669 | −27.236 | 1.00 | 17.83 | C |
| ATOM | 648 | C | GLY | A | 71 | 22.510 | −47.450 | −28.479 | 1.00 | 19.25 | C |
| ATOM | 649 | O | GLY | A | 71 | 23.059 | −46.371 | −28.671 | 1.00 | 20.19 | O |
| ATOM | 650 | N | ILE | A | 72 | 22.635 | −48.482 | −29.296 | 1.00 | 19.23 | N |
| ATOM | 651 | CA | AILE | A | 72 | 23.436 | −48.393 | −30.511 | 0.50 | 19.12 | C |
| ATOM | 652 | CA | BILE | A | 72 | 23.421 | −48.451 | −30.530 | 0.50 | 19.30 | C |
| ATOM | 653 | CB | AILE | A | 72 | 24.036 | −49.785 | −30.871 | 0.50 | 19.29 | C |
| ATOM | 654 | CB | BILE | A | 72 | 23.875 | −49.933 | −30.876 | 0.50 | 19.24 | C |
| ATOM | 655 | CG1 | AILE | A | 72 | 24.957 | −50.247 | −29.729 | 0.50 | 19.85 | C |
| ATOM | 656 | CG1 | BILE | A | 72 | 24.767 | −50.461 | −29.740 | 0.50 | 21.26 | C |
| ATOM | 657 | CD1 | AILE | A | 72 | 25.481 | −51.642 | −29.945 | 0.50 | 17.89 | C |
| ATOM | 658 | CD1 | BILE | A | 72 | 26.064 | −49.729 | −29.586 | 0.50 | 22.94 | C |
| ATOM | 659 | CG2 | AILE | A | 72 | 24.757 | −49.748 | −32.234 | 0.50 | 21.32 | C |
| ATOM | 660 | CG2 | BILE | A | 72 | 24.551 | −50.049 | −32.259 | 0.50 | 21.24 | C |
| ATOM | 661 | C | ILE | A | 72 | 22.624 | −47.830 | −31.687 | 1.00 | 18.16 | C |
| ATOM | 662 | O | ILE | A | 72 | 23.164 | −47.034 | −32.485 | 1.00 | 18.23 | O |
| ATOM | 663 | N | PHE | A | 73 | 21.349 | −48.190 | −32.817 | 1.00 | 17.72 | N |
| ATOM | 664 | CA | PHE | A | 73 | 20.570 | −47.769 | −33.014 | 1.00 | 18.09 | C |
| ATOM | 665 | CB | PHE | A | 73 | 19.699 | −48.942 | −33.564 | 1.00 | 17.61 | C |
| ATOM | 666 | CG | PHE | A | 73 | 20.535 | −49.944 | −34.310 | 1.00 | 18.14 | C |
| ATOM | 667 | CD1 | PHE | A | 73 | 20.775 | −49.756 | −35.678 | 1.00 | 17.06 | C |
| ATOM | 668 | CE1 | PHE | A | 73 | 21.596 | −50.670 | −36.419 | 1.00 | 19.45 | C |
| ATOM | 669 | CZ | PHE | A | 73 | 22.259 | −51.718 | −35.738 | 1.00 | 17.54 | C |
| ATOM | 670 | CE2 | PHE | A | 73 | 22.093 | −51.883 | −34.328 | 1.00 | 19.22 | C |
| ATOM | 671 | CD2 | PHE | A | 73 | 21.224 | −50.966 | −33.615 | 1.00 | 17.60 | C |
| ATOM | 672 | C | PHE | A | 73 | 19.753 | −46.491 | −32.795 | 1.00 | 19.06 | C |
| ATOM | 673 | O | PHE | A | 73 | 19.325 | −45.855 | −33.776 | 1.00 | 19.49 | O |
| ATOM | 674 | N | PHE | A | 74 | 19.468 | −46.143 | −31.545 | 1.00 | 17.92 | N |
| ATOM | 675 | CA | PHE | A | 74 | 18.567 | −45.017 | −31.312 | 1.00 | 18.88 | C |
| ATOM | 676 | CB | PHE | A | 74 | 17.286 | −45.372 | −30.483 | 1.00 | 18.92 | C |
| ATOM | 677 | CG | PHE | A | 74 | 16.403 | −44.178 | −30.278 | 1.00 | 21.26 | C |
| ATOM | 678 | CD1 | PHE | A | 74 | 15.452 | −43.848 | −31.227 | 1.00 | 18.16 | C |
| ATOM | 679 | CE1 | PHE | A | 74 | 14.635 | −42.687 | −31.085 | 1.00 | 23.68 | C |
| ATOM | 680 | CZ | PHE | A | 74 | 14.830 | −41.837 | −30.005 | 1.00 | 18.69 | C |
| ATOM | 681 | CE2 | PHE | A | 74 | 15.764 | −42.156 | −29.019 | 1.00 | 20.85 | C |
| ATOM | 682 | CD2 | PHE | A | 74 | 16.559 | −43.345 | −29.147 | 1.00 | 22.36 | C |
| ATOM | 683 | C | PHE | A | 74 | 19.359 | −43.868 | −30.671 | 1.00 | 19.91 | C |
| ATOM | 684 | O | PHE | A | 74 | 19.571 | −42.826 | −31.322 | 1.00 | 19.59 | O |
| ATOM | 685 | N | HIS | A | 75 | 19.847 | −44.049 | −29.421 | 1.00 | 17.73 | N |
| ATOM | 686 | CA | HIS | A | 75 | 20.561 | −42.964 | −28.708 | 1.00 | 17.98 | C |
| ATOM | 687 | CB | HIS | A | 75 | 19.591 | −41.844 | −28.301 | 1.00 | 18.20 | C |
| ATOM | 688 | CG | HIS | A | 75 | 20.251 | −40.633 | −27.705 | 1.00 | 20.13 | C |
| ATOM | 689 | ND1 | HIS | A | 75 | 20.387 | −40.452 | −26.345 | 1.00 | 19.72 | N |
| ATOM | 690 | CE1 | HIS | A | 75 | 21.005 | −39.300 | −26.107 | 1.00 | 21.80 | C |
| ATOM | 691 | NE2 | HIS | A | 75 | 21.249 | −38.708 | −27.269 | 1.00 | 19.17 | N |
| ATOM | 692 | CD2 | HIS | A | 75 | 20.799 | −39.520 | −28.288 | 1.00 | 22.06 | C |
| ATOM | 693 | C | HIS | A | 75 | 21.108 | −43.601 | −27.456 | 1.00 | 19.24 | C |
| ATOM | 694 | O | HIS | A | 75 | 20.307 | −44.145 | −26.645 | 1.00 | 17.21 | O |
| ATOM | 695 | N | GLU | A | 76 | 22.408 | −43.451 | −27.222 | 1.00 | 18.92 | N |
| ATOM | 696 | CA | GLU | A | 76 | 23.028 | −44.115 | −26.080 | 1.00 | 20.30 | C |
| ATOM | 697 | CB | GLU | A | 76 | 24.554 | −43.999 | −26.070 | 1.00 | 20.71 | C |
| ATOM | 698 | CG | GLU | A | 76 | 25.095 | −45.064 | −25.104 | 1.00 | 20.24 | C |
| ATOM | 699 | CD | GLU | A | 76 | 26.505 | −44.772 | −24.564 | 1.00 | 26.40 | C |
| ATOM | 700 | OE1 | GLU | A | 76 | 27.141 | −43.750 | −24.865 | 1.00 | 20.94 | O |
| ATOM | 701 | OE2 | GLU | A | 76 | 27.032 | −45.619 | −23.889 | 1.00 | 22.43 | O |
| ATOM | 702 | C | GLU | A | 76 | 22.481 | −43.617 | −24.744 | 1.00 | 22.00 | C |
| ATOM | 703 | O | GLU | A | 76 | 22.127 | −44.438 | −23.872 | 1.00 | 19.31 | O |
| ATOM | 704 | N | GLY | A | 77 | 22.470 | −42.275 | −24.560 | 1.00 | 18.68 | N |
| ATOM | 705 | CA | GLY | A | 77 | 21.991 | −41.657 | −23.331 | 1.00 | 20.17 | C |
| ATOM | 706 | C | GLY | A | 77 | 20.542 | −42.015 | −22.977 | 1.00 | 20.69 | C |
| ATOM | 707 | O | GLY | A | 77 | 20.233 | −42.338 | −21.809 | 1.00 | 18.70 | O |
| ATOM | 708 | N | ALA | A | 78 | 19.636 | −41.856 | −23.951 | 1.00 | 20.03 | N |
| ATOM | 709 | CA | ALA | A | 78 | 18.229 | −42.204 | −23.728 | 1.00 | 19.13 | C |
| ATOM | 710 | CB | ALA | A | 78 | 17.390 | −41.956 | −25.019 | 1.00 | 18.42 | C |
| ATOM | 711 | C | ALA | A | 78 | 18.094 | −43.694 | −23.277 | 1.00 | 19.09 | C |
| ATOM | 712 | O | ALA | A | 78 | 17.273 | −43.990 | −22.394 | 1.00 | 18.88 | O |
| ATOM | 713 | N | ALA | A | 79 | 18.811 | −44.605 | −23.941 | 1.00 | 17.24 | N |
| ATOM | 714 | CA | ALA | A | 79 | 18.690 | −46.040 | −23.694 | 1.00 | 18.98 | C |
| ATOM | 715 | CB | ALA | A | 79 | 19.520 | −46.852 | −24.712 | 1.00 | 16.12 | C |
| ATOM | 716 | C | ALA | A | 79 | 19.193 | −46.296 | −22.256 | 1.00 | 19.35 | C |
| ATOM | 717 | O | ALA | A | 79 | 18.562 | −47.018 | −21.493 | 1.00 | 17.91 | O |
| ATOM | 718 | N | ALA | A | 80 | 20.308 | −45.661 | −21.894 | 1.00 | 19.28 | N |
| ATOM | 719 | CA | ALA | A | 80 | 20.904 | −45.847 | −20.572 | 1.00 | 19.17 | C |
| ATOM | 720 | CB | ALA | A | 80 | 22.267 | −45.170 | −20.490 | 1.00 | 19.18 | C |
| ATOM | 721 | C | ALA | A | 80 | 19.961 | −45.307 | −19.481 | 1.00 | 20.41 | C |
| ATOM | 722 | O | ALA | A | 80 | 19.808 | −45.969 | −18.429 | 1.00 | 18.59 | O |
| ATOM | 723 | N | LEU | A | 81 | 19.299 | −44.164 | −19.727 | 1.00 | 20.48 | N |
| ATOM | 724 | CA | ALEU | A | 81 | 18.362 | −43.581 | −18.756 | 0.50 | 20.31 | C |
| ATOM | 725 | CA | BLEU | A | 81 | 18.374 | −43.598 | −18.738 | 0.50 | 20.74 | C |
| ATOM | 726 | CB | ALEU | A | 81 | 17.846 | −42.205 | −19.235 | 0.50 | 20.84 | C |
| ATOM | 727 | CB | BLEU | A | 81 | 17.895 | −42.193 | −19.152 | 0.50 | 21.74 | C |
| ATOM | 728 | CG | ALEU | A | 81 | 16.961 | −41.487 | −18.213 | 0.50 | 21.25 | C |
| ATOM | 729 | CG | BLEU | A | 81 | 18.856 | −41.043 | −18.838 | 0.50 | 23.64 | C |
| ATOM | 730 | CD1 | ALEU | A | 81 | 17.748 | −41.264 | −16.909 | 0.50 | 21.72 | C |
| ATOM | 731 | CD1 | BLEU | A | 81 | 18.247 | −39.683 | −19.256 | 0.50 | 26.66 | C |
| ATOM | 732 | CD2 | ALEU | A | 81 | 16.382 | −40.154 | −18.738 | 0.50 | 20.22 | C |
| ATOM | 733 | CD2 | BLEU | A | 81 | 19.273 | −41.055 | −17.353 | 0.50 | 26.64 | C |
| ATOM | 734 | C | LEU | A | 81 | 17.191 | −44.526 | −18.545 | 1.00 | 20.66 | C |
| ATOM | 735 | O | LEU | A | 81 | 16.793 | −44.804 | −17.415 | 1.00 | 19.85 | O |
| ATOM | 736 | N | CYS | A | 82 | 16.640 | −45.025 | −19.651 | 1.00 | 21.19 | N |
| ATOM | 737 | CA | ACYS | A | 82 | 15.521 | −45.972 | −19.573 | 0.50 | 20.47 | C |
| ATOM | 738 | CA | BCYS | A | 82 | 15.521 | −45.926 | −19.562 | 0.50 | 22.15 | C |
| ATOM | 739 | CB | ACYS | A | 82 | 15.060 | −46.422 | −20.950 | 0.50 | 20.52 | C |
| ATOM | 740 | CB | BCYS | A | 82 | 15.015 | −46.191 | −20.972 | 0.50 | 22.49 | C |
| ATOM | 741 | SG | ACYS | A | 82 | 14.219 | −45.219 | −21.911 | 0.50 | 16.00 | S |
| ATOM | 742 | SG | BCYS | A | 82 | 13.427 | −46.924 | −21.064 | 0.50 | 28.68 | S |
| ATOM | 743 | C | CYS | A | 82 | 15.971 | −47.228 | −18.829 | 1.00 | 21.10 | C |
| ATOM | 744 | O | CYS | A | 82 | 15.219 | −47.789 | −18.035 | 1.00 | 21.11 | O |
| ATOM | 745 | N | GLY | A | 83 | 17.200 | −47.672 | −19.104 | 1.00 | 21.21 | N |
| ATOM | 746 | CA | GLY | A | 83 | 17.796 | −48.814 | −18.408 | 1.00 | 19.98 | C |
| ATOM | 747 | C | GLY | A | 83 | 17.875 | −48.634 | −16.895 | 1.00 | 20.49 | C |
| ATOM | 748 | O | GLY | A | 83 | 17.550 | −49.564 | −16.120 | 1.00 | 19.00 | O |
| ATOM | 749 | N | LEU | A | 84 | 18.291 | −47.445 | −16.459 | 1.00 | 20.81 | N |
| ATOM | 750 | CA | ALEU | A | 84 | 18.331 | −47.116 | −15.020 | 0.50 | 20.55 | C |
| ATOM | 751 | CA | BLEU | A | 84 | 18.322 | −47.086 | −15.043 | 0.50 | 21.28 | C |
| ATOM | 752 | CB | ALEU | A | 84 | 18.934 | −45.734 | −14.767 | 0.50 | 20.70 | C |
| ATOM | 753 | CB | BLEU | A | 84 | 18.824 | −45.664 | −14.914 | 0.50 | 22.11 | C |
| ATOM | 754 | CG | ALEU | A | 84 | 20.399 | −45.516 | −15.112 | 0.50 | 16.86 | C |
| ATOM | 755 | CG | BLEU | A | 84 | 19.428 | −45.297 | −13.591 | 0.50 | 21.14 | C |
| ATOM | 756 | CD1 | ALEU | A | 84 | 20.705 | −44.026 | −14.878 | 0.50 | 15.98 | C |
| ATOM | 757 | CD1 | BLEU | A | 84 | 20.548 | −46.316 | −13.252 | 0.50 | 22.18 | C |
| ATOM | 758 | CD2 | ALEU | A | 84 | 21.264 | −46.391 | −14.256 | 0.50 | 16.03 | C |
| ATOM | 759 | CD2 | BLEU | A | 84 | 19.947 | −43.868 | −13.748 | 0.50 | 25.13 | C |
| ATOM | 760 | C | LEU | A | 84 | 16.942 | −47.154 | −14.375 | 1.00 | 22.12 | C |
| ATOM | 761 | O | LEU | A | 84 | 16.780 | −47.699 | −13.264 | 1.00 | 20.87 | O |
| ATOM | 762 | N | VAL | A | 85 | 15.969 | −46.566 | −15.055 | 1.00 | 20.92 | N |
| ATOM | 763 | CA | VAL | A | 85 | 14.598 | −46.584 | −14.585 | 1.00 | 21.52 | C |
| ATOM | 764 | CB | VAL | A | 85 | 13.678 | −45.769 | −15.556 | 1.00 | 21.13 | C |
| ATOM | 765 | CG1 | VAL | A | 85 | 12.215 | −46.002 | −15.196 | 1.00 | 22.01 | C |
| ATOM | 766 | CG2 | VAL | A | 85 | 14.041 | −44.266 | −15.486 | 1.00 | 21.32 | C |
| ATOM | 767 | C | VAL | A | 85 | 14.080 | −48.042 | −14.482 | 1.00 | 19.58 | C |
| ATOM | 768 | O | VAL | A | 85 | 13.444 | −48.401 | −13.507 | 1.00 | 18.76 | O |
| ATOM | 769 | N | TYR | A | 86 | 14.366 | −48.867 | −15.496 | 1.00 | 18.46 | N |
| ATOM | 770 | CA | TYR | A | 86 | 13.984 | −50.292 | −15.451 | 1.00 | 18.04 | C |
| ATOM | 771 | CB | TYR | A | 86 | 14.445 | −50.999 | −16.750 | 1.00 | 17.97 | C |
| ATOM | 772 | CG | TYR | A | 86 | 14.235 | −52.487 | −16.675 | 1.00 | 17.31 | C |
| ATOM | 773 | CD1 | TYR | A | 86 | 12.950 | −53.046 | −16.892 | 1.00 | 17.48 | C |
| ATOM | 774 | CE1 | TYR | A | 86 | 12.724 | −54.431 | −16.799 | 1.00 | 19.08 | C |
| ATOM | 775 | CZ | TYR | A | 86 | 13.762 | −55.232 | −16.405 | 1.00 | 17.56 | C |
| ATOM | 776 | OH | TYR | A | 86 | 13.568 | −56.602 | −16.275 | 1.00 | 18.91 | O |
| ATOM | 777 | CE2 | TYR | A | 86 | 15.047 | −54.700 | −16.142 | 1.00 | 18.55 | C |
| ATOM | 778 | CD2 | TYR | A | 86 | 15.278 | −53.329 | −16.268 | 1.00 | 15.18 | C |
| ATOM | 779 | C | TYR | A | 86 | 14.609 | −50.995 | −14.226 | 1.00 | 18.86 | C |
| ATOM | 780 | O | TYR | A | 86 | 13.933 | −51.744 | −13.480 | 1.00 | 16.48 | O |
| ATOM | 781 | N | LEU | A | 87 | 15.914 | −50.802 | −14.045 | 1.00 | 17.24 | N |
| ATOM | 782 | CA | LEU | A | 87 | 16.581 | −51.474 | −12.947 | 1.00 | 18.96 | C |
| ATOM | 783 | CB | LEU | A | 87 | 18.103 | −51.270 | −13.039 | 1.00 | 17.19 | C |
| ATOM | 784 | CG | LEU | A | 87 | 18.752 | −52.019 | −14.252 | 1.00 | 19.85 | C |
| ATOM | 785 | CD1 | LEU | A | 87 | 20.275 | −51.618 | −14.296 | 1.00 | 17.06 | C |
| ATOM | 786 | CD2 | LEU | A | 87 | 18.554 | −53.597 | −14.221 | 1.00 | 17.77 | C |
| ATOM | 787 | C | LEU | A | 87 | 16.093 | −51.031 | −11.540 | 1.00 | 19.19 | C |
| ATOM | 788 | O | LEU | A | 87 | 15.971 | −51.870 | −10.629 | 1.00 | 19.36 | O |
| ATOM | 789 | N | PHE | A | 88 | 15.903 | −49.733 | −11.360 | 1.00 | 17.43 | N |
| ATOM | 790 | CA | PHE | A | 88 | 15.302 | −49.234 | −10.120 | 1.00 | 20.02 | C |
| ATOM | 791 | CB | PHE | A | 88 | 15.242 | −47.702 | −10.128 | 1.00 | 20.93 | C |
| ATOM | 792 | CG | PHE | A | 88 | 14.782 | −47.131 | −8.810 | 1.00 | 26.16 | C |
| ATOM | 793 | CD1 | PHE | A | 88 | 15.341 | −47.584 | −7.608 | 1.00 | 29.81 | C |
| ATOM | 794 | CE1 | PHE | A | 88 | 14.913 | −47.067 | −6.370 | 1.00 | 32.36 | C |
| ATOM | 795 | CZ | PHE | A | 88 | 13.932 | −46.088 | −6.332 | 1.00 | 28.54 | C |
| ATOM | 796 | CE2 | PHE | A | 88 | 13.356 | −45.636 | −7.507 | 1.00 | 30.57 | C |
| ATOM | 797 | CD2 | PHE | A | 88 | 13.787 | −46.157 | −8.749 | 1.00 | 32.31 | C |
| ATOM | 798 | C | PHE | A | 88 | 13.899 | −49.812 | −9.884 | 1.00 | 18.74 | C |
| ATOM | 799 | O | PHE | A | 88 | 13.556 | −50.179 | −8.773 | 1.00 | 18.39 | O |
| ATOM | 800 | N | ALA | A | 89 | 13.093 | −49.866 | −10.940 | 1.00 | 19.03 | N |
| ATOM | 801 | CA | ALA | A | 89 | 11.801 | −50.497 | −10.885 | 1.00 | 19.00 | C |
| ATOM | 802 | CB | ALA | A | 89 | 10.988 | −50.290 | −12.207 | 1.00 | 18.02 | C |
| ATOM | 803 | C | ALA | A | 89 | 11.912 | −51.968 | −10.499 | 1.00 | 19.43 | C |
| ATOM | 804 | O | ALA | A | 89 | 11.092 | −52.410 | −9.705 | 1.00 | 19.80 | O |
| ATOM | 805 | N | ARG | A | 90 | 12.899 | −52.706 | −11.042 | 1.00 | 18.14 | N |
| ATOM | 806 | CA | ARG | A | 90 | 13.061 | −54.099 | −10.671 | 1.00 | 17.88 | C |
| ATOM | 807 | CB | ARG | A | 90 | 14.020 | −54.851 | −11.588 | 1.00 | 16.67 | C |
| ATOM | 808 | CG | ARG | A | 90 | 13.362 | −55.176 | −12.931 | 1.00 | 17.81 | C |
| ATOM | 809 | CD | ARG | A | 90 | 12.329 | −56.235 | −12.639 | 1.00 | 21.67 | C |
| ATOM | 810 | NE | ARG | A | 90 | 11.629 | −56.725 | −13.814 | 1.00 | 21.80 | N |
| ATOM | 811 | CZ | ARG | A | 90 | 10.778 | −57.759 | −13.798 | 1.00 | 23.73 | C |
| ATOM | 812 | NH1 | ARG | A | 90 | 10.586 | −58.469 | −12.696 | 1.00 | 26.84 | N |
| ATOM | 813 | NH2 | ARG | A | 90 | 10.147 | −58.132 | −14.917 | 1.00 | 28.04 | N |
| ATOM | 814 | C | ARG | A | 90 | 13.496 | −54.276 | −9.213 | 1.00 | 17.36 | C |
| ATOM | 815 | O | ARG | A | 90 | 13.073 | −55.240 | −8.593 | 1.00 | 17.83 | O |
| ATOM | 816 | N | LEU | A | 91 | 14.372 | −53.404 | −8.716 | 1.00 | 17.15 | N |
| ATOM | 817 | CA | LEU | A | 91 | 14.734 | −53.365 | −7.308 | 1.00 | 17.59 | C |
| ATOM | 818 | CB | LEU | A | 91 | 15.761 | −52.272 | −7.001 | 1.00 | 17.32 | C |
| ATOM | 819 | CG | LEU | A | 91 | 16.100 | −52.060 | −5.499 | 1.00 | 20.87 | C |
| ATOM | 820 | CD1 | LEU | A | 91 | 16.577 | −53.397 | −4.813 | 1.00 | 19.29 | C |
| ATOM | 821 | CD2 | LEU | A | 91 | 17.184 | −51.001 | −5.297 | 1.00 | 19.59 | C |
| ATOM | 822 | C | LEU | A | 91 | 13.474 | −53.170 | −6.397 | 1.00 | 18.14 | C |
| ATOM | 823 | O | LEU | A | 91 | 13.311 | −53.901 | −5.401 | 1.00 | 16.88 | O |
| ATOM | 824 | N | ARG | A | 92 | 12.613 | −52.201 | −6.747 | 1.00 | 17.24 | N |
| ATOM | 825 | CA | ARG | A | 92 | 11.374 | −51.943 | −5.980 | 1.00 | 19.42 | C |
| ATOM | 826 | CB | ARG | A | 92 | 10.675 | −50.669 | −6.469 | 1.00 | 19.24 | C |
| ATOM | 827 | CG | ARG | A | 92 | 11.494 | −49.394 | −6.194 | 1.00 | 21.86 | C |
| ATOM | 828 | CD | ARG | A | 92 | 10.623 | −48.083 | −6.361 | 1.00 | 24.79 | C |
| ATOM | 829 | NE | ARG | A | 92 | 9.774 | −48.029 | −7.583 | 1.00 | 34.60 | N |
| ATOM | 830 | CZ | ARG | A | 92 | 10.178 | −47.615 | −8.798 | 1.00 | 35.55 | C |
| ATOM | 831 | NH1 | ARG | A | 92 | 11.433 | −47.250 | −9.005 | 1.00 | 36.03 | N |
| ATOM | 832 | NH2 | ARG | A | 92 | 9.325 | −47.563 | −9.812 | 1.00 | 38.08 | N |
| ATOM | 833 | C | ARG | A | 92 | 10.429 | −53.142 | −6.110 | 1.00 | 18.23 | C |
| ATOM | 834 | O | ARG | A | 92 | 9.799 | −53.542 | −5.139 | 1.00 | 17.11 | O |
| ATOM | 835 | N | TYR | A | 93 | 10.346 | −53.733 | −7.303 | 1.00 | 15.50 | N |
| ATOM | 836 | CA | TYR | A | 93 | 9.538 | −54.955 | −7.502 | 1.00 | 16.06 | C |
| ATOM | 837 | CB | TYR | A | 93 | 9.711 | −55.405 | −8.970 | 1.00 | 15.77 | C |
| ATOM | 838 | CG | TYR | A | 93 | 9.227 | −56.828 | −9.260 | 1.00 | 15.29 | C |
| ATOM | 839 | CD1 | TYR | A | 93 | 7.864 | −57.082 | −9.563 | 1.00 | 15.92 | C |
| ATOM | 840 | CE1 | TYR | A | 93 | 7.400 | −58.395 | −9.819 | 1.00 | 15.43 | C |
| ATOM | 841 | CZ | TYR | A | 93 | 8.309 | −59.447 | −9.815 | 1.00 | 16.21 | C |
| ATOM | 842 | OH | TYR | A | 93 | 7.857 | −60.705 | −10.136 | 1.00 | 18.01 | O |
| ATOM | 843 | CE2 | TYR | A | 93 | 9.638 | −59.243 | −9.529 | 1.00 | 15.76 | C |
| ATOM | 844 | CD2 | TYR | A | 93 | 10.116 | −57.906 | −9.270 | 1.00 | 15.19 | C |
| ATOM | 845 | C | TYR | A | 93 | 9.977 | −56.096 | −6.543 | 1.00 | 16.71 | C |
| ATOM | 846 | O | TYR | A | 93 | 9.161 | −56.769 | −5.872 | 1.00 | 16.64 | O |
| ATOM | 847 | N | PHE | A | 94 | 11.277 | −56.369 | −6.549 | 1.00 | 17.13 | N |
| ATOM | 848 | CA | PHE | A | 94 | 11.841 | −57.432 | −5.675 | 1.00 | 17.54 | C |
| ATOM | 849 | CB | PHE | A | 94 | 13.356 | −57.500 | −5.868 | 1.00 | 18.07 | C |
| ATOM | 850 | CG | PHE | A | 94 | 13.980 | −58.638 | −5.153 | 1.00 | 17.06 | C |
| ATOM | 851 | CD1 | PHE | A | 94 | 14.087 | −59.878 | −5.784 | 1.00 | 20.58 | C |
| ATOM | 852 | CE1 | PHE | A | 94 | 14.713 | −60.987 | −5.106 | 1.00 | 21.45 | C |
| ATOM | 853 | CZ | PHE | A | 94 | 15.148 | −60.800 | −3.786 | 1.00 | 20.36 | C |
| ATOM | 854 | CE2 | PHE | A | 94 | 15.048 | −59.556 | −3.165 | 1.00 | 21.74 | C |
| ATOM | 855 | CD2 | PHE | A | 94 | 14.465 | −58.473 | −3.842 | 1.00 | 19.21 | C |
| ATOM | 856 | C | PHE | A | 94 | 11.561 | −57.205 | −4.195 | 1.00 | 18.80 | C |
| ATOM | 857 | O | PHE | A | 94 | 11.087 | −58.117 | −3.490 | 1.00 | 17.16 | O |
| ATOM | 858 | N | GLN | A | 95 | 11.892 | −55.999 | −3.723 | 1.00 | 19.26 | N |
| ATOM | 859 | CA | GLN | A | 95 | 11.555 | −55.546 | −2.341 | 1.00 | 21.36 | C |
| ATOM | 860 | CB | GLN | A | 95 | 12.069 | −54.112 | −2.119 | 1.00 | 21.28 | C |
| ATOM | 861 | CG | GLN | A | 95 | 13.606 | −54.041 | −2.241 | 1.00 | 24.86 | C |
| ATOM | 862 | CD | GLN | A | 95 | 14.153 | −52.624 | −2.124 | 1.00 | 25.49 | C |
| ATOM | 863 | OE1 | GLN | A | 95 | 13.545 | −51.641 | −2.598 | 1.00 | 29.36 | O |
| ATOM | 864 | NE2 | GLN | A | 95 | 15.321 | −52.516 | −1.501 | 1.00 | 33.43 | N |
| ATOM | 865 | C | GLN | A | 95 | 10.057 | −55.589 | −1.981 | 1.00 | 20.51 | C |
| ATOM | 866 | O | GLN | A | 95 | 9.703 | −56.042 | −0.895 | 1.00 | 19.37 | O |
| ATOM | 867 | N | GLY | A | 96 | 9.196 | −55.152 | −2.909 | 1.00 | 20.23 | N |
| ATOM | 868 | CA | GLY | A | 96 | 7.735 | −55.219 | −2.724 | 1.00 | 19.67 | C |
| ATOM | 869 | C | GLY | A | 96 | 7.279 | −56.661 | −2.575 | 1.00 | 18.78 | C |
| ATOM | 870 | O | GLY | A | 96 | 6.615 | −57.030 | −1.609 | 1.00 | 18.34 | O |
| ATOM | 871 | N | TYR | A | 97 | 7.653 | −57.466 | −3.563 | 1.00 | 17.46 | N |
| ATOM | 872 | CA | TYR | A | 97 | 7.246 | −58.852 | −3.674 | 1.00 | 17.69 | C |
| ATOM | 873 | CB | TYR | A | 97 | 7.879 | −59.508 | −4.946 | 1.00 | 17.70 | C |
| ATOM | 874 | CG | TYR | A | 97 | 7.122 | −60.683 | −5.537 | 1.00 | 18.83 | C |
| ATOM | 875 | CD1 | TYR | A | 97 | 6.134 | −61.358 | −4.803 | 1.00 | 20.76 | C |
| ATOM | 876 | CE1 | TYR | A | 97 | 5.424 | −62.435 | −5.353 | 1.00 | 19.31 | C |
| ATOM | 877 | CZ | TYR | A | 97 | 5.754 | −62.912 | −6.599 | 1.00 | 20.19 | C |
| ATOM | 878 | OH | TYR | A | 97 | 5.063 | −63.998 | −7.107 | 1.00 | 21.90 | O |
| ATOM | 879 | CE2 | TYR | A | 97 | 6.762 | −62.279 | −7.363 | 1.00 | 17.58 | C |
| ATOM | 880 | CD2 | TYR | A | 97 | 7.451 | −61.186 | −6.816 | 1.00 | 18.08 | C |
| ATOM | 881 | C | TYR | A | 97 | 7.680 | −59.597 | −2.412 | 1.00 | 17.60 | C |
| ATOM | 882 | O | TYR | A | 97 | 6.902 | −60.400 | −1.879 | 1.00 | 16.26 | O |
| ATOM | 883 | N | ALA | A | 98 | 8.904 | −59.346 | −1.928 | 1.00 | 17.96 | N |
| ATOM | 884 | CA | ALA | A | 98 | 9.366 | −60.011 | −0.697 | 1.00 | 19.75 | C |
| ATOM | 885 | CB | ALA | A | 98 | 10.796 | −59.562 | −0.346 | 1.00 | 19.79 | C |
| ATOM | 886 | C | ALA | A | 98 | 8.372 | −59.767 | 0.502 | 1.00 | 20.41 | C |
| ATOM | 887 | O | ALA | A | 98 | 8.128 | −60.678 | 1.345 | 1.00 | 21.07 | O |
| ATOM | 888 | N | ARG | A | 99 | 7.777 | −58.568 | 0.551 | 1.00 | 19.91 | N |
| ATOM | 889 | CA | ARG | A | 99 | 6.821 | −58.186 | 1.614 | 1.00 | 21.60 | C |
| ATOM | 890 | CB | ARG | A | 99 | 6.761 | −56.663 | 1.835 | 1.00 | 21.87 | C |
| ATOM | 891 | CG | ARG | A | 99 | 8.070 | −56.072 | 2.335 | 1.00 | 25.72 | C |
| ATOM | 892 | CD | ARG | A | 99 | 8.051 | −54.516 | 2.237 | 1.00 | 26.32 | C |
| ATOM | 893 | NE | ARG | A | 99 | 6.967 | −53.939 | 3.035 | 1.00 | 35.80 | N |
| ATOM | 894 | CZ | ARG | A | 99 | 6.330 | −52.802 | 2.734 | 1.00 | 39.54 | C |
| ATOM | 895 | NH1 | ARG | A | 99 | 6.666 | −52.122 | 1.636 | 1.00 | 40.73 | N |
| ATOM | 896 | NH2 | ARG | A | 99 | 5.351 | −52.349 | 3.528 | 1.00 | 39.32 | N |
| ATOM | 897 | C | ARG | A | 99 | 5.418 | −58.683 | 1.312 | 1.00 | 19.55 | C |
| ATOM | 898 | O | ARG | A | 99 | 4.750 | −59.212 | 2.197 | 1.00 | 18.16 | O |
| ATOM | 899 | N | SER | A | 100 | 4.988 | −58.563 | 0.051 | 1.00 | 18.32 | N |
| ATOM | 900 | CA | ASER | A | 100 | 3.640 | −58.976 | −0.318 | 0.50 | 17.53 | C |
| ATOM | 901 | CA | BSER | A | 100 | 3.687 | −59.100 | −0.332 | 0.50 | 17.23 | C |
| ATOM | 902 | CB | ASER | A | 100 | 2.651 | −57.928 | 0.238 | 0.50 | 16.94 | C |
| ATOM | 903 | CB | BSER | A | 100 | 2.535 | −58.308 | 0.306 | 0.50 | 16.59 | C |
| ATOM | 904 | OG | ASER | A | 100 | 1.577 | −57.637 | −0.624 | 0.50 | 14.44 | O |
| ATOM | 905 | OG | BSER | A | 100 | 2.369 | −57.063 | −0.329 | 0.50 | 12.87 | O |
| ATOM | 906 | C | SER | A | 100 | 3.524 | −59.151 | −1.834 | 1.00 | 17.22 | C |
| ATOM | 907 | O | SER | A | 100 | 4.073 | −58.318 | −2.586 | 1.00 | 17.03 | O |
| ATOM | 908 | N | ALA | A | 101 | 2.818 | −60.190 | −2.281 | 1.00 | 16.93 | N |
| ATOM | 909 | CA | ALA | A | 101 | 2.537 | −60.338 | −3.706 | 1.00 | 17.51 | C |
| ATOM | 910 | CB | ALA | A | 101 | 1.614 | −61.491 | −3.959 | 1.00 | 16.81 | C |
| ATOM | 911 | C | ALA | A | 101 | 1.943 | −59.037 | −4.234 | 1.00 | 18.49 | C |
| ATOM | 912 | O | ALA | A | 101 | 2.372 | −58.526 | −5.280 | 1.00 | 18.21 | O |
| ATOM | 913 | N | GLN | A | 102 | 0.979 | −58.472 | −3.515 | 1.00 | 17.00 | N |
| ATOM | 914 | CA | GLN | A | 102 | 0.317 | −57.230 | −3.992 | 1.00 | 18.27 | C |
| ATOM | 915 | CB | GLN | A | 102 | −0.888 | −56.897 | −3.103 | 1.00 | 17.88 | C |
| ATOM | 916 | CG | GLN | A | 102 | −1.859 | −55.885 | −3.700 | 1.00 | 19.53 | C |
| ATOM | 917 | CD | GLN | A | 102 | −1.444 | −54.431 | −3.510 | 1.00 | 23.04 | C |
| ATOM | 918 | OE1 | GLN | A | 102 | −0.656 | −54.092 | −2.616 | 1.00 | 23.83 | O |
| ATOM | 919 | NE2 | GLN | A | 102 | −1.993 | −53.554 | −4.353 | 1.00 | 28.57 | N |
| ATOM | 920 | C | GLN | A | 102 | 1.251 | −56.007 | −4.142 | 1.00 | 17.78 | C |
| ATOM | 921 | O | GLN | A | 102 | 1.079 | −55.172 | −5.050 | 1.00 | 17.61 | O |
| ATOM | 922 | N | LEU | A | 103 | 2.226 | −55.886 | −3.254 | 1.00 | 18.32 | N |
| ATOM | 923 | CA | LEU | A | 103 | 3.163 | −54.764 | −3.327 | 1.00 | 18.80 | C |
| ATOM | 924 | CB | LEU | A | 103 | 3.917 | −54.569 | −1.994 | 1.00 | 19.13 | C |
| ATOM | 925 | CG | LEU | A | 103 | 3.130 | −54.030 | −0.778 | 1.00 | 20.28 | C |
| ATOM | 926 | CD1 | LEU | A | 103 | 4.002 | −54.062 | 0.452 | 1.00 | 21.70 | C |
| ATOM | 927 | CD2 | LEU | A | 103 | 2.522 | −52.644 | −0.962 | 1.00 | 20.12 | C |
| ATOM | 928 | C | LEU | A | 103 | 4.153 | −54.877 | −4.508 | 1.00 | 18.54 | C |
| ATOM | 929 | O | LEU | A | 103 | 4.830 | −53.903 | −4.832 | 1.00 | 19.62 | O |
| ATOM | 930 | N | ARG | A | 104 | 4.231 | −56.041 | −5.149 | 1.00 | 17.51 | N |
| ATOM | 931 | CA | ARG | A | 104 | 5.091 | −56.176 | −6.345 | 1.00 | 18.29 | C |
| ATOM | 932 | CB | ARG | A | 104 | 5.338 | −57.633 | −6.721 | 1.00 | 16.19 | C |
| ATOM | 933 | CG | ARG | A | 104 | 4.289 | −58.190 | −7.703 | 1.00 | 16.36 | C |
| ATOM | 934 | CD | ARG | A | 104 | 4.403 | −59.709 | −7.817 | 1.00 | 18.46 | C |
| ATOM | 935 | NE | ARG | A | 104 | 3.564 | −60.300 | −8.867 | 1.00 | 17.38 | N |
| ATOM | 936 | CZ | ARG | A | 104 | 2.255 | −60.495 | −8.789 | 1.00 | 22.43 | C |
| ATOM | 937 | NH1 | ARG | A | 104 | 1.620 | −61.043 | −9.802 | 1.00 | 21.31 | N |
| ATOM | 938 | NH2 | ARG | A | 104 | 1.572 | −60.145 | −7.699 | 1.00 | 22.90 | N |
| ATOM | 939 | C | ARG | A | 104 | 4.469 | −55.453 | −7.549 | 1.00 | 19.41 | C |
| ATOM | 940 | O | ARG | A | 104 | 5.187 | −55.104 | −8.501 | 1.00 | 19.36 | O |
| ATOM | 941 | N | LEU | A | 105 | 3.166 | −55.174 | −7.492 | 1.00 | 18.48 | N |
| ATOM | 942 | CA | LEU | A | 105 | 2.430 | −54.780 | −8.716 | 1.00 | 18.92 | C |
| ATOM | 943 | CB | LEU | A | 105 | 0.894 | −55.010 | −8.612 | 1.00 | 18.99 | C |
| ATOM | 944 | CG | LEU | A | 105 | 0.383 | −56.466 | −8.488 | 1.00 | 20.55 | C |
| ATOM | 945 | CD1 | LEU | A | 105 | −1.127 | −56.595 | −7.931 | 1.00 | 19.75 | C |
| ATOM | 946 | CD2 | LEU | A | 105 | 0.626 | −57.244 | −9.826 | 1.00 | 20.09 | C |
| ATOM | 947 | C | LEU | A | 105 | 2.757 | −53.386 | −9.275 | 1.00 | 19.99 | C |
| ATOM | 948 | O | LEU | A | 105 | 3.012 | −53.267 | −10.479 | 1.00 | 17.61 | O |
| ATOM | 949 | N | ALA | A | 106 | 2.720 | −52.339 | −8.440 | 1.00 | 19.37 | N |
| ATOM | 950 | CA | ALA | A | 106 | 3.105 | −51.001 | −8.960 | 1.00 | 20.85 | C |
| ATOM | 951 | CB | ALA | A | 106 | 2.943 | −49.860 | −7.898 | 1.00 | 20.53 | C |
| ATOM | 952 | C | ALA | A | 106 | 4.516 | −50.985 | −9.587 | 1.00 | 20.65 | C |
| ATOM | 953 | O | ALA | A | 106 | 4.650 | −50.533 | −10.723 | 1.00 | 21.70 | O |
| ATOM | 954 | N | PRO | A | 107 | 5.576 | −51.428 | −8.856 | 1.00 | 20.32 | N |
| ATOM | 955 | CA | PRO | A | 107 | 6.868 | −51.411 | −9.562 | 1.00 | 20.33 | C |
| ATOM | 956 | CB | PRO | A | 107 | 7.908 | −51.757 | −8.463 | 1.00 | 21.24 | C |
| ATOM | 957 | CG | PRO | A | 107 | 7.078 | −52.441 | −7.373 | 1.00 | 21.13 | C |
| ATOM | 958 | CD | PRO | A | 107 | 5.711 | −51.828 | −7.431 | 1.00 | 20.65 | C |
| ATOM | 959 | C | PRO | A | 107 | 6.978 | −52.377 | −10.751 | 1.00 | 19.27 | C |
| ATOM | 960 | O | PRO | A | 107 | 7.735 | −52.069 | −11.672 | 1.00 | 19.36 | O |
| ATOM | 961 | N | LEU | A | 108 | 6.253 | −53.494 | −10.752 | 1.00 | 18.15 | N |
| ATOM | 962 | CA | LEU | A | 108 | 6.191 | −54.318 | −11.939 | 1.00 | 18.98 | C |
| ATOM | 963 | CB | LEU | A | 108 | 5.346 | −55.580 | −11.762 | 1.00 | 18.90 | C |
| ATOM | 964 | CG | LEU | A | 108 | 5.147 | −56.518 | −12.971 | 1.00 | 18.87 | C |
| ATOM | 965 | CD1 | LEU | A | 108 | 6.538 | −57.138 | −13.476 | 1.00 | 16.05 | C |
| ATOM | 966 | CD2 | LEU | A | 108 | 4.151 | −57.637 | −12.549 | 1.00 | 18.53 | C |
| ATOM | 967 | C | LEU | A | 108 | 5.708 | −53.531 | −13.158 | 1.00 | 19.14 | C |
| ATOM | 968 | O | LEU | A | 108 | 6.321 | −53.632 | −14.231 | 1.00 | 19.08 | O |
| ATOM | 969 | N | TYR | A | 109 | 4.631 | −52.776 | −13.001 | 1.00 | 19.18 | N |
| ATOM | 970 | CA | TYR | A | 109 | 4.086 | −51.963 | −14.086 | 1.00 | 20.04 | C |
| ATOM | 971 | CB | TYR | A | 109 | 2.753 | −51.239 | −13.692 | 1.00 | 21.52 | C |
| ATOM | 972 | CG | TYR | A | 109 | 1.644 | −52.092 | −13.134 | 1.00 | 25.19 | C |
| ATOM | 973 | CD1 | TYR | A | 109 | 1.370 | −53.364 | −13.646 | 1.00 | 25.04 | C |
| ATOM | 974 | CE1 | TYR | A | 109 | 0.321 | −54.135 | −13.136 | 1.00 | 28.04 | C |
| ATOM | 975 | CZ | TYR | A | 109 | −0.479 | −53.630 | −12.128 | 1.00 | 25.98 | C |
| ATOM | 976 | OH | TYR | A | 109 | −1.497 | −54.404 | −11.622 | 1.00 | 30.37 | O |
| ATOM | 977 | CE2 | TYR | A | 109 | −0.271 | −52.356 | −11.615 | 1.00 | 26.89 | C |
| ATOM | 978 | CD2 | TYR | A | 109 | 0.802 | −51.589 | −12.114 | 1.00 | 23.64 | C |
| ATOM | 979 | C | TYR | A | 109 | 5.090 | −50.903 | −14.499 | 1.00 | 19.77 | C |
| ATOM | 980 | O | TYR | A | 109 | 5.223 | −50.618 | −15.703 | 1.00 | 19.68 | O |
| ATOM | 981 | N | ALA | A | 110 | 5.801 | −50.302 | −13.536 | 1.00 | 18.69 | N |
| ATOM | 982 | CA | ALA | A | 110 | 6.864 | −49.350 | −13.917 | 1.00 | 18.23 | C |
| ATOM | 983 | CB | ALA | A | 110 | 7.408 | −48.650 | −12.670 | 1.00 | 17.00 | C |
| ATOM | 984 | C | ALA | A | 110 | 8.009 | −50.006 | −14.733 | 1.00 | 19.53 | C |
| ATOM | 985 | O | ALA | A | 110 | 8.533 | −49.404 | −15.727 | 1.00 | 19.35 | O |
| ATOM | 986 | N | SER | A | 111 | 8.394 | −51.228 | −14.358 | 1.00 | 18.40 | N |
| ATOM | 987 | CA | SER | A | 111 | 9.478 | −51.923 | −15.049 | 1.00 | 19.16 | C |
| ATOM | 988 | CB | SER | A | 111 | 9.909 | −53.150 | −14.234 | 1.00 | 18.49 | C |
| ATOM | 989 | OG | SER | A | 111 | 9.017 | −54.219 | −14.473 | 1.00 | 21.25 | O |
| ATOM | 990 | C | SER | A | 111 | 8.990 | −52.274 | −16.484 | 1.00 | 19.02 | C |
| ATOM | 991 | O | SER | A | 111 | 9.772 | −52.220 | −17.446 | 1.00 | 19.51 | O |
| ATOM | 992 | N | ALA | A | 112 | 7.717 | −52.654 | −16.629 | 1.00 | 19.25 | N |
| ATOM | 993 | CA | ALA | A | 112 | 7.179 | −52.979 | −17.960 | 1.00 | 19.05 | C |
| ATOM | 994 | CB | ALA | A | 112 | 5.777 | −53.639 | −17.893 | 1.00 | 20.36 | C |
| ATOM | 995 | C | ALA | A | 112 | 7.122 | −51.700 | −18.836 | 1.00 | 19.81 | C |
| ATOM | 996 | O | ALA | A | 112 | 7.477 | −51.768 | −20.021 | 1.00 | 19.05 | O |
| ATOM | 997 | N | ARG | A | 113 | 6.673 | −50.563 | −18.287 | 1.00 | 17.74 | N |
| ATOM | 998 | CA | ARG | A | 113 | 6.652 | −49.353 | −19.087 | 1.00 | 17.00 | C |
| ATOM | 999 | CB | ARG | A | 113 | 6.061 | −48.196 | −18.298 | 1.00 | 17.26 | C |
| ATOM | 1000 | CG | ARG | A | 113 | 4.568 | −48.332 | −18.063 | 1.00 | 16.75 | C |
| ATOM | 1001 | CD | ARG | A | 113 | 3.935 | −47.099 | −17.409 | 1.00 | 19.58 | C |
| ATOM | 1002 | NE | ARG | A | 113 | 4.479 | −46.774 | −16.091 | 1.00 | 22.43 | N |
| ATOM | 1003 | CZ | ARG | A | 113 | 3.924 | −47.134 | −14.924 | 1.00 | 26.06 | C |
| ATOM | 1004 | NH1 | ARG | A | 113 | 2.805 | −47.874 | −14.895 | 1.00 | 25.88 | N |
| ATOM | 1005 | NH2 | ARG | A | 113 | 4.490 | −46.752 | −13.777 | 1.00 | 20.88 | N |
| ATOM | 1006 | C | ARG | A | 113 | 8.118 | −49.034 | −19.561 | 1.00 | 17.10 | C |
| ATOM | 1007 | O | ARG | A | 113 | 8.351 | −48.662 | −20.728 | 1.00 | 15.69 | O |
| ATOM | 1008 | N | ALA | A | 114 | 9.104 | −49.167 | −18.656 | 1.00 | 16.30 | N |
| ATOM | 1009 | CA | ALA | A | 114 | 10.484 | −48.825 | −19.025 | 1.00 | 17.08 | C |
| ATOM | 1010 | CB | ALA | A | 114 | 11.411 | −48.740 | −17.775 | 1.00 | 16.19 | C |
| ATOM | 1011 | C | ALA | A | 114 | 11.025 | −49.835 | −20.072 | 1.00 | 16.54 | C |
| ATOM | 1012 | O | ALA | A | 114 | 11.700 | −49.443 | −21.067 | 1.00 | 16.39 | O |
| ATOM | 1013 | N | LEU | A | 115 | 10.703 | −51.114 | −19.904 | 1.00 | 15.42 | N |
| ATOM | 1014 | CA | LEU | A | 115 | 11.185 | −52.091 | −20.882 | 1.00 | 16.02 | C |
| ATOM | 1015 | CB | LEU | A | 115 | 10.997 | −53.512 | −20.375 | 1.00 | 14.14 | C |
| ATOM | 1016 | CG | LEU | A | 115 | 11.579 | −54.643 | −21.234 | 1.00 | 16.65 | C |
| ATOM | 1017 | CD1 | LEU | A | 115 | 13.111 | −54.447 | −21.524 | 1.00 | 18.28 | C |
| ATOM | 1018 | CD2 | LEU | A | 115 | 11.299 | −55.970 | −20.544 | 1.00 | 16.70 | C |
| ATOM | 1019 | C | LEU | A | 115 | 10.522 | −51.870 | −22.268 | 1.00 | 16.10 | C |
| ATOM | 1020 | O | LEU | A | 115 | 11.187 | −51.971 | −23.332 | 1.00 | 13.56 | O |
| ATOM | 1021 | N | TRP | A | 116 | 9.215 | −51.586 | −22.266 | 1.00 | 15.42 | N |
| ATOM | 1022 | CA | TRP | A | 116 | 8.561 | −51.387 | −23.532 | 1.00 | 16.32 | C |
| ATOM | 1023 | CB | TRP | A | 116 | 7.023 | −51.421 | −23.390 | 1.00 | 17.44 | C |
| ATOM | 1024 | CG | TRP | A | 116 | 6.522 | −52.852 | −23.299 | 1.00 | 21.79 | C |
| ATOM | 1025 | CD1 | TRP | A | 116 | 6.052 | −53.495 | −22.178 | 1.00 | 26.43 | C |
| ATOM | 1026 | NE1 | TRP | A | 116 | 5.682 | −54.789 | −22.487 | 1.00 | 26.72 | N |
| ATOM | 1027 | CE2 | TRP | A | 116 | 5.942 | −55.019 | −23.816 | 1.00 | 27.72 | C |
| ATOM | 1028 | CD2 | TRP | A | 116 | 6.474 | −53.819 | −24.362 | 1.00 | 24.70 | C |
| ATOM | 1029 | CE3 | TRP | A | 116 | 6.818 | −53.785 | −25.726 | 1.00 | 25.83 | C |
| ATOM | 1030 | CZ3 | TRP | A | 116 | 6.614 | −54.934 | −26.507 | 1.00 | 24.24 | C |
| ATOM | 1031 | CH2 | TRP | A | 116 | 6.076 | −56.123 | −25.939 | 1.00 | 24.26 | C |
| ATOM | 1032 | CZ2 | TRP | A | 116 | 5.730 | −56.187 | −24.606 | 1.00 | 25.52 | C |
| ATOM | 1033 | C | TRP | A | 116 | 9.037 | −50.107 | −24.220 | 1.00 | 15.53 | C |
| ATOM | 1034 | O | TRP | A | 116 | 9.086 | −50.051 | −25.449 | 1.00 | 14.21 | O |
| ATOM | 1035 | N | LEU | A | 117 | 9.399 | −49.089 | −23.445 | 1.00 | 15.82 | N |
| ATOM | 1036 | CA | LEU | A | 117 | 9.986 | −47.877 | −24.042 | 1.00 | 17.22 | C |
| ATOM | 1037 | CB | LEU | A | 117 | 10.197 | −46.796 | −22.970 | 1.00 | 17.54 | C |
| ATOM | 1038 | CG | LEU | A | 117 | 10.651 | −45.398 | −23.417 | 1.00 | 21.03 | C |
| ATOM | 1039 | CD1 | LEU | A | 117 | 9.805 | −44.830 | −24.609 | 1.00 | 20.36 | C |
| ATOM | 1040 | CD2 | LEU | A | 117 | 10.638 | −44.437 | −22.221 | 1.00 | 19.16 | C |
| ATOM | 1041 | C | LEU | A | 117 | 11.328 | −48.244 | −24.712 | 1.00 | 17.27 | C |
| ATOM | 1042 | O | LEU | A | 117 | 11.609 | −47.797 | −25.846 | 1.00 | 15.68 | O |
| ATOM | 1043 | N | LEU | A | 118 | 12.142 | −49.059 | −24.041 | 1.00 | 16.55 | N |
| ATOM | 1044 | CA | LEU | A | 118 | 13.392 | −49.546 | −24.663 | 1.00 | 17.55 | C |
| ATOM | 1045 | CB | LEU | A | 118 | 14.273 | −50.408 | −23.731 | 1.00 | 18.46 | C |
| ATOM | 1046 | CG | LEU | A | 118 | 15.191 | −49.703 | −22.756 | 1.00 | 21.32 | C |
| ATOM | 1047 | CD1 | LEU | A | 118 | 15.748 | −50.742 | −21.729 | 1.00 | 19.72 | C |
| ATOM | 1048 | CD2 | LEU | A | 118 | 16.305 | −48.908 | −23.516 | 1.00 | 16.31 | C |
| ATOM | 1049 | C | LEU | A | 118 | 13.138 | −50.336 | −25.925 | 1.00 | 17.98 | C |
| ATOM | 1050 | O | LEU | A | 118 | 13.866 | −50.163 | −26.874 | 1.00 | 17.15 | O |
| ATOM | 1051 | N | VAL | A | 119 | 12.117 | −51.209 | −25.924 | 1.00 | 15.44 | N |
| ATOM | 1052 | CA | VAL | A | 119 | 11.753 | −51.942 | −27.113 | 1.00 | 15.10 | C |
| ATOM | 1053 | CB | VAL | A | 119 | 10.651 | −52.958 | −26.806 | 1.00 | 15.45 | C |
| ATOM | 1054 | CG1 | VAL | A | 119 | 9.996 | −53.488 | −28.104 | 1.00 | 16.21 | C |
| ATOM | 1055 | CG2 | VAL | A | 119 | 11.233 | −54.153 | −25.931 | 1.00 | 14.62 | C |
| ATOM | 1056 | C | VAL | A | 119 | 11.370 | −50.934 | −28.255 | 1.00 | 15.50 | C |
| ATOM | 1057 | O | VAL | A | 119 | 11.804 | −51.079 | −29.425 | 1.00 | 15.89 | O |
| ATOM | 1058 | N | ALA | A | 120 | 10.595 | −49.917 | −27.920 | 1.00 | 14.80 | N |
| ATOM | 1059 | CA | ALA | A | 120 | 10.122 | −48.958 | −28.923 | 1.00 | 15.99 | C |
| ATOM | 1060 | CB | ALA | A | 120 | 9.072 | −47.985 | −28.303 | 1.00 | 14.80 | C |
| ATOM | 1061 | C | ALA | A | 120 | 11.318 | −48.179 | −29.504 | 1.00 | 16.00 | C |
| ATOM | 1062 | O | ALA | A | 120 | 11.379 | −47.945 | −30.720 | 1.00 | 16.41 | O |
| ATOM | 1063 | N | LEU | A | 121 | 12.283 | −47.816 | −28.654 | 1.00 | 16.45 | N |
| ATOM | 1064 | CA | LEU | A | 121 | 13.465 | −47.091 | −29.135 | 1.00 | 17.42 | C |
| ATOM | 1065 | CB | LEU | A | 121 | 14.317 | −46.544 | −27.976 | 1.00 | 16.44 | C |
| ATOM | 1066 | CG | LEU | A | 121 | 13.680 | −45.572 | −26.984 | 1.00 | 15.90 | C |
| ATOM | 1067 | CD1 | LEU | A | 121 | 14.771 | −44.996 | −26.054 | 1.00 | 18.04 | C |
| ATOM | 1068 | CD2 | LEU | A | 121 | 12.933 | −44.435 | −27.714 | 1.00 | 18.56 | C |
| ATOM | 1069 | C | LEU | A | 121 | 14.324 | −47.982 | −30.022 | 1.00 | 17.33 | C |
| ATOM | 1070 | O | LEU | A | 121 | 14.815 | −47.523 | −31.078 | 1.00 | 16.03 | O |
| ATOM | 1071 | N | ALA | A | 122 | 14.514 | −49.231 | −29.597 | 1.00 | 16.06 | N |
| ATOM | 1072 | CA | ALA | A | 122 | 15.196 | −50.217 | −30.436 | 1.00 | 16.34 | C |
| ATOM | 1073 | CB | ALA | A | 122 | 15.286 | −51.566 | −29.722 | 1.00 | 14.36 | C |
| ATOM | 1074 | C | ALA | A | 122 | 14.495 | −50.351 | −31.826 | 1.00 | 16.12 | C |
| ATOM | 1075 | O | ALA | A | 122 | 15.183 | −50.332 | −32.849 | 1.00 | 17.12 | O |
| ATOM | 1076 | N | ALA | A | 123 | 13.182 | −50.506 | −31.829 | 1.00 | 14.68 | N |
| ATOM | 1077 | CA | ALA | A | 123 | 12.384 | −50.662 | −33.065 | 1.00 | 16.66 | C |
| ATOM | 1078 | CB | ALA | A | 123 | 10.879 | −50.939 | −32.782 | 1.00 | 15.93 | C |
| ATOM | 1079 | C | ALA | A | 123 | 12.513 | −49.414 | −33.952 | 1.00 | 16.24 | C |
| ATOM | 1080 | O | ALA | A | 123 | 12.775 | −49.555 | −35.136 | 1.00 | 14.50 | O |
| ATOM | 1081 | N | LEU | A | 124 | 12.434 | −48.218 | −33.350 | 1.00 | 16.22 | N |
| ATOM | 1082 | CA | LEU | A | 124 | 12.528 | −47.002 | −34.144 | 1.00 | 16.16 | C |
| ATOM | 1083 | CB | LEU | A | 124 | 12.214 | −45.765 | −33.268 | 1.00 | 16.21 | C |
| ATOM | 1084 | CG | LEU | A | 124 | 10.745 | −45.643 | −32.820 | 1.00 | 18.15 | C |
| ATOM | 1085 | CD1 | LEU | A | 124 | 10.578 | −44.457 | −31.794 | 1.00 | 20.40 | C |
| ATOM | 1086 | CD2 | LEU | A | 124 | 9.857 | −45.414 | −34.070 | 1.00 | 19.87 | C |
| ATOM | 1087 | C | LEU | A | 124 | 13.941 | −46.862 | −34.724 | 1.00 | 17.00 | C |
| ATOM | 1088 | O | LEU | A | 124 | 14.077 | −46.444 | −35.850 | 1.00 | 17.42 | O |
| ATOM | 1089 | N | GLY | A | 125 | 14.982 | −47.169 | −33.940 | 1.00 | 16.35 | N |
| ATOM | 1090 | CA | GLY | A | 125 | 16.341 | −47.076 | −34.412 | 1.00 | 16.72 | C |
| ATOM | 1091 | C | GLY | A | 125 | 16.601 | −48.052 | −35.568 | 1.00 | 16.98 | C |
| ATOM | 1092 | O | GLY | A | 125 | 17.245 | −47.704 | −36.594 | 1.00 | 16.12 | O |
| ATOM | 1093 | N | LEU | A | 126 | 16.099 | −49.275 | −35.434 | 1.00 | 15.83 | N |
| ATOM | 1094 | CA | LEU | A | 126 | 16.178 | −50.251 | −36.556 | 1.00 | 15.68 | C |
| ATOM | 1095 | CB | LEU | A | 126 | 15.763 | −51.657 | −36.095 | 1.00 | 14.31 | C |
| ATOM | 1096 | CG | LEU | A | 126 | 16.811 | −52.334 | −35.153 | 1.00 | 12.27 | C |
| ATOM | 1097 | CD1 | LEU | A | 126 | 16.224 | −53.623 | −34.530 | 1.00 | 12.70 | C |
| ATOM | 1098 | CD2 | LEU | A | 126 | 18.123 | −52.643 | −35.856 | 1.00 | 14.00 | C |
| ATOM | 1099 | C | LEU | A | 126 | 15.379 | −49.819 | −37.820 | 1.00 | 17.51 | C |
| ATOM | 1100 | O | LEU | A | 126 | 15.847 | −49.981 | −38.942 | 1.00 | 16.62 | O |
| ATOM | 1101 | N | LEU | A | 127 | 14.175 | −49.296 | −37.622 | 1.00 | 16.40 | N |
| ATOM | 1102 | CA | LEU | A | 127 | 13.392 | −48.803 | −38.711 | 1.00 | 19.64 | C |
| ATOM | 1103 | CB | LEU | A | 127 | 12.060 | −48.301 | −38.175 | 1.00 | 20.16 | C |
| ATOM | 1104 | CG | LEU | A | 127 | 10.743 | −48.841 | −38.708 | 1.00 | 31.03 | C |
| ATOM | 1105 | CD1 | LEU | A | 127 | 9.840 | −49.172 | −37.438 | 1.00 | 34.65 | C |
| ATOM | 1106 | CD2 | LEU | A | 127 | 10.780 | −50.067 | −39.690 | 1.00 | 35.41 | C |
| ATOM | 1107 | C | LEU | A | 127 | 14.166 | −47.668 | −39.451 | 1.00 | 18.74 | C |
| ATOM | 1108 | O | LEU | A | 127 | 14.200 | −47.671 | −40.691 | 1.00 | 16.96 | O |
| ATOM | 1109 | N | ALA | A | 128 | 14.796 | −46.760 | −38.687 | 1.00 | 18.28 | N |
| ATOM | 1110 | CA | ALA | A | 128 | 15.575 | −45.653 | −39.225 | 1.00 | 20.21 | C |
| ATOM | 1111 | CB | ALA | A | 128 | 16.082 | −44.713 | −38.128 | 1.00 | 18.89 | C |
| ATOM | 1112 | C | ALA | A | 128 | 16.748 | −46.184 | −40.034 | 1.00 | 19.78 | C |
| ATOM | 1113 | O | ALA | A | 128 | 17.078 | −45.631 | −41.090 | 1.00 | 19.15 | O |
| ATOM | 1114 | N | HIS | A | 129 | 17.334 | −47.281 | −39.569 | 1.00 | 18.53 | N |
| ATOM | 1115 | CA | HIS | A | 129 | 18.390 | −47.936 | −40.278 | 1.00 | 17.66 | C |
| ATOM | 1116 | CB | HIS | A | 129 | 19.128 | −48.951 | −39.368 | 1.00 | 17.32 | C |
| ATOM | 1117 | CG | HIS | A | 129 | 20.207 | −49.688 | −40.083 | 1.00 | 15.76 | C |
| ATOM | 1118 | ND1 | HIS | A | 129 | 21.499 | −49.207 | −40.181 | 1.00 | 18.37 | N |
| ATOM | 1119 | CE1 | HIS | A | 129 | 22.208 | −50.012 | −40.951 | 1.00 | 17.41 | C |
| ATOM | 1120 | NE2 | HIS | A | 129 | 21.433 | −51.011 | −41.335 | 1.00 | 17.22 | N |
| ATOM | 1121 | CD2 | HIS | A | 129 | 20.174 | −50.830 | −40.812 | 1.00 | 16.46 | C |
| ATOM | 1122 | C | HIS | A | 129 | 17.959 | −48.598 | −41.615 | 1.00 | 18.81 | C |
| ATOM | 1123 | O | HIS | A | 129 | 18.658 | −48.456 | −42.604 | 1.00 | 18.01 | O |
| ATOM | 1124 | N | PHE | A | 130 | 16.883 | −49.375 | −41.618 | 1.00 | 16.57 | N |
| ATOM | 1125 | CA | PHE | A | 130 | 16.515 | −50.193 | −42.766 | 1.00 | 17.26 | C |
| ATOM | 1126 | CB | PHE | A | 130 | 15.903 | −51.507 | −42.328 | 1.00 | 16.04 | C |
| ATOM | 1127 | CG | PHE | A | 130 | 16.898 | −52.486 | −41.755 | 1.00 | 16.37 | C |
| ATOM | 1128 | CD1 | PHE | A | 130 | 17.796 | −53.141 | −42.603 | 1.00 | 16.48 | C |
| ATOM | 1129 | CE1 | PHE | A | 130 | 18.698 | −54.070 | −42.097 | 1.00 | 17.18 | C |
| ATOM | 1130 | CZ | PHE | A | 130 | 18.712 | −54.361 | −40.741 | 1.00 | 13.56 | C |
| ATOM | 1131 | CE2 | PHE | A | 130 | 17.855 | −53.709 | −39.877 | 1.00 | 13.23 | C |
| ATOM | 1132 | CD2 | PHE | A | 130 | 16.906 | −52.788 | −40.389 | 1.00 | 16.06 | C |
| ATOM | 1133 | C | PHE | A | 130 | 15.513 | −49.549 | −43.767 | 1.00 | 18.83 | C |
| ATOM | 1134 | O | PHE | A | 130 | 15.552 | −49.888 | −44.943 | 1.00 | 17.40 | O |
| ATOM | 1135 | N | LEU | A | 131 | 14.608 | −48.708 | −43.275 | 1.00 | 18.27 | N |
| ATOM | 1136 | CA | LEU | A | 131 | 13.485 | −48.269 | −44.080 | 1.00 | 21.82 | C |
| ATOM | 1137 | CB | LEU | A | 131 | 12.370 | −47.660 | −43.205 | 1.00 | 21.96 | C |
| ATOM | 1138 | CG | LEU | A | 131 | 11.015 | −47.296 | −43.820 | 1.00 | 26.12 | C |
| ATOM | 1139 | CD1 | LEU | A | 131 | 10.370 | −48.451 | −44.570 | 1.00 | 24.52 | C |
| ATOM | 1140 | CD2 | LEU | A | 131 | 10.118 | −46.786 | −42.672 | 1.00 | 25.75 | C |
| ATOM | 1141 | C | LEU | A | 131 | 13.907 | −47.372 | −45.270 | 1.00 | 20.69 | C |
| ATOM | 1142 | O | LEU | A | 131 | 13.422 | −47.579 | −46.363 | 1.00 | 19.41 | O |
| ATOM | 1143 | N | PRO | A | 132 | 14.807 | −46.397 | −45.065 | 1.00 | 20.35 | N |
| ATOM | 1144 | CA | PRO | A | 132 | 15.179 | −45.565 | −46.237 | 1.00 | 21.17 | C |
| ATOM | 1145 | CB | PRO | A | 132 | 16.169 | −44.527 | −45.642 | 1.00 | 20.75 | C |
| ATOM | 1146 | CG | PRO | A | 132 | 15.712 | −44.417 | −44.183 | 1.00 | 21.79 | C |
| ATOM | 1147 | CD | PRO | A | 132 | 15.431 | −45.898 | −43.838 | 1.00 | 21.43 | C |
| ATOM | 1148 | C | PRO | A | 132 | 15.787 | −46.378 | −47.393 | 1.00 | 21.10 | C |
| ATOM | 1149 | O | PRO | A | 132 | 15.299 | −46.253 | −48.544 | 1.00 | 21.92 | O |
| ATOM | 1150 | N | ALA | A | 133 | 16.818 | −47.201 | −47.138 | 1.00 | 19.00 | N |
| ATOM | 1151 | CA | ALA | A | 133 | 17.344 | −48.048 | −48.217 | 1.00 | 18.47 | C |
| ATOM | 1152 | CB | ALA | A | 133 | 18.518 | −48.909 | −47.744 | 1.00 | 17.70 | C |
| ATOM | 1153 | C | ALA | A | 133 | 16.269 | −48.954 | −48.827 | 1.00 | 17.62 | C |
| ATOM | 1154 | O | ALA | A | 133 | 16.280 | −49.222 | −50.033 | 1.00 | 17.85 | O |
| ATOM | 1155 | N | ALA | A | 134 | 15.365 | −49.477 | −48.006 | 1.00 | 16.95 | N |
| ATOM | 1156 | CA | ALA | A | 134 | 14.323 | −50.370 | −48.539 | 1.00 | 16.86 | C |
| ATOM | 1157 | CB | ALA | A | 134 | 13.561 | −51.110 | −47.364 | 1.00 | 16.97 | C |
| ATOM | 1158 | C | ALA | A | 134 | 13.333 | −49.605 | −49.454 | 1.00 | 16.39 | C |
| ATOM | 1159 | O | ALA | A | 134 | 12.941 | −50.098 | −50.545 | 1.00 | 16.55 | O |
| ATOM | 1160 | N | LEU | A | 135 | 12.933 | −48.408 | −49.034 | 1.00 | 15.20 | N |
| ATOM | 1161 | CA | LEU | A | 135 | 12.029 | −47.603 | −49.846 | 1.00 | 15.77 | C |
| ATOM | 1162 | CB | LEU | A | 135 | 11.573 | −46.361 | −49.084 | 1.00 | 16.04 | C |
| ATOM | 1163 | CG | LEU | A | 135 | 10.594 | −46.641 | −47.903 | 1.00 | 19.35 | C |
| ATOM | 1164 | CD1 | LEU | A | 135 | 10.386 | −45.419 | −46.960 | 1.00 | 18.88 | C |
| ATOM | 1165 | CD2 | LEU | A | 135 | 9.207 | −47.183 | −48.417 | 1.00 | 22.05 | C |
| ATOM | 1166 | C | LEU | A | 135 | 12.709 | −47.210 | −51.162 | 1.00 | 16.61 | C |
| ATOM | 1167 | O | LEU | A | 135 | 12.070 | −47.243 | −52.241 | 1.00 | 16.10 | O |
| ATOM | 1168 | N | ARG | A | 136 | 13.991 | −46.804 | −51.075 | 1.00 | 15.57 | N |
| ATOM | 1169 | CA | ARG | A | 136 | 14.763 | −46.510 | −52.269 | 1.00 | 16.20 | C |
| ATOM | 1170 | CB | ARG | A | 136 | 16.161 | −46.028 | −51.936 | 1.00 | 15.74 | C |
| ATOM | 1171 | CG | ARG | A | 136 | 16.849 | −45.714 | −53.265 | 1.00 | 20.45 | C |
| ATOM | 1172 | CD | ARG | A | 136 | 18.125 | −45.020 | −53.101 | 1.00 | 23.94 | C |
| ATOM | 1173 | NE | ARG | A | 136 | 19.055 | −45.805 | −52.341 | 1.00 | 24.94 | N |
| ATOM | 1174 | CZ | ARG | A | 136 | 20.337 | −45.497 | −52.189 | 1.00 | 33.93 | C |
| ATOM | 1175 | NH1 | ARG | A | 136 | 20.852 | −44.420 | −52.788 | 1.00 | 30.17 | N |
| ATOM | 1176 | NH2 | ARG | A | 136 | 21.112 | −46.263 | −51.406 | 1.00 | 36.07 | N |
| ATOM | 1177 | C | ARG | A | 136 | 14.862 | −47.649 | −53.268 | 1.00 | 16.02 | C |
| ATOM | 1178 | O | ARG | A | 136 | 14.647 | −47.428 | −54.447 | 1.00 | 16.39 | O |
| ATOM | 1179 | N | ALA | A | 137 | 15.139 | −48.875 | −52.795 | 1.00 | 16.10 | N |
| ATOM | 1180 | CA | ALA | A | 137 | 15.273 | −50.026 | −53.676 | 1.00 | 17.40 | C |
| ATOM | 1181 | CB | ALA | A | 137 | 15.646 | −51.280 | −52.866 | 1.00 | 17.86 | C |
| ATOM | 1182 | C | ALA | A | 137 | 13.966 | −50.271 | −54.414 | 1.00 | 17.27 | C |
| ATOM | 1183 | O | ALA | A | 137 | 13.955 | −50.569 | −55.602 | 1.00 | 19.04 | O |
| ATOM | 1184 | N | ALA | A | 138 | 12.874 | −50.115 | −53.692 | 1.00 | 17.64 | N |
| ATOM | 1185 | CA | ALA | A | 138 | 11.538 | −50.310 | −54.227 | 1.00 | 18.93 | C |
| ATOM | 1186 | CB | ALA | A | 138 | 10.521 | −50.282 | −53.087 | 1.00 | 17.91 | C |
| ATOM | 1187 | C | ALA | A | 138 | 11.184 | −49.267 | −55.271 | 1.00 | 18.76 | C |
| ATOM | 1188 | O | ALA | A | 138 | 10.620 | −49.579 | −56.337 | 1.00 | 18.73 | O |
| ATOM | 1189 | N | LEU | A | 139 | 11.487 | −48.015 | −54.963 | 1.00 | 19.53 | N |
| ATOM | 1190 | CA | LEU | A | 139 | 11.342 | −46.931 | −55.938 | 1.00 | 20.25 | C |
| ATOM | 1191 | CB | LEU | A | 139 | 11.686 | −45.590 | −55.301 | 1.00 | 21.38 | C |
| ATOM | 1192 | CG | LEU | A | 139 | 10.641 | −45.145 | −54.268 | 1.00 | 25.15 | C |
| ATOM | 1193 | CD1 | LEU | A | 139 | 11.216 | −44.148 | −53.214 | 1.00 | 28.08 | C |
| ATOM | 1194 | CD2 | LEU | A | 139 | 9.405 | −44.534 | −55.026 | 1.00 | 30.13 | C |
| ATOM | 1195 | C | LEU | A | 139 | 12.173 | −47.132 | −57.186 | 1.00 | 20.70 | C |
| ATOM | 1196 | O | LEU | A | 139 | 11.669 | −46.939 | −58.309 | 1.00 | 20.05 | O |
| ATOM | 1197 | N | LEU | A | 140 | 13.437 | −47.507 | −57.015 | 1.00 | 21.05 | N |
| ATOM | 1198 | CA | LEU | A | 140 | 14.324 | −47.812 | −58.147 | 1.00 | 21.95 | C |
| ATOM | 1199 | CB | LEU | A | 140 | 15.742 | −48.164 | −57.659 | 1.00 | 20.37 | C |
| ATOM | 1200 | CG | LEU | A | 140 | 16.573 | −47.022 | −57.030 | 1.00 | 21.37 | C |
| ATOM | 1201 | CD1 | LEU | A | 140 | 17.914 | −47.544 | −56.486 | 1.00 | 19.31 | C |
| ATOM | 1202 | CD2 | LEU | A | 140 | 16.833 | −45.831 | −57.959 | 1.00 | 18.14 | C |
| ATOM | 1203 | C | LEU | A | 140 | 13.763 | −48.910 | −59.082 | 1.00 | 24.36 | C |
| ATOM | 1204 | O | LEU | A | 140 | 13.821 | −48.756 | −60.316 | 1.00 | 25.16 | O |
| ATOM | 1205 | N | GLY | A | 141 | 13.186 | −49.966 | −58.502 | 1.00 | 26.81 | N |
| ATOM | 1206 | CA | GLY | A | 141 | 12.579 | −51.064 | −59.253 | 1.00 | 31.10 | C |
| ATOM | 1207 | C | GLY | A | 141 | 11.416 | −50.610 | −60.122 | 1.00 | 34.70 | C |
| ATOM | 1208 | O | GLY | A | 141 | 11.243 | −51.084 | −61.225 | 1.00 | 32.81 | O |
| ATOM | 1209 | N | ARG | A | 142 | 10.618 | −49.699 | −59.590 | 1.00 | 39.98 | N |
| ATOM | 1210 | CA | ARG | A | 142 | 9.636 | −48.912 | −60.339 | 1.00 | 47.21 | C |
| ATOM | 1211 | CB | ARG | A | 142 | 8.992 | −47.999 | −59.301 | 1.00 | 46.48 | C |
| ATOM | 1212 | CG | ARG | A | 142 | 7.530 | −47.769 | −59.365 | 1.00 | 47.57 | C |
| ATOM | 1213 | CD | ARG | A | 142 | 6.880 | −48.470 | −58.163 | 1.00 | 49.36 | C |
| ATOM | 1214 | NE | ARG | A | 142 | 7.334 | −49.859 | −58.068 | 1.00 | 48.64 | N |
| ATOM | 1215 | CZ | ARG | A | 142 | 7.128 | −50.786 | −59.006 | 1.00 | 46.60 | C |
| ATOM | 1216 | NH1 | ARG | A | 142 | 7.592 | −52.021 | −58.821 | 1.00 | 45.63 | N |
| ATOM | 1217 | NH2 | ARG | A | 142 | 6.478 | −50.476 | −60.127 | 1.00 | 44.87 | N |
| ATOM | 1218 | C | ARG | A | 142 | 10.209 | −47.987 | −61.453 | 1.00 | 52.70 | C |
| ATOM | 1219 | O | ARG | A | 142 | 9.432 | −47.576 | −62.295 | 1.00 | 52.86 | O |
| ATOM | 1220 | N | LEU | A | 143 | 11.525 | −47.593 | −61.427 | 1.00 | 62.69 | N |
| ATOM | 1221 | CA | LEU | A | 143 | 11.920 | −46.384 | −62.212 | 1.00 | 64.17 | C |
| ATOM | 1222 | CB | LEU | A | 143 | 13.239 | −45.745 | −61.870 | 1.00 | 65.77 | C |
| ATOM | 1223 | CG | LEU | A | 143 | 13.363 | −44.199 | −62.043 | 1.00 | 66.48 | C |
| ATOM | 1224 | CD1 | LEU | A | 143 | 14.866 | −43.842 | −62.267 | 1.00 | 64.58 | C |
| ATOM | 1225 | CD2 | LEU | A | 143 | 12.594 | −43.410 | −60.921 | 1.00 | 67.46 | C |
| ATOM | 1226 | C | LEU | A | 143 | 11.538 | −46.348 | −63.776 | 1.00 | 64.64 | C |
| ATOM | 1227 | O | LEU | A | 143 | 10.895 | −45.373 | −64.173 | 1.00 | 63.76 | O |
| ATOM | 1228 | N | ARG | A | 144 | 11.711 | −47.340 | −64.674 | 1.00 | 65.81 | N |
| ATOM | 1229 | CA | ARG | A | 144 | 11.472 | −48.800 | −64.608 | 1.00 | 67.60 | C |
| ATOM | 1230 | CB | ARG | A | 144 | 12.270 | −49.486 | −63.489 | 1.00 | 67.22 | C |
| ATOM | 1231 | CG | ARG | A | 144 | 13.611 | −50.012 | −63.961 | 1.00 | 67.14 | C |
| ATOM | 1232 | CD | ARG | A | 144 | 14.762 | −49.098 | −63.576 | 1.00 | 66.63 | C |
| ATOM | 1233 | NE | ARG | A | 144 | 15.617 | −49.646 | −62.511 | 1.00 | 66.32 | N |
| ATOM | 1234 | CZ | ARG | A | 144 | 16.045 | −50.908 | −62.426 | 1.00 | 65.42 | C |
| ATOM | 1235 | NH1 | ARG | A | 144 | 15.727 | −51.797 | −63.358 | 1.00 | 65.86 | N |
| ATOM | 1236 | NH2 | ARG | A | 144 | 16.814 | −51.284 | −61.406 | 1.00 | 64.70 | N |
| ATOM | 1237 | C | ARG | A | 144 | 9.976 | −49.283 | −64.765 | 1.00 | 68.80 | C |
| ATOM | 1238 | O | ARG | A | 144 | 9.690 | −50.525 | −64.722 | 1.00 | 70.00 | O |
| ATOM | 1239 | N | THR | A | 145 | 9.034 | −48.412 | −65.177 | 1.00 | 69.68 | N |
| ATOM | 1240 | CA | THR | A | 145 | 9.096 | −47.477 | −66.337 | 1.00 | 69.98 | C |
| ATOM | 1241 | CB | THR | A | 145 | 8.231 | −46.175 | −66.181 | 1.00 | 70.25 | C |
| ATOM | 1242 | OG1 | THR | A | 145 | 8.896 | −45.301 | −65.144 | 1.00 | 66.72 | O |
| ATOM | 1243 | CG2 | THR | A | 145 | 6.614 | −46.590 | −65.622 | 1.00 | 72.03 | C |
| ATOM | 1244 | C | THR | A | 145 | 10.431 | −47.269 | −67.053 | 1.00 | 69.95 | C |
| ATOM | 1245 | O | THR | A | 145 | 11.307 | −48.148 | −67.025 | 1.00 | 70.10 | O |
| ATOM | 1246 | N | GLY | A | 146 | 10.558 | −46.129 | −67.737 | 1.00 | 69.96 | N |
| ATOM | 1247 | CA | GLY | A | 146 | 11.813 | −45.762 | −68.406 | 1.00 | 69.80 | C |
| ATOM | 1248 | C | GLY | A | 146 | 12.044 | −46.547 | −69.680 | 1.00 | 69.62 | C |
| ATOM | 1249 | O | GLY | A | 146 | 11.374 | −47.551 | −69.941 | 1.00 | 69.65 | O |
| ATOM | 1250 | N | GLY | A | 147 | 13.010 | −46.095 | −70.472 | 1.00 | 69.53 | N |
| ATOM | 1251 | CA | GLY | A | 147 | 13.159 | −46.584 | −71.841 | 1.00 | 69.36 | C |
| ATOM | 1252 | C | GLY | A | 147 | 12.007 | −46.020 | −72.654 | 1.00 | 69.18 | C |
| ATOM | 1253 | O | GLY | A | 147 | 11.626 | −46.574 | −73.694 | 1.00 | 69.16 | O |
| ATOM | 1254 | N | GLY | A | 148 | 11.456 | −44.913 | −72.149 | 1.00 | 69.04 | N |
| ATOM | 1255 | CA | GLY | A | 148 | 10.290 | −44.249 | −72.722 | 1.00 | 68.96 | C |
| ATOM | 1256 | C | GLY | A | 148 | 9.878 | −43.109 | −71.809 | 1.00 | 68.92 | C |
| ATOM | 1257 | O | GLY | A | 148 | 10.465 | −42.027 | −71.867 | 1.00 | 68.87 | O |
| ATOM | 1258 | N | ALA | A | 149 | 8.866 | −43.372 | −70.973 | 1.00 | 68.88 | N |
| ATOM | 1259 | CA | ALA | A | 149 | 8.372 | −42.473 | −69.898 | 1.00 | 68.77 | C |
| ATOM | 1260 | CB | ALA | A | 149 | 9.305 | −42.526 | −68.667 | 1.00 | 68.75 | C |
| ATOM | 1261 | C | ALA | A | 149 | 7.990 | −41.014 | −70.252 | 1.00 | 68.67 | C |
| ATOM | 1262 | O | ALA | A | 149 | 8.352 | −40.418 | −71.288 | 1.00 | 68.52 | O |
| ATOM | 1263 | OXT | ALA | A | 149 | 7.277 | −40.367 | −69.463 | 1.00 | 68.63 | O |
| ATOM | 1264 | NI | NI | B | 1 | 36.095 | −48.980 | −36.056 | 0.33 | 33.22 | NI |
| ATOM | 1265 | NI | NI | B | 2 | 49.606 | −49.606 | −35.233 | 0.30 | 36.96 | NI |
| ATOM | 1266 | NI | NI | B | 3 | 42.873 | −48.586 | −37.142 | 1.00 | 99.29 | NI |
| ATOM | 1267 | O6′ | LMT | C | 1 | −3.605 | −54.087 | −16.040 | 1.00 | 111.04 | O |
| ATOM | 1268 | C6′ | LMT | C | 1 | −3.173 | −52.979 | −16.843 | 1.00 | 111.18 | C |
| ATOM | 1269 | C5′ | LMT | C | 1 | −1.887 | −52.371 | −16.274 | 1.00 | 110.81 | C |
| ATOM | 1270 | C4′ | LMT | C | 1 | −2.229 | −51.126 | −15.451 | 1.00 | 111.06 | C |
| ATOM | 1271 | C3′ | LMT | C | 1 | −1.040 | −50.157 | −15.294 | 1.00 | 110.38 | C |
| ATOM | 1272 | O3′ | LMT | C | 1 | −1.525 | −48.852 | −14.957 | 1.00 | 110.45 | O |
| ATOM | 1273 | C2′ | LMT | C | 1 | −0.198 | −50.035 | −16.572 | 1.00 | 109.55 | C |
| ATOM | 1274 | O2′ | LMT | C | 1 | 0.917 | −49.129 | −16.433 | 1.00 | 108.76 | O |
| ATOM | 1275 | O1* | LMT | C | 1 | −2.731 | −51.685 | −14.228 | 1.00 | 112.51 | O |
| ATOM | 1276 | C1* | LMT | C | 1 | −3.206 | −50.871 | −13.139 | 1.00 | 114.57 | C |
| ATOM | 1277 | O5* | LMT | C | 1 | −3.559 | −49.507 | −13.446 | 1.00 | 115.19 | O |
| ATOM | 1278 | C5* | LMT | C | 1 | −3.688 | −48.684 | −12.266 | 1.00 | 116.27 | C |
| ATOM | 1279 | C6* | LMT | C | 1 | −2.468 | −47.764 | −12.142 | 1.00 | 116.61 | C |
| ATOM | 1280 | O6* | LMT | C | 1 | −2.877 | −46.401 | −11.930 | 1.00 | 117.41 | O |
| ATOM | 1281 | C4* | LMT | C | 1 | −3.875 | −49.535 | −10.989 | 1.00 | 116.40 | C |
| ATOM | 1282 | O4* | LMT | C | 1 | −4.351 | −48.741 | −9.886 | 1.00 | 116.75 | O |
| ATOM | 1283 | C3* | LMT | C | 1 | −4.784 | −50.759 | −11.194 | 1.00 | 115.88 | C |
| ATOM | 1284 | O3* | LMT | C | 1 | −4.708 | −51.608 | −10.038 | 1.00 | 116.21 | O |
| ATOM | 1285 | C2* | LMT | C | 1 | −4.383 | −51.559 | −12.435 | 1.00 | 115.09 | C |
| ATOM | 1286 | O2* | LMT | C | 1 | −5.498 | −51.680 | −13.327 | 1.00 | 115.04 | O |
| ATOM | 1287 | O5′ | LMT | C | 1 | −0.953 | −52.135 | −17.348 | 1.00 | 110.41 | O |
| ATOM | 1288 | C1′ | LMT | C | 1 | 0.250 | −51.426 | −17.017 | 1.00 | 108.78 | C |
| ATOM | 1289 | O1′ | LMT | C | 1 | 1.106 | −51.303 | −18.164 | 1.00 | 106.47 | O |
| ATOM | 1290 | C1 | LMT | C | 1 | 2.109 | −52.311 | −18.332 | 1.00 | 102.89 | C |
| ATOM | 1291 | C2 | LMT | C | 1 | 2.537 | −52.390 | −19.803 | 1.00 | 100.07 | C |
| ATOM | 1292 | C3 | LMT | C | 1 | 2.902 | −51.031 | −20.400 | 1.00 | 98.03 | C |
| ATOM | 1293 | C4 | LMT | C | 1 | 3.459 | −51.122 | −21.819 | 1.00 | 96.60 | C |
| ATOM | 1294 | C5 | LMT | C | 1 | 2.360 | −51.137 | −22.884 | 1.00 | 96.20 | C |
| ATOM | 1295 | C6 | LMT | C | 1 | 2.922 | −51.294 | −24.299 | 1.00 | 95.35 | C |
| ATOM | 1296 | C7 | LMT | C | 1 | 1.961 | −50.786 | −25.367 | 1.00 | 93.83 | C |
| ATOM | 1302 | O6′ | LMT | C | 2 | −2.031 | −55.756 | −17.808 | 1.00 | 80.58 | O |
| ATOM | 1303 | C6′ | LMT | C | 2 | −0.935 | −55.782 | −16.876 | 1.00 | 81.63 | C |
| ATOM | 1304 | C5′ | LMT | C | 2 | 0.251 | −56.588 | −17.425 | 1.00 | 81.73 | C |
| ATOM | 1305 | C4′ | LMT | C | 2 | 1.210 | −57.054 | −16.314 | 1.00 | 80.51 | C |
| ATOM | 1306 | C3′ | LMT | C | 2 | 2.449 | −57.718 | −16.932 | 1.00 | 80.88 | C |
| ATOM | 1307 | O3′ | LMT | C | 2 | 3.394 | −58.131 | −15.930 | 1.00 | 79.99 | O |
| ATOM | 1308 | C2′ | LMT | C | 2 | 3.110 | −56.879 | −18.031 | 1.00 | 81.76 | C |
| ATOM | 1309 | O2′ | LMT | C | 2 | 4.179 | −57.638 | −18.607 | 1.00 | 79.74 | O |
| ATOM | 1310 | O1* | LMT | C | 2 | 0.544 | −58.048 | −15.535 | 1.00 | 78.22 | O |
| ATOM | 1311 | C1* | LMT | C | 2 | 0.619 | −57.870 | −14.117 | 1.00 | 76.69 | C |
| ATOM | 1312 | O5* | LMT | C | 2 | −0.584 | −57.225 | −13.653 | 1.00 | 77.46 | O |
| ATOM | 1313 | C5* | LMT | C | 2 | −1.784 | −58.030 | −13.671 | 1.00 | 77.02 | C |
| ATOM | 1314 | C6* | LMT | C | 2 | −2.979 | −57.176 | −13.253 | 1.00 | 76.34 | C |
| ATOM | 1315 | O6* | LMT | C | 2 | −2.670 | −56.552 | −12.006 | 1.00 | 73.92 | O |
| ATOM | 1316 | C4* | LMT | C | 2 | −1.561 | −59.263 | −12.785 | 1.00 | 76.97 | C |
| ATOM | 1317 | O4* | LMT | C | 2 | −2.735 | −60.071 | −12.643 | 1.00 | 78.21 | O |
| ATOM | 1318 | C3* | LMT | C | 2 | −0.438 | −60.065 | −13.427 | 1.00 | 75.75 | C |
| ATOM | 1319 | O3* | LMT | C | 2 | −0.293 | −61.311 | −12.744 | 1.00 | 76.68 | O |
| ATOM | 1320 | C2* | LMT | C | 2 | 0.844 | −59.217 | −13.410 | 1.00 | 75.44 | C |
| ATOM | 1321 | O2* | LMT | C | 2 | 1.963 | −59.916 | −13.988 | 1.00 | 70.89 | O |
| ATOM | 1322 | O5′ | LMT | C | 2 | 0.957 | −55.816 | −18.416 | 1.00 | 83.00 | O |
| ATOM | 1323 | C1′ | LMT | C | 2 | 2.039 | −56.516 | −19.082 | 1.00 | 83.33 | C |
| ATOM | 1324 | O1′ | LMT | C | 2 | 2.579 | −55.760 | −20.194 | 1.00 | 84.31 | O |
| ATOM | 1325 | C1 | LMT | C | 2 | 1.663 | −55.598 | −21.289 | 1.00 | 85.13 | C |
| ATOM | 1326 | C2 | LMT | C | 2 | 2.370 | −55.721 | −22.634 | 1.00 | 85.59 | C |
| ATOM | 1327 | C3 | LMT | C | 2 | 1.395 | −55.459 | −23.782 | 1.00 | 86.75 | C |
| ATOM | 1328 | C4 | LMT | C | 2 | 2.041 | −55.667 | −25.155 | 1.00 | 87.26 | C |
| ATOM | 1329 | C5 | LMT | C | 2 | 2.231 | −54.342 | −25.894 | 1.00 | 87.53 | C |
| ATOM | 1330 | C6 | LMT | C | 2 | 3.114 | −54.507 | −27.130 | 1.00 | 88.69 | C |
| ATOM | 1331 | C7 | LMT | C | 2 | 2.602 | −53.707 | −28.327 | 1.00 | 89.75 | C |
| ATOM | 1332 | C8 | LMT | C | 2 | 3.551 | −53.780 | −29.524 | 1.00 | 90.13 | C |
| ATOM | 1333 | C9 | LMT | C | 2 | 3.066 | −54.744 | −30.606 | 1.00 | 90.48 | C |
| ATOM | 1334 | C10 | LMT | C | 2 | 4.237 | −55.559 | −31.157 | 1.00 | 91.10 | C |
| ATOM | 1335 | C11 | LMT | C | 2 | 3.783 | −56.629 | −32.150 | 1.00 | 91.45 | C |
| ATOM | 1336 | C12 | LMT | C | 2 | 4.278 | −56.320 | −33.547 | 1.00 | 91.61 | C |
| ATOM | 1349 | C11 | ACD | D | 1 | 12.505 | −43.590 | −40.585 | 1.00 | 54.69 | C |
| ATOM | 1350 | C12 | ACD | D | 1 | 12.648 | −43.056 | −39.353 | 1.00 | 54.82 | C |
| ATOM | 1351 | C13 | ACD | D | 1 | 11.694 | −43.364 | −38.211 | 1.00 | 50.69 | C |
| ATOM | 1352 | C14 | ACD | D | 1 | 12.041 | −42.637 | −36.904 | 1.00 | 49.48 | C |
| ATOM | 1353 | C15 | ACD | D | 1 | 13.312 | −42.542 | −36.529 | 1.00 | 47.66 | C |
| ATOM | 1354 | C16 | ACD | D | 1 | 13.824 | −41.869 | −35.272 | 1.00 | 47.27 | C |
| ATOM | 1355 | C17 | ACD | D | 1 | 15.059 | −42.648 | −34.847 | 1.00 | 41.50 | C |
| ATOM | 1356 | C18 | ACD | D | 1 | 16.371 | −41.947 | −35.187 | 1.00 | 43.04 | C |
| ATOM | 1357 | C19 | ACD | D | 1 | 17.554 | −42.814 | −34.886 | 1.00 | 39.17 | C |
| ATOM | 1358 | C20 | ACD | D | 1 | 18.724 | −42.571 | −35.826 | 1.00 | 42.68 | C |
| ATOM | 1362 | C2 | PLM | E | 1 | 20.760 | −40.781 | −11.317 | 1.00 | 74.16 | C |
| ATOM | 1363 | C3 | PLM | E | 1 | 21.878 | −41.789 | −11.595 | 1.00 | 74.65 | C |
| ATOM | 1364 | C4 | PLM | E | 1 | 22.104 | −42.705 | −10.387 | 1.00 | 74.01 | C |
| ATOM | 1365 | C5 | PLM | E | 1 | 23.002 | −43.919 | −10.663 | 1.00 | 72.47 | C |
| ATOM | 1366 | C6 | PLM | E | 1 | 22.740 | −45.015 | −9.621 | 1.00 | 71.56 | C |
| ATOM | 1367 | C7 | PLM | E | 1 | 23.972 | −45.825 | −9.214 | 1.00 | 70.48 | C |
| ATOM | 1378 | C1 | PLM | E | 2 | 9.374 | −55.594 | −40.579 | 1.00 | 71.72 | C |
| ATOM | 1380 | C2 | PLM | E | 2 | 10.009 | −56.966 | −40.680 | 1.00 | 72.26 | C |
| ATOM | 1381 | C3 | PLM | E | 2 | 9.761 | −57.587 | −42.054 | 1.00 | 73.03 | C |
| ATOM | 1382 | C4 | PLM | E | 2 | 11.014 | −57.512 | −42.926 | 1.00 | 74.51 | C |
| ATOM | 1383 | C5 | PLM | E | 2 | 11.788 | −58.832 | −43.027 | 1.00 | 75.54 | C |
| ATOM | 1384 | C6 | PLM | E | 2 | 12.096 | −59.186 | −44.489 | 1.00 | 75.76 | C |
| ATOM | 1385 | C7 | PLM | E | 2 | 13.499 | −59.769 | −44.661 | 1.00 | 75.86 | C |
| ATOM | 1386 | C8 | PLM | E | 2 | 13.586 | −60.801 | −45.788 | 1.00 | 76.18 | C |
| ATOM | 1387 | C9 | PLM | E | 2 | 14.405 | −60.291 | −46.976 | 1.00 | 74.91 | C |
| ATOM | 1396 | C1 | PLM | E | 3 | 13.409 | −59.091 | −38.243 | 1.00 | 43.40 | C |
| ATOM | 1398 | C2 | PLM | E | 3 | 13.872 | −59.588 | −39.613 | 1.00 | 47.64 | C |
| ATOM | 1399 | C3 | PLM | E | 3 | 15.354 | −59.947 | −39.674 | 1.00 | 45.49 | C |
| ATOM | 1400 | C4 | PLM | E | 3 | 15.691 | −60.300 | −41.115 | 1.00 | 44.84 | C |
| ATOM | 1401 | C5 | PLM | E | 3 | 17.055 | −60.944 | −41.231 | 1.00 | 45.47 | C |
| ATOM | 1402 | C6 | PLM | E | 3 | 17.125 | −61.793 | −42.488 | 1.00 | 45.61 | C |
| ATOM | 1403 | C7 | PLM | E | 3 | 18.434 | −61.525 | −43.213 | 1.00 | 50.54 | C |
| ATOM | 1404 | C8 | PLM | E | 3 | 18.567 | −62.151 | −44.598 | 1.00 | 50.19 | C |
| ATOM | 1405 | C9 | PLM | E | 3 | 19.881 | −62.927 | −44.668 | 1.00 | 51.53 | C |
| ATOM | 1406 | C10 | PLM | E | 3 | 20.304 | −63.298 | −46.092 | 1.00 | 54.50 | C |
| ATOM | 1414 | C1 | PLM | E | 4 | 11.495 | −55.573 | −46.829 | 1.00 | 60.10 | C |
| ATOM | 1416 | C2 | PLM | E | 4 | 12.312 | −54.868 | −45.758 | 1.00 | 60.16 | C |
| ATOM | 1417 | C3 | PLM | E | 4 | 11.480 | −53.831 | −44.993 | 1.00 | 58.00 | C |
| ATOM | 1418 | C4 | PLM | E | 4 | 12.404 | −53.062 | −44.045 | 1.00 | 57.41 | C |
| ATOM | 1419 | C5 | PLM | E | 4 | 11.701 | −52.049 | −43.146 | 1.00 | 55.27 | C |
| ATOM | 1432 | C1 | PLM | E | 5 | 23.241 | −52.587 | −6.604 | 1.00 | 64.05 | C |
| ATOM | 1434 | C2 | PLM | E | 5 | 22.971 | −53.290 | −5.290 | 1.00 | 65.83 | C |
| ATOM | 1435 | C3 | PLM | E | 5 | 21.603 | −52.879 | −4.748 | 1.00 | 66.72 | C |
| ATOM | 1436 | C4 | PLM | E | 5 | 21.522 | −52.787 | −3.227 | 1.00 | 66.62 | C |
| ATOM | 1437 | C5 | PLM | E | 5 | 20.079 | −52.504 | −2.830 | 1.00 | 67.23 | C |
| ATOM | 1438 | C6 | PLM | E | 5 | 19.977 | −51.825 | −1.479 | 1.00 | 68.89 | C |
| ATOM | 1439 | C7 | PLM | E | 5 | 18.520 | −51.679 | −1.062 | 1.00 | 70.98 | C |
| ATOM | 1440 | C8 | PLM | E | 5 | 18.079 | −50.209 | −1.116 | 1.00 | 72.90 | C |
| ATOM | 1441 | C9 | PLM | E | 5 | 16.809 | −49.942 | −0.304 | 1.00 | 72.41 | C |
| ATOM | 1442 | C10 | PLM | E | 5 | 16.133 | −48.629 | −0.701 | 1.00 | 74.15 | C |
| ATOM | 1443 | C11 | PLM | E | 5 | 15.343 | −48.738 | −2.009 | 1.00 | 74.42 | C |
| ATOM | 1444 | C12 | PLM | E | 5 | 14.304 | −47.628 | −2.145 | 1.00 | 75.37 | C |
| ATOM | 1445 | C13 | PLM | E | 5 | 13.225 | −48.052 | −3.137 | 1.00 | 75.74 | C |
| ATOM | 1446 | C14 | PLM | E | 5 | 11.990 | −47.156 | −3.065 | 1.00 | 75.82 | C |
| ATOM | 1450 | C1 | PLM | E | 6 | 20.482 | −51.252 | −45.234 | 1.00 | 57.66 | C |
| ATOM | 1452 | C2 | PLM | E | 6 | 19.557 | −52.371 | −45.650 | 1.00 | 60.20 | C |
| ATOM | 1453 | C3 | PLM | E | 6 | 20.274 | −53.611 | −46.205 | 1.00 | 64.91 | C |
| ATOM | 1454 | C4 | PLM | E | 6 | 20.408 | −54.765 | −45.201 | 1.00 | 66.16 | C |
| ATOM | 1455 | C5 | PLM | E | 6 | 21.817 | −55.381 | −45.206 | 1.00 | 68.53 | C |
| ATOM | 1456 | C6 | PLM | E | 6 | 21.905 | −56.912 | −45.227 | 1.00 | 68.79 | C |
| ATOM | 1457 | C7 | PLM | E | 6 | 21.712 | −57.577 | −46.596 | 1.00 | 70.69 | C |
| ATOM | 1458 | C8 | PLM | E | 6 | 20.949 | −58.908 | −46.519 | 1.00 | 70.78 | C |
| ATOM | 1459 | C9 | PLM | E | 6 | 20.505 | −59.466 | −47.876 | 1.00 | 71.83 | C |
| ATOM | 1468 | C1 | PLM | E | 7 | 18.225 | −45.029 | −10.107 | 1.00 | 74.92 | C |
| ATOM | 1470 | C2 | PLM | E | 7 | 19.181 | −46.059 | −9.549 | 1.00 | 75.43 | C |
| ATOM | 1471 | C3 | PLM | E | 7 | 18.786 | −47.453 | −10.029 | 1.00 | 76.14 | C |
| ATOM | 1472 | C4 | PLM | E | 7 | 19.832 | −48.520 | −9.706 | 1.00 | 76.87 | C |
| ATOM | 1473 | C5 | PLM | E | 7 | 19.375 | −49.504 | −8.633 | 1.00 | 76.41 | C |
| ATOM | 1474 | C6 | PLM | E | 7 | 20.305 | −49.510 | −7.413 | 1.00 | 77.88 | C |
| ATOM | 1475 | C7 | PLM | E | 7 | 20.259 | −48.203 | −6.604 | 1.00 | 78.46 | C |
| ATOM | 1476 | C8 | PLM | E | 7 | 20.055 | −48.371 | −5.094 | 1.00 | 77.69 | C |
| ATOM | 1477 | C9 | PLM | E | 7 | 18.984 | −47.397 | −4.584 | 1.00 | 78.07 | C |
| ATOM | 1486 | C1 | PLM | E | 8 | 18.445 | −56.498 | −47.334 | 1.00 | 62.14 | C |
| ATOM | 1488 | C2 | PLM | E | 8 | 17.684 | −57.340 | −46.328 | 1.00 | 60.03 | C |
| ATOM | 1489 | C3 | PLM | E | 8 | 17.050 | −56.385 | −45.330 | 1.00 | 57.68 | C |
| ATOM | 1490 | C4 | PLM | E | 8 | 16.340 | −57.092 | −44.184 | 1.00 | 54.58 | C |
| ATOM | 1491 | C5 | PLM | E | 8 | 15.228 | −56.195 | −43.633 | 1.00 | 54.88 | C |
| ATOM | 1492 | C6 | PLM | E | 8 | 15.387 | −56.057 | −42.120 | 1.00 | 52.46 | C |
| ATOM | 1493 | C7 | PLM | E | 8 | 14.116 | −55.964 | −41.306 | 1.00 | 51.86 | C |
| ATOM | 1494 | C8 | PLM | E | 8 | 14.015 | −54.610 | −40.626 | 1.00 | 51.66 | C |
| ATOM | 1495 | C9 | PLM | E | 8 | 13.654 | −54.634 | −39.146 | 1.00 | 54.74 | C |
| ATOM | 1496 | C10 | PLM | E | 8 | 12.638 | −53.542 | −38.802 | 1.00 | 56.92 | C |
| ATOM | 1497 | C11 | PLM | E | 8 | 12.602 | −53.118 | −37.326 | 1.00 | 60.52 | C |
| ATOM | 1498 | C12 | PLM | E | 8 | 11.662 | −53.898 | −36.395 | 1.00 | 61.15 | C |
| ATOM | 1499 | C13 | PLM | E | 8 | 12.396 | −55.029 | −35.639 | 1.00 | 63.48 | C |
| ATOM | 1500 | C14 | PLM | E | 8 | 11.653 | −56.371 | −35.496 | 1.00 | 61.80 | C |
| ATOM | 1507 | C3 | PLM | E | 9 | −3.388 | −49.728 | −1.656 | 1.00 | 77.81 | C |
| ATOM | 1508 | C4 | PLM | E | 9 | −3.796 | −49.452 | −3.103 | 1.00 | 77.86 | C |
| ATOM | 1509 | C5 | PLM | E | 9 | −2.831 | −50.073 | −4.115 | 1.00 | 77.90 | C |
| ATOM | 1510 | C6 | PLM | E | 9 | −3.415 | −50.033 | −5.526 | 1.00 | 77.11 | C |
| ATOM | 1522 | C1 | PLM | E | 10 | 7.838 | −53.489 | −32.633 | 1.00 | 66.07 | C |
| ATOM | 1524 | C2 | PLM | E | 10 | 6.779 | −52.797 | −31.806 | 1.00 | 66.68 | C |
| ATOM | 1525 | C3 | PLM | E | 10 | 7.412 | −52.127 | −30.594 | 1.00 | 66.75 | C |
| ATOM | 1526 | C4 | PLM | E | 10 | 6.522 | −50.998 | −30.056 | 1.00 | 67.37 | C |
| ATOM | 1527 | C5 | PLM | E | 10 | 6.098 | −51.170 | −28.596 | 1.00 | 65.79 | C |
| ATOM | 1528 | C6 | PLM | E | 10 | 5.278 | −49.972 | −28.109 | 1.00 | 65.57 | C |
| ATOM | 1529 | C7 | PLM | E | 10 | 5.403 | −49.732 | −26.605 | 1.00 | 64.10 | C |
| ATOM | 1530 | C8 | PLM | E | 10 | 5.017 | −48.304 | −26.179 | 1.00 | 64.01 | C |
| ATOM | 1531 | C9 | PLM | E | 10 | 6.061 | −47.628 | −25.289 | 1.00 | 60.09 | C |
| ATOM | 1532 | C10 | PLM | E | 10 | 5.576 | −47.317 | −23.876 | 1.00 | 60.70 | C |
| ATOM | 1543 | C3 | PLM | E | 11 | 17.260 | −46.767 | −62.233 | 1.00 | 77.12 | C |
| ATOM | 1544 | C4 | PLM | E | 11 | 18.724 | −47.213 | −62.167 | 1.00 | 77.11 | C |
| ATOM | 1545 | C5 | PLM | E | 11 | 18.843 | −48.711 | −61.868 | 1.00 | 76.32 | C |
| ATOM | 1546 | C6 | PLM | E | 11 | 19.190 | −49.036 | −60.407 | 1.00 | 74.86 | C |
| ATOM | 1547 | C7 | PLM | E | 11 | 20.474 | −49.859 | −60.243 | 1.00 | 72.18 | C |
| ATOM | 1548 | C8 | PLM | E | 11 | 21.720 | −49.012 | −59.917 | 1.00 | 69.63 | C |
| ATOM | 1549 | C9 | PLM | E | 11 | 22.043 | −47.969 | −60.999 | 1.00 | 66.36 | C |
| ATOM | 1550 | C10 | PLM | E | 11 | 23.529 | −47.634 | −61.123 | 1.00 | 60.96 | C |
| ATOM | 1551 | C11 | PLM | E | 11 | 23.744 | −46.131 | −60.959 | 1.00 | 53.56 | C |
| ATOM | 1552 | C12 | PLM | E | 11 | 24.390 | −45.814 | −59.592 | 1.00 | 51.09 | C |
| ATOM | 1553 | C13 | PLM | E | 11 | 24.083 | −44.426 | −59.014 | 1.00 | 35.34 | C |
| ATOM | 1554 | C14 | PLM | E | 11 | 24.712 | −44.175 | −57.646 | 1.00 | 45.65 | C |
| ATOM | 1555 | C15 | PLM | E | 11 | 23.750 | −43.713 | −56.535 | 1.00 | 41.07 | C |
| ATOM | 1556 | C16 | PLM | E | 11 | 24.270 | −42.627 | −55.639 | 1.00 | 32.17 | C |
| ATOM | 1557 | O2 | PLM | E | 12 | 26.718 | −44.330 | −29.929 | 1.00 | 63.05 | O |
| ATOM | 1558 | C1 | PLM | E | 12 | 26.490 | −45.566 | −30.045 | 1.00 | 58.42 | C |
| ATOM | 1559 | O1 | PLM | E | 12 | 26.039 | −46.107 | −29.007 | 1.00 | 50.98 | O |
| ATOM | 1560 | C2 | PLM | E | 12 | 26.737 | −46.349 | −31.356 | 1.00 | 53.64 | C |
| ATOM | 1561 | C3 | PLM | E | 12 | 28.168 | −46.863 | −31.443 | 1.00 | 52.96 | C |
| ATOM | 1562 | C4 | PLM | E | 12 | 28.421 | −48.250 | −32.051 | 1.00 | 54.56 | C |
| ATOM | 1563 | C5 | PLM | E | 12 | 29.529 | −49.030 | −31.332 | 1.00 | 54.51 | C |
| ATOM | 1564 | C6 | PLM | E | 12 | 29.975 | −50.290 | −32.098 | 1.00 | 57.32 | C |
| ATOM | 1565 | C7 | PLM | E | 12 | 29.451 | −51.569 | −31.440 | 1.00 | 55.35 | C |
| ATOM | 1566 | C8 | PLM | E | 12 | 28.761 | −52.503 | −32.438 | 1.00 | 57.24 | C |
| ATOM | 1567 | C9 | PLM | E | 12 | 28.307 | −53.855 | −31.866 | 1.00 | 53.71 | C |
| ATOM | 1568 | C10 | PLM | E | 12 | 27.896 | −53.890 | −30.387 | 1.00 | 55.00 | C |
| ATOM | 1569 | C11 | PLM | E | 12 | 26.788 | −54.929 | −30.188 | 1.00 | 52.46 | C |
| ATOM | 1570 | C12 | PLM | E | 12 | 26.524 | −55.352 | −28.761 | 1.00 | 51.60 | C |
| ATOM | 1571 | C13 | PLM | E | 12 | 25.142 | −55.976 | −28.756 | 1.00 | 49.17 | C |
| ATOM | 1572 | C14 | PLM | E | 12 | 24.950 | −57.102 | −27.764 | 1.00 | 49.70 | C |
| ATOM | 1573 | C15 | PLM | E | 12 | 23.480 | −57.546 | −27.752 | 1.00 | 50.03 | C |
| ATOM | 1574 | C16 | PLM | E | 12 | 23.218 | −58.681 | −26.752 | 1.00 | 50.32 | C |
| ATOM | 1583 | C7 | PLM | E | 13 | 22.537 | −35.853 | −28.273 | 1.00 | 81.67 | C |
| ATOM | 1584 | C8 | PLM | E | 13 | 22.546 | −36.528 | −29.642 | 1.00 | 83.70 | C |
| ATOM | 1585 | C9 | PLM | E | 13 | 23.842 | −37.316 | −29.873 | 1.00 | 84.89 | C |
| ATOM | 1586 | C10 | PLM | E | 13 | 23.607 | −38.545 | −30.753 | 1.00 | 85.74 | C |
| ATOM | 1587 | C11 | PLM | E | 13 | 24.896 | −39.126 | −31.341 | 1.00 | 87.42 | C |
| ATOM | 1588 | C12 | PLM | E | 13 | 24.627 | −40.414 | −32.117 | 1.00 | 87.87 | C |
| ATOM | 1589 | C13 | PLM | E | 13 | 25.824 | −40.848 | −32.964 | 1.00 | 88.32 | C |
| ATOM | 1590 | C14 | PLM | E | 13 | 25.502 | −42.108 | −33.768 | 1.00 | 88.31 | C |
| ATOM | 1591 | C15 | PLM | E | 13 | 26.127 | −43.352 | −33.133 | 1.00 | 88.25 | C |
| ATOM | 1592 | C16 | PLM | E | 13 | 26.062 | −44.553 | −34.073 | 1.00 | 88.09 | C |
| ATOM | 1593 | S | SO4 | F | 1 | 0.371 | −60.843 | 0.415 | 0.50 | 33.47 | S |
| ATOM | 1594 | O1 | SO4 | F | 1 | 0.768 | −60.079 | 1.619 | 0.50 | 27.24 | O |
| ATOM | 1595 | O2 | SO4 | F | 1 | 0.045 | −59.903 | −0.677 | 0.50 | 34.07 | O |
| ATOM | 1596 | O3 | SO4 | F | 1 | 1.523 | −61.656 | −0.012 | 0.50 | 31.62 | O |
| ATOM | 1597 | O4 | SO4 | F | 1 | −0.770 | −61.686 | 0.706 | 0.50 | 31.33 | O |
| ATOM | 1598 | S | SO4 | F | 2 | 4.740 | −61.413 | −12.145 | 0.70 | 35.78 | S |
| ATOM | 1599 | O1 | SO4 | F | 2 | 5.350 | −60.844 | −13.352 | 0.70 | 36.38 | O |
| ATOM | 1600 | O2 | SO4 | F | 2 | 3.349 | −61.045 | −12.328 | 0.70 | 37.26 | O |
| ATOM | 1601 | O3 | SO4 | F | 2 | 4.899 | −62.845 | −12.206 | 0.70 | 35.60 | O |
| ATOM | 1602 | O4 | SO4 | F | 2 | 5.375 | −61.026 | −10.910 | 0.70 | 25.11 | O |
| ATOM | 1603 | O | HOH | G | 1 | 15.375 | −58.000 | −15.073 | 1.00 | 22.82 | O |
| ATOM | 1604 | O | HOH | G | 2 | 19.531 | −46.287 | −36.566 | 1.00 | 27.78 | O |
| ATOM | 1605 | O | HOH | G | 3 | 29.517 | −43.658 | −23.829 | 1.00 | 30.07 | O |
| ATOM | 1606 | O | HOH | G | 4 | −0.278 | −64.451 | −2.346 | 1.00 | 70.05 | O |
| ATOM | 1607 | O | HOH | G | 5 | 9.052 | −51.779 | −3.324 | 1.00 | 38.80 | O |
| ATOM | 1608 | O | HOH | G | 6 | 16.885 | −52.027 | −46.008 | 1.00 | 36.95 | O |
| ATOM | 1609 | O | HOH | G | 7 | 3.471 | −48.240 | −11.420 | 1.00 | 40.77 | O |
| ATOM | 1610 | O | HOH | G | 8 | 20.174 | −63.744 | −13.015 | 1.00 | 38.14 | O |
| ATOM | 1611 | O | HOH | G | 9 | 2.276 | −60.434 | 2.999 | 1.00 | 39.02 | O |
| ATOM | 1612 | O | HOH | G | 10 | −0.002 | −55.693 | 0.000 | 0.50 | 40.02 | O |
| ATOM | 1613 | O | HOH | G | 11 | 5.511 | −58.682 | 4.679 | 1.00 | 51.03 | O |
| ATOM | 1614 | O | HOH | G | 12 | 9.721 | −52.208 | −56.796 | 1.00 | 56.88 | O |
| ATOM | 1615 | O | HOH | G | 13 | 18.599 | −48.824 | −51.587 | 1.00 | 45.31 | O |
| ATOM | 1616 | O | HOH | G | 14 | 14.316 | −60.310 | 0.899 | 1.00 | 51.21 | O |
| ATOM | 1617 | O | HOH | G | 15 | 12.024 | −46.710 | −11.705 | 1.00 | 39.84 | O |
| ATOM | 1618 | O | HOH | G | 16 | 19.768 | −63.332 | −9.672 | 1.00 | 34.13 | O |
| ATOM | 1619 | O | HOH | G | 17 | 11.715 | −77.776 | −5.495 | 1.00 | 60.38 | O |
| ATOM | 1620 | O | HOH | G | 18 | 11.013 | −76.333 | 2.582 | 1.00 | 56.66 | O |
| ATOM | 1621 | O | HOH | G | 19 | 12.691 | −69.042 | −15.651 | 1.00 | 42.53 | O |
| ATOM | 1622 | O | HOH | G | 20 | 16.776 | −57.005 | −13.259 | 1.00 | 46.26 | O |
| ATOM | 1623 | O | HOH | G | 21 | 11.581 | −56.209 | 1.044 | 1.00 | 42.54 | O |
| ATOM | 1624 | O | HOH | G | 22 | 15.940 | −62.084 | 1.839 | 1.00 | 50.28 | O |
| ATOM | 1625 | O | HOH | G | 23 | 6.480 | −48.205 | −7.968 | 1.00 | 57.43 | O |
| ATOM | 1626 | O | HOH | G | 24 | 39.197 | −43.575 | −30.519 | 1.00 | 42.04 | O |
| ATOM | 1627 | O | HOH | G | 25 | 6.027 | −62.306 | −15.788 | 1.00 | 55.55 | O |
| ATOM | 1628 | O | HOH | G | 26 | −3.802 | −54.617 | −6.208 | 1.00 | 62.40 | O |
| ATOM | 1629 | O | HOH | G | 27 | 28.505 | −47.616 | −34.361 | 1.00 | 65.78 | O |
| ATOM | 1630 | OW0 | HOH | G | 28 | −1.801 | −59.317 | −5.575 | 1.00 | 44.97 | O |
| ATOM | 1631 | OW0 | HOH | G | 29 | 22.373 | −53.791 | −42.746 | 1.00 | 56.53 | O |
| ATOM | 1632 | O | HOH | G | 30 | 20.463 | −67.986 | −5.108 | 1.00 | 49.86 | O |
| ATOM | 1633 | OW0 | HOH | G | 31 | 1.458 | −52.674 | −5.845 | 1.00 | 35.48 | O |
| ATOM | 1634 | OW0 | HOH | G | 32 | −0.601 | −51.527 | −2.029 | 1.00 | 56.79 | O |
| ATOM | 1635 | OW0 | HOH | G | 33 | −0.725 | −51.609 | −7.206 | 1.00 | 46.34 | O |
| ATOM | 1636 | OW0 | HOH | G | 34 | 52.333 | −52.336 | −32.501 | 0.33 | 62.20 | O |
| ATOM | 1637 | OW0 | HOH | G | 35 | 40.005 | −46.339 | −20.411 | 1.00 | 51.61 | O |
| ATOM | 1638 | OW0 | HOH | G | 36 | 7.348 | −45.031 | −11.009 | 1.00 | 67.53 | O |
| ATOM | 1639 | OW0 | HOH | G | 37 | 18.638 | −47.155 | −44.937 | 1.00 | 42.78 | O |
| ATOM | 1640 | OW0 | HOH | G | 38 | 42.297 | −46.265 | −36.401 | 1.00 | 59.51 | O |
| ATOM | 1641 | OW0 | HOH | G | 39 | 19.312 | −49.538 | −53.973 | 1.00 | 63.11 | O |
| ATOM | 1642 | OW0 | HOH | G | 40 | 12.092 | −61.018 | 2.812 | 1.00 | 57.91 | O |
| ATOM | 1643 | OW0 | HOH | G | 41 | 9.109 | −62.096 | 4.871 | 1.00 | 64.80 | O |
| ATOM | 1644 | OW0 | HOH | G | 42 | 22.011 | −46.105 | −39.823 | 1.00 | 57.86 | O |
| ATOM | 1645 | OW0 | HOH | G | 43 | 46.973 | −55.321 | −35.496 | 1.00 | 69.49 | O |
| ATOM | 1646 | OW0 | HOH | G | 44 | 5.887 | −57.239 | −20.655 | 1.00 | 60.45 | O |
| ATOM | 1647 | OW0 | HOH | G | 45 | 3.287 | −69.706 | −1.459 | 1.00 | 73.57 | O |
| ATOM | 1648 | OW0 | HOH | G | 46 | 49.236 | −55.611 | −31.052 | 1.00 | 63.34 | O |
| ATOM | 1649 | O | HOH | G | 47 | 14.454 | −67.301 | −14.382 | 1.00 | 34.21 | O |
| ATOM | 1650 | OW0 | HOH | G | 48 | 9.238 | −74.111 | −6.684 | 1.00 | 57.89 | O |
| ATOM | 1651 | OW0 | HOH | G | 49 | 21.087 | −49.042 | −50.373 | 1.00 | 56.92 | O |
| ATOM | 1652 | OW0 | HOH | G | 50 | 2.130 | −50.654 | −4.091 | 1.00 | 57.03 | O |
| ATOM | 1653 | OW0 | HOH | G | 51 | 6.952 | −49.477 | −5.064 | 1.00 | 65.41 | O |
| ATOM | 1654 | OW0 | HOH | G | 52 | 5.018 | −51.449 | −3.965 | 1.00 | 48.62 | O |
| ATOM | 1655 | OW0 | HOH | G | 53 | 9.445 | −55.961 | −16.924 | 1.00 | 35.37 | O |
| ATOM | 1656 | OW0 | HOH | G | 54 | 8.917 | −72.072 | −5.125 | 1.00 | 50.93 | O |
| ATOM | 1657 | OW0 | HOH | G | 55 | 10.673 | −58.107 | 3.325 | 1.00 | 64.30 | O |
| ATOM | 1658 | OW0 | HOH | G | 56 | 4.127 | −64.321 | 4.681 | 1.00 | 55.09 | O |
| ATOM | 1659 | OW0 | HOH | G | 57 | 14.339 | −57.713 | 0.092 | 1.00 | 64.04 | O |
| ATOM | 1660 | O | HOH | G | 58 | 15.953 | −51.995 | −59.077 | 1.00 | 72.95 | O |
| ATOM | 1661 | OW0 | HOH | G | 59 | 1.655 | −62.800 | −12.959 | 1.00 | 51.50 | O |
| ATOM | 1662 | OW0 | HOH | G | 60 | 8.303 | −67.621 | −12.206 | 1.00 | 46.64 | O |
| ATOM | 1663 | OW0 | HOH | G | 61 | 11.164 | −52.340 | 1.370 | 1.00 | 66.16 | O |
| ATOM | 1664 | OW0 | HOH | G | 62 | 7.352 | −56.599 | −17.979 | 1.00 | 53.67 | O |
| ATOM | 1665 | OW0 | HOH | G | 63 | 12.595 | −76.194 | 9.847 | 0.33 | 37.05 | O |
| ATOM | 1666 | OW0 | HOH | G | 64 | 23.621 | −44.970 | −49.242 | 1.00 | 67.29 | O |
| ATOM | 1667 | O | HOH | G | 65 | 16.972 | −53.121 | −66.418 | 1.00 | 85.95 | O |
| ATOM | 1668 | OW0 | HOH | G | 66 | 3.059 | −73.144 | 3.863 | 1.00 | 62.75 | O |
| ATOM | 1669 | O | HOH | G | 67 | 7.859 | −48.330 | −32.956 | 1.00 | 65.26 | O |
| ATOM | 1670 | O | HOH | G | 68 | 4.156 | −58.856 | −27.809 | 1.00 | 57.36 | O |
| ATOM | 1671 | O | HOH | G | 69 | 4.551 | −59.395 | −29.922 | 1.00 | 69.10 | O |
| ATOM | 1672 | O | HOH | G | 70 | 6.260 | −47.543 | −31.586 | 1.00 | 68.61 | O |
| ATOM | 1673 | O | HOH | G | 71 | 7.840 | −44.030 | −28.246 | 1.00 | 62.70 | O |
| ATOM | 1674 | O | HOH | G | 72 | 8.079 | −41.411 | −33.214 | 1.00 | 72.05 | O |
| ATOM | 1675 | O | HOH | G | 73 | 5.110 | −39.116 | −28.229 | 1.00 | 87.18 | O |
| ATOM | 1676 | O | HOH | G | 74 | 4.809 | −40.681 | −30.664 | 1.00 | 89.65 | O |
| ATOM | 1677 | O | HOH | G | 75 | 1.648 | −46.036 | −34.526 | 1.00 | 82.70 | O |
| ATOM | 1678 | O | HOH | G | 76 | 22.388 | −44.026 | −33.664 | 1.00 | 58.55 | O |
| ATOM | 1679 | O | HOH | G | 77 | 29.957 | −41.672 | −33.880 | 1.00 | 62.69 | O |
| ATOM | 1680 | O | HOH | G | 78 | 5.207 | −64.324 | −10.143 | 1.00 | 37.85 | O |
| ATOM | 1681 | O | HOH | G | 79 | 12.766 | −53.130 | −50.860 | 1.00 | 46.09 | O |
| ATOM | 1682 | O | HOH | G | 80 | 15.500 | −53.938 | −50.088 | 1.00 | 60.49 | O |
| ATOM | 1683 | O | HOH | G | 81 | 8.596 | −38.206 | −69.322 | 1.00 | 46.24 | O |
| ATOM | 1684 | O | HOH | G | 82 | 15.672 | −67.848 | −17.578 | 1.00 | 40.99 | O |
| ATOM | 1685 | O | HOH | G | 83 | 19.615 | −63.969 | −15.881 | 1.00 | 30.41 | O |
| ATOM | 1686 | O | HOH | G | 84 | 18.094 | −66.902 | −18.047 | 0.33 | 37.23 | O |
| ATOM | 1687 | OW0 | HOH | G | 85 | 11.250 | −67.176 | −16.147 | 1.00 | 44.94 | O |
| ATOM | 1688 | OW0 | HOH | G | 86 | 25.393 | −63.667 | −6.384 | 1.00 | 67.71 | O |
| ATOM | 1689 | OW0 | HOH | G | 87 | −1.170 | −60.898 | −7.473 | 1.00 | 60.19 | O |
| ATOM | 1690 | OW0 | HOH | G | 88 | −1.923 | −60.821 | −9.510 | 1.00 | 55.99 | O |
| ATOM | 1691 | O | HOH | G | 89 | 5.204 | −75.045 | −0.805 | 1.00 | 66.56 | O |
| ATOM | 1692 | O | HOH | G | 90 | 16.440 | −51.952 | −56.650 | 1.00 | 53.69 | O |
| ATOM | 1693 | O | HOH | G | 91 | 17.250 | −43.149 | −41.268 | 1.00 | 40.84 | O |
| ATOM | 1694 | O | HOH | G | 92 | 20.290 | −55.786 | 2.319 | 1.00 | 69.16 | O |
| ATOM | 1695 | O | HOH | G | 93 | −3.677 | −57.411 | −6.324 | 1.00 | 55.59 | O |
| ATOM | 1696 | O | HOH | G | 94 | 6.045 | −75.775 | 1.785 | 1.00 | 89.59 | O |
| ATOM | 1697 | O | HOH | G | 95 | 4.531 | −53.450 | −35.339 | 1.00 | 92.62 | O |
| ATOM | 1698 | O | HOH | G | 96 | 4.130 | −51.404 | −33.734 | 1.00 | 82.09 | O |
| ATOM | 1699 | O | HOH | G | 97 | 12.647 | −72.205 | 12.644 | 0.33 | 78.15 | O |
| ATOM | 1700 | O | HOH | G | 98 | 2.444 | −62.657 | 4.285 | 1.00 | 59.37 | O |
| ATOM | 1701 | O | HOH | G | 99 | 7.480 | −44.064 | −63.567 | 1.00 | 62.36 | O |
| ATOM | 1702 | O | HOH | H | 1 | 10.235 | −73.923 | −12.362 | 1.00 | 54.56 | O |
| ATOM | 1703 | O | HOH | H | 2 | 10.853 | −75.041 | −11.353 | 0.33 | 24.04 | O |
| ATOM | 1704 | O | HOH | H | 3 | 15.894 | −53.237 | 1.991 | 1.00 | 93.97 | O |
| ATOM | 1705 | O | HOH | H | 4 | 39.197 | −44.633 | −37.978 | 1.00 | 63.14 | O |
| ATOM | 1706 | O | HOH | H | 5 | 44.373 | −55.060 | −35.376 | 1.00 | 52.81 | O |
| ATOM | 1707 | O | HOH | H | 6 | 17.661 | −71.389 | −5.588 | 1.00 | 43.35 | O |
| ATOM | 1708 | O | HOH | H | 7 | 12.422 | −42.244 | −65.541 | 1.00 | 57.13 | O |
| ATOM | 1709 | O | HOH | H | 8 | 6.038 | −58.327 | −16.631 | 1.00 | 68.30 | O |
| ATOM | 1710 | O | HOH | H | 9 | 5.301 | −73.350 | 5.416 | 1.00 | 76.92 | O |
| ATOM | 1711 | O | HOH | H | 10 | 23.492 | −43.612 | −31.618 | 1.00 | 75.36 | O |
| ATOM | 1712 | O | HOH | H | 11 | 26.685 | −40.784 | −24.862 | 1.00 | 60.70 | O |
| ATOM | 1713 | O | HOH | H | 12 | 19.317 | −64.666 | 6.838 | 1.00 | 65.00 | O |
| ATOM | 1714 | O | HOH | H | 13 | 27.223 | −42.773 | −28.000 | 1.00 | 68.99 | O |
| ATOM | 1715 | O | HOH | H | 14 | 1.887 | −64.499 | −0.652 | 1.00 | 57.61 | O |
| END | | | | | | | | | | | |

TABLE II
co-ordinates with GSH

| | | |
|---|---|---|
| HEADER | --- XX-XXX-9- | dasm |

| | |
|---|---|
| COMPND | --- |

| | | | |
|---|---|---|---|
| REMARK | 3 | | |
| REMARK | 3 | REFINEMENT. | |
| REMARK | 3 | PROGRAM | : REFMAC 5.2.0019 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.15 |
| REMARK | 3 | RESOLUTION RANGE LOW | (ANGSTROMS) | : 49.03 |
| REMARK | 3 | DATA CUTOFF | (SIGMA(F)) | : NONE |
| REMARK | 3 | COMPLETENESS FOR RANGE | (%) | : 99.66 |
| REMARK | 3 | NUMBER OF REFLECTIONS | | : 20920 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.18490 |
| REMARK | 3 | R VALUE | (WORKING SET) | : 0.18301 |

| | | | | |
|---|---|---|---|---|
| REMARK | 3 | FREE R VALUE | | : 0.22024 |
| REMARK | 3 | FREE R VALUE TEST SET SIZE | (%) | : 5.1 |
| REMARK | 3 | FREE R VALUE TEST SET COUNT | | : 1129 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 2.153 |
| REMARK | 3 | BIN RESOLUTION RANGE LOW | | : 2.209 |
| REMARK | 3 | REFLECTION IN BIN | (WORKING SET) | : 1544 |
| REMARK | 3 | BIN COMPLETENESS | (WORKING + TEST) (%) | : 100.00 |
| REMARK | 3 | BIN R VALUE | (WORKING SET) | : 0.245 |

| | | | |
|---|---|---|---|
| REMARK | 3 | BIN FREE R VALUE SET COUNT | : 93 |
| REMARK | 3 | BIN FREE R VALUE | : 0.264 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 1444 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 21.523 |

| REMARK | 3 | OVERALL ANISOTROPIC B VALUE. | | |
|---|---|---|---|---|
| REMARK | 3 | B11 (A**2) : NULL | | |
| REMARK | 3 | B22 (A**2) : NULL | | |
| REMARK | 3 | B33 (A**2) : NULL | | |
| REMARK | 3 | B12 (A**2) : NULL | | |
| REMARK | 3 | B13 (A**2) : NULL | | |
| REMARK | 3 | B23 (A**2) : NULL | | |
| REMARK | 3 | | | |
| REMARK | 3 | ESTIMATED OVERALL COORDINATE ERROR. | | |
| REMARK | 3 | ESU BASED ON R VALUE | (A): | 0.124 |
| REMARK | 3 | ESU BASED ON FREE R VALUE | (A): | 0.127 |
| REMARK | 3 | ESU BASED ON MAXIMUM LIKELIHOOD | (A): | 0.099 |
| REMARK | 3 | ESU FOR B VALUES BASED ON MAXIMUM LIKELIHOOD | (A**2): | 7.431 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.942 |
| REMARK | 3 | CORRELATION COEFFICIENT FO-FC FREE | : 0.921 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 1346 | ; 0.025 | ; 0.021 |
| REMARK | 3 | BOND ANGLES REFINED ATOMS | (DEGREES): | 1811 | ; 2.485 | ; 2.033 |
| REMARK | 3 | TORSION ANGLES, PERIOD 1 | (DEGREES): | 153 | ; 6.375 | ; 5.000 |
| REMARK | 3 | TORSION ANGLES, PERIOD 2 | (DEGREES): | 52 | ;29.194 | ;19.808 |
| REMARK | 3 | TORSION ANGLES, PERIOD 3 | (DEGREES): | 182 | ;17.505 | ;15.000 |
| REMARK | 3 | TORSION ANGLES, PERIOD 4 | (DEGREES): | 13 | ;19.279 | ;15.000 |
| REMARK | 3 | CHIRAL-CENTER RESTRAINTS | (A**3): | 205 | ; 0.216 | ; 0.200 |

| | | | | | | |
|---|---|---|---|---|---|---|
| REMARK | 3 | GENERAL PLANES REFINED ATOMS | (A): | 963 | ; 0.011 | ; 0.020 |
| REMARK | 3 | NON-BONDED CONTACTS REFINED ATOMS | (A): | 583 | ; 0.238 | ; 0.200 |
| REMARK | 3 | NON-BONDED TORSION REFINED ATOMS | (A): | 892 | ; 0.317 | ; 0.200 |
| REMARK | 3 | H-BOND (X...Y) REFINED ATOMS | (A): | 75 | ; 0.383 | ; 0.200 |
| REMARK | 3 | SYMMETRY VDW REFINED ATOMS | (A): | 102 | ; 0.227 | ; 0.200 |
| REMARK | 3 | SYMMETRY H-BOND REFINED ATOMS | (A): | 33 | ; 0.213 | ; 0.200 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 777 | ; 1.310 | ; 1.500 |
| REMARK | 3 | MAIN-CHAIN ANGLE REFINED ATOMS | (A**2): | 1199 | ; 1.935 | ; 2.000 |
| REMARK | 3 | SIDE-CHAIN BOND REFINED ATOMS | (A**2): | 637 | ; 3.354 | ; 3.000 |
| REMARK | 3 | SIDE-CHAIN ANGLE REFINED ATOMS | (A**2): | 610 | ; 4.793 | ; 4.500 |
| REMARK | 3 | | | | | |

| REMARK | 3 | NCS RESTRAINTS STATISTICS | | | | | |
|---|---|---|---|---|---|---|---|
| REMARK | 3 | NUMBER OF NCS GROUPS : NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS : 4 | | | | | |
| REMARK | 3 | ATOM RECORD CONTAINS RESIDUAL B FACTORS ONLY | | | | | |
| REMARK | 3 | | | | | | |
| REMARK | 3 | TLS GROUP : 1 | | | | | |
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | −4 | | A | 4 |

| REMARK | 3 | ORIGIN FOR THE GROUP (A): 38.7610 30.7943 37.1686 | | | | | |
|---|---|---|---|---|---|---|---|
| REMARK | 3 | T TENSOR | | | | | |
| REMARK | 3 | T11: | 0.0263 | T22: | 0.0132 | | |
| REMARK | 3 | T33: | −0.0026 | T12: | −0.0504 | | |
| REMARK | 3 | T13: | −0.1743 | T23: | 0.0166 | | |
| REMARK | 3 | L TENSOR | | | | | |
| REMARK | 3 | L11: | 43.7540 | L22: | 14.5336 | | |
| REMARK | 3 | L33: | 8.4265 | L12: | 22.5082 | | |
| REMARK | 3 | L13: | −4.8297 | L23: | −2.5635 | | |
| REMARK | 3 | S TENSOR | | | | | |
| REMARK | 3 | S11: | 0.4125 | S12: | 0.0342 | S13: | 0.0464 |
| REMARK | 3 | S21: | 0.5494 | S22: | −0.3780 | S23: | −0.3859 |
| REMARK | 3 | S31: | 0.4718 | S32: | 0.7522 | S33: | −0.0345 |
| REMARK | 3 | | | | | | |

| REMARK | 3 | TLS GROUP : 2 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 5 | | A | 47 |

| REMARK | 3 | ORIGIN FOR THE GROUP (A): 18.6917 4.7881 21.1784 | | | | | |
|---|---|---|---|---|---|---|---|
| REMARK | 3 | T TENSOR | | | | | |
| REMARK | 3 | T11: | 0.0778 | T22: | 0.0210 | | |
| REMARK | 3 | T33: | 0.0780 | T12: | −0.0304 | | |
| REMARK | 3 | T13: | 0.0112 | T23: | −0.0223 | | |
| REMARK | 3 | L TENSOR | | | | | |
| REMARK | 3 | L11: | 1.6369 | L22: | 2.6248 | | |
| REMARK | 3 | L33: | 3.7651 | L12: | 1.4943 | | |
| REMARK | 3 | L13: | 1.4586 | L23: | 1.8919 | | |
| REMARK | 3 | S TENSOR | | | | | |
| REMARK | 3 | S11: | 0.0517 | S12: | −0.0060 | S13: | −0.2508 |
| REMARK | 3 | S21: | 0.1396 | S22: | −0.0663 | S23: | −0.0501 |
| REMARK | 3 | S31: | 0.5843 | S32: | −0.1539 | S33: | 0.0146 |
| REMARK | 3 | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 48 | | A | 108 |

| REMARK | 3 | ORIGIN FOR THE GROUP (A): 13.6553 14.2035 29.6632 | | | | | |
|---|---|---|---|---|---|---|---|
| REMARK | 3 | T TENSOR | | | | | |
| REMARK | 3 | T11: | 0.0402 | T22: | 0.0368 | | |
| REMARK | 3 | T33: | 0.0374 | T12: | −0.0427 | | |
| REMARK | 3 | T13: | −0.0076 | T23: | −0.0064 | | |
| REMARK | 3 | L TENSOR | | | | | |
| REMARK | 3 | L11: | 1.3293 | L22: | 2.4856 | | |
| REMARK | 3 | L33: | 1.2819 | L12: | 1.1540 | | |
| REMARK | 3 | L13: | 0.8262 | L23: | 1.2899 | | |
| REMARK | 3 | S TENSOR | | | | | |
| REMARK | 3 | S11: | 0.0470 | S12: | −0.1247 | S13: | −0.0343 |
| REMARK | 3 | S21: | 0.1694 | S22: | −0.0326 | S23: | 0.0912 |
| REMARK | 3 | S31: | 0.1021 | S32: | −0.1147 | S33: | −0.0143 |
| REMARK | 3 | | | | | | |

| REMARK | 3 | TLS GROUP : 4 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 109 | | A | 145 |

| REMARK | 3 | ORIGIN FOR THE GROUP (A): 11.7672 39.2439 35.7931 | | | | | |
|---|---|---|---|---|---|---|---|
| REMARK | 3 | T TENSOR | | | | | |
| REMARK | 3 | T11: | 0.0948 | T22: | 0.1006 | | |
| REMARK | 3 | T33: | 0.1232 | T12: | 0.0341 | | |
| REMARK | 3 | T13: | −0.0316 | T23: | −0.0639 | | |
| REMARK | 3 | L TENSOR | | | | | |
| REMARK | 3 | L11: | 0.7275 | L22: | 19.7339 | | |
| REMARK | 3 | L33: | 1.6059 | L12: | 3.7802 | | |
| REMARK | 3 | L13: | 0.6232 | L23: | 2.9231 | | |
| REMARK | 3 | S TENSOR | | | | | |
| REMARK | 3 | S11: | −0.1412 | S12: | −0.0355 | S13: | 0.2319 |
| REMARK | 3 | S21: | 0.0911 | S22: | −0.0196 | S23: | 1.1691 |
| REMARK | 3 | S31: | −0.2654 | S32: | −0.1500 | S33: | 0.1608 |
| REMARK | 3 | | | | | | |
| REMARK | 3 | | | | | | |

| REMARK | 3 | BULK SOLVENT MODELLING. | | | | | | |
|---|---|---|---|---|---|---|---|---|
| REMARK | 3 | METHOD USED : MASK | | | | | | |
| REMARK | 3 | PARAMETERS FOR MASK CALCULATION | | | | | | |
| REMARK | 3 | VDW PROBE RADIUS | | | | : 1.20 | | |
| REMARK | 3 | ION PROBE RADIUS | | | | : 0.80 | | |
| REMARK | 3 | SHRINKAGE RADIUS | | | | : 0.80 | | |
| REMARK | 3 | | | | | | | |
| REMARK | 3 | OTHER REFINEMENT REMARKS: | | | | | | |
| REMARK | 3 | HYDROGENS HAVE BEEN ADDED IN THE RIDING POSITIONS | | | | | | |
| REMARK | 3 | | | | | | | |
| CISPEP | 1 | HIS | A | −4 | HIS | A | −3 | 0.00 |
| CISPEP | 2 | PRO | A | 37 | PRO | A | 38 | 0.00 |

| | | | | | |
|---|---|---|---|---|---|
| CRYST1 | 169.940 | 169.940 | 169.940 | 90.00 90.00 90.00 | F 2 3 |

| | | | | |
|---|---|---|---|---|
| SCALE1 | 0.005884 | 0.000000 | 0.000000 | 0.00000 |
| SCALE2 | 0.000000 | 0.005884 | 0.000000 | 0.00000 |
| SCALE3 | 0.000000 | 0.000000 | 0.005884 | 0.00000 |

| | | | | | | | | | | | |
|---|---|---|---|---|---|---|---|---|---|---|---|
| ATOM | 1 | N | HIS | A | −4 | 45.102 | 31.739 | 30.742 | 1.00 | 43.85 | N |
| ATOM | 2 | CA | HIS | A | −4 | 44.912 | 32.035 | 32.222 | 1.00 | 44.81 | C |
| ATOM | 3 | CB | HIS | A | −4 | 46.047 | 31.383 | 33.039 | 1.00 | 43.72 | C |
| ATOM | 4 | CG | HIS | A | −4 | 47.334 | 32.147 | 32.989 | 1.00 | 42.63 | C |
| ATOM | 5 | ND1 | HIS | A | −4 | 48.139 | 32.175 | 31.872 | 1.00 | 41.37 | N |
| ATOM | 6 | CE1 | HIS | A | −4 | 49.193 | 32.937 | 32.107 | 1.00 | 39.60 | C |
| ATOM | 7 | NE2 | HIS | A | −4 | 49.092 | 33.413 | 33.334 | 1.00 | 39.25 | N |
| ATOM | 8 | CD2 | HIS | A | −4 | 47.941 | 32.935 | 33.911 | 1.00 | 39.51 | C |
| ATOM | 9 | C | HIS | A | −4 | 44.952 | 33.568 | 32.414 | 1.00 | 45.14 | C |
| ATOM | 10 | O | HIS | A | −4 | 45.605 | 34.238 | 31.607 | 1.00 | 46.36 | O |
| ATOM | 11 | N | HIS | A | −3 | 44.343 | 34.189 | 33.435 | 1.00 | 45.00 | N |
| ATOM | 12 | CA | HIS | A | −3 | 43.545 | 33.720 | 34.595 | 1.00 | 44.05 | C |
| ATOM | 13 | CB | HIS | A | −3 | 42.075 | 33.507 | 34.265 | 1.00 | 43.76 | C |
| ATOM | 14 | CG | HIS | A | −3 | 41.624 | 34.373 | 33.110 | 1.00 | 45.31 | C |
| ATOM | 15 | ND1 | HIS | A | −3 | 41.585 | 35.756 | 33.185 | 1.00 | 44.37 | N |
| ATOM | 16 | CE1 | HIS | A | −3 | 41.234 | 36.246 | 32.006 | 1.00 | 44.90 | C |
| ATOM | 17 | NE2 | HIS | A | −3 | 41.048 | 35.237 | 31.168 | 1.00 | 41.90 | N |
| ATOM | 18 | CD2 | HIS | A | −3 | 41.330 | 34.060 | 31.815 | 1.00 | 42.23 | C |
| ATOM | 19 | C | HIS | A | −3 | 44.138 | 33.078 | 35.875 | 1.00 | 44.08 | C |
| ATOM | 20 | O | HIS | A | −3 | 44.669 | 31.928 | 35.919 | 1.00 | 44.84 | O |
| ATOM | 21 | N | HIS | A | −2 | 44.069 | 33.881 | 36.929 | 1.00 | 41.86 | N |
| ATOM | 22 | CA | HIS | A | −2 | 44.313 | 33.388 | 38.250 | 1.00 | 40.21 | C |
| ATOM | 23 | CB | HIS | A | −2 | 45.268 | 34.314 | 38.943 | 1.00 | 39.35 | C |
| ATOM | 24 | CG | HIS | A | −2 | 46.603 | 34.281 | 38.312 | 1.00 | 38.72 | C |
| ATOM | 25 | ND1 | HIS | A | −2 | 47.659 | 33.571 | 38.846 | 1.00 | 40.91 | N |
| ATOM | 26 | CE1 | HIS | A | −2 | 48.706 | 33.695 | 38.054 | 1.00 | 39.34 | C |
| ATOM | 27 | NE2 | HIS | A | −2 | 48.346 | 34.412 | 37.003 | 1.00 | 40.80 | N |
| ATOM | 28 | CD2 | HIS | A | −2 | 47.036 | 34.792 | 37.139 | 1.00 | 36.98 | C |
| ATOM | 29 | C | HIS | A | −2 | 43.028 | 33.183 | 39.024 | 1.00 | 39.93 | C |
| ATOM | 30 | O | HIS | A | −2 | 43.058 | 33.196 | 40.260 | 1.00 | 40.14 | O |
| ATOM | 31 | N | HIS | A | −1 | 41.913 | 33.024 | 38.295 | 1.00 | 38.27 | N |
| ATOM | 32 | CA | HIS | A | −1 | 40.687 | 32.426 | 38.860 | 1.00 | 38.31 | C |
| ATOM | 33 | CB | HIS | A | −1 | 39.796 | 33.479 | 39.494 | 1.00 | 37.44 | C |
| ATOM | 34 | CG | HIS | A | −1 | 39.148 | 34.364 | 38.481 | 1.00 | 37.49 | C |
| ATOM | 35 | ND1 | HIS | A | −1 | 39.820 | 35.414 | 37.880 | 1.00 | 32.64 | N |
| ATOM | 36 | CE1 | HIS | A | −1 | 39.015 | 35.995 | 37.011 | 1.00 | 31.75 | C |
| ATOM | 37 | NE2 | HIS | A | −1 | 37.858 | 35.346 | 37.008 | 1.00 | 34.18 | N |
| ATOM | 38 | CD2 | HIS | A | −1 | 37.926 | 34.302 | 37.900 | 1.00 | 31.68 | C |
| ATOM | 39 | C | HIS | A | −1 | 39.917 | 31.681 | 37.776 | 1.00 | 37.15 | C |
| ATOM | 40 | O | HIS | A | −1 | 40.095 | 31.915 | 36.568 | 1.00 | 38.15 | O |
| ATOM | 41 | N | HIS | A | 0 | 39.041 | 30.787 | 38.195 | 1.00 | 37.14 | N |
| ATOM | 42 | CA | HIS | A | 0 | 38.185 | 30.098 | 37.211 | 1.00 | 36.62 | C |
| ATOM | 43 | CB | HIS | A | 0 | 38.679 | 28.671 | 36.986 | 1.00 | 37.80 | C |
| ATOM | 44 | CG | HIS | A | 0 | 38.516 | 27.796 | 38.199 | 1.00 | 42.93 | C |
| ATOM | 45 | ND1 | HIS | A | 0 | 39.575 | 27.442 | 39.018 | 1.00 | 48.02 | N |
| ATOM | 46 | CE1 | HIS | A | 0 | 39.128 | 26.685 | 40.012 | 1.00 | 50.57 | C |
| ATOM | 47 | NE2 | HIS | A | 0 | 37.812 | 26.555 | 39.882 | 1.00 | 49.00 | N |
| ATOM | 48 | CD2 | HIS | A | 0 | 37.404 | 27.257 | 38.769 | 1.00 | 45.61 | C |
| ATOM | 49 | C | HIS | A | 0 | 36.760 | 30.045 | 37.746 | 1.00 | 35.43 | C |
| ATOM | 50 | O | HIS | A | 0 | 36.554 | 30.184 | 38.965 | 1.00 | 36.72 | O |
| ATOM | 51 | N | HIS | A | 1 | 35.775 | 29.837 | 36.882 | 1.00 | 32.02 | N |
| ATOM | 52 | CA | HIS | A | 1 | 34.422 | 29.832 | 37.358 | 1.00 | 30.71 | C |
| ATOM | 53 | CB | HIS | A | 1 | 33.572 | 30.843 | 36.606 | 1.00 | 30.18 | C |
| ATOM | 54 | CG | HIS | A | 1 | 33.987 | 32.271 | 36.831 | 1.00 | 30.69 | C |
| ATOM | 55 | ND1 | HIS | A | 1 | 33.424 | 33.069 | 37.801 | 1.00 | 30.91 | N |
| ATOM | 56 | CE1 | HIS | A | 1 | 33.990 | 34.258 | 37.790 | 1.00 | 25.35 | C |
| ATOM | 57 | NE2 | HIS | A | 1 | 34.887 | 34.273 | 36.824 | 1.00 | 30.30 | N |
| ATOM | 58 | CD2 | HIS | A | 1 | 34.892 | 33.046 | 36.197 | 1.00 | 25.86 | C |
| ATOM | 59 | C | HIS | A | 1 | 33.806 | 28.427 | 37.249 | 1.00 | 30.57 | C |
| ATOM | 60 | O | HIS | A | 1 | 32.588 | 28.307 | 37.257 | 1.00 | 29.93 | O |
| ATOM | 61 | N | LYS | A | 2 | 34.646 | 27.397 | 37.102 | 1.00 | 30.64 | N |
| ATOM | 62 | CA | LYS | A | 2 | 34.236 | 26.005 | 37.051 | 1.00 | 31.42 | C |
| ATOM | 63 | CB | LYS | A | 2 | 35.434 | 25.072 | 37.010 | 1.00 | 30.60 | C |
| ATOM | 64 | CG | LYS | A | 2 | 36.165 | 24.993 | 35.743 | 1.00 | 29.46 | C |
| ATOM | 65 | CD | LYS | A | 2 | 37.227 | 23.927 | 35.908 | 1.00 | 26.47 | C |
| ATOM | 66 | CE | LYS | A | 2 | 38.440 | 24.557 | 36.561 | 1.00 | 26.15 | C |
| ATOM | 67 | NZ | LYS | A | 2 | 39.414 | 23.476 | 36.821 | 1.00 | 28.93 | N |
| ATOM | 68 | C | LYS | A | 2 | 33.467 | 25.601 | 38.282 | 1.00 | 31.29 | C |
| ATOM | 69 | O | LYS | A | 2 | 32.553 | 24.762 | 38.175 | 1.00 | 30.92 | O |
| ATOM | 70 | N | ASP | A | 3 | 33.856 | 26.142 | 39.447 | 1.00 | 30.40 | N |
| ATOM | 71 | CA | ASP | A | 3 | 33.168 | 25.781 | 40.677 | 1.00 | 31.15 | C |
| ATOM | 72 | CB | ASP | A | 3 | 34.018 | 26.017 | 41.953 | 1.00 | 32.15 | C |
| ATOM | 73 | CG | ASP | A | 3 | 34.650 | 27.445 | 42.017 | 1.00 | 39.24 | C |
| ATOM | 74 | OD1 | ASP | A | 3 | 34.454 | 28.282 | 41.071 | 1.00 | 38.35 | O |
| ATOM | 75 | OD2 | ASP | A | 3 | 35.390 | 27.696 | 43.034 | 1.00 | 45.40 | O |
| ATOM | 76 | C | ASP | A | 3 | 31.764 | 26.410 | 40.747 | 1.00 | 29.46 | C |
| ATOM | 77 | O | ASP | A | 3 | 30.992 | 26.115 | 41.626 | 1.00 | 28.62 | O |
| ATOM | 78 | N | GLU | A | 4 | 31.409 | 27.245 | 39.789 | 1.00 | 28.05 | N |
| ATOM | 79 | CA | GLU | A | 4 | 30.030 | 27.750 | 39.760 | 1.00 | 27.71 | C |
| ATOM | 80 | CB | GLU | A | 4 | 30.027 | 29.190 | 39.258 | 1.00 | 29.09 | C |
| ATOM | 81 | CG | GLU | A | 4 | 31.112 | 30.021 | 39.957 | 1.00 | 34.58 | C |
| ATOM | 82 | CD | GLU | A | 4 | 30.816 | 31.477 | 39.858 | 1.00 | 41.79 | C |
| ATOM | 83 | OE1 | GLU | A | 4 | 29.818 | 31.830 | 40.492 | 1.00 | 47.49 | O |
| ATOM | 84 | OE2 | GLU | A | 4 | 31.547 | 32.257 | 39.179 | 1.00 | 41.63 | O |
| ATOM | 85 | C | GLU | A | 4 | 29.073 | 26.917 | 38.913 | 1.00 | 25.13 | C |
| ATOM | 86 | O | GLU | A | 4 | 27.869 | 27.176 | 38.941 | 1.00 | 25.62 | O |
| ATOM | 87 | N | VAL | A | 5 | 29.605 | 25.972 | 38.122 | 1.00 | 21.53 | N |
| ATOM | 88 | CA | VAL | A | 5 | 28.807 | 25.259 | 37.104 | 1.00 | 18.86 | C |
| ATOM | 89 | CB | VAL | A | 5 | 28.989 | 25.880 | 35.687 | 1.00 | 18.68 | C |
| ATOM | 90 | CG1 | VAL | A | 5 | 28.636 | 27.341 | 35.687 | 1.00 | 18.92 | C |
| ATOM | 91 | CG2 | VAL | A | 5 | 30.479 | 25.719 | 35.207 | 1.00 | 15.79 | C |
| ATOM | 92 | C | VAL | A | 5 | 29.099 | 23.739 | 37.029 | 1.00 | 16.85 | C |
| ATOM | 93 | O | VAL | A | 5 | 28.712 | 23.086 | 36.068 | 1.00 | 16.80 | O |
| ATOM | 94 | N | ALA | A | 6 | 29.807 | 23.186 | 38.015 | 1.00 | 15.69 | N |
| ATOM | 95 | CA | ALA | A | 6 | 30.302 | 21.815 | 37.911 | 1.00 | 14.08 | C |
| ATOM | 96 | CB | ALA | A | 6 | 31.464 | 21.535 | 38.950 | 1.00 | 11.02 | C |
| ATOM | 97 | C | ALA | A | 6 | 29.156 | 20.840 | 38.117 | 1.00 | 13.46 | C |
| ATOM | 98 | O | ALA | A | 6 | 29.185 | 19.787 | 37.499 | 1.00 | 14.39 | O |
| ATOM | 99 | N | LEU | A | 7 | 28.173 | 21.164 | 38.968 | 1.00 | 13.42 | N |
| ATOM | 100 | CA | LEU | A | 7 | 26.929 | 20.360 | 39.068 | 1.00 | 13.82 | C |
| ATOM | 101 | CB | LEU | A | 7 | 25.970 | 20.812 | 40.176 | 1.00 | 13.27 | C |
| ATOM | 102 | CG | LEU | A | 7 | 26.539 | 20.898 | 41.588 | 1.00 | 13.97 | C |
| ATOM | 103 | CD1 | LEU | A | 7 | 25.510 | 21.510 | 42.558 | 1.00 | 11.74 | C |
| ATOM | 104 | CD2 | LEU | A | 7 | 26.941 | 19.510 | 42.092 | 1.00 | 14.06 | C |
| ATOM | 105 | C | LEU | A | 7 | 26.157 | 20.363 | 37.773 | 1.00 | 14.03 | C |
| ATOM | 106 | O | LEU | A | 7 | 25.619 | 19.330 | 37.352 | 1.00 | 13.31 | O |
| ATOM | 107 | N | LEU | A | 8 | 26.044 | 21.537 | 37.145 | 1.00 | 14.06 | N |
| ATOM | 108 | CA | LEU | A | 8 | 25.348 | 21.637 | 35.864 | 1.00 | 13.18 | C |
| ATOM | 109 | CB | LEU | A | 8 | 25.335 | 23.115 | 35.414 | 1.00 | 13.77 | C |
| ATOM | 110 | CG | LEU | A | 8 | 24.715 | 24.164 | 36.346 | 1.00 | 14.55 | C |
| ATOM | 111 | CD1 | LEU | A | 8 | 24.831 | 25.603 | 35.752 | 1.00 | 8.94 | C |
| ATOM | 112 | CD2 | LEU | A | 8 | 23.234 | 23.801 | 36.798 | 1.00 | 7.45 | C |
| ATOM | 113 | C | LEU | A | 8 | 26.074 | 20.800 | 34.774 | 1.00 | 13.11 | C |
| ATOM | 114 | O | LEU | A | 8 | 25.471 | 20.178 | 33.900 | 1.00 | 13.24 | O |
| ATOM | 115 | N | ALA | A | 9 | 27.404 | 20.811 | 34.848 | 1.00 | 14.33 | N |
| ATOM | 116 | CA | ALA | A | 9 | 28.227 | 20.131 | 33.903 | 1.00 | 14.51 | C |
| ATOM | 117 | CB | ALA | A | 9 | 29.656 | 20.601 | 34.001 | 1.00 | 12.38 | C |
| ATOM | 118 | C | ALA | A | 9 | 28.086 | 18.596 | 34.127 | 1.00 | 14.24 | C |
| ATOM | 119 | O | ALA | A | 9 | 28.023 | 17.845 | 33.123 | 1.00 | 12.19 | O |
| ATOM | 120 | N | ALA | A | 10 | 28.057 | 18.157 | 35.399 | 1.00 | 14.37 | N |
| ATOM | 121 | CA | ALA | A | 10 | 27.853 | 16.756 | 35.760 | 1.00 | 14.67 | C |
| ATOM | 122 | CB | ALA | A | 10 | 28.007 | 16.541 | 37.242 | 1.00 | 13.11 | C |
| ATOM | 123 | C | ALA | A | 10 | 26.473 | 16.242 | 35.283 | 1.00 | 15.35 | C |
| ATOM | 124 | O | ALA | A | 10 | 26.348 | 15.103 | 34.823 | 1.00 | 15.31 | O |
| ATOM | 125 | N | VAL | A | 11 | 25.432 | 17.036 | 35.483 | 1.00 | 15.32 | N |
| ATOM | 126 | CA | VAL | A | 11 | 24.056 | 16.637 | 35.058 | 1.00 | 14.33 | C |
| ATOM | 127 | CB | VAL | A | 11 | 22.961 | 17.570 | 35.666 | 1.00 | 12.15 | C |
| ATOM | 128 | CG1 | VAL | A | 11 | 21.549 | 17.348 | 35.037 | 1.00 | 10.49 | C |
| ATOM | 129 | CG2 | VAL | A | 11 | 22.898 | 17.323 | 37.227 | 1.00 | 12.81 | C |
| ATOM | 130 | C | VAL | A | 11 | 23.989 | 16.614 | 33.528 | 1.00 | 17.02 | C |
| ATOM | 131 | O | VAL | A | 11 | 23.308 | 15.756 | 32.932 | 1.00 | 16.98 | O |
| ATOM | 132 | N | THR | A | 12 | 24.708 | 17.563 | 32.894 | 1.00 | 15.77 | N |
| ATOM | 133 | CA | THR | A | 12 | 24.798 | 17.603 | 31.426 | 1.00 | 16.21 | C |
| ATOM | 134 | CB | THR | A | 12 | 25.658 | 18.831 | 30.969 | 1.00 | 16.10 | C |
| ATOM | 135 | OG1 | THR | A | 12 | 24.986 | 20.020 | 31.391 | 1.00 | 13.83 | O |
| ATOM | 136 | CG2 | THR | A | 12 | 25.864 | 18.845 | 29.446 | 1.00 | 15.32 | C |
| ATOM | 137 | C | THR | A | 12 | 25.452 | 16.327 | 30.874 | 1.00 | 16.28 | C |
| ATOM | 138 | O | THR | A | 12 | 24.927 | 15.733 | 29.922 | 1.00 | 16.08 | O |
| ATOM | 139 | N | LEU | A | 13 | 26.563 | 15.918 | 31.478 | 1.00 | 13.10 | N |
| ATOM | 140 | CA | LEU | A | 13 | 27.250 | 14.704 | 31.126 | 1.00 | 14.57 | C |
| ATOM | 141 | CB | LEU | A | 13 | 28.630 | 14.631 | 31.829 | 1.00 | 12.99 | C |
| ATOM | 142 | CG | LEU | A | 13 | 29.387 | 13.383 | 31.346 | 1.00 | 18.22 | C |
| ATOM | 143 | CD1 | LEU | A | 13 | 29.645 | 13.517 | 29.809 | 1.00 | 15.91 | C |
| ATOM | 144 | CD2 | LEU | A | 13 | 30.658 | 13.091 | 32.099 | 1.00 | 18.98 | C |
| ATOM | 145 | C | LEU | A | 13 | 26.400 | 13.425 | 31.344 | 1.00 | 15.67 | C |
| ATOM | 146 | O | LEU | A | 13 | 26.334 | 12.543 | 30.496 | 1.00 | 16.15 | O |
| ATOM | 147 | N | LEU | A | 14 | 25.744 | 13.339 | 32.467 | 1.00 | 16.66 | N |
| ATOM | 148 | CA | LEU | A | 14 | 24.770 | 12.300 | 32.680 | 1.00 | 18.79 | C |
| ATOM | 149 | CB | LEU | A | 14 | 24.153 | 12.435 | 34.071 | 1.00 | 18.03 | C |
| ATOM | 150 | CG | LEU | A | 14 | 23.125 | 11.381 | 34.470 | 1.00 | 24.54 | C |
| ATOM | 151 | CD1 | LEU | A | 14 | 23.749 | 9.964 | 34.585 | 1.00 | 25.47 | C |
| ATOM | 152 | CD2 | LEU | A | 14 | 22.496 | 11.762 | 35.815 | 1.00 | 22.08 | C |
| ATOM | 153 | C | LEU | A | 14 | 23.688 | 12.262 | 31.600 | 1.00 | 19.00 | C |
| ATOM | 154 | O | LEU | A | 14 | 23.298 | 11.169 | 31.197 | 1.00 | 19.10 | O |
| ATOM | 155 | N | GLY | A | 15 | 23.167 | 13.433 | 31.199 | 1.00 | 19.54 | N |
| ATOM | 156 | CA | GLY | A | 15 | 22.243 | 13.576 | 30.078 | 1.00 | 18.51 | C |
| ATOM | 157 | C | GLY | A | 15 | 22.743 | 12.983 | 28.750 | 1.00 | 18.18 | C |
| ATOM | 158 | O | GLY | A | 15 | 21.981 | 12.279 | 28.077 | 1.00 | 18.00 | O |
| ATOM | 159 | N | VAL | A | 16 | 24.009 | 13.250 | 28.391 | 1.00 | 14.62 | N |
| ATOM | 160 | CA | VAL | A | 16 | 24.608 | 12.764 | 27.214 | 1.00 | 14.16 | C |
| ATOM | 161 | CB | VAL | A | 16 | 26.071 | 13.433 | 26.960 | 1.00 | 15.20 | C |
| ATOM | 162 | CG1 | VAL | A | 16 | 26.823 | 12.670 | 25.920 | 1.00 | 12.26 | C |
| ATOM | 163 | CG2 | VAL | A | 16 | 25.907 | 14.937 | 26.582 | 1.00 | 12.65 | C |
| ATOM | 164 | C | VAL | A | 16 | 24.735 | 11.252 | 27.250 | 1.00 | 13.94 | C |
| ATOM | 165 | O | VAL | A | 16 | 24.485 | 10.577 | 26.225 | 1.00 | 13.45 | O |
| ATOM | 166 | N | LEU | A | 17 | 25.155 | 10.713 | 28.413 | 1.00 | 12.68 | N |
| ATOM | 167 | CA | LEU | A | 17 | 25.238 | 9.280 | 28.592 | 1.00 | 13.23 | C |
| ATOM | 168 | CB | LEU | A | 17 | 25.798 | 8.914 | 29.967 | 1.00 | 11.56 | C |
| ATOM | 169 | CG | LEU | A | 17 | 27.292 | 9.195 | 30.095 | 1.00 | 16.23 | C |
| ATOM | 170 | CD1 | LEU | A | 17 | 27.690 | 9.247 | 31.608 | 1.00 | 14.52 | C |
| ATOM | 171 | CD2 | LEU | A | 17 | 28.121 | 8.165 | 29.346 | 1.00 | 14.49 | C |
| ATOM | 172 | C | LEU | A | 17 | 23.905 | 8.591 | 28.455 | 1.00 | 12.70 | C |
| ATOM | 173 | O | LEU | A | 17 | 23.843 | 7.464 | 27.959 | 1.00 | 11.56 | O |
| ATOM | 174 | N | LEU | A | 18 | 22.845 | 9.204 | 29.014 | 1.00 | 12.69 | N |
| ATOM | 175 | CA | LEU | A | 18 | 21.508 | 8.664 | 28.834 | 1.00 | 14.20 | C |
| ATOM | 176 | CB | LEU | A | 18 | 20.522 | 9.499 | 29.635 | 1.00 | 13.39 | C |
| ATOM | 177 | CG | LEU | A | 18 | 19.122 | 8.880 | 29.823 | 1.00 | 19.24 | C |
| ATOM | 178 | CD1 | LEU | A | 18 | 19.090 | 7.475 | 30.542 | 1.00 | 18.85 | C |
| ATOM | 179 | CD2 | LEU | A | 18 | 18.112 | 9.948 | 30.464 | 1.00 | 16.88 | C |
| ATOM | 180 | C | LEU | A | 18 | 21.088 | 8.637 | 27.323 | 1.00 | 12.86 | C |
| ATOM | 181 | O | LEU | A | 18 | 20.470 | 7.683 | 26.877 | 1.00 | 14.91 | O |
| ATOM | 182 | N | GLN | A | 19 | 21.372 | 9.701 | 26.567 | 1.00 | 12.79 | N |
| ATOM | 183 | CA | GLN | A | 19 | 21.004 | 9.815 | 25.141 | 1.00 | 14.03 | C |
| ATOM | 184 | CB | GLN | A | 19 | 21.365 | 11.183 | 24.568 | 1.00 | 13.85 | C |
| ATOM | 185 | CG | GLN | A | 19 | 20.385 | 12.290 | 25.041 | 1.00 | 13.14 | C |
| ATOM | 186 | CD | GLN | A | 19 | 18.902 | 12.058 | 24.662 | 1.00 | 17.44 | C |
| ATOM | 187 | OE1 | GLN | A | 19 | 18.030 | 12.114 | 25.511 | 1.00 | 18.58 | O |
| ATOM | 188 | NE2 | GLN | A | 19 | 18.636 | 11.780 | 23.422 | 1.00 | 11.71 | N |
| ATOM | 189 | C | GLN | A | 19 | 21.823 | 8.766 | 24.366 | 1.00 | 13.92 | C |
| ATOM | 190 | O | GLN | A | 19 | 21.270 | 8.116 | 23.515 | 1.00 | 15.23 | O |
| ATOM | 191 | N | ALA | A | 20 | 23.085 | 8.528 | 24.761 | 1.00 | 13.14 | N |
| ATOM | 192 | CA | ALA | A | 20 | 23.928 | 7.510 | 24.165 | 1.00 | 13.90 | C |
| ATOM | 193 | CB | ALA | A | 20 | 25.334 | 7.530 | 24.801 | 1.00 | 12.18 | C |
| ATOM | 194 | C | ALA | A | 20 | 23.260 | 6.095 | 24.341 | 1.00 | 14.12 | C |
| ATOM | 195 | O | ALA | A | 20 | 23.175 | 5.317 | 23.410 | 1.00 | 13.96 | O |
| ATOM | 196 | N | TYR | A | 21 | 22.827 | 5.807 | 25.553 | 1.00 | 15.22 | N |
| ATOM | 197 | CA | TYR | A | 21 | 22.074 | 4.571 | 25.895 | 1.00 | 14.63 | C |
| ATOM | 198 | CB | TYR | A | 21 | 21.726 | 4.595 | 27.394 | 1.00 | 12.88 | C |
| ATOM | 199 | CG | TYR | A | 21 | 20.766 | 3.477 | 27.812 | 1.00 | 14.08 | C |
| ATOM | 200 | CD1 | TYR | A | 21 | 21.226 | 2.173 | 27.952 | 1.00 | 12.81 | C |
| ATOM | 201 | CE1 | TYR | A | 21 | 20.384 | 1.131 | 28.285 | 1.00 | 13.55 | C |
| ATOM | 202 | CZ | TYR | A | 21 | 19.041 | 1.375 | 28.478 | 1.00 | 14.21 | C |
| ATOM | 203 | OH | TYR | A | 21 | 18.284 | 0.289 | 28.855 | 1.00 | 17.59 | O |
| ATOM | 204 | CE2 | TYR | A | 21 | 18.511 | 2.666 | 28.341 | 1.00 | 14.36 | C |
| ATOM | 205 | CD2 | TYR | A | 21 | 19.404 | 3.716 | 27.983 | 1.00 | 12.11 | C |
| ATOM | 206 | C | TYR | A | 21 | 20.815 | 4.357 | 24.999 | 1.00 | 14.79 | C |
| ATOM | 207 | O | TYR | A | 21 | 20.563 | 3.241 | 24.524 | 1.00 | 15.79 | O |
| ATOM | 208 | N | PHE | A | 22 | 20.020 | 5.419 | 24.820 | 1.00 | 13.65 | N |
| ATOM | 209 | CA | PHE | A | 22 | 18.812 | 5.396 | 24.001 | 1.00 | 13.77 | C |
| ATOM | 210 | CB | PHE | A | 22 | 18.101 | 6.784 | 23.979 | 1.00 | 12.79 | C |
| ATOM | 211 | CG | PHE | A | 22 | 17.533 | 7.202 | 25.310 | 1.00 | 13.01 | C |
| ATOM | 212 | CD1 | PHE | A | 22 | 17.200 | 6.234 | 26.299 | 1.00 | 9.44 | C |
| ATOM | 213 | CE1 | PHE | A | 22 | 16.662 | 6.615 | 27.542 | 1.00 | 10.73 | C |
| ATOM | 214 | CZ | PHE | A | 22 | 16.457 | 7.957 | 27.798 | 1.00 | 12.97 | C |
| ATOM | 215 | CE2 | PHE | A | 22 | 16.818 | 8.940 | 26.800 | 1.00 | 9.75 | C |
| ATOM | 216 | CD2 | PHE | A | 22 | 17.326 | 8.556 | 25.595 | 1.00 | 9.89 | C |
| ATOM | 217 | C | PHE | A | 22 | 19.225 | 5.028 | 22.563 | 1.00 | 14.12 | C |
| ATOM | 218 | O | PHE | A | 22 | 18.553 | 4.202 | 21.895 | 1.00 | 12.02 | O |
| ATOM | 219 | N | SER | A | 23 | 20.328 | 5.633 | 22.115 | 1.00 | 13.90 | N |
| ATOM | 220 | CA | SER | A | 23 | 20.858 | 5.344 | 20.792 | 1.00 | 14.33 | C |
| ATOM | 221 | CB | SER | A | 23 | 22.038 | 6.244 | 20.365 | 1.00 | 13.68 | C |
| ATOM | 222 | OG | SER | A | 23 | 21.537 | 7.538 | 20.302 | 1.00 | 22.60 | O |
| ATOM | 223 | C | SER | A | 23 | 21.331 | 3.934 | 20.671 | 1.00 | 12.41 | C |
| ATOM | 224 | O | SER | A | 23 | 21.106 | 3.315 | 19.649 | 1.00 | 12.71 | O |
| ATOM | 225 | N | LEU | A | 24 | 22.063 | 3.424 | 21.637 | 1.00 | 11.34 | N |
| ATOM | 226 | CA | LEU | A | 24 | 22.516 | 2.029 | 21.489 | 1.00 | 9.87 | C |
| ATOM | 227 | CB | LEU | A | 24 | 23.407 | 1.702 | 22.679 | 1.00 | 10.30 | C |
| ATOM | 228 | CG | LEU | A | 24 | 24.725 | 2.524 | 22.673 | 1.00 | 13.60 | C |
| ATOM | 229 | CD1 | LEU | A | 24 | 25.540 | 2.351 | 24.000 | 1.00 | 11.42 | C |
| ATOM | 230 | CD2 | LEU | A | 24 | 25.599 | 2.005 | 21.456 | 1.00 | 13.75 | C |
| ATOM | 231 | C | LEU | A | 24 | 21.303 | 1.081 | 21.505 | 1.00 | 10.73 | C |
| ATOM | 232 | O | LEU | A | 24 | 21.342 | −0.016 | 20.919 | 1.00 | 9.21 | O |
| ATOM | 233 | N | GLN | A | 25 | 20.235 | 1.479 | 22.228 | 1.00 | 10.44 | N |
| ATOM | 234 | CA | GLN | A | 25 | 18.962 | 0.658 | 22.266 | 1.00 | 11.89 | C |
| ATOM | 235 | CB | GLN | A | 25 | 18.054 | 1.091 | 23.443 | 1.00 | 12.09 | C |
| ATOM | 236 | CG | GLN | A | 25 | 18.702 | 0.839 | 24.798 | 1.00 | 12.91 | C |
| ATOM | 237 | CD | GLN | A | 25 | 18.745 | −0.651 | 25.075 | 1.00 | 20.45 | C |
| ATOM | 238 | OE1 | GLN | A | 25 | 19.774 | −1.298 | 24.951 | 1.00 | 22.86 | O |
| ATOM | 239 | NE2 | GLN | A | 25 | 17.606 | −1.207 | 25.352 | 1.00 | 20.18 | N |
| ATOM | 240 | C | GLN | A | 25 | 18.208 | 0.659 | 20.949 | 1.00 | 11.61 | C |
| ATOM | 241 | O | GLN | A | 25 | 17.641 | −0.349 | 20.541 | 1.00 | 14.14 | O |
| ATOM | 242 | N | VAL | A | 26 | 18.185 | 1.786 | 20.257 | 1.00 | 13.41 | N |
| ATOM | 243 | CA | VAL | A | 26 | 17.685 | 1.765 | 18.887 | 1.00 | 12.07 | C |
| ATOM | 244 | CB | VAL | A | 26 | 17.628 | 3.193 | 18.290 | 1.00 | 13.82 | C |
| ATOM | 245 | CG1 | VAL | A | 26 | 17.136 | 3.074 | 16.788 | 1.00 | 9.67 | C |
| ATOM | 246 | CG2 | VAL | A | 26 | 16.680 | 4.017 | 19.099 | 1.00 | 12.85 | C |
| ATOM | 247 | C | VAL | A | 26 | 18.531 | 0.829 | 17.964 | 1.00 | 11.93 | C |
| ATOM | 248 | O | VAL | A | 26 | 17.966 | 0.078 | 17.174 | 1.00 | 11.16 | O |
| ATOM | 249 | N | ILE | A | 27 | 19.874 | 0.927 | 18.006 | 1.00 | 10.57 | N |
| ATOM | 250 | CA | ILE | A | 27 | 20.744 | 0.022 | 17.221 | 1.00 | 9.41 | C |
| ATOM | 251 | CB | ILE | A | 27 | 22.218 | 0.318 | 17.510 | 1.00 | 8.62 | C |
| ATOM | 252 | CG1 | ILE | A | 27 | 22.529 | 1.749 | 17.019 | 1.00 | 7.59 | C |
| ATOM | 253 | CD1 | ILE | A | 27 | 23.899 | 2.314 | 17.344 | 1.00 | 10.48 | C |
| ATOM | 254 | CG2 | ILE | A | 27 | 23.119 | −0.792 | 16.846 | 1.00 | 5.42 | C |
| ATOM | 255 | C | ILE | A | 27 | 20.391 | −1.464 | 17.507 | 1.00 | 10.44 | C |
| ATOM | 256 | O | ILE | A | 27 | 20.296 | −2.326 | 16.600 | 1.00 | 11.32 | O |
| ATOM | 257 | N | SER | A | 28 | 20.157 | −1.748 | 18.771 | 1.00 | 10.48 | N |
| ATOM | 258 | CA | SER | A | 28 | 19.829 | −3.060 | 19.189 | 1.00 | 12.55 | C |
| ATOM | 259 | CB | SER | A | 28 | 19.932 | −3.099 | 20.723 | 1.00 | 12.29 | C |
| ATOM | 260 | OG | SER | A | 28 | 19.487 | −4.370 | 21.191 | 1.00 | 17.87 | O |
| ATOM | 261 | C | SER | A | 28 | 18.422 | −3.448 | 18.660 | 1.00 | 11.75 | C |
| ATOM | 262 | O | SER | A | 28 | 18.199 | −4.572 | 18.248 | 1.00 | 10.69 | O |
| ATOM | 263 | N | ALA | A | 29 | 17.475 | −2.515 | 18.644 | 1.00 | 12.74 | N |
| ATOM | 264 | CA | ALA | A | 29 | 16.139 | −2.833 | 18.067 | 1.00 | 13.62 | C |
| ATOM | 265 | CB | ALA | A | 29 | 15.133 | −1.670 | 18.399 | 1.00 | 13.35 | C |
| ATOM | 266 | C | ALA | A | 29 | 16.229 | −3.069 | 16.521 | 1.00 | 12.51 | C |
| ATOM | 267 | O | ALA | A | 29 | 15.497 | −3.835 | 15.967 | 1.00 | 12.55 | O |
| ATOM | 268 | N | ARG | A | 30 | 17.148 | −2.420 | 15.848 | 1.00 | 11.66 | N |
| ATOM | 269 | CA | ARG | A | 30 | 17.317 | −2.575 | 14.409 | 1.00 | 13.71 | C |
| ATOM | 270 | CB | ARG | A | 30 | 18.356 | −1.560 | 13.883 | 1.00 | 13.34 | C |
| ATOM | 271 | CG | ARG | A | 30 | 17.788 | −0.190 | 13.607 | 1.00 | 12.61 | C |
| ATOM | 272 | CD | ARG | A | 30 | 18.745 | 0.766 | 12.913 | 1.00 | 13.07 | C |
| ATOM | 273 | NE | ARG | A | 30 | 18.157 | 2.108 | 12.760 | 1.00 | 12.05 | N |
| ATOM | 274 | CZ | ARG | A | 30 | 17.255 | 2.449 | 11.830 | 1.00 | 12.21 | C |
| ATOM | 275 | NH1 | ARG | A | 30 | 16.827 | 1.523 | 10.929 | 1.00 | 7.86 | N |
| ATOM | 276 | NH2 | ARG | A | 30 | 16.792 | 3.727 | 11.747 | 1.00 | 5.61 | N |
| ATOM | 277 | C | ARG | A | 30 | 17.766 | −3.994 | 14.130 | 1.00 | 15.25 | C |
| ATOM | 278 | O | ARG | A | 30 | 17.277 | −4.635 | 13.202 | 1.00 | 15.66 | O |
| ATOM | 279 | N | ARG | A | 31 | 18.698 | −4.480 | 14.957 | 1.00 | 17.22 | N |
| ATOM | 280 | CA | ARG | A | 31 | 19.152 | −5.856 | 14.911 | 1.00 | 20.02 | C |
| ATOM | 281 | CB | ARG | A | 31 | 20.351 | −6.058 | 15.855 | 1.00 | 18.29 | C |
| ATOM | 282 | CG | ARG | A | 31 | 21.084 | −7.465 | 15.656 | 1.00 | 26.40 | C |
| ATOM | 283 | CD | ARG | A | 31 | 22.466 | −7.540 | 16.418 | 1.00 | 28.12 | C |
| ATOM | 284 | NE | ARG | A | 31 | 22.986 | −6.152 | 16.672 | 1.00 | 44.00 | N |
| ATOM | 285 | CZ | ARG | A | 31 | 23.816 | −5.440 | 15.868 | 1.00 | 46.92 | C |
| ATOM | 286 | NH1 | ARG | A | 31 | 24.296 | −5.990 | 14.717 | 1.00 | 49.61 | N |
| ATOM | 287 | NH2 | ARG | A | 31 | 24.186 | −4.189 | 16.220 | 1.00 | 41.52 | N |
| ATOM | 288 | C | ARG | A | 31 | 18.015 | −6.885 | 15.203 | 1.00 | 18.00 | C |
| ATOM | 289 | O | ARG | A | 31 | 17.697 | −7.700 | 14.339 | 1.00 | 16.67 | O |
| ATOM | 290 | N | ALA | A | 32 | 17.409 | −6.835 | 16.403 | 1.00 | 17.87 | N |
| ATOM | 291 | CA | ALA | A | 32 | 16.280 | −7.703 | 16.761 | 1.00 | 17.39 | C |
| ATOM | 292 | CB | ALA | A | 32 | 15.704 | −7.291 | 18.143 | 1.00 | 15.94 | C |
| ATOM | 293 | C | ALA | A | 32 | 15.170 | −7.750 | 15.685 | 1.00 | 17.99 | C |
| ATOM | 294 | O | ALA | A | 32 | 14.678 | −8.814 | 15.388 | 1.00 | 18.15 | O |
| ATOM | 295 | N | PHE | A | 33 | 14.785 | −6.609 | 15.104 | 1.00 | 18.07 | N |
| ATOM | 296 | CA | PHE | A | 33 | 13.641 | −6.533 | 14.201 | 1.00 | 20.32 | C |
| ATOM | 297 | CB | PHE | A | 33 | 12.694 | −5.399 | 14.592 | 1.00 | 21.89 | C |
| ATOM | 298 | CG | PHE | A | 33 | 12.162 | −5.541 | 15.978 | 1.00 | 23.17 | C |
| ATOM | 299 | CD1 | PHE | A | 33 | 11.065 | −6.357 | 16.230 | 1.00 | 28.57 | C |
| ATOM | 300 | CE1 | PHE | A | 33 | 10.593 | −6.518 | 17.547 | 1.00 | 29.89 | C |
| ATOM | 301 | CZ | PHE | A | 33 | 11.239 | −5.884 | 18.591 | 1.00 | 27.38 | C |
| ATOM | 302 | CE2 | PHE | A | 33 | 12.311 | −5.029 | 18.334 | 1.00 | 28.15 | C |
| ATOM | 303 | CD2 | PHE | A | 33 | 12.773 | −4.889 | 17.038 | 1.00 | 25.87 | C |
| ATOM | 304 | C | PHE | A | 33 | 14.036 | −6.423 | 12.746 | 1.00 | 21.44 | C |
| ATOM | 305 | O | PHE | A | 33 | 13.177 | −6.317 | 11.857 | 1.00 | 22.66 | O |
| ATOM | 306 | N | ARG | A | 34 | 15.339 | −6.506 | 12.484 | 1.00 | 21.20 | N |
| ATOM | 307 | CA | ARG | A | 34 | 15.819 | −6.527 | 11.098 | 1.00 | 21.81 | C |
| ATOM | 308 | CB | ARG | A | 34 | 15.556 | −7.892 | 10.425 | 1.00 | 21.90 | C |
| ATOM | 309 | CG | ARG | A | 34 | 16.064 | −9.089 | 11.279 | 1.00 | 25.86 | C |
| ATOM | 310 | CD | ARG | A | 34 | 16.624 | −10.245 | 10.465 | 1.00 | 31.94 | C |
| ATOM | 311 | NE | ARG | A | 34 | 15.585 | −11.210 | 10.111 | 1.00 | 37.48 | N |
| ATOM | 312 | CZ | ARG | A | 34 | 14.997 | −12.057 | 10.967 | 1.00 | 42.81 | C |
| ATOM | 313 | NH1 | ARG | A | 34 | 15.336 | −12.039 | 12.268 | 1.00 | 45.32 | N |
| ATOM | 314 | NH2 | ARG | A | 34 | 14.046 | −12.913 | 10.528 | 1.00 | 43.06 | N |
| ATOM | 315 | C | ARG | A | 34 | 15.291 | −5.334 | 10.267 | 1.00 | 20.00 | C |
| ATOM | 316 | O | ARG | A | 34 | 14.887 | −5.493 | 9.105 | 1.00 | 22.72 | O |
| ATOM | 317 | N | VAL | A | 35 | 15.324 | −4.153 | 10.882 | 1.00 | 18.69 | N |
| ATOM | 318 | CA | VAL | A | 35 | 14.985 | −2.885 | 10.239 | 1.00 | 15.42 | C |
| ATOM | 319 | CB | VAL | A | 35 | 14.071 | −2.014 | 11.137 | 1.00 | 13.95 | C |
| ATOM | 320 | CG1 | VAL | A | 35 | 13.696 | −0.739 | 10.374 | 1.00 | 12.35 | C |
| ATOM | 321 | CG2 | VAL | A | 35 | 12.796 | −2.771 | 11.580 | 1.00 | 14.58 | C |
| ATOM | 322 | C | VAL | A | 35 | 16.252 | −2.122 | 9.891 | 1.00 | 15.66 | C |
| ATOM | 323 | O | VAL | A | 35 | 16.864 | −1.503 | 10.748 | 1.00 | 12.69 | O |
| ATOM | 324 | N | SER | A | 36 | 16.661 | −2.172 | 8.625 | 1.00 | 15.31 | N |
| ATOM | 325 | CA | SER | A | 36 | 17.901 | −1.496 | 8.223 | 1.00 | 17.02 | C |
| ATOM | 326 | CB | SER | A | 36 | 18.550 | −2.218 | 7.034 | 1.00 | 16.98 | C |
| ATOM | 327 | OG | SER | A | 36 | 18.318 | −3.599 | 7.163 | 1.00 | 26.41 | O |
| ATOM | 328 | C | SER | A | 36 | 17.764 | −0.054 | 7.778 | 1.00 | 15.01 | C |
| ATOM | 329 | O | SER | A | 36 | 16.839 | 0.285 | 7.027 | 1.00 | 15.40 | O |
| ATOM | 330 | N | PRO | A | 37 | 18.760 | 0.779 | 8.139 | 1.00 | 14.72 | N |
| ATOM | 331 | CA | PRO | A | 37 | 18.760 | 2.133 | 7.551 | 1.00 | 13.18 | C |
| ATOM | 332 | CB | PRO | A | 37 | 20.082 | 2.753 | 8.103 | 1.00 | 13.40 | C |
| ATOM | 333 | CG | PRO | A | 37 | 20.325 | 2.015 | 9.380 | 1.00 | 12.81 | C |
| ATOM | 334 | CD | PRO | A | 37 | 19.905 | 0.558 | 9.058 | 1.00 | 12.35 | C |
| ATOM | 335 | C | PRO | A | 37 | 18.699 | 2.072 | 5.987 | 1.00 | 12.15 | C |
| ATOM | 336 | O | PRO | A | 37 | 19.263 | 1.162 | 5.413 | 1.00 | 13.45 | O |
| ATOM | 337 | N | PRO | A | 38 | 18.069 | 3.059 | 5.291 | 1.00 | 12.51 | N |
| ATOM | 338 | CA | PRO | A | 38 | 17.526 | 4.340 | 5.802 | 1.00 | 11.81 | C |
| ATOM | 339 | CB | PRO | A | 38 | 17.449 | 5.201 | 4.549 | 1.00 | 10.25 | C |
| ATOM | 340 | CG | PRO | A | 38 | 17.147 | 4.182 | 3.365 | 1.00 | 9.40 | C |
| ATOM | 341 | CD | PRO | A | 38 | 17.900 | 2.913 | 3.810 | 1.00 | 10.84 | C |
| ATOM | 342 | C | PRO | A | 38 | 16.175 | 3.938 | 6.400 | 1.00 | 13.91 | C |
| ATOM | 343 | O | PRO | A | 38 | 15.905 | 2.742 | 6.564 | 1.00 | 14.88 | O |
| ATOM | 344 | N | LEU | A | 39 | 15.219 | 4.705 | 6.803 | 1.00 | 15.38 | N |
| ATOM | 345 | CA | LEU | A | 39 | 14.063 | 3.608 | 7.197 | 1.00 | 15.49 | C |
| ATOM | 346 | CB | LEU | A | 39 | 14.154 | 2.190 | 6.569 | 1.00 | 14.92 | C |
| ATOM | 347 | CG | LEU | A | 39 | 12.971 | 1.265 | 5.976 | 1.00 | 18.58 | C |
| ATOM | 348 | CD1 | LEU | A | 39 | 13.255 | −0.195 | 5.671 | 1.00 | 19.59 | C |
| ATOM | 349 | CD2 | LEU | A | 39 | 12.324 | 1.770 | 4.637 | 1.00 | 11.24 | C |
| ATOM | 350 | C | LEU | A | 39 | 13.975 | 3.353 | 8.661 | 1.00 | 14.01 | C |
| ATOM | 351 | O | LEU | A | 39 | 14.878 | 2.784 | 9.319 | 1.00 | 11.41 | O |
| ATOM | 352 | N | THR | A | 40 | 12.827 | 3.808 | 9.110 | 1.00 | 14.12 | N |
| ATOM | 353 | CA | THR | A | 40 | 12.557 | 4.170 | 10.483 | 1.00 | 15.41 | C |
| ATOM | 354 | CB | THR | A | 40 | 12.758 | 5.677 | 10.653 | 1.00 | 16.77 | C |
| ATOM | 355 | OG1 | THR | A | 40 | 11.901 | 6.359 | 9.732 | 1.00 | 15.44 | O |
| ATOM | 356 | CG2 | THR | A | 40 | 14.229 | 6.045 | 10.340 | 1.00 | 11.31 | C |
| ATOM | 357 | C | THR | A | 40 | 11.120 | 3.663 | 10.757 | 1.00 | 16.03 | C |
| ATOM | 358 | O | THR | A | 40 | 10.531 | 3.951 | 11.780 | 1.00 | 17.93 | O |
| ATOM | 359 | N | THR | A | 41 | 10.637 | 2.788 | 9.875 | 1.00 | 14.57 | N |
| ATOM | 360 | CA | THR | A | 41 | 9.354 | 2.105 | 10.052 | 1.00 | 15.88 | C |
| ATOM | 361 | CB | THR | A | 41 | 8.300 | 2.555 | 8.982 | 1.00 | 14.57 | C |
| ATOM | 362 | OG1 | THR | A | 41 | 8.861 | 2.299 | 7.688 | 1.00 | 17.40 | O |
| ATOM | 363 | CG2 | THR | A | 41 | 7.934 | 4.044 | 9.185 | 1.00 | 14.46 | C |
| ATOM | 364 | C | THR | A | 41 | 9.575 | 0.583 | 10.018 | 1.00 | 15.83 | C |
| ATOM | 365 | O | THR | A | 41 | 10.563 | 0.097 | 9.430 | 1.00 | 14.69 | O |
| ATOM | 366 | N | GLY | A | 42 | 8.639 | −0.152 | 10.613 | 1.00 | 16.94 | N |
| ATOM | 367 | CA | GLY | A | 42 | 8.766 | −1.596 | 10.828 | 1.00 | 18.20 | C |
| ATOM | 368 | C | GLY | A | 42 | 7.644 | −1.976 | 11.763 | 1.00 | 19.62 | C |
| ATOM | 369 | O | GLY | A | 42 | 6.656 | −1.278 | 11.839 | 1.00 | 20.86 | O |
| ATOM | 370 | N | PRO | A | 43 | 7.770 | −3.095 | 12.471 | 1.00 | 20.21 | N |
| ATOM | 371 | CA | PRO | A | 43 | 6.810 | −3.419 | 13.539 | 1.00 | 20.36 | C |
| ATOM | 372 | CB | PRO | A | 43 | 7.399 | −4.691 | 14.171 | 1.00 | 21.77 | C |
| ATOM | 373 | CG | PRO | A | 43 | 8.766 | −4.877 | 13.585 | 1.00 | 20.01 | C |
| ATOM | 374 | CD | PRO | A | 43 | 8.835 | −4.097 | 12.314 | 1.00 | 19.47 | C |
| ATOM | 375 | C | PRO | A | 43 | 6.598 | −2.319 | 14.631 | 1.00 | 19.46 | C |
| ATOM | 376 | O | PRO | A | 43 | 7.561 | −1.625 | 14.985 | 1.00 | 20.21 | O |
| ATOM | 377 | N | PRO | A | 44 | 5.348 | −2.153 | 15.133 | 1.00 | 19.30 | N |
| ATOM | 378 | CA | PRO | A | 44 | 5.052 | −1.157 | 16.164 | 1.00 | 19.49 | C |
| ATOM | 379 | CB | PRO | A | 44 | 3.669 | −1.559 | 16.689 | 1.00 | 19.77 | C |
| ATOM | 380 | CG | PRO | A | 44 | 3.014 | −2.130 | 15.453 | 1.00 | 19.90 | C |
| ATOM | 381 | CD | PRO | A | 44 | 4.126 | −2.906 | 14.744 | 1.00 | 18.39 | C |
| ATOM | 382 | C | PRO | A | 44 | 6.136 | −1.167 | 17.259 | 1.00 | 19.75 | C |
| ATOM | 383 | O | PRO | A | 44 | 6.612 | −0.113 | 17.698 | 1.00 | 19.34 | O |
| ATOM | 384 | N | GLU | A | 45 | 6.611 | −2.347 | 17.607 | 1.00 | 19.40 | N |
| ATOM | 385 | CA | GLU | A | 45 | 7.605 | −2.462 | 18.654 | 1.00 | 19.71 | C |
| ATOM | 386 | CB | GLU | A | 45 | 7.680 | −3.903 | 19.123 | 1.00 | 21.07 | C |
| ATOM | 387 | CG | GLU | A | 45 | 8.633 | −4.041 | 20.294 | 1.00 | 27.54 | C |
| ATOM | 388 | CD | GLU | A | 45 | 8.408 | −5.319 | 21.094 | 1.00 | 34.48 | C |
| ATOM | 389 | OE1 | GLU | A | 45 | 7.879 | −6.318 | 20.492 | 1.00 | 36.28 | O |
| ATOM | 390 | OE2 | GLU | A | 45 | 8.768 | −5.279 | 22.307 | 1.00 | 33.16 | O |
| ATOM | 391 | C | GLU | A | 45 | 9.001 | −1.915 | 18.283 | 1.00 | 18.56 | C |
| ATOM | 392 | O | GLU | A | 45 | 9.719 | −1.429 | 19.156 | 1.00 | 18.28 | O |
| ATOM | 393 | N | PHE | A | 46 | 9.380 | −1.963 | 16.995 | 1.00 | 16.03 | N |
| ATOM | 394 | CA | PHE | A | 46 | 10.560 | −1.264 | 16.602 | 1.00 | 15.23 | C |
| ATOM | 395 | CB | PHE | A | 46 | 11.099 | −1.721 | 15.239 | 1.00 | 14.81 | C |
| ATOM | 396 | CG | PHE | A | 46 | 12.066 | −0.764 | 14.700 | 1.00 | 17.22 | C |
| ATOM | 397 | CD1 | PHE | A | 46 | 13.390 | −0.756 | 15.153 | 1.00 | 14.12 | C |
| ATOM | 398 | CE1 | PHE | A | 46 | 14.295 | 0.242 | 14.732 | 1.00 | 19.07 | C |
| ATOM | 399 | CZ | PHE | A | 46 | 13.884 | 1.233 | 13.890 | 1.00 | 17.14 | C |
| ATOM | 400 | CE2 | PHE | A | 46 | 12.537 | 1.232 | 13.428 | 1.00 | 17.68 | C |
| ATOM | 401 | CD2 | PHE | A | 46 | 11.637 | 0.260 | 13.860 | 1.00 | 18.51 | C |
| ATOM | 402 | C | PHE | A | 46 | 10.299 | 0.296 | 16.601 | 1.00 | 13.78 | C |
| ATOM | 403 | O | PHE | A | 46 | 11.123 | 1.038 | 17.040 | 1.00 | 12.23 | O |
| ATOM | 404 | N | GLU | A | 47 | 9.143 | 0.739 | 16.117 | 1.00 | 12.97 | N |
| ATOM | 405 | CA | GLU | A | 47 | 8.795 | 2.144 | 15.986 | 1.00 | 11.61 | C |
| ATOM | 406 | CB | GLU | A | 47 | 7.517 | 2.295 | 15.156 | 1.00 | 11.60 | C |
| ATOM | 407 | CG | GLU | A | 47 | 7.725 | 1.795 | 13.706 | 1.00 | 10.58 | C |
| ATOM | 408 | CD | GLU | A | 47 | 6.603 | 2.205 | 12.721 | 1.00 | 14.43 | C |
| ATOM | 409 | OE1 | GLU | A | 47 | 5.642 | 2.946 | 13.076 | 1.00 | 16.62 | O |
| ATOM | 410 | OE2 | GLU | A | 47 | 6.744 | 1.801 | 11.554 | 1.00 | 15.88 | O |
| ATOM | 411 | C | GLU | A | 47 | 8.691 | 2.868 | 17.305 | 1.00 | 13.07 | C |
| ATOM | 412 | O | GLU | A | 47 | 9.172 | 4.010 | 17.440 | 1.00 | 11.47 | O |
| ATOM | 413 | N | ARG | A | 48 | 8.105 | 2.205 | 18.290 | 1.00 | 13.97 | N |
| ATOM | 414 | CA | ARG | A | 48 | 8.188 | 2.722 | 19.665 | 1.00 | 15.04 | C |
| ATOM | 415 | CB | ARG | A | 48 | 7.347 | 1.874 | 20.639 | 1.00 | 15.02 | C |
| ATOM | 416 | CG | ARG | A | 48 | 5.844 | 1.967 | 20.364 | 1.00 | 14.43 | C |
| ATOM | 417 | CD | ARG | A | 48 | 4.990 | 1.353 | 21.493 | 1.00 | 13.52 | C |
| ATOM | 418 | NE | ARG | A | 48 | 5.592 | 0.097 | 21.947 | 1.00 | 13.44 | N |
| ATOM | 419 | CZ | ARG | A | 48 | 5.269 | −1.127 | 21.503 | 1.00 | 13.23 | C |
| ATOM | 420 | NH1 | ARG | A | 48 | 4.309 | −1.328 | 20.596 | 1.00 | 10.55 | N |
| ATOM | 421 | NH2 | ARG | A | 48 | 5.929 | −2.138 | 21.938 | 1.00 | 7.17 | N |
| ATOM | 422 | C | ARG | A | 48 | 9.590 | 3.022 | 20.224 | 1.00 | 15.81 | C |
| ATOM | 423 | O | ARG | A | 48 | 9.760 | 4.044 | 20.845 | 1.00 | 16.42 | O |
| ATOM | 424 | N | VAL | A | 49 | 10.582 | 2.136 | 20.024 | 1.00 | 16.51 | N |
| ATOM | 425 | CA | VAL | A | 49 | 11.923 | 2.367 | 20.523 | 1.00 | 14.75 | C |
| ATOM | 426 | CB | VAL | A | 49 | 12.849 | 1.114 | 20.324 | 1.00 | 16.09 | C |
| ATOM | 427 | CG1 | VAL | A | 49 | 14.301 | 1.370 | 20.855 | 1.00 | 14.60 | C |
| ATOM | 428 | CG2 | VAL | A | 49 | 12.303 | −0.088 | 21.055 | 1.00 | 16.72 | C |
| ATOM | 429 | C | VAL | A | 49 | 12.553 | 3.508 | 19.743 | 1.00 | 14.72 | C |
| ATOM | 430 | O | VAL | A | 49 | 13.198 | 4.372 | 20.338 | 1.00 | 12.53 | O |
| ATOM | 431 | N | TYR | A | 50 | 12.468 | 3.449 | 18.406 | 1.00 | 12.52 | N |
| ATOM | 432 | CA | TYR | A | 50 | 12.897 | 4.549 | 17.557 | 1.00 | 12.27 | C |
| ATOM | 433 | CB | TYR | A | 50 | 12.485 | 4.247 | 16.125 | 1.00 | 13.15 | C |
| ATOM | 434 | CG | TYR | A | 50 | 12.675 | 5.414 | 15.251 | 1.00 | 10.62 | C |
| ATOM | 435 | CD1 | TYR | A | 50 | 13.959 | 5.777 | 14.880 | 1.00 | 12.53 | C |
| ATOM | 436 | CE1 | TYR | A | 50 | 14.173 | 6.843 | 14.100 | 1.00 | 12.06 | C |
| ATOM | 437 | CZ | TYR | A | 50 | 13.113 | 7.644 | 13.676 | 1.00 | 9.95 | C |
| ATOM | 438 | OH | TYR | A | 50 | 13.469 | 8.739 | 12.908 | 1.00 | 11.26 | O |
| ATOM | 439 | CE2 | TYR | A | 50 | 11.795 | 7.350 | 13.982 | 1.00 | 9.79 | C |
| ATOM | 440 | CD2 | TYR | A | 50 | 11.581 | 6.177 | 14.801 | 1.00 | 13.35 | C |
| ATOM | 441 | C | TYR | A | 50 | 12.312 | 5.904 | 18.006 | 1.00 | 12.31 | C |
| ATOM | 442 | O | TYR | A | 50 | 13.030 | 6.911 | 18.241 | 1.00 | 13.28 | O |
| ATOM | 443 | N | ARG | A | 51 | 11.001 | 5.928 | 18.188 | 1.00 | 12.34 | N |
| ATOM | 444 | CA | ARG | A | 51 | 10.303 | 7.144 | 18.496 | 1.00 | 12.42 | C |
| ATOM | 445 | CB | ARG | A | 51 | 8.795 | 6.869 | 18.371 | 1.00 | 14.04 | C |
| ATOM | 446 | CG | ARG | A | 51 | 8.261 | 7.145 | 16.923 | 1.00 | 14.11 | C |
| ATOM | 447 | CD | ARG | A | 51 | 7.721 | 8.577 | 17.058 | 1.00 | 23.55 | C |
| ATOM | 448 | NE | ARG | A | 51 | 8.521 | 9.271 | 16.203 | 1.00 | 17.63 | N |
| ATOM | 449 | CZ | ARG | A | 51 | 9.060 | 10.459 | 16.315 | 1.00 | 12.66 | C |
| ATOM | 450 | NH1 | ARG | A | 51 | 8.852 | 11.367 | 17.275 | 1.00 | 13.62 | N |
| ATOM | 451 | NH2 | ARG | A | 51 | 9.843 | 10.700 | 15.290 | 1.00 | 13.25 | N |
| ATOM | 452 | C | ARG | A | 51 | 10.614 | 7.604 | 19.922 | 1.00 | 14.15 | C |
| ATOM | 453 | O | ARG | A | 51 | 10.651 | 8.808 | 20.144 | 1.00 | 11.71 | O |
| ATOM | 454 | N | ALA | A | 52 | 10.766 | 6.663 | 20.901 | 1.00 | 14.36 | N |
| ATOM | 455 | CA | ALA | A | 52 | 11.112 | 7.066 | 22.267 | 1.00 | 13.96 | C |
| ATOM | 456 | CB | ALA | A | 52 | 11.095 | 5.836 | 23.217 | 1.00 | 14.72 | C |
| ATOM | 457 | C | ALA | A | 52 | 12.463 | 7.826 | 22.265 | 1.00 | 13.65 | C |
| ATOM | 458 | O | ALA | A | 52 | 12.655 | 8.817 | 22.954 | 1.00 | 12.08 | O |
| ATOM | 459 | N | GLN | A | 53 | 13.398 | 7.361 | 21.418 | 1.00 | 14.50 | N |
| ATOM | 460 | CA | GLN | A | 53 | 14.706 | 7.937 | 21.347 | 1.00 | 13.04 | C |
| ATOM | 461 | CB | GLN | A | 53 | 15.704 | 6.986 | 20.619 | 1.00 | 12.84 | C |
| ATOM | 462 | CG | GLN | A | 53 | 17.113 | 7.675 | 20.304 | 1.00 | 12.80 | C |
| ATOM | 463 | CD | GLN | A | 53 | 17.181 | 8.182 | 18.877 | 1.00 | 13.94 | C |
| ATOM | 464 | OE1 | GLN | A | 53 | 17.004 | 7.424 | 17.939 | 1.00 | 16.24 | O |
| ATOM | 465 | NE2 | GLN | A | 53 | 17.427 | 9.502 | 18.693 | 1.00 | 15.50 | N |
| ATOM | 466 | C | GLN | A | 53 | 14.642 | 9.283 | 20.637 | 1.00 | 13.51 | C |
| ATOM | 467 | O | GLN | A | 53 | 15.246 | 10.255 | 21.097 | 1.00 | 12.36 | O |
| ATOM | 468 | N | VAL | A | 54 | 13.916 | 9.355 | 19.505 | 1.00 | 12.90 | N |
| ATOM | 469 | CA | VAL | A | 54 | 13.851 | 10.610 | 18.783 | 1.00 | 10.80 | C |
| ATOM | 470 | CB | VAL | A | 54 | 13.092 | 10.425 | 17.479 | 1.00 | 11.95 | C |
| ATOM | 471 | CG1 | VAL | A | 54 | 12.779 | 11.766 | 16.825 | 1.00 | 7.46 | C |
| ATOM | 472 | CG2 | VAL | A | 54 | 13.891 | 9.504 | 16.524 | 1.00 | 9.26 | C |
| ATOM | 473 | C | VAL | A | 54 | 13.176 | 11.675 | 19.671 | 1.00 | 11.94 | C |
| ATOM | 474 | O | VAL | A | 54 | 13.554 | 12.809 | 19.642 | 1.00 | 12.72 | O |
| ATOM | 475 | N | ASN | A | 55 | 12.156 | 11.297 | 20.443 | 1.00 | 11.98 | N |
| ATOM | 476 | CA | ASN | A | 55 | 11.465 | 12.261 | 21.301 | 1.00 | 12.46 | C |
| ATOM | 477 | CB | ASN | A | 55 | 10.179 | 11.650 | 21.950 | 1.00 | 9.74 | C |
| ATOM | 478 | CG | ASN | A | 55 | 9.332 | 12.678 | 22.694 | 1.00 | 13.90 | C |
| ATOM | 479 | OD1 | ASN | A | 55 | 9.691 | 13.107 | 23.815 | 1.00 | 15.04 | O |
| ATOM | 480 | ND2 | ASN | A | 55 | 8.231 | 13.151 | 22.050 | 1.00 | 6.77 | N |
| ATOM | 481 | C | ASN | A | 55 | 12.403 | 12.810 | 22.353 | 1.00 | 11.37 | C |
| ATOM | 482 | O | ASN | A | 55 | 12.440 | 14.025 | 22.586 | 1.00 | 11.95 | O |
| ATOM | 483 | N | CYS | A | 56 | 13.137 | 11.906 | 23.022 | 1.00 | 11.84 | N |
| ATOM | 484 | CA | CYS | A | 56 | 14.116 | 12.344 | 24.024 | 1.00 | 12.82 | C |
| ATOM | 485 | CB | CYS | A | 56 | 14.794 | 11.145 | 24.729 | 1.00 | 11.54 | C |
| ATOM | 486 | SG | CYS | A | 56 | 13.622 | 10.159 | 25.693 | 1.00 | 18.21 | S |
| ATOM | 487 | C | CYS | A | 56 | 15.150 | 13.273 | 23.368 | 1.00 | 11.89 | C |
| ATOM | 488 | O | CYS | A | 56 | 15.511 | 14.275 | 23.950 | 1.00 | 9.84 | O |
| ATOM | 489 | N | SER | A | 57 | 15.634 | 12.918 | 22.172 | 1.00 | 12.11 | N |
| ATOM | 490 | CA | SER | A | 57 | 16.655 | 13.730 | 21.479 | 1.00 | 13.22 | C |
| ATOM | 491 | CB | SER | A | 57 | 17.141 | 13.114 | 20.150 | 1.00 | 13.25 | C |
| ATOM | 492 | OG | SER | A | 57 | 17.733 | 11.872 | 20.383 | 1.00 | 17.22 | O |
| ATOM | 493 | C | SER | A | 57 | 16.088 | 15.106 | 21.137 | 1.00 | 14.26 | C |
| ATOM | 494 | O | SER | A | 57 | 16.810 | 16.074 | 21.196 | 1.00 | 12.95 | O |
| ATOM | 495 | N | GLU | A | 58 | 14.813 | 15.207 | 20.733 | 1.00 | 15.19 | N |
| ATOM | 496 | CA | GLU | A | 58 | 14.374 | 16.567 | 20.393 | 1.00 | 15.85 | C |
| ATOM | 497 | CB | GLU | A | 58 | 13.209 | 16.584 | 19.423 | 1.00 | 18.23 | C |
| ATOM | 498 | CG | GLU | A | 58 | 11.967 | 16.232 | 20.029 | 1.00 | 19.53 | C |
| ATOM | 499 | CD | GLU | A | 58 | 10.859 | 15.915 | 18.951 | 1.00 | 24.35 | C |
| ATOM | 500 | OE1 | GLU | A | 58 | 11.115 | 16.019 | 17.730 | 1.00 | 22.29 | O |
| ATOM | 501 | OE2 | GLU | A | 58 | 9.771 | 15.494 | 19.374 | 1.00 | 19.32 | O |
| ATOM | 502 | C | GLU | A | 58 | 14.124 | 17.480 | 21.575 | 1.00 | 15.55 | C |
| ATOM | 503 | O | GLU | A | 58 | 14.276 | 18.702 | 21.414 | 1.00 | 13.27 | O |
| ATOM | 504 | N | TYR | A | 59 | 13.818 | 16.912 | 22.762 | 1.00 | 13.72 | N |
| ATOM | 505 | CA | TYR | A | 59 | 13.730 | 17.730 | 23.973 | 1.00 | 13.56 | C |
| ATOM | 506 | CB | TYR | A | 59 | 12.756 | 17.146 | 25.011 | 1.00 | 14.18 | C |
| ATOM | 507 | CG | TYR | A | 59 | 11.268 | 17.317 | 24.653 | 1.00 | 15.13 | C |
| ATOM | 508 | CD1 | TYR | A | 59 | 10.517 | 18.401 | 25.180 | 1.00 | 16.03 | C |
| ATOM | 509 | CE1 | TYR | A | 59 | 9.142 | 18.585 | 24.842 | 1.00 | 15.15 | C |
| ATOM | 510 | CZ | TYR | A | 59 | 8.536 | 17.605 | 24.039 | 1.00 | 15.61 | C |
| ATOM | 511 | OH | TYR | A | 59 | 7.218 | 17.734 | 23.715 | 1.00 | 15.78 | O |
| ATOM | 512 | CE2 | TYR | A | 59 | 9.278 | 16.585 | 23.470 | 1.00 | 11.76 | C |
| ATOM | 513 | CD2 | TYR | A | 59 | 10.620 | 16.427 | 23.781 | 1.00 | 12.40 | C |
| ATOM | 514 | C | TYR | A | 59 | 15.062 | 17.965 | 24.656 | 1.00 | 13.24 | C |
| ATOM | 515 | O | TYR | A | 59 | 15.168 | 18.830 | 25.518 | 1.00 | 11.08 | O |
| ATOM | 516 | N | PHE | A | 60 | 16.064 | 17.161 | 24.322 | 1.00 | 13.07 | N |
| ATOM | 517 | CA | PHE | A | 60 | 17.376 | 17.305 | 24.919 | 1.00 | 13.02 | C |
| ATOM | 518 | CB | PHE | A | 60 | 18.365 | 16.191 | 24.437 | 1.00 | 13.22 | C |
| ATOM | 519 | CG | PHE | A | 60 | 19.663 | 16.094 | 25.237 | 1.00 | 11.22 | C |
| ATOM | 520 | CD1 | PHE | A | 60 | 19.651 | 15.779 | 26.614 | 1.00 | 17.34 | C |
| ATOM | 521 | CE1 | PHE | A | 60 | 20.828 | 15.682 | 27.352 | 1.00 | 12.55 | C |
| ATOM | 522 | CZ | PHE | A | 60 | 22.034 | 15.892 | 26.714 | 1.00 | 12.20 | C |
| ATOM | 523 | CE2 | PHE | A | 60 | 22.090 | 16.245 | 25.410 | 1.00 | 14.53 | C |
| ATOM | 524 | CD2 | PHE | A | 60 | 20.866 | 16.303 | 24.630 | 1.00 | 13.00 | C |
| ATOM | 525 | C | PHE | A | 60 | 17.985 | 18.741 | 24.907 | 1.00 | 13.07 | C |
| ATOM | 526 | O | PHE | A | 60 | 18.493 | 19.159 | 25.922 | 1.00 | 13.53 | O |
| ATOM | 527 | N | PRO | A | 61 | 18.016 | 19.461 | 23.745 | 1.00 | 14.18 | N |
| ATOM | 528 | CA | PRO | A | 61 | 18.541 | 20.831 | 23.851 | 1.00 | 13.05 | C |
| ATOM | 529 | CB | PRO | A | 61 | 18.561 | 21.353 | 22.380 | 1.00 | 12.70 | C |
| ATOM | 530 | CG | PRO | A | 61 | 18.455 | 20.061 | 21.536 | 1.00 | 12.61 | C |
| ATOM | 531 | CD | PRO | A | 61 | 17.683 | 19.070 | 22.339 | 1.00 | 10.04 | C |
| ATOM | 532 | C | PRO | A | 61 | 17.685 | 21.789 | 24.685 | 1.00 | 14.93 | C |
| ATOM | 533 | O | PRO | A | 61 | 18.217 | 22.775 | 25.147 | 1.00 | 14.83 | O |
| ATOM | 534 | N | LEU | A | 62 | 16.366 | 21.542 | 24.812 | 1.00 | 15.61 | N |
| ATOM | 535 | CA | LEU | A | 62 | 15.537 | 22.402 | 25.615 | 1.00 | 15.15 | C |
| ATOM | 536 | CB | LEU | A | 62 | 14.084 | 22.004 | 25.437 | 1.00 | 15.98 | C |
| ATOM | 537 | CG | LEU | A | 62 | 13.346 | 22.529 | 24.238 | 1.00 | 18.01 | C |
| ATOM | 538 | CD1 | LEU | A | 62 | 14.129 | 22.750 | 22.948 | 1.00 | 19.31 | C |
| ATOM | 539 | CD2 | LEU | A | 62 | 12.063 | 21.759 | 24.065 | 1.00 | 20.44 | C |
| ATOM | 540 | C | LEU | A | 62 | 15.960 | 22.178 | 27.052 | 1.00 | 15.52 | C |
| ATOM | 541 | O | LEU | A | 62 | 16.059 | 23.105 | 27.849 | 1.00 | 13.51 | O |
| ATOM | 542 | N | PHE | A | 63 | 16.183 | 20.916 | 27.413 | 1.00 | 13.75 | N |
| ATOM | 543 | CA | PHE | A | 63 | 16.612 | 20.651 | 28.733 | 1.00 | 14.28 | C |
| ATOM | 544 | CB | PHE | A | 63 | 16.639 | 19.132 | 28.947 | 1.00 | 14.43 | C |
| ATOM | 545 | CG | PHE | A | 63 | 17.597 | 18.678 | 30.045 | 1.00 | 18.25 | C |
| ATOM | 546 | CD1 | PHE | A | 63 | 17.305 | 18.899 | 31.394 | 1.00 | 19.16 | C |
| ATOM | 547 | CE1 | PHE | A | 63 | 18.201 | 18.419 | 32.424 | 1.00 | 17.16 | C |
| ATOM | 548 | CZ | PHE | A | 63 | 19.352 | 17.753 | 32.057 | 1.00 | 15.03 | C |
| ATOM | 549 | CE2 | PHE | A | 63 | 19.674 | 17.571 | 30.735 | 1.00 | 22.75 | C |
| ATOM | 550 | CD2 | PHE | A | 63 | 18.775 | 18.003 | 29.710 | 1.00 | 17.50 | C |
| ATOM | 551 | C | PHE | A | 63 | 17.982 | 21.334 | 29.046 | 1.00 | 14.58 | C |
| ATOM | 552 | O | PHE | A | 63 | 18.143 | 21.911 | 30.093 | 1.00 | 14.07 | O |
| ATOM | 553 | N | LEU | A | 64 | 18.983 | 21.165 | 28.181 | 1.00 | 14.45 | N |
| ATOM | 554 | CA | LEU | A | 64 | 20.322 | 21.755 | 28.402 | 1.00 | 15.20 | C |
| ATOM | 555 | CB | LEU | A | 64 | 21.284 | 21.419 | 27.246 | 1.00 | 15.87 | C |
| ATOM | 556 | CG | LEU | A | 64 | 21.739 | 19.946 | 27.212 | 1.00 | 22.75 | C |
| ATOM | 557 | CD1 | LEU | A | 64 | 22.914 | 19.754 | 26.320 | 1.00 | 27.77 | C |
| ATOM | 558 | CD2 | LEU | A | 64 | 22.123 | 19.428 | 28.622 | 1.00 | 22.63 | C |
| ATOM | 559 | C | LEU | A | 64 | 20.222 | 23.255 | 28.525 | 1.00 | 13.47 | C |
| ATOM | 560 | O | LEU | A | 64 | 20.840 | 23.833 | 29.401 | 1.00 | 14.28 | O |
| ATOM | 561 | N | ALA | A | 65 | 19.477 | 23.884 | 27.630 | 1.00 | 12.85 | N |
| ATOM | 562 | CA | ALA | A | 65 | 19.400 | 25.358 | 27.619 | 1.00 | 13.24 | C |
| ATOM | 563 | CB | ALA | A | 65 | 18.501 | 25.842 | 26.473 | 1.00 | 10.80 | C |
| ATOM | 564 | C | ALA | A | 65 | 18.867 | 25.866 | 28.935 | 1.00 | 13.87 | C |
| ATOM | 565 | O | ALA | A | 65 | 19.429 | 26.822 | 29.568 | 1.00 | 12.95 | O |
| ATOM | 566 | N | THR | A | 66 | 17.780 | 25.212 | 29.360 | 1.00 | 12.63 | N |
| ATOM | 567 | CA | ATHR | A | 66 | 17.011 | 25.635 | 30.507 | 0.50 | 13.55 | C |
| ATOM | 568 | CA | BTHR | A | 66 | 17.089 | 25.720 | 30.525 | 0.50 | 13.85 | C |
| ATOM | 569 | CB | ATHR | A | 66 | 15.614 | 24.923 | 30.414 | 0.50 | 12.60 | C |
| ATOM | 570 | CB | BTHR | A | 66 | 15.597 | 25.358 | 30.552 | 0.50 | 13.34 | C |
| ATOM | 571 | OG1 | ATHR | A | 66 | 14.877 | 25.415 | 29.278 | 0.50 | 8.39 | O |
| ATOM | 572 | OG1 | BTHR | A | 66 | 14.995 | 26.151 | 31.573 | 0.50 | 15.39 | O |
| ATOM | 573 | CG2 | ATHR | A | 66 | 14.823 | 25.160 | 31.625 | 0.50 | 14.87 | C |
| ATOM | 574 | CG2 | BTHR | A | 66 | 15.390 | 23.907 | 30.905 | 0.50 | 10.32 | C |
| ATOM | 575 | C | THR | A | 66 | 17.785 | 25.363 | 31.852 | 1.00 | 14.13 | C |
| ATOM | 576 | O | THR | A | 66 | 17.830 | 26.178 | 32.763 | 1.00 | 14.63 | O |
| ATOM | 577 | N | LEU | A | 67 | 18.372 | 24.179 | 31.939 | 1.00 | 14.49 | N |
| ATOM | 578 | CA | LEU | A | 67 | 19.321 | 23.795 | 33.002 | 1.00 | 12.69 | C |
| ATOM | 579 | CB | LEU | A | 67 | 19.996 | 22.449 | 32.629 | 1.00 | 13.54 | C |
| ATOM | 580 | CG | LEU | A | 67 | 21.083 | 21.928 | 33.570 | 1.00 | 8.83 | C |
| ATOM | 581 | CD1 | LEU | A | 67 | 20.388 | 21.495 | 34.959 | 1.00 | 7.51 | C |
| ATOM | 582 | CD2 | LEU | A | 67 | 21.921 | 20.772 | 32.962 | 1.00 | 12.44 | C |
| ATOM | 583 | C | LEU | A | 67 | 20.391 | 24.883 | 33.263 | 1.00 | 14.47 | C |
| ATOM | 584 | O | LEU | A | 67 | 20.612 | 25.276 | 34.428 | 1.00 | 13.40 | O |
| ATOM | 585 | N | TRP | A | 68 | 21.090 | 25.295 | 32.203 | 1.00 | 12.00 | N |
| ATOM | 586 | CA | TRP | A | 68 | 22.181 | 26.233 | 32.303 | 1.00 | 13.04 | C |
| ATOM | 587 | CB | TRP | A | 68 | 23.044 | 26.221 | 31.005 | 1.00 | 10.77 | C |
| ATOM | 588 | CG | TRP | A | 68 | 23.990 | 25.029 | 31.064 | 1.00 | 13.93 | C |
| ATOM | 589 | CD1 | TRP | A | 68 | 23.748 | 23.718 | 30.618 | 1.00 | 12.43 | C |
| ATOM | 590 | NE1 | TRP | A | 68 | 24.832 | 22.926 | 30.908 | 1.00 | 9.53 | N |
| ATOM | 591 | CE2 | TRP | A | 68 | 25.808 | 23.698 | 31.500 | 1.00 | 12.27 | C |
| ATOM | 592 | CD2 | TRP | A | 68 | 25.309 | 25.016 | 31.630 | 1.00 | 11.94 | C |
| ATOM | 593 | CE3 | TRP | A | 68 | 26.126 | 26.006 | 32.229 | 1.00 | 16.53 | C |
| ATOM | 594 | CZ3 | TRP | A | 68 | 27.362 | 25.658 | 32.651 | 1.00 | 15.09 | C |
| ATOM | 595 | CH2 | TRP | A | 68 | 27.847 | 24.291 | 32.521 | 1.00 | 11.03 | C |
| ATOM | 596 | CZ2 | TRP | A | 68 | 27.082 | 23.334 | 31.954 | 1.00 | 11.78 | C |
| ATOM | 597 | C | TRP | A | 68 | 21.678 | 27.629 | 32.676 | 1.00 | 12.25 | C |
| ATOM | 598 | O | TRP | A | 68 | 22.271 | 28.259 | 33.527 | 1.00 | 12.94 | O |
| ATOM | 599 | N | VAL | A | 69 | 20.585 | 28.087 | 32.052 | 1.00 | 11.26 | N |
| ATOM | 600 | CA | VAL | A | 69 | 20.049 | 29.385 | 32.383 | 1.00 | 10.51 | C |
| ATOM | 601 | CB | VAL | A | 69 | 18.965 | 29.886 | 31.365 | 1.00 | 10.37 | C |
| ATOM | 602 | CG1 | VAL | A | 69 | 18.396 | 31.246 | 31.893 | 1.00 | 7.69 | C |
| ATOM | 603 | CG2 | VAL | A | 69 | 19.635 | 30.133 | 29.934 | 1.00 | 9.36 | C |
| ATOM | 604 | C | VAL | A | 69 | 19.513 | 29.373 | 33.820 | 1.00 | 11.73 | C |
| ATOM | 605 | O | VAL | A | 69 | 19.782 | 30.272 | 34.550 | 1.00 | 11.65 | O |
| ATOM | 606 | N | ALA | A | 70 | 18.744 | 28.347 | 34.227 | 1.00 | 11.50 | N |
| ATOM | 607 | CA | ALA | A | 70 | 18.267 | 28.297 | 35.620 | 1.00 | 11.42 | C |
| ATOM | 608 | CB | ALA | A | 70 | 17.266 | 27.106 | 35.883 | 1.00 | 10.19 | C |
| ATOM | 609 | C | ALA | A | 70 | 19.443 | 28.184 | 36.596 | 1.00 | 12.67 | C |
| ATOM | 610 | O | ALA | A | 70 | 19.405 | 28.803 | 37.682 | 1.00 | 11.04 | O |
| ATOM | 611 | N | GLY | A | 71 | 20.440 | 27.364 | 36.258 | 1.00 | 12.51 | N |
| ATOM | 612 | CA | GLY | A | 71 | 21.555 | 27.166 | 37.157 | 1.00 | 13.85 | C |
| ATOM | 613 | C | GLY | A | 71 | 22.394 | 28.419 | 37.321 | 1.00 | 14.72 | C |
| ATOM | 614 | O | GLY | A | 71 | 22.945 | 28.625 | 38.359 | 1.00 | 15.04 | O |
| ATOM | 615 | N | ILE | A | 72 | 22.547 | 29.224 | 36.284 | 1.00 | 15.76 | N |
| ATOM | 616 | CA | ILE | A | 72 | 23.340 | 30.476 | 36.402 | 1.00 | 15.94 | C |
| ATOM | 617 | CB | ILE | A | 72 | 23.940 | 30.843 | 35.025 | 1.00 | 17.01 | C |
| ATOM | 618 | CG1 | ILE | A | 72 | 25.052 | 29.829 | 34.772 | 1.00 | 20.65 | C |
| ATOM | 619 | CD1 | ILE | A | 72 | 25.365 | 29.700 | 33.330 | 1.00 | 25.78 | C |
| ATOM | 620 | CG2 | ILE | A | 72 | 24.568 | 32.263 | 35.001 | 1.00 | 15.51 | C |
| ATOM | 621 | C | ILE | A | 72 | 22.594 | 31.656 | 37.060 | 1.00 | 16.58 | C |
| ATOM | 622 | O | ILE | A | 72 | 23.148 | 32.394 | 37.845 | 1.00 | 17.07 | O |
| ATOM | 623 | N | PHE | A | 73 | 21.304 | 31.798 | 36.753 | 1.00 | 16.25 | N |
| ATOM | 624 | CA | PHE | A | 73 | 20.539 | 32.993 | 37.092 | 1.00 | 13.94 | C |
| ATOM | 625 | CB | PHE | A | 73 | 19.718 | 33.521 | 35.865 | 1.00 | 12.32 | C |
| ATOM | 626 | CG | PHE | A | 73 | 20.570 | 34.265 | 34.861 | 1.00 | 15.58 | C |
| ATOM | 627 | CD1 | PHE | A | 73 | 20.818 | 35.669 | 34.997 | 1.00 | 13.34 | C |
| ATOM | 628 | CE1 | PHE | A | 73 | 21.671 | 36.343 | 34.056 | 1.00 | 14.64 | C |
| ATOM | 629 | CZ | PHE | A | 73 | 22.312 | 35.551 | 33.034 | 1.00 | 10.84 | C |
| ATOM | 630 | CE2 | PHE | A | 73 | 22.033 | 34.161 | 32.972 | 1.00 | 13.67 | C |
| ATOM | 631 | CD2 | PHE | A | 73 | 21.209 | 33.557 | 33.842 | 1.00 | 12.49 | C |
| ATOM | 632 | C | PHE | A | 73 | 19.695 | 32.702 | 38.323 | 1.00 | 13.02 | C |
| ATOM | 633 | O | PHE | A | 73 | 19.265 | 33.650 | 39.004 | 1.00 | 12.63 | O |
| ATOM | 634 | N | PHE | A | 74 | 19.458 | 31.442 | 38.659 | 1.00 | 10.90 | N |
| ATOM | 635 | CA | PHE | A | 74 | 18.542 | 31.234 | 39.809 | 1.00 | 11.36 | C |
| ATOM | 636 | CB | PHE | A | 74 | 17.222 | 30.468 | 39.467 | 1.00 | 11.30 | C |
| ATOM | 637 | CG | PHE | A | 74 | 16.323 | 30.260 | 40.693 | 1.00 | 11.70 | C |
| ATOM | 638 | CD1 | PHE | A | 74 | 15.372 | 31.209 | 41.062 | 1.00 | 10.85 | C |
| ATOM | 639 | CE1 | PHE | A | 74 | 14.543 | 31.064 | 42.226 | 1.00 | 12.62 | C |
| ATOM | 640 | CZ | PHE | A | 74 | 14.753 | 30.010 | 43.087 | 1.00 | 11.26 | C |
| ATOM | 641 | CE2 | PHE | A | 74 | 15.719 | 29.035 | 42.746 | 1.00 | 15.01 | C |
| ATOM | 642 | CD2 | PHE | A | 74 | 16.509 | 29.179 | 41.535 | 1.00 | 16.36 | C |
| ATOM | 643 | C | PHE | A | 74 | 19.325 | 30.583 | 40.967 | 1.00 | 13.78 | C |
| ATOM | 644 | O | PHE | A | 74 | 19.557 | 31.229 | 41.991 | 1.00 | 13.01 | O |
| ATOM | 645 | N | HIS | A | 75 | 19.806 | 29.341 | 40.806 | 1.00 | 13.91 | N |
| ATOM | 646 | CA | HIS | A | 75 | 20.512 | 28.633 | 41.902 | 1.00 | 13.91 | C |
| ATOM | 647 | CB | HIS | A | 75 | 19.539 | 28.274 | 43.039 | 1.00 | 13.67 | C |
| ATOM | 648 | CG | HIS | A | 75 | 20.189 | 27.633 | 44.229 | 1.00 | 12.20 | C |
| ATOM | 649 | ND1 | HIS | A | 75 | 20.328 | 26.273 | 44.346 | 1.00 | 11.93 | N |
| ATOM | 650 | CE1 | HIS | A | 75 | 20.913 | 25.981 | 45.493 | 1.00 | 13.03 | C |
| ATOM | 651 | NE2 | HIS | A | 75 | 21.173 | 27.114 | 46.125 | 1.00 | 10.22 | N |
| ATOM | 652 | CD2 | HIS | A | 75 | 20.704 | 28.162 | 45.369 | 1.00 | 10.73 | C |
| ATOM | 653 | C | HIS | A | 75 | 21.003 | 27.332 | 41.272 | 1.00 | 14.40 | C |
| ATOM | 654 | O | HIS | A | 75 | 20.201 | 26.578 | 40.723 | 1.00 | 14.22 | O |
| ATOM | 655 | N | GLU | A | 76 | 22.298 | 27.058 | 41.383 | 1.00 | 13.47 | N |
| ATOM | 656 | CA | GLU | A | 76 | 22.903 | 25.862 | 40.777 | 1.00 | 15.28 | C |
| ATOM | 657 | CB | GLU | A | 76 | 24.467 | 25.933 | 40.797 | 1.00 | 13.94 | C |
| ATOM | 658 | CG | GLU | A | 76 | 25.036 | 24.964 | 39.771 | 1.00 | 16.66 | C |
| ATOM | 659 | CD | GLU | A | 76 | 26.464 | 24.478 | 40.055 | 1.00 | 19.99 | C |
| ATOM | 660 | OE1 | GLU | A | 76 | 27.005 | 24.723 | 41.125 | 1.00 | 23.00 | O |
| ATOM | 661 | OE2 | GLU | A | 76 | 27.078 | 23.817 | 39.202 | 1.00 | 20.47 | O |
| ATOM | 662 | C | GLU | A | 76 | 22.398 | 24.522 | 41.294 | 1.00 | 14.98 | C |
| ATOM | 663 | O | GLU | A | 76 | 22.126 | 23.669 | 40.492 | 1.00 | 17.45 | O |
| ATOM | 664 | N | GLY | A | 77 | 22.300 | 24.309 | 42.614 | 1.00 | 14.20 | N |
| ATOM | 665 | CA | GLY | A | 77 | 21.830 | 23.076 | 43.192 | 1.00 | 12.60 | C |
| ATOM | 666 | C | GLY | A | 77 | 20.403 | 22.759 | 42.849 | 1.00 | 14.63 | C |
| ATOM | 667 | O | GLY | A | 77 | 20.048 | 21.592 | 42.545 | 1.00 | 13.52 | O |
| ATOM | 668 | N | ALA | A | 78 | 19.539 | 23.768 | 42.898 | 1.00 | 14.71 | N |
| ATOM | 669 | CA | ALA | A | 78 | 18.139 | 23.580 | 42.513 | 1.00 | 13.75 | C |
| ATOM | 670 | CB | ALA | A | 78 | 17.294 | 24.901 | 42.768 | 1.00 | 13.00 | C |
| ATOM | 671 | C | ALA | A | 78 | 17.986 | 23.131 | 41.089 | 1.00 | 13.82 | C |
| ATOM | 672 | O | ALA | A | 78 | 17.144 | 22.201 | 40.763 | 1.00 | 14.20 | O |
| ATOM | 673 | N | ALA | A | 79 | 18.743 | 23.781 | 40.204 | 1.00 | 14.04 | N |
| ATOM | 674 | CA | ALA | A | 79 | 18.618 | 23.502 | 38.775 | 1.00 | 12.36 | C |
| ATOM | 675 | CB | ALA | A | 79 | 19.402 | 24.496 | 37.934 | 1.00 | 9.24 | C |
| ATOM | 676 | C | ALA | A | 79 | 19.112 | 22.081 | 38.495 | 1.00 | 13.04 | C |
| ATOM | 677 | O | ALA | A | 79 | 18.448 | 21.302 | 37.774 | 1.00 | 13.24 | O |
| ATOM | 678 | N | ALA | A | 80 | 20.260 | 21.735 | 39.093 | 1.00 | 13.27 | N |
| ATOM | 679 | CA | ALA | A | 80 | 20.877 | 20.407 | 38.954 | 1.00 | 11.95 | C |
| ATOM | 680 | CB | ALA | A | 80 | 22.173 | 20.315 | 39.729 | 1.00 | 10.13 | C |
| ATOM | 681 | C | ALA | A | 80 | 19.932 | 19.320 | 39.445 | 1.00 | 13.89 | C |
| ATOM | 682 | O | ALA | A | 80 | 19.749 | 18.306 | 38.774 | 1.00 | 13.84 | O |
| ATOM | 683 | N | LEU | A | 81 | 19.306 | 19.564 | 40.587 | 1.00 | 14.77 | N |
| ATOM | 684 | CA | LEU | A | 81 | 18.326 | 18.657 | 41.154 | 1.00 | 16.92 | C |
| ATOM | 685 | CB | LEU | A | 81 | 17.851 | 19.276 | 42.444 | 1.00 | 17.72 | C |
| ATOM | 686 | CG | LEU | A | 81 | 17.596 | 18.454 | 43.677 | 1.00 | 26.70 | C |
| ATOM | 687 | CD1 | LEU | A | 81 | 18.461 | 17.115 | 43.668 | 1.00 | 30.82 | C |
| ATOM | 688 | CD2 | LEU | A | 81 | 17.947 | 19.438 | 44.895 | 1.00 | 31.30 | C |
| ATOM | 689 | C | LEU | A | 81 | 17.090 | 18.465 | 40.237 | 1.00 | 15.28 | C |
| ATOM | 690 | O | LEU | A | 81 | 16.691 | 17.354 | 40.014 | 1.00 | 13.83 | O |
| ATOM | 691 | N | CYS | A | 82 | 16.448 | 19.550 | 39.774 | 1.00 | 15.11 | N |
| ATOM | 692 | CA | CYS | A | 82 | 15.398 | 19.411 | 38.722 | 1.00 | 16.94 | C |
| ATOM | 693 | CB | CYS | A | 82 | 14.737 | 20.724 | 38.254 | 1.00 | 16.28 | C |
| ATOM | 694 | SG | CYS | A | 82 | 14.157 | 21.763 | 39.515 | 1.00 | 23.74 | S |
| ATOM | 695 | C | CYS | A | 82 | 15.873 | 18.668 | 37.480 | 1.00 | 16.03 | C |
| ATOM | 696 | O | CYS | A | 82 | 15.072 | 17.958 | 36.840 | 1.00 | 16.87 | O |
| ATOM | 697 | N | GLY | A | 83 | 17.133 | 18.922 | 37.105 | 1.00 | 16.23 | N |
| ATOM | 698 | CA | GLY | A | 83 | 17.811 | 18.200 | 36.056 | 1.00 | 14.48 | C |
| ATOM | 699 | C | GLY | A | 83 | 17.856 | 16.677 | 36.180 | 1.00 | 14.60 | C |
| ATOM | 700 | O | GLY | A | 83 | 17.528 | 15.959 | 35.215 | 1.00 | 13.92 | O |
| ATOM | 701 | N | LEU | A | 84 | 18.246 | 16.177 | 37.359 | 1.00 | 14.89 | N |
| ATOM | 702 | CA | LEU | A | 84 | 18.247 | 14.778 | 37.632 | 1.00 | 15.09 | C |
| ATOM | 703 | CB | LEU | A | 84 | 18.742 | 14.472 | 39.034 | 1.00 | 16.29 | C |
| ATOM | 704 | CG | LEU | A | 84 | 20.232 | 14.789 | 39.235 | 1.00 | 17.97 | C |
| ATOM | 705 | CD1 | LEU | A | 84 | 20.546 | 14.556 | 40.725 | 1.00 | 20.25 | C |
| ATOM | 706 | CD2 | LEU | A | 84 | 21.082 | 13.828 | 38.252 | 1.00 | 18.58 | C |
| ATOM | 707 | C | LEU | A | 84 | 16.867 | 14.225 | 37.577 | 1.00 | 14.51 | C |
| ATOM | 708 | O | LEU | A | 84 | 16.672 | 13.152 | 37.000 | 1.00 | 13.34 | O |
| ATOM | 709 | N | VAL | A | 85 | 15.933 | 14.918 | 38.208 | 1.00 | 12.53 | N |
| ATOM | 710 | CA | VAL | A | 85 | 14.516 | 14.476 | 38.156 | 1.00 | 13.74 | C |
| ATOM | 711 | CB | VAL | A | 85 | 13.536 | 15.399 | 39.001 | 1.00 | 13.07 | C |
| ATOM | 712 | CG1 | VAL | A | 85 | 12.020 | 15.035 | 38.687 | 1.00 | 15.53 | C |
| ATOM | 713 | CG2 | VAL | A | 85 | 13.866 | 15.270 | 40.449 | 1.00 | 14.98 | C |
| ATOM | 714 | C | VAL | A | 85 | 13.991 | 14.357 | 36.692 | 1.00 | 12.69 | C |
| ATOM | 715 | O | VAL | A | 85 | 13.370 | 13.360 | 36.371 | 1.00 | 11.34 | O |
| ATOM | 716 | N | TYR | A | 86 | 14.279 | 15.363 | 35.839 | 1.00 | 11.34 | N |
| ATOM | 717 | CA | TYR | A | 86 | 13.941 | 15.315 | 34.419 | 1.00 | 11.49 | C |
| ATOM | 718 | CB | TYR | A | 86 | 14.351 | 16.657 | 33.721 | 1.00 | 10.90 | C |
| ATOM | 719 | CG | TYR | A | 86 | 14.211 | 16.591 | 32.237 | 1.00 | 11.85 | C |
| ATOM | 720 | CD1 | TYR | A | 86 | 12.977 | 16.837 | 31.634 | 1.00 | 11.77 | C |
| ATOM | 721 | CE1 | TYR | A | 86 | 12.822 | 16.718 | 30.228 | 1.00 | 12.10 | C |
| ATOM | 722 | CZ | TYR | A | 86 | 13.885 | 16.330 | 29.484 | 1.00 | 14.05 | C |
| ATOM | 723 | OH | TYR | A | 86 | 13.726 | 16.166 | 28.114 | 1.00 | 14.10 | O |
| ATOM | 724 | CE2 | TYR | A | 86 | 15.107 | 16.003 | 30.089 | 1.00 | 13.31 | C |
| ATOM | 725 | CD2 | TYR | A | 86 | 15.266 | 16.116 | 31.436 | 1.00 | 7.03 | C |
| ATOM | 726 | C | TYR | A | 86 | 14.592 | 14.107 | 33.733 | 1.00 | 12.33 | C |
| ATOM | 727 | O | TYR | A | 86 | 13.939 | 13.382 | 32.993 | 1.00 | 13.31 | O |
| ATOM | 728 | N | LEU | A | 87 | 15.882 | 13.859 | 33.979 | 1.00 | 13.85 | N |
| ATOM | 729 | CA | LEU | A | 87 | 16.568 | 12.763 | 33.317 | 1.00 | 13.13 | C |
| ATOM | 730 | CB | LEU | A | 87 | 18.103 | 12.881 | 33.500 | 1.00 | 14.25 | C |
| ATOM | 731 | CG | LEU | A | 87 | 18.784 | 14.080 | 32.824 | 1.00 | 13.79 | C |
| ATOM | 732 | CD1 | LEU | A | 87 | 20.269 | 14.022 | 33.173 | 1.00 | 10.18 | C |
| ATOM | 733 | CD2 | LEU | A | 87 | 18.543 | 13.997 | 31.274 | 1.00 | 8.93 | C |
| ATOM | 734 | C | LEU | A | 87 | 16.080 | 11.363 | 33.762 | 1.00 | 13.61 | C |
| ATOM | 735 | O | LEU | A | 87 | 15.955 | 10.441 | 32.943 | 1.00 | 12.97 | O |
| ATOM | 736 | N | PHE | A | 88 | 15.801 | 11.216 | 35.052 | 1.00 | 12.37 | N |
| ATOM | 737 | CA | PHE | A | 88 | 15.252 | 10.022 | 35.518 | 1.00 | 13.75 | C |
| ATOM | 738 | CB | PHE | A | 88 | 15.275 | 9.996 | 37.049 | 1.00 | 15.47 | C |
| ATOM | 739 | CG | PHE | A | 88 | 14.693 | 8.736 | 37.611 | 1.00 | 21.57 | C |
| ATOM | 740 | CD1 | PHE | A | 88 | 15.121 | 7.488 | 37.145 | 1.00 | 29.43 | C |
| ATOM | 741 | CE1 | PHE | A | 88 | 14.558 | 6.304 | 37.655 | 1.00 | 31.14 | C |
| ATOM | 742 | CZ | PHE | A | 88 | 13.568 | 6.367 | 38.664 | 1.00 | 28.33 | C |
| ATOM | 743 | CE2 | PHE | A | 88 | 13.146 | 7.597 | 39.131 | 1.00 | 30.00 | C |
| ATOM | 744 | CD2 | PHE | A | 88 | 13.682 | 8.773 | 38.580 | 1.00 | 27.56 | C |
| ATOM | 745 | C | PHE | A | 88 | 13.839 | 9.782 | 34.943 | 1.00 | 12.86 | C |
| ATOM | 746 | O | PHE | A | 88 | 13.523 | 8.684 | 34.608 | 1.00 | 12.67 | O |
| ATOM | 747 | N | ALA | A | 89 | 13.001 | 10.814 | 34.845 | 1.00 | 13.33 | N |
| ATOM | 748 | CA | ALA | A | 89 | 11.741 | 10.695 | 34.191 | 1.00 | 14.62 | C |
| ATOM | 749 | CB | ALA | A | 89 | 10.903 | 12.040 | 34.308 | 1.00 | 12.92 | C |
| ATOM | 750 | C | ALA | A | 89 | 11.903 | 10.316 | 32.702 | 1.00 | 14.55 | C |
| ATOM | 751 | O | ALA | A | 89 | 11.016 | 9.643 | 32.169 | 1.00 | 16.54 | O |
| ATOM | 752 | N | ARG | A | 90 | 12.951 | 10.820 | 32.015 | 1.00 | 14.18 | N |
| ATOM | 753 | CA | ARG | A | 90 | 13.209 | 10.490 | 30.581 | 1.00 | 14.57 | C |
| ATOM | 754 | CB | ARG | A | 90 | 14.205 | 11.435 | 29.923 | 1.00 | 14.34 | C |
| ATOM | 755 | CG | ARG | A | 90 | 13.597 | 12.752 | 29.498 | 1.00 | 19.24 | C |
| ATOM | 756 | CD | ARG | A | 90 | 12.678 | 12.414 | 28.351 | 1.00 | 24.50 | C |
| ATOM | 757 | NE | ARG | A | 90 | 11.899 | 13.519 | 27.802 | 1.00 | 24.92 | N |
| ATOM | 758 | CZ | ARG | A | 90 | 11.128 | 13.469 | 26.705 | 1.00 | 23.57 | C |
| ATOM | 759 | NH1 | ARG | A | 90 | 11.019 | 12.380 | 25.951 | 1.00 | 18.81 | N |
| ATOM | 760 | NH2 | ARG | A | 90 | 10.432 | 14.527 | 26.354 | 1.00 | 23.30 | N |
| ATOM | 761 | C | ARG | A | 90 | 13.599 | 9.013 | 30.390 | 1.00 | 12.53 | C |
| ATOM | 762 | O | ARG | A | 90 | 13.096 | 8.382 | 29.473 | 1.00 | 13.13 | O |
| ATOM | 763 | N | LEU | A | 91 | 14.397 | 8.451 | 31.299 | 1.00 | 14.15 | N |
| ATOM | 764 | CA | LEU | A | 91 | 14.721 | 7.011 | 31.322 | 1.00 | 13.85 | C |
| ATOM | 765 | CB | LEU | A | 91 | 15.567 | 6.688 | 32.558 | 1.00 | 16.27 | C |
| ATOM | 766 | CG | LEU | A | 91 | 16.522 | 5.488 | 32.568 | 1.00 | 17.38 | C |
| ATOM | 767 | CD1 | LEU | A | 91 | 16.961 | 4.874 | 33.956 | 1.00 | 14.17 | C |
| ATOM | 768 | CD2 | LEU | A | 91 | 16.258 | 4.538 | 31.433 | 1.00 | 13.85 | C |
| ATOM | 769 | C | LEU | A | 91 | 13.393 | 6.232 | 31.422 | 1.00 | 13.93 | C |
| ATOM | 770 | O | LEU | A | 91 | 13.171 | 5.310 | 30.670 | 1.00 | 12.31 | O |
| ATOM | 771 | N | ARG | A | 92 | 12.527 | 6.602 | 32.394 | 1.00 | 13.99 | N |
| ATOM | 772 | CA | ARG | A | 92 | 11.224 | 5.918 | 32.592 | 1.00 | 14.17 | C |
| ATOM | 773 | CB | ARG | A | 92 | 10.455 | 6.385 | 33.832 | 1.00 | 11.97 | C |
| ATOM | 774 | CG | ARG | A | 92 | 11.188 | 6.019 | 35.131 | 1.00 | 15.16 | C |
| ATOM | 775 | CD | ARG | A | 92 | 10.294 | 6.149 | 36.413 | 1.00 | 19.31 | C |
| ATOM | 776 | NE | ARG | A | 92 | 9.417 | 7.356 | 36.441 | 1.00 | 31.40 | N |
| ATOM | 777 | CZ | ARG | A | 92 | 9.736 | 8.616 | 36.839 | 1.00 | 33.81 | C |
| ATOM | 778 | NH1 | ARG | A | 92 | 10.963 | 8.960 | 37.316 | 1.00 | 30.93 | N |
| ATOM | 779 | NH2 | ARG | A | 92 | 8.771 | 9.547 | 36.787 | 1.00 | 33.74 | N |
| ATOM | 780 | C | ARG | A | 92 | 10.360 | 6.086 | 31.350 | 1.00 | 14.55 | C |
| ATOM | 781 | O | ARG | A | 92 | 9.654 | 5.153 | 30.999 | 1.00 | 13.96 | O |
| ATOM | 782 | N | TYR | A | 93 | 10.450 | 7.243 | 30.669 | 1.00 | 14.13 | N |
| ATOM | 783 | CA | TYR | A | 93 | 9.653 | 7.465 | 29.440 | 1.00 | 13.44 | C |
| ATOM | 784 | CB | TYR | A | 93 | 9.700 | 8.948 | 29.034 | 1.00 | 13.24 | C |
| ATOM | 785 | CG | TYR | A | 93 | 9.181 | 9.284 | 27.632 | 1.00 | 11.08 | C |
| ATOM | 786 | CD1 | TYR | A | 93 | 7.854 | 9.650 | 27.433 | 1.00 | 9.64 | C |
| ATOM | 787 | CE1 | TYR | A | 93 | 7.381 | 10.010 | 26.158 | 1.00 | 7.71 | C |
| ATOM | 788 | CZ | TYR | A | 93 | 8.223 | 9.909 | 25.055 | 1.00 | 9.98 | C |
| ATOM | 789 | OH | TYR | A | 93 | 7.720 | 10.213 | 23.847 | 1.00 | 12.50 | O |
| ATOM | 790 | CE2 | TYR | A | 93 | 9.534 | 9.496 | 25.163 | 1.00 | 8.83 | C |
| ATOM | 791 | CD2 | TYR | A | 93 | 10.021 | 9.185 | 26.505 | 1.00 | 9.40 | C |
| ATOM | 792 | C | TYR | A | 93 | 10.056 | 6.511 | 28.328 | 1.00 | 14.35 | C |
| ATOM | 793 | O | TYR | A | 93 | 9.205 | 5.836 | 27.684 | 1.00 | 12.63 | O |
| ATOM | 794 | N | PHE | A | 94 | 11.374 | 6.416 | 28.152 | 1.00 | 13.74 | N |
| ATOM | 795 | CA | PHE | A | 94 | 11.932 | 5.588 | 27.161 | 1.00 | 13.21 | C |
| ATOM | 796 | CB | PHE | A | 94 | 13.442 | 5.809 | 27.057 | 1.00 | 13.53 | C |
| ATOM | 797 | CG | PHE | A | 94 | 14.078 | 4.939 | 26.031 | 1.00 | 14.20 | C |
| ATOM | 798 | CD1 | PHE | A | 94 | 14.260 | 5.418 | 24.711 | 1.00 | 15.87 | C |
| ATOM | 799 | CE1 | PHE | A | 94 | 14.840 | 4.569 | 23.706 | 1.00 | 15.83 | C |
| ATOM | 800 | CZ | PHE | A | 94 | 15.205 | 3.258 | 24.062 | 1.00 | 14.61 | C |
| ATOM | 801 | CE2 | PHE | A | 94 | 15.065 | 2.795 | 25.382 | 1.00 | 14.02 | C |
| ATOM | 802 | CD2 | PHE | A | 94 | 14.478 | 3.634 | 26.358 | 1.00 | 13.17 | C |
| ATOM | 803 | C | PHE | A | 94 | 11.609 | 4.111 | 27.407 | 1.00 | 14.91 | C |
| ATOM | 804 | O | PHE | A | 94 | 11.120 | 3.418 | 26.468 | 1.00 | 12.70 | O |
| ATOM | 805 | N | GLN | A | 95 | 11.872 | 3.630 | 28.639 | 1.00 | 14.32 | N |
| ATOM | 806 | CA | GLN | A | 95 | 11.529 | 2.241 | 29.005 | 1.00 | 16.12 | C |
| ATOM | 807 | CB | GLN | A | 95 | 11.958 | 1.928 | 30.442 | 1.00 | 14.85 | C |
| ATOM | 808 | CG | GLN | A | 95 | 13.515 | 2.021 | 30.619 | 1.00 | 20.59 | C |
| ATOM | 809 | CD | GLN | A | 95 | 13.962 | 1.866 | 32.106 | 1.00 | 21.10 | C |
| ATOM | 810 | OE1 | GLN | A | 95 | 13.324 | 2.408 | 33.060 | 1.00 | 26.27 | O |
| ATOM | 811 | NE2 | GLN | A | 95 | 15.066 | 1.122 | 32.308 | 1.00 | 26.90 | N |
| ATOM | 812 | C | GLN | A | 95 | 10.018 | 1.907 | 28.844 | 1.00 | 15.85 | C |
| ATOM | 813 | O | GLN | A | 95 | 9.665 | 0.796 | 28.412 | 1.00 | 17.07 | O |
| ATOM | 814 | N | GLY | A | 96 | 9.143 | 2.829 | 29.253 | 1.00 | 14.21 | N |
| ATOM | 815 | CA | GLY | A | 96 | 7.705 | 2.678 | 29.142 | 1.00 | 14.91 | C |
| ATOM | 816 | C | GLY | A | 96 | 7.181 | 2.653 | 27.705 | 1.00 | 14.07 | C |
| ATOM | 817 | O | GLY | A | 96 | 6.546 | 1.698 | 27.323 | 1.00 | 15.31 | O |
| ATOM | 818 | N | TYR | A | 97 | 7.453 | 3.708 | 26.946 | 1.00 | 14.51 | N |
| ATOM | 819 | CA | TYR | A | 97 | 7.156 | 3.837 | 25.515 | 1.00 | 14.17 | C |
| ATOM | 820 | CB | TYR | A | 97 | 7.808 | 5.115 | 24.960 | 1.00 | 13.01 | C |
| ATOM | 821 | CG | TYR | A | 97 | 7.242 | 5.618 | 23.664 | 1.00 | 12.30 | C |
| ATOM | 822 | CD1 | TYR | A | 97 | 6.292 | 4.857 | 22.919 | 1.00 | 13.51 | C |
| ATOM | 823 | CE1 | TYR | A | 97 | 5.750 | 5.377 | 21.710 | 1.00 | 11.95 | C |
| ATOM | 824 | CZ | TYR | A | 97 | 6.256 | 6.590 | 21.200 | 1.00 | 9.43 | C |
| ATOM | 825 | OH | TYR | A | 97 | 5.790 | 7.153 | 20.039 | 1.00 | 10.23 | O |
| ATOM | 826 | CE2 | TYR | A | 97 | 7.177 | 7.350 | 21.930 | 1.00 | 11.59 | C |
| ATOM | 827 | CD2 | TYR | A | 97 | 7.675 | 6.844 | 23.142 | 1.00 | 15.09 | C |
| ATOM | 828 | C | TYR | A | 97 | 7.624 | 2.557 | 24.780 | 1.00 | 15.40 | C |
| ATOM | 829 | O | TYR | A | 97 | 6.858 | 1.983 | 24.054 | 1.00 | 13.84 | O |
| ATOM | 830 | N | ALA | A | 98 | 8.830 | 2.044 | 25.069 | 1.00 | 15.47 | N |
| ATOM | 831 | CA | ALA | A | 98 | 9.261 | 0.782 | 24.447 | 1.00 | 15.13 | C |
| ATOM | 832 | CB | ALA | A | 98 | 10.685 | 0.411 | 24.828 | 1.00 | 14.78 | C |
| ATOM | 833 | C | ALA | A | 98 | 8.294 | −0.390 | 24.683 | 1.00 | 15.60 | C |
| ATOM | 834 | O | ALA | A | 98 | 8.005 | −1.120 | 23.757 | 1.00 | 15.78 | O |
| ATOM | 835 | N | ARG | A | 99 | 7.782 | −0.544 | 25.888 | 1.00 | 14.89 | N |
| ATOM | 836 | CA | ARG | A | 99 | 6.758 | −1.550 | 26.186 | 1.00 | 18.61 | C |
| ATOM | 837 | CB | ARG | A | 99 | 6.544 | −1.754 | 27.710 | 1.00 | 17.12 | C |
| ATOM | 838 | CG | ARG | A | 99 | 7.711 | −2.393 | 28.353 | 1.00 | 22.91 | C |
| ATOM | 839 | CD | ARG | A | 99 | 7.615 | −2.367 | 29.913 | 1.00 | 26.13 | C |
| ATOM | 840 | NE | ARG | A | 99 | 6.527 | −3.239 | 30.478 | 1.00 | 31.86 | N |
| ATOM | 841 | CZ | ARG | A | 99 | 5.962 | −3.001 | 31.691 | 1.00 | 36.64 | C |
| ATOM | 842 | NH1 | ARG | A | 99 | 6.377 | −1.933 | 32.430 | 1.00 | 36.40 | N |
| ATOM | 843 | NH2 | ARG | A | 99 | 4.983 | −3.793 | 32.183 | 1.00 | 35.37 | N |
| ATOM | 844 | C | ARG | A | 99 | 5.395 | −1.259 | 25.586 | 1.00 | 16.35 | C |
| ATOM | 845 | O | ARG | A | 99 | 4.706 | −2.178 | 25.184 | 1.00 | 18.90 | O |
| ATOM | 846 | N | SER | A | 100 | 4.994 | −0.004 | 25.582 | 1.00 | 14.50 | N |
| ATOM | 847 | CA | SER | A | 100 | 3.634 | 0.330 | 25.256 | 1.00 | 15.14 | C |
| ATOM | 848 | CB | SER | A | 100 | 2.827 | −0.085 | 26.473 | 1.00 | 14.49 | C |
| ATOM | 849 | OG | SER | A | 100 | 1.516 | 0.163 | 26.252 | 1.00 | 14.92 | O |
| ATOM | 850 | C | SER | A | 100 | 3.514 | 1.857 | 25.011 | 1.00 | 15.28 | C |
| ATOM | 851 | O | SER | A | 100 | 4.074 | 2.660 | 25.765 | 1.00 | 15.11 | O |
| ATOM | 852 | N | ALA | A | 101 | 2.790 | 2.275 | 23.976 | 1.00 | 16.34 | N |
| ATOM | 853 | CA | ALA | A | 101 | 2.545 | 3.706 | 23.806 | 1.00 | 16.79 | C |
| ATOM | 854 | CB | ALA | A | 101 | 1.726 | 3.988 | 22.569 | 1.00 | 17.05 | C |
| ATOM | 855 | C | ALA | A | 101 | 1.878 | 4.293 | 25.066 | 1.00 | 17.22 | C |
| ATOM | 856 | O | ALA | A | 101 | 2.270 | 5.358 | 25.535 | 1.00 | 18.57 | O |
| ATOM | 857 | N | GLN | A | 102 | 0.925 | 3.583 | 25.652 | 1.00 | 15.32 | N |
| ATOM | 858 | CA | GLN | A | 102 | 0.214 | 4.095 | 26.836 | 1.00 | 15.90 | C |
| ATOM | 859 | CB | GLN | A | 102 | −1.049 | 3.224 | 27.162 | 1.00 | 14.84 | C |
| ATOM | 860 | CG | GLN | A | 102 | −1.968 | 3.823 | 28.277 | 1.00 | 13.81 | C |
| ATOM | 861 | CD | GLN | A | 102 | −1.476 | 3.444 | 29.699 | 1.00 | 17.70 | C |
| ATOM | 862 | OE1 | GLN | A | 102 | −0.779 | 2.433 | 29.897 | 1.00 | 19.22 | O |
| ATOM | 863 | NE2 | GLN | A | 102 | −1.861 | 4.222 | 30.674 | 1.00 | 17.49 | N |
| ATOM | 864 | C | GLN | A | 102 | 1.169 | 4.293 | 28.057 | 1.00 | 16.02 | C |
| ATOM | 865 | O | GLN | A | 102 | 1.001 | 5.238 | 28.792 | 1.00 | 17.70 | O |
| ATOM | 866 | N | LEU | A | 103 | 2.206 | 3.452 | 28.200 | 1.00 | 15.32 | N |
| ATOM | 867 | CA | LEU | A | 103 | 3.155 | 3.553 | 29.292 | 1.00 | 15.42 | C |
| ATOM | 868 | CB | LEU | A | 103 | 3.926 | 2.251 | 29.531 | 1.00 | 13.30 | C |
| ATOM | 869 | CG | LEU | A | 103 | 3.111 | 1.164 | 30.209 | 1.00 | 16.00 | C |
| ATOM | 870 | CD1 | LEU | A | 103 | 3.808 | −0.159 | 29.972 | 1.00 | 17.16 | C |
| ATOM | 871 | CD2 | LEU | A | 103 | 2.796 | 1.400 | 31.705 | 1.00 | 8.03 | C |
| ATOM | 872 | C | LEU | A | 103 | 4.142 | 4.698 | 29.166 | 1.00 | 15.78 | C |
| ATOM | 873 | O | LEU | A | 103 | 4.889 | 4.946 | 30.094 | 1.00 | 17.02 | O |
| ATOM | 874 | N | ARG | A | 104 | 4.172 | 5.390 | 28.034 | 1.00 | 16.09 | N |
| ATOM | 875 | CA | ARG | A | 104 | 4.968 | 6.627 | 27.928 | 1.00 | 14.49 | C |
| ATOM | 876 | CB | ARG | A | 104 | 5.161 | 7.073 | 26.470 | 1.00 | 15.67 | C |
| ATOM | 877 | CG | ARG | A | 104 | 4.087 | 8.047 | 25.918 | 1.00 | 12.76 | C |
| ATOM | 878 | CD | ARG | A | 104 | 4.194 | 8.213 | 24.407 | 1.00 | 12.98 | C |
| ATOM | 879 | NE | ARG | A | 104 | 3.190 | 9.183 | 23.965 | 1.00 | 11.68 | N |
| ATOM | 880 | CZ | ARG | A | 104 | 1.884 | 8.907 | 23.798 | 1.00 | 16.02 | C |
| ATOM | 881 | NH1 | ARG | A | 104 | 1.066 | 9.887 | 23.448 | 1.00 | 16.52 | N |
| ATOM | 882 | NH2 | ARG | A | 104 | 1.364 | 7.674 | 24.026 | 1.00 | 14.91 | N |
| ATOM | 883 | C | ARG | A | 104 | 4.372 | 7.836 | 28.724 | 1.00 | 14.93 | C |
| ATOM | 884 | O | ARG | A | 104 | 5.115 | 8.780 | 29.046 | 1.00 | 14.63 | O |
| ATOM | 885 | N | LEU | A | 105 | 3.075 | 7.793 | 29.029 | 1.00 | 13.25 | N |
| ATOM | 886 | CA | LEU | A | 105 | 2.340 | 8.984 | 29.475 | 1.00 | 12.90 | C |
| ATOM | 887 | CB | LEU | A | 105 | 0.837 | 8.801 | 29.283 | 1.00 | 13.12 | C |
| ATOM | 888 | CG | LEU | A | 105 | 0.401 | 8.723 | 27.814 | 1.00 | 13.13 | C |
| ATOM | 889 | CD1 | LEU | A | 105 | −1.124 | 8.236 | 27.794 | 1.00 | 14.35 | C |
| ATOM | 890 | CD2 | LEU | A | 105 | 0.669 | 10.072 | 27.072 | 1.00 | 11.38 | C |
| ATOM | 891 | C | LEU | A | 105 | 2.661 | 9.452 | 30.893 | 1.00 | 12.95 | C |
| ATOM | 892 | O | LEU | A | 105 | 2.952 | 10.665 | 31.098 | 1.00 | 12.05 | O |
| ATOM | 893 | N | ALA | A | 106 | 2.626 | 8.544 | 31.874 | 1.00 | 12.66 | N |
| ATOM | 894 | CA | ALA | A | 106 | 2.898 | 8.971 | 33.269 | 1.00 | 13.15 | C |
| ATOM | 895 | CB | ALA | A | 106 | 2.773 | 7.778 | 34.293 | 1.00 | 10.91 | C |
| ATOM | 896 | C | ALA | A | 106 | 4.273 | 9.643 | 33.355 | 1.00 | 13.75 | C |
| ATOM | 897 | O | ALA | A | 106 | 4.414 | 10.774 | 33.861 | 1.00 | 15.38 | O |
| ATOM | 898 | N | PRO | A | 107 | 5.323 | 8.962 | 32.876 | 1.00 | 15.07 | N |
| ATOM | 899 | CA | PRO | A | 107 | 6.636 | 9.690 | 32.873 | 1.00 | 13.91 | C |
| ATOM | 900 | CB | PRO | A | 107 | 7.648 | 8.579 | 32.608 | 1.00 | 14.31 | C |
| ATOM | 901 | CG | PRO | A | 107 | 6.837 | 7.480 | 31.988 | 1.00 | 16.27 | C |
| ATOM | 902 | CD | PRO | A | 107 | 5.458 | 7.541 | 32.492 | 1.00 | 14.07 | C |
| ATOM | 903 | C | PRO | A | 107 | 6.867 | 10.846 | 31.914 | 1.00 | 12.66 | C |
| ATOM | 904 | O | PRO | A | 107 | 7.740 | 11.686 | 32.213 | 1.00 | 13.50 | O |
| ATOM | 905 | N | LEU | A | 108 | 6.192 | 10.905 | 30.773 | 1.00 | 10.99 | N |
| ATOM | 906 | CA | LEU | A | 108 | 6.188 | 12.141 | 29.991 | 1.00 | 12.51 | C |
| ATOM | 907 | CB | LEU | A | 108 | 5.261 | 12.056 | 28.762 | 1.00 | 12.84 | C |
| ATOM | 908 | CG | LEU | A | 108 | 5.153 | 13.340 | 27.937 | 1.00 | 14.17 | C |
| ATOM | 909 | CD1 | LEU | A | 108 | 6.553 | 13.818 | 27.293 | 1.00 | 10.33 | C |
| ATOM | 910 | CD2 | LEU | A | 108 | 4.084 | 13.124 | 26.905 | 1.00 | 14.83 | C |
| ATOM | 911 | C | LEU | A | 108 | 5.711 | 13.302 | 30.876 | 1.00 | 13.04 | C |
| ATOM | 912 | O | LEU | A | 108 | 6.333 | 14.373 | 30.888 | 1.00 | 15.26 | O |
| ATOM | 913 | N | TYR | A | 109 | 4.604 | 13.104 | 31.594 | 1.00 | 13.82 | N |
| ATOM | 914 | CA | TYR | A | 109 | 4.106 | 14.116 | 32.509 | 1.00 | 13.76 | C |
| ATOM | 915 | CB | TYR | A | 109 | 2.756 | 13.735 | 33.137 | 1.00 | 15.29 | C |
| ATOM | 916 | CG | TYR | A | 109 | 1.689 | 13.437 | 32.178 | 1.00 | 17.61 | C |
| ATOM | 917 | CD1 | TYR | A | 109 | 1.481 | 14.243 | 31.086 | 1.00 | 21.26 | C |
| ATOM | 918 | CE1 | TYR | A | 109 | 0.472 | 13.981 | 30.196 | 1.00 | 20.34 | C |
| ATOM | 919 | CZ | TYR | A | 109 | −0.325 | 12.920 | 30.394 | 1.00 | 19.53 | C |
| ATOM | 920 | OH | TYR | A | 109 | −1.302 | 12.681 | 29.487 | 1.00 | 22.88 | O |
| ATOM | 921 | CE2 | TYR | A | 109 | −0.169 | 12.078 | 31.487 | 1.00 | 21.61 | C |
| ATOM | 922 | CD2 | TYR | A | 109 | 0.852 | 12.360 | 32.382 | 1.00 | 17.80 | C |
| ATOM | 923 | C | TYR | A | 109 | 5.073 | 14.453 | 33.648 | 1.00 | 12.22 | C |
| ATOM | 924 | O | TYR | A | 109 | 5.205 | 15.627 | 33.951 | 1.00 | 11.21 | O |
| ATOM | 925 | N | ALA | A | 110 | 5.701 | 13.448 | 34.301 | 1.00 | 11.08 | N |
| ATOM | 926 | CA | ALA | A | 110 | 6.776 | 13.771 | 35.262 | 1.00 | 11.48 | C |
| ATOM | 927 | CB | ALA | A | 110 | 7.328 | 12.511 | 35.988 | 1.00 | 8.23 | C |
| ATOM | 928 | C | ALA | A | 110 | 7.888 | 14.600 | 34.593 | 1.00 | 11.82 | C |
| ATOM | 929 | O | ALA | A | 110 | 8.368 | 15.531 | 35.163 | 1.00 | 14.31 | O |
| ATOM | 930 | N | SER | A | 111 | 8.307 | 14.265 | 33.378 | 1.00 | 12.96 | N |
| ATOM | 931 | CA | SER | A | 111 | 9.421 | 14.988 | 32.747 | 1.00 | 13.27 | C |
| ATOM | 932 | CB | SER | A | 111 | 9.991 | 14.210 | 31.527 | 1.00 | 10.94 | C |
| ATOM | 933 | OG | SER | A | 111 | 9.145 | 14.441 | 30.410 | 1.00 | 16.40 | O |
| ATOM | 934 | C | SER | A | 111 | 8.994 | 16.478 | 32.380 | 1.00 | 13.84 | C |
| ATOM | 935 | O | SER | A | 111 | 9.766 | 17.421 | 32.529 | 1.00 | 14.85 | O |
| ATOM | 936 | N | ALA | A | 112 | 7.745 | 16.670 | 31.963 | 1.00 | 13.58 | N |
| ATOM | 937 | CA | ALA | A | 112 | 7.219 | 17.993 | 31.703 | 1.00 | 11.84 | C |
| ATOM | 938 | CB | ALA | A | 112 | 5.838 | 17.898 | 31.051 | 1.00 | 10.37 | C |
| ATOM | 939 | C | ALA | A | 112 | 7.167 | 18.881 | 32.974 | 1.00 | 11.97 | C |
| ATOM | 940 | O | ALA | A | 112 | 7.481 | 20.102 | 32.900 | 1.00 | 10.21 | O |
| ATOM | 941 | N | ARG | A | 113 | 6.738 | 18.312 | 34.105 | 1.00 | 12.79 | N |
| ATOM | 942 | CA | ARG | A | 113 | 6.711 | 19.070 | 35.368 | 1.00 | 13.87 | C |
| ATOM | 943 | CB | ARG | A | 113 | 6.120 | 18.266 | 36.537 | 1.00 | 13.69 | C |
| ATOM | 944 | CG | ARG | A | 113 | 4.596 | 18.128 | 36.421 | 1.00 | 15.95 | C |
| ATOM | 945 | CD | ARG | A | 113 | 3.932 | 17.387 | 37.599 | 1.00 | 18.47 | C |
| ATOM | 946 | NE | ARG | A | 113 | 4.346 | 15.979 | 37.685 | 1.00 | 25.47 | N |
| ATOM | 947 | CZ | ARG | A | 113 | 3.610 | 14.918 | 37.306 | 1.00 | 26.94 | C |
| ATOM | 948 | NH1 | ARG | A | 113 | 2.359 | 15.082 | 36.820 | 1.00 | 27.58 | N |
| ATOM | 949 | NH2 | ARG | A | 113 | 4.129 | 13.691 | 37.430 | 1.00 | 23.12 | N |
| ATOM | 950 | C | ARG | A | 113 | 8.133 | 19.554 | 35.685 | 1.00 | 13.41 | C |
| ATOM | 951 | O | ARG | A | 113 | 8.300 | 20.667 | 36.101 | 1.00 | 13.78 | O |
| ATOM | 952 | N | ALA | A | 114 | 9.136 | 18.699 | 35.485 | 1.00 | 12.35 | N |
| ATOM | 953 | CA | ALA | A | 114 | 10.477 | 19.029 | 35.873 | 1.00 | 11.15 | C |
| ATOM | 954 | CB | ALA | A | 114 | 11.362 | 17.762 | 35.926 | 1.00 | 10.92 | C |
| ATOM | 955 | C | ALA | A | 114 | 11.048 | 20.101 | 34.903 | 1.00 | 11.85 | C |
| ATOM | 956 | O | ALA | A | 114 | 11.667 | 21.066 | 35.366 | 1.00 | 9.24 | O |
| ATOM | 957 | N | LEU | A | 115 | 10.772 | 19.976 | 33.598 | 1.00 | 10.88 | N |
| ATOM | 958 | CA | LEU | A | 115 | 11.277 | 20.980 | 32.619 | 1.00 | 13.38 | C |
| ATOM | 959 | CB | LEU | A | 115 | 11.005 | 20.488 | 31.163 | 1.00 | 12.74 | C |
| ATOM | 960 | CG | LEU | A | 115 | 11.715 | 21.035 | 29.972 | 1.00 | 13.69 | C |
| ATOM | 961 | CD1 | LEU | A | 115 | 13.238 | 21.172 | 30.317 | 1.00 | 16.09 | C |
| ATOM | 962 | CD2 | LEU | A | 115 | 11.542 | 20.106 | 28.770 | 1.00 | 14.91 | C |
| ATOM | 963 | C | LEU | A | 115 | 10.541 | 22.315 | 32.866 | 1.00 | 13.80 | C |
| ATOM | 964 | O | LEU | A | 115 | 11.171 | 23.365 | 32.900 | 1.00 | 16.02 | O |
| ATOM | 965 | N | TRP | A | 116 | 9.221 | 22.271 | 33.054 | 1.00 | 13.64 | N |
| ATOM | 966 | CA | TRP | A | 116 | 8.506 | 23.480 | 33.356 | 1.00 | 13.88 | C |
| ATOM | 967 | CB | TRP | A | 116 | 7.016 | 23.231 | 33.466 | 1.00 | 13.31 | C |
| ATOM | 968 | CG | TRP | A | 116 | 6.262 | 23.224 | 32.097 | 1.00 | 16.85 | C |
| ATOM | 969 | CD1 | TRP | A | 116 | 5.436 | 22.187 | 31.593 | 1.00 | 13.15 | C |
| ATOM | 970 | NE1 | TRP | A | 116 | 4.893 | 22.561 | 30.385 | 1.00 | 14.92 | N |
| ATOM | 971 | CE2 | TRP | A | 116 | 5.315 | 23.822 | 30.064 | 1.00 | 14.46 | C |
| ATOM | 972 | CD2 | TRP | A | 116 | 6.161 | 24.293 | 31.133 | 1.00 | 15.63 | C |
| ATOM | 973 | CE3 | TRP | A | 116 | 6.702 | 25.593 | 31.054 | 1.00 | 16.77 | C |
| ATOM | 974 | CZ3 | TRP | A | 116 | 6.375 | 26.372 | 29.943 | 1.00 | 16.02 | C |
| ATOM | 975 | CH2 | TRP | A | 116 | 5.537 | 25.863 | 28.890 | 1.00 | 14.85 | C |
| ATOM | 976 | CZ2 | TRP | A | 116 | 5.012 | 24.596 | 28.944 | 1.00 | 15.66 | C |
| ATOM | 977 | C | TRP | A | 116 | 9.001 | 24.198 | 34.620 | 1.00 | 13.49 | C |
| ATOM | 978 | O | TRP | A | 116 | 9.011 | 25.426 | 34.672 | 1.00 | 12.26 | O |
| ATOM | 979 | N | LEU | A | 117 | 9.353 | 23.448 | 35.655 | 1.00 | 14.45 | N |
| ATOM | 980 | CA | LEU | A | 117 | 9.980 | 24.047 | 36.825 | 1.00 | 15.88 | C |
| ATOM | 981 | CB | LEU | A | 117 | 10.101 | 23.020 | 37.961 | 1.00 | 15.64 | C |
| ATOM | 982 | CG | LEU | A | 117 | 10.651 | 23.436 | 39.298 | 1.00 | 20.17 | C |
| ATOM | 983 | CD1 | LEU | A | 117 | 9.846 | 24.607 | 39.951 | 1.00 | 21.46 | C |
| ATOM | 984 | CD2 | LEU | A | 117 | 10.601 | 22.165 | 40.221 | 1.00 | 18.31 | C |
| ATOM | 985 | C | LEU | A | 117 | 11.317 | 24.684 | 36.465 | 1.00 | 15.24 | C |
| ATOM | 986 | O | LEU | A | 117 | 11.608 | 25.778 | 36.937 | 1.00 | 16.29 | O |
| ATOM | 987 | N | LEU | A | 118 | 12.133 | 24.047 | 35.643 | 1.00 | 14.71 | N |
| ATOM | 988 | CA | LEU | A | 118 | 13.381 | 24.693 | 35.193 | 1.00 | 15.37 | C |
| ATOM | 989 | CB | LEU | A | 118 | 14.264 | 23.778 | 34.337 | 1.00 | 14.41 | C |
| ATOM | 990 | CG | LEU | A | 118 | 15.072 | 22.673 | 35.008 | 1.00 | 15.73 | C |
| ATOM | 991 | CD1 | LEU | A | 118 | 15.681 | 21.656 | 33.970 | 1.00 | 13.78 | C |
| ATOM | 992 | CD2 | LEU | A | 118 | 16.144 | 23.343 | 35.876 | 1.00 | 10.27 | C |
| ATOM | 993 | C | LEU | A | 118 | 13.079 | 25.990 | 34.420 | 1.00 | 14.94 | C |
| ATOM | 994 | O | LEU | A | 118 | 13.794 | 26.958 | 34.586 | 1.00 | 15.78 | O |
| ATOM | 995 | N | VAL | A | 119 | 12.047 | 25.991 | 33.566 | 1.00 | 14.65 | N |
| ATOM | 996 | CA | VAL | A | 119 | 11.725 | 27.163 | 32.816 | 1.00 | 11.84 | C |
| ATOM | 997 | CB | VAL | A | 119 | 10.594 | 26.871 | 31.799 | 1.00 | 11.96 | C |
| ATOM | 998 | CG1 | VAL | A | 119 | 9.910 | 28.176 | 31.352 | 1.00 | 10.15 | C |
| ATOM | 999 | CG2 | VAL | A | 119 | 11.076 | 26.026 | 30.581 | 1.00 | 8.10 | C |
| ATOM | 1000 | C | VAL | A | 119 | 11.326 | 28.296 | 33.813 | 1.00 | 13.56 | C |
| ATOM | 1001 | O | VAL | A | 119 | 11.699 | 29.443 | 33.624 | 1.00 | 14.32 | O |
| ATOM | 1002 | N | ALA | A | 120 | 10.551 | 27.964 | 34.851 | 1.00 | 13.23 | N |
| ATOM | 1003 | CA | ALA | A | 120 | 10.071 | 28.922 | 35.801 | 1.00 | 13.63 | C |
| ATOM | 1004 | CB | ALA | A | 120 | 9.006 | 28.329 | 36.725 | 1.00 | 13.60 | C |
| ATOM | 1005 | C | ALA | A | 120 | 11.226 | 29.453 | 36.597 | 1.00 | 13.56 | C |
| ATOM | 1006 | O | ALA | A | 120 | 11.254 | 30.632 | 36.874 | 1.00 | 13.30 | O |
| ATOM | 1007 | N | LEU | A | 121 | 12.174 | 28.602 | 36.977 | 1.00 | 13.08 | N |
| ATOM | 1008 | CA | LEU | A | 121 | 13.325 | 29.118 | 37.697 | 1.00 | 14.20 | C |
| ATOM | 1009 | CB | LEU | A | 121 | 14.181 | 28.004 | 38.352 | 1.00 | 13.22 | C |
| ATOM | 1010 | CG | LEU | A | 121 | 13.491 | 27.071 | 39.344 | 1.00 | 13.38 | C |
| ATOM | 1011 | CD1 | LEU | A | 121 | 14.549 | 26.035 | 39.797 | 1.00 | 11.02 | C |
| ATOM | 1012 | CD2 | LEU | A | 121 | 12.805 | 27.806 | 40.508 | 1.00 | 13.39 | C |
| ATOM | 1013 | C | LEU | A | 121 | 14.221 | 29.994 | 36.809 | 1.00 | 13.20 | C |
| ATOM | 1014 | O | LEU | A | 121 | 14.803 | 30.929 | 37.305 | 1.00 | 14.45 | O |
| ATOM | 1015 | N | ALA | A | 122 | 14.340 | 29.662 | 35.520 | 1.00 | 14.22 | N |
| ATOM | 1016 | CA | ALA | A | 122 | 15.103 | 30.471 | 34.574 | 1.00 | 14.30 | C |
| ATOM | 1017 | CB | ALA | A | 122 | 15.233 | 29.771 | 33.270 | 1.00 | 12.02 | C |
| ATOM | 1018 | C | ALA | A | 122 | 14.452 | 31.876 | 34.419 | 1.00 | 14.09 | C |
| ATOM | 1019 | O | ALA | A | 122 | 15.144 | 32.866 | 34.502 | 1.00 | 15.03 | O |
| ATOM | 1020 | N | ALA | A | 123 | 13.127 | 31.932 | 34.286 | 1.00 | 15.23 | N |
| ATOM | 1021 | CA | ALA | A | 123 | 12.365 | 33.196 | 34.181 | 1.00 | 14.48 | C |
| ATOM | 1022 | CB | ALA | A | 123 | 10.905 | 32.912 | 33.818 | 1.00 | 13.53 | C |
| ATOM | 1023 | C | ALA | A | 123 | 12.478 | 34.103 | 35.445 | 1.00 | 15.58 | C |
| ATOM | 1024 | O | ALA | A | 123 | 12.692 | 35.291 | 35.312 | 1.00 | 15.67 | O |
| ATOM | 1025 | N | LEU | A | 124 | 12.328 | 33.521 | 36.639 | 1.00 | 14.48 | N |
| ATOM | 1026 | CA | LEU | A | 124 | 12.529 | 34.188 | 37.883 | 1.00 | 16.04 | C |
| ATOM | 1027 | CB | LEU | A | 124 | 12.177 | 33.310 | 39.120 | 1.00 | 15.29 | C |
| ATOM | 1028 | CG | LEU | A | 124 | 10.724 | 32.882 | 39.232 | 1.00 | 17.42 | C |
| ATOM | 1029 | CD1 | LEU | A | 124 | 10.507 | 31.731 | 40.252 | 1.00 | 15.84 | C |
| ATOM | 1030 | CD2 | LEU | A | 124 | 9.740 | 34.103 | 39.462 | 1.00 | 17.06 | C |
| ATOM | 1031 | C | LEU | A | 124 | 13.940 | 34.692 | 38.002 | 1.00 | 15.91 | C |
| ATOM | 1032 | O | LEU | A | 124 | 14.103 | 35.816 | 38.407 | 1.00 | 17.85 | O |
| ATOM | 1033 | N | GLY | A | 125 | 14.959 | 33.925 | 37.633 | 1.00 | 15.26 | N |
| ATOM | 1034 | CA | GLY | A | 125 | 16.266 | 34.465 | 37.742 | 1.00 | 14.67 | C |
| ATOM | 1035 | C | GLY | A | 125 | 16.490 | 35.642 | 36.785 | 1.00 | 15.34 | C |
| ATOM | 1036 | O | GLY | A | 125 | 17.205 | 36.605 | 37.148 | 1.00 | 13.70 | O |
| ATOM | 1037 | N | LEU | A | 126 | 16.005 | 35.516 | 35.535 | 1.00 | 15.03 | N |
| ATOM | 1038 | CA | LEU | A | 126 | 16.145 | 36.573 | 34.547 | 1.00 | 14.38 | C |
| ATOM | 1039 | CB | LEU | A | 126 | 15.715 | 36.133 | 33.141 | 1.00 | 12.20 | C |
| ATOM | 1040 | CG | LEU | A | 126 | 16.729 | 35.176 | 32.490 | 1.00 | 12.52 | C |
| ATOM | 1041 | CD1 | LEU | A | 126 | 16.040 | 34.599 | 31.253 | 1.00 | 9.80 | C |
| ATOM | 1042 | CD2 | LEU | A | 126 | 18.055 | 35.833 | 32.135 | 1.00 | 2.00 | C |
| ATOM | 1043 | C | LEU | A | 126 | 15.308 | 37.807 | 35.002 | 1.00 | 15.97 | C |
| ATOM | 1044 | O | LEU | A | 126 | 15.780 | 38.957 | 34.864 | 1.00 | 16.88 | O |
| ATOM | 1045 | N | LEU | A | 127 | 14.107 | 37.604 | 35.537 | 1.00 | 14.84 | N |
| ATOM | 1046 | CA | LEU | A | 127 | 13.376 | 38.715 | 36.090 | 1.00 | 17.03 | C |
| ATOM | 1047 | CB | LEU | A | 127 | 12.068 | 38.264 | 36.654 | 1.00 | 17.65 | C |
| ATOM | 1048 | CG | LEU | A | 127 | 10.992 | 38.413 | 35.627 | 1.00 | 26.91 | C |
| ATOM | 1049 | CD1 | LEU | A | 127 | 9.886 | 37.317 | 35.933 | 1.00 | 32.60 | C |
| ATOM | 1050 | CD2 | LEU | A | 127 | 10.454 | 39.885 | 35.581 | 1.00 | 29.73 | C |
| ATOM | 1051 | C | LEU | A | 127 | 14.177 | 39.431 | 37.221 | 1.00 | 16.39 | C |
| ATOM | 1052 | O | LEU | A | 127 | 14.205 | 40.641 | 37.238 | 1.00 | 16.26 | O |
| ATOM | 1053 | N | ALA | A | 128 | 14.745 | 38.683 | 38.181 | 1.00 | 16.11 | N |
| ATOM | 1054 | CA | ALA | A | 128 | 15.560 | 39.301 | 39.247 | 1.00 | 15.78 | C |
| ATOM | 1055 | CB | ALA | A | 128 | 16.001 | 38.292 | 40.264 | 1.00 | 15.58 | C |
| ATOM | 1056 | C | ALA | A | 128 | 16.761 | 40.032 | 38.638 | 1.00 | 15.63 | C |
| ATOM | 1057 | O | ALA | A | 128 | 17.159 | 41.070 | 39.122 | 1.00 | 17.53 | O |
| ATOM | 1058 | N | HIS | A | 129 | 17.294 | 39.556 | 37.535 | 1.00 | 16.41 | N |
| ATOM | 1059 | CA | HIS | A | 129 | 18.379 | 40.271 | 36.890 | 1.00 | 16.54 | C |
| ATOM | 1060 | CB | HIS | A | 129 | 19.118 | 39.371 | 35.890 | 1.00 | 17.13 | C |
| ATOM | 1061 | CG | HIS | A | 129 | 20.251 | 40.065 | 35.217 | 1.00 | 17.82 | C |
| ATOM | 1062 | ND1 | HIS | A | 129 | 21.496 | 40.185 | 35.797 | 1.00 | 17.71 | N |
| ATOM | 1063 | CE1 | HIS | A | 129 | 22.282 | 40.910 | 35.018 | 1.00 | 17.15 | C |
| ATOM | 1064 | NE2 | HIS | A | 129 | 21.590 | 41.268 | 33.950 | 1.00 | 17.75 | N |
| ATOM | 1065 | CD2 | HIS | A | 129 | 20.323 | 40.731 | 34.035 | 1.00 | 17.93 | C |
| ATOM | 1066 | C | HIS | A | 129 | 17.954 | 41.614 | 36.223 | 1.00 | 17.26 | C |
| ATOM | 1067 | O | HIS | A | 129 | 18.664 | 42.605 | 36.407 | 1.00 | 17.60 | O |
| ATOM | 1068 | N | PHE | A | 130 | 16.823 | 41.646 | 35.480 | 1.00 | 15.37 | N |
| ATOM | 1069 | CA | PHE | A | 130 | 16.438 | 42.789 | 34.671 | 1.00 | 15.70 | C |
| ATOM | 1070 | CB | PHE | A | 130 | 15.861 | 42.365 | 33.292 | 1.00 | 14.48 | C |
| ATOM | 1071 | CG | PHE | A | 130 | 16.875 | 41.811 | 32.333 | 1.00 | 14.26 | C |
| ATOM | 1072 | CD1 | PHE | A | 130 | 17.803 | 42.661 | 31.695 | 1.00 | 12.46 | C |
| ATOM | 1073 | CE1 | PHE | A | 130 | 18.726 | 42.117 | 30.736 | 1.00 | 14.66 | C |
| ATOM | 1074 | CZ | PHE | A | 130 | 18.683 | 40.747 | 30.426 | 1.00 | 11.28 | C |
| ATOM | 1075 | CE2 | PHE | A | 130 | 17.741 | 39.898 | 31.074 | 1.00 | 9.83 | C |
| ATOM | 1076 | CD2 | PHE | A | 130 | 16.863 | 40.436 | 32.007 | 1.00 | 9.24 | C |
| ATOM | 1077 | C | PHE | A | 130 | 15.468 | 43.808 | 35.339 | 1.00 | 16.84 | C |
| ATOM | 1078 | O | PHE | A | 130 | 15.513 | 44.975 | 34.988 | 1.00 | 15.03 | O |
| ATOM | 1079 | N | LEU | A | 131 | 14.599 | 43.368 | 36.263 | 1.00 | 18.31 | N |
| ATOM | 1080 | CA | LEU | A | 131 | 13.485 | 44.202 | 36.725 | 1.00 | 19.33 | C |
| ATOM | 1081 | CB | LEU | A | 131 | 12.471 | 43.345 | 37.475 | 1.00 | 21.55 | C |
| ATOM | 1082 | CG | LEU | A | 131 | 11.072 | 43.868 | 37.754 | 1.00 | 24.78 | C |
| ATOM | 1083 | CD1 | LEU | A | 131 | 10.360 | 44.192 | 36.448 | 1.00 | 24.49 | C |
| ATOM | 1084 | CD2 | LEU | A | 131 | 10.345 | 42.759 | 38.512 | 1.00 | 27.31 | C |
| ATOM | 1085 | C | LEU | A | 131 | 13.941 | 45.434 | 37.535 | 1.00 | 19.75 | C |
| ATOM | 1086 | O | LEU | A | 131 | 13.441 | 46.532 | 37.272 | 1.00 | 17.96 | O |
| ATOM | 1087 | N | PRO | A | 132 | 14.886 | 45.269 | 38.500 | 1.00 | 19.97 | N |
| ATOM | 1088 | CA | PRO | A | 132 | 15.345 | 46.415 | 39.333 | 1.00 | 20.32 | C |
| ATOM | 1089 | CB | PRO | A | 132 | 16.443 | 45.791 | 40.248 | 1.00 | 20.94 | C |
| ATOM | 1090 | CG | PRO | A | 132 | 15.921 | 44.302 | 40.402 | 1.00 | 19.47 | C |
| ATOM | 1091 | CD | PRO | A | 132 | 15.533 | 44.012 | 38.944 | 1.00 | 21.77 | C |
| ATOM | 1092 | C | PRO | A | 132 | 15.823 | 47.580 | 38.503 | 1.00 | 19.76 | C |
| ATOM | 1093 | O | PRO | A | 132 | 15.265 | 48.671 | 38.616 | 1.00 | 20.31 | O |
| ATOM | 1094 | N | ALA | A | 133 | 16.756 | 47.318 | 37.595 | 1.00 | 18.26 | N |
| ATOM | 1095 | CA | ALA | A | 133 | 17.251 | 48.361 | 36.768 | 1.00 | 16.31 | C |
| ATOM | 1096 | CB | ALA | A | 133 | 18.521 | 47.924 | 36.014 | 1.00 | 14.32 | C |
| ATOM | 1097 | C | ALA | A | 133 | 16.206 | 48.935 | 35.832 | 1.00 | 14.97 | C |
| ATOM | 1098 | O | ALA | A | 133 | 16.216 | 50.119 | 35.606 | 1.00 | 16.49 | O |
| ATOM | 1099 | N | ALA | A | 134 | 15.304 | 48.136 | 35.273 | 1.00 | 14.39 | N |
| ATOM | 1100 | CA | ALA | A | 134 | 14.215 | 48.705 | 34.473 | 1.00 | 13.98 | C |
| ATOM | 1101 | CB | ALA | A | 134 | 13.452 | 47.588 | 33.660 | 1.00 | 13.38 | C |
| ATOM | 1102 | C | ALA | A | 134 | 13.250 | 49.577 | 35.280 | 1.00 | 14.32 | C |
| ATOM | 1103 | O | ALA | A | 134 | 12.835 | 50.671 | 34.786 | 1.00 | 14.38 | O |
| ATOM | 1104 | N | LEU | A | 135 | 12.894 | 49.147 | 36.508 | 1.00 | 14.05 | N |
| ATOM | 1105 | CA | LEU | A | 135 | 11.992 | 49.957 | 37.317 | 1.00 | 15.12 | C |
| ATOM | 1106 | CB | LEU | A | 135 | 11.510 | 49.203 | 38.549 | 1.00 | 16.20 | C |
| ATOM | 1107 | CG | LEU | A | 135 | 10.601 | 47.982 | 38.289 | 1.00 | 18.97 | C |
| ATOM | 1108 | CD1 | LEU | A | 135 | 10.218 | 47.302 | 39.601 | 1.00 | 19.42 | C |
| ATOM | 1109 | CD2 | LEU | A | 135 | 9.329 | 48.362 | 37.460 | 1.00 | 19.36 | C |
| ATOM | 1110 | C | LEU | A | 135 | 12.655 | 51.301 | 37.733 | 1.00 | 15.26 | C |
| ATOM | 1111 | O | LEU | A | 135 | 12.018 | 52.382 | 37.734 | 1.00 | 15.72 | O |
| ATOM | 1112 | N | ARG | A | 136 | 13.938 | 51.210 | 38.077 | 1.00 | 14.02 | N |
| ATOM | 1113 | CA | ARG | A | 136 | 14.716 | 52.358 | 38.455 | 1.00 | 13.62 | C |
| ATOM | 1114 | CB | ARG | A | 136 | 16.122 | 51.952 | 38.796 | 1.00 | 11.70 | C |
| ATOM | 1115 | CG | ARG | A | 136 | 16.799 | 53.193 | 39.199 | 1.00 | 17.23 | C |
| ATOM | 1116 | CD | ARG | A | 136 | 17.999 | 53.021 | 39.977 | 1.00 | 21.91 | C |
| ATOM | 1117 | NE | ARG | A | 136 | 19.048 | 52.500 | 39.145 | 1.00 | 27.85 | N |
| ATOM | 1118 | CZ | ARG | A | 136 | 20.322 | 52.512 | 39.502 | 1.00 | 32.97 | C |
| ATOM | 1119 | NH1 | ARG | A | 136 | 20.721 | 53.038 | 40.656 | 1.00 | 29.38 | N |
| ATOM | 1120 | NH2 | ARG | A | 136 | 21.211 | 51.993 | 38.692 | 1.00 | 38.10 | N |
| ATOM | 1121 | C | ARG | A | 136 | 14.766 | 53.387 | 37.340 | 1.00 | 12.04 | C |
| ATOM | 1122 | O | ARG | A | 136 | 14.522 | 54.551 | 37.573 | 1.00 | 13.00 | O |
| ATOM | 1123 | N | ALA | A | 137 | 15.048 | 52.934 | 36.118 | 1.00 | 13.52 | N |
| ATOM | 1124 | CA | ALA | A | 137 | 15.091 | 53.810 | 34.952 | 1.00 | 13.62 | C |
| ATOM | 1125 | CB | ALA | A | 137 | 15.637 | 53.013 | 33.709 | 1.00 | 12.85 | C |
| ATOM | 1126 | C | ALA | A | 137 | 13.718 | 54.411 | 34.661 | 1.00 | 13.19 | C |
| ATOM | 1127 | O | ALA | A | 137 | 13.621 | 55.597 | 34.361 | 1.00 | 13.65 | O |
| ATOM | 1128 | N | ALA | A | 138 | 12.660 | 53.598 | 34.724 | 1.00 | 13.79 | N |
| ATOM | 1129 | CA | ALA | A | 138 | 11.287 | 54.111 | 34.538 | 1.00 | 13.40 | C |
| ATOM | 1130 | CB | ALA | A | 138 | 10.256 | 52.966 | 34.705 | 1.00 | 12.19 | C |
| ATOM | 1131 | C | ALA | A | 138 | 11.048 | 55.265 | 35.564 | 1.00 | 14.42 | C |
| ATOM | 1132 | O | ALA | A | 138 | 10.663 | 56.419 | 35.209 | 1.00 | 14.09 | O |
| ATOM | 1133 | N | LEU | A | 139 | 11.329 | 54.972 | 36.833 | 1.00 | 15.58 | N |
| ATOM | 1134 | CA | LEU | A | 139 | 11.163 | 55.955 | 37.911 | 1.00 | 16.41 | C |
| ATOM | 1135 | CB | LEU | A | 139 | 11.494 | 55.301 | 39.237 | 1.00 | 17.46 | C |
| ATOM | 1136 | CG | LEU | A | 139 | 10.332 | 54.701 | 40.025 | 1.00 | 20.93 | C |
| ATOM | 1137 | CD1 | LEU | A | 139 | 10.774 | 53.393 | 40.715 | 1.00 | 19.31 | C |
| ATOM | 1138 | CD2 | LEU | A | 139 | 9.858 | 55.847 | 41.000 | 1.00 | 24.20 | C |
| ATOM | 1139 | C | LEU | A | 139 | 12.007 | 57.215 | 37.782 | 1.00 | 16.65 | C |
| ATOM | 1140 | O | LEU | A | 139 | 11.516 | 58.304 | 38.011 | 1.00 | 16.20 | O |
| ATOM | 1141 | N | LEU | A | 140 | 13.298 | 57.083 | 37.466 | 1.00 | 17.70 | N |
| ATOM | 1142 | CA | LEU | A | 140 | 14.119 | 58.296 | 37.257 | 1.00 | 17.48 | C |
| ATOM | 1143 | CB | LEU | A | 140 | 15.571 | 57.980 | 36.905 | 1.00 | 17.06 | C |
| ATOM | 1144 | CG | LEU | A | 140 | 16.413 | 57.309 | 38.015 | 1.00 | 18.21 | C |
| ATOM | 1145 | CD1 | LEU | A | 140 | 17.753 | 56.754 | 37.439 | 1.00 | 18.45 | C |
| ATOM | 1146 | CD2 | LEU | A | 140 | 16.610 | 58.209 | 39.236 | 1.00 | 15.29 | C |
| ATOM | 1147 | C | LEU | A | 140 | 13.532 | 59.196 | 36.165 | 1.00 | 18.39 | C |
| ATOM | 1148 | O | LEU | A | 140 | 13.619 | 60.408 | 36.287 | 1.00 | 18.39 | O |
| ATOM | 1149 | N | GLY | A | 141 | 12.962 | 58.573 | 35.123 | 1.00 | 19.17 | N |
| ATOM | 1150 | CA | GLY | A | 141 | 12.262 | 59.211 | 34.040 | 1.00 | 20.51 | C |
| ATOM | 1151 | C | GLY | A | 141 | 11.116 | 60.082 | 34.528 | 1.00 | 21.91 | C |
| ATOM | 1152 | O | GLY | A | 141 | 10.976 | 61.202 | 34.066 | 1.00 | 22.14 | O |
| ATOM | 1153 | N | ARG | A | 142 | 10.313 | 59.579 | 35.468 | 1.00 | 23.33 | N |
| ATOM | 1154 | CA | ARG | A | 142 | 9.240 | 60.350 | 36.091 | 1.00 | 25.41 | C |
| ATOM | 1155 | CB | ARG | A | 142 | 8.284 | 59.477 | 36.901 | 1.00 | 24.99 | C |
| ATOM | 1156 | CG | ARG | A | 142 | 7.928 | 58.177 | 36.272 | 1.00 | 30.09 | C |
| ATOM | 1157 | CD | ARG | A | 142 | 6.477 | 58.109 | 35.945 | 1.00 | 38.07 | C |
| ATOM | 1158 | NE | ARG | A | 142 | 6.024 | 59.304 | 35.245 | 1.00 | 44.22 | N |
| ATOM | 1159 | CZ | ARG | A | 142 | 6.452 | 59.669 | 34.041 | 1.00 | 47.61 | C |
| ATOM | 1160 | NH1 | ARG | A | 142 | 5.970 | 60.777 | 33.474 | 1.00 | 48.87 | N |
| ATOM | 1161 | NH2 | ARG | A | 142 | 7.364 | 58.928 | 33.411 | 1.00 | 47.82 | N |
| ATOM | 1162 | C | ARG | A | 142 | 9.754 | 61.399 | 37.049 | 1.00 | 26.59 | C |
| ATOM | 1163 | O | ARG | A | 142 | 9.043 | 62.348 | 37.292 | 1.00 | 26.95 | O |
| ATOM | 1164 | N | LEU | A | 143 | 10.960 | 61.245 | 37.602 | 1.00 | 27.81 | N |
| ATOM | 1165 | CA | ALEU | A | 143 | 11.450 | 62.097 | 38.712 | 0.50 | 28.31 | C |
| ATOM | 1166 | CA | BLEU | A | 143 | 11.319 | 62.085 | 38.717 | 0.50 | 28.43 | C |
| ATOM | 1167 | CB | ALEU | A | 143 | 12.899 | 61.769 | 39.167 | 0.50 | 28.08 | C |
| ATOM | 1168 | CB | BLEU | A | 143 | 12.548 | 61.549 | 39.462 | 0.50 | 27.84 | C |
| ATOM | 1169 | CG | ALEU | A | 143 | 13.450 | 62.537 | 40.406 | 0.50 | 27.82 | C |
| ATOM | 1170 | CG | BLEU | A | 143 | 12.219 | 60.664 | 40.682 | 0.50 | 26.49 | C |
| ATOM | 1171 | CD1 | ALEU | A | 143 | 13.158 | 61.781 | 41.696 | 0.50 | 27.48 | C |
| ATOM | 1172 | CD1 | BLEU | A | 143 | 12.798 | 61.289 | 41.938 | 0.50 | 26.64 | C |
| ATOM | 1173 | CD2 | ALEU | A | 143 | 14.929 | 62.898 | 40.358 | 0.50 | 26.74 | C |
| ATOM | 1174 | CD2 | BLEU | A | 143 | 10.719 | 60.404 | 40.870 | 0.50 | 24.63 | C |
| ATOM | 1175 | C | LEU | A | 143 | 11.353 | 63.574 | 38.394 | 1.00 | 29.56 | C |
| ATOM | 1176 | O | LEU | A | 143 | 11.038 | 64.372 | 39.274 | 1.00 | 29.09 | O |
| ATOM | 1177 | N | ARG | A | 144 | 11.652 | 63.945 | 37.143 | 1.00 | 32.10 | N |
| ATOM | 1178 | CA | ARG | A | 144 | 11.623 | 65.397 | 36.701 | 1.00 | 35.18 | C |
| ATOM | 1179 | CB | ARG | A | 144 | 11.710 | 65.523 | 35.170 | 1.00 | 35.69 | C |
| ATOM | 1180 | CG | ARG | A | 144 | 10.819 | 64.496 | 34.440 | 1.00 | 36.66 | C |
| ATOM | 1181 | CD | ARG | A | 144 | 9.426 | 65.058 | 34.107 | 1.00 | 38.59 | C |
| ATOM | 1182 | NE | ARG | A | 144 | 8.348 | 64.080 | 34.345 | 1.00 | 38.54 | N |
| ATOM | 1183 | CZ | ARG | A | 144 | 7.336 | 64.243 | 35.208 | 1.00 | 38.75 | C |
| ATOM | 1184 | NH1 | ARG | A | 144 | 7.220 | 65.351 | 35.923 | 1.00 | 38.78 | N |
| ATOM | 1185 | NH2 | ARG | A | 144 | 6.417 | 63.300 | 35.361 | 1.00 | 39.05 | N |
| ATOM | 1186 | C | ARG | A | 144 | 10.392 | 66.179 | 37.226 | 1.00 | 36.66 | C |
| ATOM | 1187 | O | ARG | A | 144 | 9.369 | 66.281 | 36.541 | 1.00 | 37.30 | O |
| ATOM | 1188 | N | THR | A | 145 | 10.553 | 66.781 | 38.414 | 1.00 | 37.68 | N |
| ATOM | 1189 | CA | THR | A | 145 | 9.473 | 67.132 | 39.352 | 1.00 | 37.91 | C |
| ATOM | 1190 | CB | THR | A | 145 | 9.161 | 68.652 | 39.248 | 1.00 | 38.56 | C |
| ATOM | 1191 | OG1 | THR | A | 145 | 10.419 | 69.339 | 39.132 | 1.00 | 40.06 | O |
| ATOM | 1192 | CG2 | THR | A | 145 | 8.349 | 69.231 | 40.487 | 1.00 | 39.02 | C |
| ATOM | 1193 | C | THR | A | 145 | 8.280 | 66.121 | 39.314 | 1.00 | 37.66 | C |
| ATOM | 1194 | O | THR | A | 145 | 8.162 | 65.179 | 40.106 | 1.00 | 36.34 | O |
| ATOM | 1195 | OXT | THR | A | 145 | 7.397 | 66.142 | 38.473 | 1.00 | 37.90 | O |
| ATOM | 1196 | NI | NI | B | 1 | 49.619 | 35.321 | 35.324 | 0.33 | 43.64 | NI |
| ATOM | 1197 | NI | NI | B | 2 | 36.032 | 36.034 | 36.032 | 0.33 | 34.18 | NI |
| ATOM | 1198 | O6′ | LMT | C | 2 | −2.641 | 14.467 | 21.544 | 1.00 | 55.59 | O |
| ATOM | 1199 | C6′ | LMT | C | 2 | −3.020 | 13.503 | 22.520 | 1.00 | 65.46 | C |
| ATOM | 1200 | C5′ | LMT | C | 2 | −1.903 | 12.590 | 23.122 | 1.00 | 68.25 | C |
| ATOM | 1201 | C4′ | LMT | C | 2 | −2.524 | 11.311 | 23.768 | 1.00 | 71.67 | C |
| ATOM | 1202 | C3′ | LMT | C | 2 | −2.897 | 11.304 | 25.278 | 1.00 | 69.12 | C |
| ATOM | 1203 | O3′ | LMT | C | 2 | −4.153 | 10.597 | 25.541 | 1.00 | 65.82 | O |
| ATOM | 1204 | C2′ | LMT | C | 2 | −2.816 | 12.699 | 25.942 | 1.00 | 68.48 | C |
| ATOM | 1205 | O2′ | LMT | C | 2 | −2.892 | 12.577 | 27.385 | 1.00 | 69.28 | O |
| ATOM | 1206 | O1* | LMT | C | 2 | −1.587 | 10.255 | 23.638 | 1.00 | 80.39 | O |
| ATOM | 1207 | C1* | LMT | C | 2 | −2.340 | 9.073 | 23.413 | 1.00 | 88.39 | C |
| ATOM | 1208 | O5* | LMT | C | 2 | −2.310 | 8.780 | 22.004 | 1.00 | 92.28 | O |
| ATOM | 1209 | C5* | LMT | C | 2 | −3.102 | 7.587 | 21.990 | 1.00 | 94.73 | C |
| ATOM | 1210 | C6* | LMT | C | 2 | −4.226 | 7.614 | 20.917 | 1.00 | 96.13 | C |
| ATOM | 1211 | O6* | LMT | C | 2 | −4.478 | 8.959 | 20.439 | 1.00 | 99.22 | O |
| ATOM | 1212 | C4* | LMT | C | 2 | −2.140 | 6.376 | 22.259 | 1.00 | 92.70 | C |
| ATOM | 1213 | O4* | LMT | C | 2 | −1.123 | 6.201 | 21.236 | 1.00 | 89.99 | O |
| ATOM | 1214 | C3* | LMT | C | 2 | −1.518 | 6.588 | 23.678 | 1.00 | 90.17 | C |
| ATOM | 1215 | O3* | LMT | C | 2 | −1.787 | 5.546 | 24.633 | 1.00 | 87.74 | O |
| ATOM | 1216 | C2* | LMT | C | 2 | −1.994 | 7.879 | 24.373 | 1.00 | 90.31 | C |
| ATOM | 1217 | O2* | LMT | C | 2 | −3.100 | 7.571 | 25.287 | 1.00 | 90.99 | O |
| ATOM | 1218 | O5′ | LMT | C | 2 | −1.035 | 13.203 | 24.103 | 1.00 | 64.23 | O |
| ATOM | 1219 | C1′ | LMT | C | 2 | −1.643 | 13.598 | 25.394 | 1.00 | 66.28 | C |
| ATOM | 1220 | O1′ | LMT | C | 2 | −0.652 | 13.915 | 26.382 | 1.00 | 61.22 | O |
| ATOM | 1221 | C1 | LMT | C | 2 | −0.324 | 15.293 | 26.271 | 1.00 | 59.74 | C |
| ATOM | 1222 | C2 | LMT | C | 2 | 0.129 | 15.941 | 27.582 | 1.00 | 58.38 | C |
| ATOM | 1223 | C3 | LMT | C | 2 | 1.652 | 16.029 | 27.567 | 1.00 | 53.65 | C |
| ATOM | 1224 | C4 | LMT | C | 2 | 2.152 | 17.260 | 28.279 | 1.00 | 47.24 | C |
| ATOM | 1225 | C5 | LMT | C | 2 | 3.653 | 17.040 | 28.379 | 1.00 | 45.00 | C |
| ATOM | 1226 | C6 | LMT | C | 2 | 4.473 | 17.301 | 27.120 | 1.00 | 40.50 | C |
| ATOM | 1227 | C7 | LMT | C | 2 | 4.391 | 18.766 | 26.805 | 1.00 | 38.80 | C |
| ATOM | 1228 | C8 | LMT | C | 2 | 5.784 | 19.270 | 26.461 | 1.00 | 39.22 | C |
| ATOM | 1229 | C9 | LMT | C | 2 | 5.984 | 20.624 | 27.142 | 1.00 | 41.72 | C |
| ATOM | 1230 | C10 | LMT | C | 2 | 7.383 | 21.257 | 27.072 | 1.00 | 40.00 | C |
| ATOM | 1231 | C11 | LMT | C | 2 | 7.796 | 21.777 | 28.445 | 1.00 | 42.73 | C |
| ATOM | 1232 | C12 | LMT | C | 2 | 8.884 | 22.860 | 28.345 | 1.00 | 45.65 | C |
| ATOM | 1233 | O31 | GTT | D | 1 | 7.260 | 11.767 | 19.720 | 1.00 | 18.18 | O |
| ATOM | 1234 | C3 | GTT | D | 1 | 6.534 | 10.745 | 19.928 | 1.00 | 23.39 | C |
| ATOM | 1235 | O32 | GTT | D | 1 | 6.794 | 9.681 | 19.307 | 1.00 | 22.21 | O |
| ATOM | 1236 | CA3 | GTT | D | 1 | 5.385 | 10.720 | 20.937 | 1.00 | 21.49 | C |
| ATOM | 1237 | N3 | GTT | D | 1 | 5.108 | 12.040 | 21.461 | 1.00 | 17.33 | N |
| ATOM | 1238 | C2 | GTT | D | 1 | 4.906 | 12.262 | 22.759 | 1.00 | 18.28 | C |
| ATOM | 1239 | O2 | GTT | D | 1 | 5.170 | 11.234 | 23.600 | 1.00 | 19.19 | O |
| ATOM | 1240 | CA2 | GTT | D | 1 | 4.330 | 13.562 | 23.268 | 1.00 | 16.91 | C |
| ATOM | 1241 | CB2 | GTT | D | 1 | 2.906 | 13.185 | 23.759 | 1.00 | 19.79 | C |
| ATOM | 1242 | SG2 | GTT | D | 1 | 1.808 | 12.751 | 22.382 | 1.00 | 19.29 | S |
| ATOM | 1243 | N2 | GTT | D | 1 | 4.231 | 14.575 | 22.163 | 1.00 | 21.49 | N |
| ATOM | 1244 | CD1 | GTT | D | 1 | 5.157 | 15.515 | 21.860 | 1.00 | 15.20 | C |
| ATOM | 1245 | OE1 | GTT | D | 1 | 6.314 | 15.422 | 22.562 | 1.00 | 19.82 | O |
| ATOM | 1246 | CG1 | GTT | D | 1 | 4.983 | 16.500 | 20.708 | 1.00 | 14.16 | C |
| ATOM | 1247 | CB1 | GTT | D | 1 | 6.308 | 16.329 | 20.014 | 1.00 | 15.79 | C |
| ATOM | 1248 | CA1 | GTT | O | 1 | 6.307 | 15.434 | 18.773 | 1.00 | 23.56 | C |
| ATOM | 1249 | N1 | GTT | D | 1 | 7.583 | 15.142 | 18.203 | 1.00 | 17.29 | N |
| ATOM | 1250 | C1 | GTT | D | 1 | 5.393 | 14.226 | 18.497 | 1.00 | 22.82 | C |
| ATOM | 1251 | O11 | GTT | D | 1 | 4.484 | 13.795 | 19.226 | 1.00 | 21.17 | O |
| ATOM | 1252 | O12 | GTT | D | 1 | 5.580 | 13.736 | 17.383 | 1.00 | 19.19 | O |
| ATOM | 1253 | O2 | PLM | E | 1 | 26.974 | 33.632 | 38.899 | 1.00 | 66.56 | O |
| ATOM | 1254 | C1 | PLM | E | 1 | 27.406 | 32.626 | 38.230 | 1.00 | 66.81 | C |
| ATOM | 1255 | O1 | PLM | E | 1 | 27.017 | 31.447 | 38.519 | 1.00 | 64.62 | O |
| ATOM | 1256 | C2 | PLM | E | 1 | 28.419 | 32.836 | 37.100 | 1.00 | 64.42 | C |
| ATOM | 1257 | C3 | PLM | E | 1 | 28.693 | 31.511 | 36.394 | 1.00 | 60.12 | C |
| ATOM | 1258 | C4 | PLM | E | 1 | 29.921 | 31.548 | 35.493 | 1.00 | 59.14 | C |
| ATOM | 1259 | C5 | PLM | E | 1 | 29.759 | 32.417 | 34.250 | 1.00 | 56.91 | C |
| ATOM | 1260 | C6 | PLM | E | 1 | 29.137 | 31.618 | 33.124 | 1.00 | 54.09 | C |
| ATOM | 1261 | C7 | PLM | E | 1 | 28.634 | 32.575 | 32.060 | 1.00 | 51.22 | C |
| ATOM | 1262 | C8 | PLM | E | 1 | 28.276 | 31.808 | 30.785 | 1.00 | 47.04 | C |
| ATOM | 1263 | C9 | PLM | E | 1 | 27.522 | 30.474 | 30.940 | 1.00 | 46.76 | C |
| ATOM | 1264 | C10 | PLM | E | 1 | 27.144 | 29.952 | 29.535 | 1.00 | 44.05 | C |
| ATOM | 1265 | C11 | PLM | E | 1 | 26.288 | 28.712 | 29.447 | 1.00 | 41.17 | C |
| ATOM | 1266 | C12 | PLM | E | 1 | 25.119 | 29.008 | 28.521 | 1.00 | 37.05 | C |
| ATOM | 1267 | C13 | PLM | E | 1 | 24.761 | 27.741 | 27.783 | 1.00 | 33.21 | C |
| ATOM | 1268 | C14 | PLM | E | 1 | 23.369 | 27.762 | 27.137 | 1.00 | 35.38 | C |
| ATOM | 1269 | C15 | PLM | E | 1 | 23.143 | 26.418 | 26.449 | 1.00 | 35.82 | C |
| ATOM | 1270 | C16 | PLM | E | 1 | 22.860 | 26.760 | 25.007 | 1.00 | 36.18 | C |
| ATOM | 1272 | C1 | PLM | E | 2 | 11.914 | 38.040 | 26.633 | 1.00 | 48.53 | C |
| ATOM | 1274 | C2 | PLM | E | 2 | 13.174 | 37.970 | 25.790 | 1.00 | 44.77 | C |
| ATOM | 1275 | C3 | PLM | E | 2 | 13.602 | 39.369 | 25.287 | 1.00 | 44.35 | C |
| ATOM | 1276 | C4 | PLM | E | 2 | 15.114 | 39.474 | 24.969 | 1.00 | 38.32 | C |
| ATOM | 1277 | C5 | PLM | E | 2 | 15.515 | 40.918 | 24.728 | 1.00 | 37.07 | C |
| ATOM | 1278 | C6 | PLM | E | 2 | 16.706 | 41.173 | 23.807 | 1.00 | 35.48 | C |
| ATOM | 1279 | C7 | PLM | E | 2 | 16.774 | 42.671 | 23.526 | 1.00 | 37.05 | C |
| ATOM | 1280 | C8 | PLM | E | 2 | 17.978 | 43.129 | 22.653 | 1.00 | 37.63 | C |
| ATOM | 1281 | C9 | PLM | E | 2 | 18.500 | 44.556 | 22.928 | 1.00 | 39.61 | C |
| ATOM | 1282 | C10 | PLM | E | 2 | 19.561 | 45.130 | 21.927 | 1.00 | 36.56 | C |
| ATOM | 1290 | C1 | PLM | E | 3 | 11.541 | 46.276 | 43.832 | 1.00 | 39.81 | C |
| ATOM | 1292 | C2 | PLM | E | 3 | 12.716 | 46.969 | 43.161 | 1.00 | 39.10 | C |
| ATOM | 1293 | C3 | PLM | E | 3 | 12.315 | 48.228 | 42.438 | 1.00 | 35.98 | C |
| ATOM | 1294 | C4 | PLM | E | 3 | 13.068 | 49.485 | 42.861 | 1.00 | 40.15 | C |
| ATOM | 1295 | C5 | PLM | E | 3 | 14.269 | 49.934 | 42.035 | 1.00 | 36.54 | C |
| ATOM | 1296 | C6 | PLM | E | 3 | 15.487 | 49.927 | 42.964 | 1.00 | 37.54 | C |
| ATOM | 1297 | C7 | PLM | E | 3 | 16.859 | 50.306 | 42.421 | 1.00 | 36.68 | C |
| ATOM | 1298 | C8 | PLM | E | 3 | 17.845 | 49.142 | 42.450 | 1.00 | 41.16 | C |
| ATOM | 1299 | C9 | PLM | E | 3 | 19.322 | 49.524 | 42.505 | 1.00 | 44.23 | C |
| ATOM | 1300 | C10 | PLM | E | 3 | 20.060 | 48.981 | 41.279 | 1.00 | 47.26 | C |
| ATOM | 1308 | C1 | PLM | E | 4 | 11.814 | 37.183 | 31.848 | 1.00 | 45.82 | C |
| ATOM | 1310 | C2 | PLM | E | 4 | 12.943 | 37.762 | 30.970 | 1.00 | 45.75 | C |
| ATOM | 1311 | C3 | PLM | E | 4 | 12.893 | 39.300 | 30.870 | 1.00 | 42.36 | C |
| ATOM | 1312 | C4 | PLM | E | 4 | 13.992 | 39.810 | 29.928 | 1.00 | 39.54 | C |
| ATOM | 1313 | C5 | PLM | E | 4 | 13.894 | 41.291 | 29.589 | 1.00 | 37.76 | C |
| ATOM | 1314 | C6 | PLM | E | 4 | 15.128 | 41.823 | 28.881 | 1.00 | 36.66 | C |
| ATOM | 1315 | C7 | PLM | E | 4 | 15.019 | 43.285 | 28.388 | 1.00 | 37.90 | C |
| ATOM | 1316 | C8 | PLM | E | 4 | 16.317 | 44.077 | 28.668 | 1.00 | 39.17 | C |
| ATOM | 1317 | C9 | PLM | E | 4 | 16.632 | 45.429 | 27.993 | 1.00 | 41.51 | C |
| ATOM | 1318 | C10 | PLM | E | 4 | 17.991 | 46.027 | 28.467 | 1.00 | 39.52 | C |
| ATOM | 1326 | C1 | PLM | E | 5 | 11.321 | 43.101 | 32.669 | 1.00 | 48.99 | C |
| ATOM | 1328 | C2 | PLM | E | 5 | 12.190 | 44.150 | 32.016 | 1.00 | 48.28 | C |
| ATOM | 1329 | C3 | PLM | E | 5 | 11.457 | 44.966 | 30.951 | 1.00 | 50.96 | C |
| ATOM | 1330 | C4 | PLM | E | 5 | 12.490 | 45.898 | 30.276 | 1.00 | 54.37 | C |
| ATOM | 1331 | C5 | PLM | E | 5 | 11.911 | 47.042 | 29.423 | 1.00 | 55.82 | C |
| ATOM | 1344 | C1 | PLM | E | 6 | 18.081 | −0.222 | 32.874 | 1.00 | 49.57 | C |
| ATOM | 1346 | C2 | PLM | E | 6 | 19.428 | 0.454 | 33.122 | 1.00 | 49.65 | C |
| ATOM | 1347 | C3 | PLM | E | 6 | 19.538 | 1.691 | 32.234 | 1.00 | 44.94 | C |
| ATOM | 1348 | C4 | PLM | E | 6 | 20.973 | 2.059 | 31.885 | 1.00 | 41.84 | C |
| ATOM | 1349 | C5 | PLM | E | 6 | 20.991 | 3.576 | 31.764 | 1.00 | 38.05 | C |
| ATOM | 1350 | C6 | PLM | E | 6 | 22.353 | 4.225 | 31.633 | 1.00 | 41.48 | C |
| ATOM | 1351 | C7 | PLM | E | 6 | 22.231 | 5.718 | 31.962 | 1.00 | 42.77 | C |
| ATOM | 1352 | C8 | PLM | E | 6 | 23.564 | 6.373 | 32.261 | 1.00 | 42.18 | C |
| ATOM | 1361 | S | SO4 | F | 1 | −0.212 | 0.680 | 23.119 | 0.50 | 38.20 | S |
| ATOM | 1362 | O1 | SO4 | F | 1 | −0.012 | −0.447 | 24.080 | 0.50 | 37.58 | O |
| ATOM | 1363 | O2 | SO4 | F | 1 | 1.038 | 0.841 | 22.328 | 0.50 | 32.09 | O |
| ATOM | 1364 | O3 | SO4 | F | 1 | −1.316 | 0.327 | 22.206 | 0.50 | 35.26 | O |
| ATOM | 1365 | O4 | SO4 | F | 1 | −0.573 | 1.819 | 23.985 | 0.50 | 30.18 | O |
| ATOM | 1366 | O | HOH | G | 1 | −0.943 | 7.401 | 32.542 | 1.00 | 37.80 | O |
| ATOM | 1367 | O | HOH | G | 2 | 23.768 | −2.017 | 23.464 | 1.00 | 50.16 | O |
| ATOM | 1368 | O | HOH | G | 3 | 24.405 | 25.638 | 44.557 | 1.00 | 30.72 | O |
| ATOM | 1369 | O | HOH | G | 4 | 8.539 | 15.180 | 38.179 | 1.00 | 35.64 | O |
| ATOM | 1370 | O | HOH | G | 5 | 12.649 | −4.140 | 6.571 | 1.00 | 32.67 | O |
| ATOM | 1371 | O | HOH | G | 6 | 49.199 | 31.327 | 29.456 | 1.00 | 42.68 | O |
| ATOM | 1372 | O | HOH | G | 7 | 19.364 | −3.454 | 3.849 | 1.00 | 35.88 | O |
| ATOM | 1373 | O | HOH | G | 8 | 22.454 | 22.448 | 22.453 | 0.33 | 27.52 | O |
| ATOM | 1374 | O | HOH | G | 9 | 24.180 | 28.825 | 42.845 | 1.00 | 31.64 | O |
| ATOM | 1375 | O | HOH | G | 10 | 15.726 | 20.889 | 19.798 | 1.00 | 28.20 | O |
| ATOM | 1376 | O | HOH | G | 11 | 4.869 | 3.908 | 32.695 | 1.00 | 23.56 | O |
| ATOM | 1377 | O | HOH | G | 12 | 12.549 | 20.952 | 20.270 | 1.00 | 26.41 | O |
| ATOM | 1378 | O | HOH | G | 13 | −0.641 | 2.101 | 32.565 | 1.00 | 30.52 | O |
| ATOM | 1379 | O | HOH | G | 14 | 1.356 | 4.200 | 33.433 | 1.00 | 41.58 | O |
| ATOM | 1380 | O | HOH | G | 15 | 23.516 | −1.627 | 20.788 | 1.00 | 27.46 | O |
| ATOM | 1381 | O | HOH | G | 16 | 12.705 | 51.071 | 31.777 | 1.00 | 39.06 | O |
| ATOM | 1382 | O | HOH | G | 17 | 19.696 | 45.528 | 32.849 | 1.00 | 46.56 | O |
| ATOM | 1383 | O | HOH | G | 18 | 20.332 | −4.259 | 11.486 | 1.00 | 56.74 | O |
| ATOM | 1384 | O | HOH | G | 19 | 6.083 | −4.312 | 9.581 | 1.00 | 52.29 | O |
| ATOM | 1385 | O | HOH | G | 20 | 10.605 | −5.685 | 10.001 | 1.00 | 45.47 | O |
| ATOM | 1386 | O | HOH | G | 21 | 9.714 | 16.613 | 28.435 | 1.00 | 30.94 | O |
| ATOM | 1387 | O | HOH | G | 22 | 29.550 | 23.798 | 41.176 | 1.00 | 24.02 | O |
| ATOM | 1388 | O | HOH | G | 23 | 19.770 | −7.178 | 19.171 | 1.00 | 43.78 | O |
| ATOM | 1389 | O | HOH | G | 24 | −0.298 | 0.404 | 28.375 | 0.50 | 17.68 | O |
| ATOM | 1390 | O | HOH | G | 25 | 19.502 | 36.373 | 38.692 | 1.00 | 28.51 | O |
| ATOM | 1391 | O | HOH | G | 26 | 18.464 | 10.008 | 38.963 | 1.00 | 47.22 | O |
| ATOM | 1392 | O | HOH | G | 27 | 17.027 | 13.019 | 27.868 | 1.00 | 23.53 | O |
| ATOM | 1393 | O | HOH | G | 28 | 16.918 | −11.145 | 14.469 | 1.00 | 45.82 | O |
| ATOM | 1394 | O | HOH | G | 29 | 3.177 | 11.600 | 36.108 | 1.00 | 30.70 | O |
| ATOM | 1395 | O | HOH | G | 30 | 16.303 | −2.200 | 22.254 | 1.00 | 35.62 | O |
| ATOM | 1396 | O | HOH | G | 31 | 3.550 | 1.641 | 14.248 | 1.00 | 38.31 | O |
| ATOM | 1397 | O | HOH | G | 32 | 8.799 | 3.171 | 32.766 | 1.00 | 31.96 | O |
| ATOM | 1398 | O | HOH | G | 33 | 6.518 | 5.654 | 13.528 | 1.00 | 25.16 | O |
| ATOM | 1399 | O | HOH | G | 34 | −0.568 | −2.259 | 15.350 | 1.00 | 38.76 | O |
| ATOM | 1400 | O | HOH | G | 35 | 11.300 | −1.161 | 28.084 | 1.00 | 30.49 | O |
| ATOM | 1401 | O | HOH | G | 36 | 18.621 | 45.148 | 37.681 | 1.00 | 34.44 | O |
| ATOM | 1402 | O | HOH | G | 37 | 8.931 | 6.171 | 12.377 | 1.00 | 37.11 | O |
| ATOM | 1403 | O | HOH | G | 38 | 19.279 | −0.636 | 3.034 | 1.00 | 40.60 | O |
| ATOM | 1404 | O | HOH | G | 39 | 7.065 | 7.091 | 7.076 | 0.33 | 38.56 | O |
| ATOM | 1405 | O | HOH | G | 40 | 5.120 | 1.278 | 9.497 | 1.00 | 47.24 | O |
| ATOM | 1406 | O | HOH | G | 41 | 3.122 | −2.767 | 34.303 | 1.00 | 55.08 | O |
| ATOM | 1407 | O | HOH | G | 42 | 16.405 | 0.431 | 35.012 | 1.00 | 60.69 | O |
| ATOM | 1408 | O | HOH | G | 43 | 9.341 | 56.829 | 32.953 | 1.00 | 47.19 | O |
| ATOM | 1409 | O | HOH | G | 44 | 13.749 | 2.875 | 35.782 | 1.00 | 46.75 | O |
| ATOM | 1410 | O | HOH | G | 45 | 21.044 | −4.632 | 24.353 | 1.00 | 48.95 | O |
| ATOM | 1411 | O | HOH | G | 46 | 17.960 | 17.972 | 18.023 | 0.33 | 21.66 | O |
| ATOM | 1412 | O | HOH | G | 47 | 23.465 | −4.351 | 19.223 | 1.00 | 46.63 | O |
| ATOM | 1413 | O | HOH | G | 48 | 22.105 | −6.228 | 19.910 | 1.00 | 40.95 | O |
| ATOM | 1414 | O | HOH | G | 49 | 13.571 | −13.572 | 13.572 | 0.33 | 39.27 | O |
| ATOM | 1415 | O | HOH | G | 50 | 7.079 | 52.775 | 47.715 | 1.00 | 43.72 | O |
| ATOM | 1416 | O | HOH | G | 51 | 9.009 | 49.996 | 33.051 | 1.00 | 51.94 | O |
| ATOM | 1417 | O | HOH | G | 52 | 10.262 | 41.231 | 27.895 | 1.00 | 49.00 | O |
| ATOM | 1418 | O | HOH | G | 53 | 13.720 | 13.669 | 12.813 | 0.33 | 18.06 | O |
| ATOM | 1419 | O | HOH | G | 54 | 16.527 | 9.719 | 14.214 | 1.00 | 41.63 | O |
| ATOM | 1420 | O | HOH | G | 55 | 19.917 | −2.461 | 29.008 | 1.00 | 41.31 | O |
| ATOM | 1421 | O | HOH | G | 56 | 26.002 | 4.113 | 27.927 | 1.00 | 43.02 | O |
| ATOM | 1422 | O | HOH | G | 57 | 42.530 | 37.631 | 35.739 | 1.00 | 44.03 | O |
| ATOM | 1423 | O | HOH | G | 58 | 0.558 | 23.097 | 29.764 | 1.00 | 38.42 | O |
| ATOM | 1424 | O | HOH | G | 59 | −0.084 | 20.766 | 28.679 | 1.00 | 42.63 | O |
| ATOM | 1425 | O | HOH | G | 60 | 7.231 | 31.657 | 32.146 | 1.00 | 49.34 | O |
| ATOM | 1426 | O | HOH | G | 61 | 6.100 | 28.757 | 33.751 | 1.00 | 48.45 | O |
| ATOM | 1427 | O | HOH | G | 62 | 22.704 | 33.262 | 41.119 | 1.00 | 43.75 | O |
| ATOM | 1428 | O | HOH | G | 63 | 23.542 | 36.678 | 45.283 | 1.00 | 56.29 | O |
| ATOM | 1429 | O | HOH | G | 64 | 13.013 | 15.648 | 16.066 | 1.00 | 33.59 | O |
| ATOM | 1430 | O | HOH | G | 65 | 11.923 | 11.539 | 37.896 | 1.00 | 32.51 | O |
| ATOM | 1431 | O | HOH | G | 66 | 23.799 | −0.422 | 30.808 | 1.00 | 42.75 | O |
| ATOM | 1432 | O | HOH | G | 67 | 19.848 | 8.641 | 34.946 | 1.00 | 45.99 | O |
| ATOM | 1433 | O | HOH | G | 68 | 6.284 | 8.088 | 36.246 | 1.00 | 32.45 | O |
| ATOM | 1434 | O | HOH | G | 69 | 16.320 | 62.608 | 37.937 | 1.00 | 54.04 | O |
| ATOM | 1435 | O | HOH | G | 70 | 17.347 | 60.408 | 34.261 | 1.00 | 51.34 | O |
| ATOM | 1436 | O | HOH | G | 71 | 23.686 | 61.097 | 38.482 | 1.00 | 47.83 | O |
| ATOM | 1437 | O | HOH | G | 72 | 1.261 | 5.929 | 31.480 | 1.00 | 26.59 | O |
| ATOM | 1438 | O | HOH | G | 73 | 15.539 | 14.906 | 26.775 | 1.00 | 15.99 | O |
| ATOM | 1439 | O | HOH | G | 74 | 14.984 | 14.225 | 17.521 | 1.00 | 25.28 | O |
| ATOM | 1440 | O | HOH | G | 75 | 10.752 | 10.753 | 10.752 | 0.33 | 44.69 | O |
| ATOM | 1441 | O | HOH | G | 76 | 7.951 | 0.926 | 3.374 | 1.00 | 52.97 | O |
| ATOM | 1442 | O | HOH | G | 77 | 5.353 | 2.129 | 6.301 | 1.00 | 46.99 | O |
| ATOM | 1443 | O | HOH | G | 78 | 3.761 | 3.646 | 2.822 | 0.33 | 23.22 | O |
| ATOM | 1444 | O | HOH | G | 79 | 5.736 | 2.198 | 2.348 | 1.00 | 56.67 | O |
| ATOM | 1445 | O | HOH | G | 80 | 16.996 | 15.731 | 17.621 | 1.00 | 32.02 | O |
| ATOM | 1446 | O | HOH | G | 81 | 9.509 | 9.336 | 12.446 | 1.00 | 27.31 | O |
| ATOM | 1447 | O | HOH | G | 82 | 7.285 | 63.628 | 41.255 | 1.00 | 50.12 | O |
| ATOM | 1448 | O | HOH | G | 83 | 11.668 | 71.674 | 37.394 | 1.00 | 58.95 | O |
| ATOM | 1449 | O | HOH | G | 84 | 52.502 | 27.817 | 39.209 | 1.00 | 54.21 | O |
| ATOM | 1450 | O | HOH | G | 85 | 22.730 | 36.647 | 42.784 | 1.00 | 52.19 | O |
| ATOM | 1451 | O | HOH | G | 86 | 17.005 | 45.986 | 32.923 | 1.00 | 30.94 | O |
| ATOM | 1452 | O | HOH | G | 87 | 9.499 | 47.980 | 33.772 | 1.00 | 40.66 | O |
| ATOM | 1453 | O | HOH | G | 88 | 5.052 | 56.415 | 38.430 | 1.00 | 55.24 | O |
| ATOM | 1454 | O | HOH | G | 89 | 22.807 | −1.144 | 25.460 | 1.00 | 42.51 | O |
| ATOM | 1455 | O | HOH | G | 90 | 25.234 | −1.919 | 25.883 | 1.00 | 52.43 | O |
| ATOM | 1456 | O | HOH | G | 91 | 21.549 | 10.612 | 19.510 | 1.00 | 36.05 | O |
| ATOM | 1457 | O | HOH | G | 92 | 8.615 | −0.822 | 6.948 | 1.00 | 49.68 | O |
| ATOM | 1458 | O | HOH | G | 93 | 11.375 | −2.265 | 2.608 | 1.00 | 28.01 | O |
| ATOM | 1459 | O | HOH | G | 94 | 10.563 | −2.134 | 8.036 | 1.00 | 27.90 | O |
| ATOM | 1460 | O | HOH | G | 95 | 3.858 | −0.505 | 12.082 | 1.00 | 37.25 | O |
| ATOM | 1461 | O | HOH | G | 96 | 17.845 | 62.184 | 36.568 | 1.00 | 60.67 | O |
| ATOM | 1462 | O | HOH | G | 97 | 19.883 | 63.500 | 38.550 | 1.00 | 51.50 | O |
| ATOM | 1463 | O | HOH | G | 98 | −3.992 | 12.182 | 33.345 | 1.00 | 47.54 | O |
| ATOM | 1464 | O | HOH | G | 99 | −2.625 | 14.172 | 34.289 | 1.00 | 57.66 | O |
| ATOM | 1465 | O | HOH | H | 1 | −1.410 | 16.222 | 32.352 | 1.00 | 51.79 | O |
| ATOM | 1466 | O | HOH | H | 2 | −2.035 | 4.920 | 4.213 | 1.00 | 56.43 | O |
| ATOM | 1467 | O | HOH | H | 3 | 1.762 | 2.181 | 3.138 | 0.33 | 9.55 | O |
| ATOM | 1468 | O | HOH | H | 4 | 4.071 | −1.142 | 2.376 | 1.00 | 70.24 | O |
| ATOM | 1469 | O | HOH | H | 5 | 2.368 | 0.085 | 1.287 | 1.00 | 56.00 | O |
| ATOM | 1470 | O | HOH | H | 6 | 3.459 | −6.491 | 15.191 | 1.00 | 43.50 | O |
| ATOM | 1471 | O | HOH | H | 7 | 2.611 | −3.264 | 23.603 | 1.00 | 27.95 | O |
| ATOM | 1472 | O | HOH | H | 8 | 2.170 | 0.591 | 19.746 | 1.00 | 52.43 | O |
| ATOM | 1473 | O | HOH | H | 9 | 2.170 | 0.591 | 19.746 | 1.00 | 52.43 | O |
| ATOM | 1474 | O | HOH | H | 10 | 22.207 | 28.317 | 48.743 | 1.00 | 45.79 | O |
| ATOM | 1475 | O | HOH | H | 11 | 14.592 | −1.036 | 24.606 | 1.00 | 38.07 | O |
| ATOM | 1476 | O | HOH | H | 12 | 4.462 | −4.943 | 21.408 | 1.00 | 41.79 | O |
| ATOM | 1477 | O | HOH | H | 13 | 15.675 | 56.885 | 33.119 | 1.00 | 46.32 | O |
| ATOM | 1478 | O | HOH | H | 14 | 18.469 | 51.763 | 36.153 | 1.00 | 38.33 | O |
| ATOM | 1479 | O | HOH | H | 15 | 12.448 | −6.521 | 8.382 | 1.00 | 45.62 | O |
| ATOM | 1480 | O | HOH | H | 16 | 12.130 | 65.814 | 42.971 | 1.00 | 38.19 | O |
| ATOM | 1481 | O | HOH | H | 17 | 25.958 | 29.229 | 39.247 | 1.00 | 44.50 | O |
| ATOM | 1482 | O | HOH | H | 18 | 26.611 | −2.745 | 16.970 | 1.00 | 51.78 | O |
| ATOM | 1483 | O | HOH | H | 19 | 9.680 | −2.081 | 21.897 | 1.00 | 38.19 | O |
| ATOM | 1484 | O | HOH | H | 20 | 5.879 | −4.835 | 25.822 | 1.00 | 40.98 | O |
| ATOM | 1485 | O | HOH | H | 21 | 22.458 | 42.679 | 31.767 | 1.00 | 40.48 | O |
| ATOM | 1486 | O | HOH | H | 22 | 12.241 | −9.427 | 8.755 | 1.00 | 54.06 | O |
| ATOM | 1487 | O | HOH | H | 23 | 5.205 | −5.013 | 17.361 | 1.00 | 35.53 | O |
| ATOM | 1488 | O | HOH | H | 24 | 16.473 | −0.862 | 4.489 | 1.00 | 41.63 | O |
| ATOM | 1489 | O | HOH | H | 25 | 7.860 | 61.368 | 30.803 | 1.00 | 53.11 | O |
| ATOM | 1490 | O | HOH | H | 26 | 13.077 | 63.130 | 35.975 | 1.00 | 48.46 | O |
| ATOM | 1491 | O | HOH | H | 27 | 42.305 | 36.490 | 37.769 | 1.00 | 45.84 | O |
| END | | | | | | | | | | | |

1-44. (canceled)

45. A method for selecting or designing a compound expected to modulate the activity of Leukotriene C4 synthase (LTC4S), the method comprising the step of using molecular modelling means to select or design a compound that is predicted to interact with the catalytic site or a substrate binding region of LTC4S, wherein a three-dimensional structure of at least a part of the catalytic site or a substrate binding region of LTC4S is compared with a three-dimensional structure of a compound, and a compound that is predicted to interact with the said catalytic site or substrate binding region is selected.

46. The method of claim 45 wherein the three-dimensional structure of at least a part of the active site or a substrate binding region of LTC4S is a three-dimensional structure (or part thereof) determined for an LTC4S polypeptide comprising an N-terminal hexahistidine tag.

47. The method of claim 45 wherein the three-dimensional structure of at least a part of the active site or a substrate binding region of LTC4S is a three-dimensional structure (or part thereof) determined for a human LTC4S polypeptide.

48. The method of claim 45 wherein the three-dimensional structure of at least a part of the active site or a substrate binding region of LTC4S is a three-dimensional structure (or part thereof) obtainable by X-ray analysis of a crystal obtainable using a mother liquor solution comprising a detergent; and optionally, the mother liquor solution comprises glutathione (GSH).

49. The method of claim 48 wherein the detergent is dodecyl maltoside (DDM).

50. The method of claim 48 wherein the three-dimensional structure of at least a part of the active site or a substrate binding region of LTC4S is that represented by the structure co-ordinates shown in Table I (or a part thereof) when the mother liquor solution does not comprise GSH, or Table II (or a part thereof) when the mother liquor solution comprises GSH; or a structure modelled on such structure co-ordinates ± the root mean square deviation from the backbone atoms of the protein of less than 2.0 Å, 1.5, Å 1.0 Å or 0.5 Å.

51. The method of claim 45 wherein the selected compound is predicted to block or bind to at least a part of a region of the structure termed the “GSH substrate binding cavity” (formed by residues including residues Arg51, Arg30, Arg104, Gln53, Asn55, Glu58, Tyr59, Tyr93, Tyr97, Ile27, Pro37, Leu108 of full length human LTC4S, or equivalent residues); the “lipophilic substrate binding crevice” (formed by residues including Ala20, Leu24, Ile27, Tyr59, Trp116, Ala112, Leu115, Leu108, Tyr109, Leu62, Val119, Thr66, Val16 and Leu17, or equivalent residues); or the “catalytic site” (formed by residues including Arg104 or Arg31, or equivalent residues).

52. The method of claim 45 comprising the step of assessing whether the compound modulates the activity of LTC4S, and selecting a compound that modulates the activity.

53. The method of claim 45 comprising the step of assessing whether the compound modulates LTC4 signalling in a whole cell, tissue or organism, and a compound that modulates the activity is selected; and, optionally further comprising the step of assessing whether the compound modulates the activity of LTC4S in the whole cell, tissue or organism, and a compound that modulates the activity is selected.

54. A three-dimensional crystalline form of LTC4S as defined in claim 45 belonging to space group F23 and/or has a unit cell containing 48 LTC4S chains and/or comprises multiple adjacent histidine tags coordinated by metal ions (for example three for each LTC4S trimer or twelve for each unit cell).

55. The three-dimensional crystalline form of claim 54 wherein the crystalline form further comprises a co-crystallised molecule; and, optionally, the co-crystallised molecule modulates LTC4S activity.

56. The three-dimensional crystalline form of claim 55 wherein the co-crystallised molecule is GSH or a detergent such as DDM.

57. Use of LTC4S as defined in claim 45 in generating a structure of the active site or a substrate binding region (or part thereof) of LTC4S; or a structure of the active site or a substrate binding region (or part thereof) of LTC4S bound to a test compound.

58. A method of predicting a three dimensional structure of a target LTC4S protein or other MAPEG family member protein or homo- or heteromultimer thereof (the target protein), the method comprises the steps of: aligning a representation of an amino acid sequence of the target protein with the amino acid sequence of the LTC4S of Table I or II, optionally varied by a root mean square deviation of not more than 2.0 Å, 1.5 Å, 1.0 Å or 0.5 Å, or selected coordinates thereof, to match homologous regions of the amino acid sequences; modelling the structure of the matched homologous regions of said target protein on the corresponding regions of the LTC4S structure as defined by Table I or II, optionally varied by a root mean square deviation of not more than 2.0 Å, 1.5 Å, 1.0 Å or 0.5 Å, or selected coordinates thereof; and determining a conformation for said target protein which substantially preserves the structure of said matched homologous regions.

59. A method for selecting or designing a compound expected to modulate the activity of a MAPEG family member protein or homo- or heteromultimer thereof, the method comprising the step of using molecular modelling means to select or design a compound that is predicted to interact with the catalytic site or a substrate binding region of the MAPEG family member protein or homo- or heteromultimer thereof, wherein a three-dimensional structure of at least a part of the catalytic site or a substrate binding region of the MAPEG family member protein or homo- or heteromultimer thereof is compared with a three-dimensional structure of a compound, and a compound that is predicted to interact with the said catalytic site or substrate binding region is selected, wherein the three-dimensional structure of at least a part of the catalytic site or a substrate binding region of the MAPEG protein or homo- or heteromultimer thereof is a three-dimensional structure (or part thereof) predicted by a method according to claim 58.

60. The method of claim 45 wherein the molecular structure to be fitted is in the form of a model of a pharmacophore.

61. A computer-based method of rational drug design comprising: (a) providing the coordinates of a LTC4S structure as defined in Table I or II optionally varied by a root mean square deviation of backbone atoms of the protein of less than 2.0 Å, 1.5 Å, 1.0 Å or 0.5 Å, or selected coordinates thereof; (b) providing the structures of a plurality of molecular fragments; (c) fitting the structure of each of the molecular fragments to the selected coordinates; and (d) assembling the molecular fragments into a single molecule to form a candidate modulator molecule; and, optionally further comprising the step of: (e) obtaining or synthesising the molecular fragment or modulator molecule; and (f) contacting the molecular fragment or modulator molecule with LTC4S to determine the ability of the molecular fragment or modulator molecule to interact with LTC4S.

62. A method of obtaining a representation of the three dimensional structure of LTC4S, which method comprises providing the data of Table I or II optionally varied by a root mean square deviation of backbone atoms of the protein of less than 2.0 Å, 1.5 Å, 1.0 Å or 0.5 Å, or selected coordinates thereof, and constructing a three-dimensional structure representing said coordinates; and, optionally presenting the structure as a (a) a wire-frame model; (b) a chicken-wire model; (c) a ball-and-stick model; (d) a space-filling model; (e) a stick-model; (f) a ribbon model; (g) a snake model; (h) an arrow and cylinder model; (i) an electron density map; (j) a molecular surface model.

63. A computer system, intended to generate structures and/or perform optimisation of compounds which interact with LTC4S or other MAPEG family member protein or homo- or heteromultimer thereof, complexes of LTC4S or other MAPEG family member protein or homo- or heteromultimer thereof with compounds, the system containing computer-readable data comprising one or more of: (a) LTC4S co-ordinate data of Tables I or II optionally varied by a root mean square deviation of backbone atoms of the protein of less than 22.0 Å, 1.5 Å, 1.0 Å or 0.5 Å, or selected coordinates thereof, said data defining the three-dimensional structure of LTC4S or said selected coordinates thereof; (b) atomic coordinate data of a target MAPEG family member protein or homo- or heteromultimer thereof generated by homology modelling of the target based on the coordinate data of Table I or II optionally varied by a root mean square deviation of backbone atoms of the protein of less than 2.0 Å, 1.5 Å, 1.0 Å or 0.5 Å, or selected coordinates thereof; (c) atomic coordinate data of a target MAPEG family member protein or homo- or heteromultimer thereof generated by interpreting X-ray crystallographic data or NMR data by reference to the coordinate data of Tables I or II optionally varied by a root mean square deviation of backbone atoms of the protein of less than 2.0 Å, 1.5 Å, 1.0 Å or 0.5 Å, or selected coordinates thereof; (d) structure factor data derivable from the atomic coordinate data of (b) or (c); and (e) atomic coordinate data of Table I or II optionally varied by a root mean square deviation of backbone atoms of the protein of less than 2.0 Å, 1.5 Å, 1.0 Å or 0.5 Å, or selected coordinates thereof, wherein, optionally, the atomic coordinate data is for at least one of the atoms provided by the residues listed in claim 51.

64. A method for selecting or designing a compound expected to modulate the activity of Leukotriene C4 synthase (LTC4S), the method comprising the step of using molecular modelling means to select or design a compound that is predicted to interact with a subunit interaction region of LTC4S, wherein a three-dimensional structure of at least a part of a subunit interaction region of LTC4S is compared with a three-dimensional structure of a compound, and a compound that is predicted to interact with the said substrate interaction region is selected.
